SEQUENCE LISTING (74 sequences)

SEQ ID NO: 1 - protein, 529 aa, synthetic construct
  source 1..529: note = Description of Artificial Sequence: Synthetic polypeptide
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 2 - protein, 18 aa, unidentified
  source 1..18: note = Description of Unknown: NLS sequence
KRPAATKKAGQAKKKKEF

SEQ ID NO: 3 - RNA, 51 nt, synthetic construct
  source 1..51: mol_type = other RNA; note = Description of Artificial Sequence: Synthetic oligonucleotide
ggccaccccaaaaatgaaggggactaaaacagtgataagtggaatgccatg

SEQ ID NO: 4 - RNA, 51 nt, synthetic construct
  source 1..51: mol_type = other RNA; note = Description of Artificial Sequence: Synthetic oligonucleotide
ggccaccccaaaaatgaaggggactaaaacacgacctactctcccatactc

SEQ ID NO: 5 - RNA, 41 nt, synthetic construct
  source 1..41: mol_type = other RNA; note = Description of Artificial Sequence: Synthetic oligonucleotide
taatttctactaagtgtagatcccccagcgcttcagcgttc

SEQ ID NO: 6 - RNA, 41 nt, synthetic construct; identical to SEQ ID NO: 5
  source 1..41: mol_type = other RNA; note = Description of Artificial Sequence: Synthetic oligonucleotide

SEQ ID NO: 7 - DNA, 46 bp, synthetic construct
  source 1..46: mol_type = other DNA; note = Description of Artificial Sequence: Synthetic oligonucleotide; note = Description of Combined DNA/RNA Molecule: Synthetic oligonucleotide
  modified_base 1: mod_base = OTHER; note = Thymine phosphorothioate
  modified_base 2: mod_base = OTHER; note = Thymine phosphorothioate
  modified_base 3: mod_base = OTHER; note = Thymine phosphorothioate
  modified_base 4: mod_base = OTHER; note = Thymine phosphorothioate
  modified_base 5: mod_base = OTHER; note = Thymine phosphorothioate
  misc_feature 1..5: note = DNA
  misc_feature 6..46: note = RNA
ttttttaatttctactaagtgtagatcccccagcgcttcagcgttc

SEQ ID NO: 8 - DNA, 51 bp, synthetic construct
  source 1..51: mol_type = other DNA; note = Description of Artificial Sequence: Synthetic oligonucleotide; note = Description of Combined DNA/RNA Molecule: Synthetic oligonucleotide
  modified_base 1: mod_base = OTHER; note = Thymine phosphorothioate
  modified_base 2: mod_base = OTHER; note = Thymine phosphorothioate
  modified_base 3: mod_base = OTHER; note = Thymine phosphorothioate
  modified_base 4: mod_base = OTHER; note = Thymine phosphorothioate
  modified_base 5: mod_base = OTHER; note = Thymine phosphorothioate
  modified_base 6: mod_base = OTHER; note = Thymine phosphorothioate
  modified_base 7: mod_base = OTHER; note = Thymine phosphorothioate
  modified_base 8: mod_base = OTHER; note = Thymine phosphorothioate
  modified_base 9: mod_base = OTHER; note = Thymine phosphorothioate
  modified_base 10: mod_base = OTHER; note = Thymine phosphorothioate
  misc_feature 1..10: note = DNA
  misc_feature 11..51: note = RNA
tttttttttttaatttctactaagtgtagatcccccagcgcttcagcgttc

SEQ ID NO: 9 - DNA, 42 bp, synthetic construct
  source 1..42: mol_type = other DNA; note = Description of Artificial Sequence: Synthetic oligonucleotide; note = Description of Combined DNA/RNA Molecule: Synthetic oligonucleotide
  misc_feature 1..9: note = RNA
  misc_feature 10: note = DNA
  misc_feature 11..42: note = RNA
taatttctatctaagtgtagatcccccagcgcttcagcgttc

SEQ ID NO: 10 - DNA, 42 bp, synthetic construct
  source 1..42: mol_type = other DNA; note = Description of Artificial Sequence: Synthetic oligonucleotide; note = Description of Combined DNA/RNA Molecule: Synthetic oligonucleotide
  misc_feature 1..22: note = RNA
  misc_feature 23: note = DNA
  misc_feature 24..42: note = RNA
taatttctactaagtgtagatctccccagcgcttcagcgttc

SEQ ID NO: 11 - DNA, 41 bp, synthetic construct
  source 1..41: mol_type = other DNA; note = Description of Artificial Sequence: Synthetic oligonucleotide; note = Description of Combined DNA/RNA Molecule: Synthetic oligonucleotide
  misc_feature 1..7: note = RNA
  misc_feature 8: note = DNA
  misc_feature 9..41: note = RNA
taatttctactaagtgtagatcccccagcgcttcagcgttc

SEQ ID NO: 12 - DNA, 41 bp, synthetic construct; identical to SEQ ID NO: 11
  source 1..41: mol_type = other DNA; note = Description of Artificial Sequence: Synthetic oligonucleotide; note = Description of Combined DNA/RNA Molecule: Synthetic oligonucleotide
  misc_feature 1..20: note = RNA
  misc_feature 21: note = DNA
  misc_feature 22..41: note = RNA

SEQ ID NO: 13 - RNA, 41 nt, synthetic construct; identical to SEQ ID NO: 5
  source 1..41: mol_type = other RNA; note = Description of Artificial Sequence: Synthetic oligonucleotide

SEQ ID NO: 14 - RNA, 41 nt, synthetic construct; identical to SEQ ID NO: 5
  source 1..41: mol_type = other RNA; note = Description of Artificial Sequence: Synthetic oligonucleotide

SEQ ID NO: 15 - RNA, 41 nt, synthetic construct; identical to SEQ ID NO: 5
  source 1..41: mol_type = other RNA; note = Description of Artificial Sequence: Synthetic oligonucleotide

SEQ ID NO: 16-17 - skipped (2 entries)

SEQ ID NO: 18 - DNA, 14 bp, synthetic construct
  source 1..14: mol_type = other DNA; note = Description of Artificial Sequence: Synthetic oligonucleotide
ttattattattatt

SEQ ID NO: 19 - DNA, 19 bp, synthetic construct
  source 1..19: mol_type = other DNA; note = Description of Artificial Sequence: Synthetic oligonucleotide
  modified_base 1: mod_base = OTHER; note = Thymine phosphorothioate
  modified_base 2: mod_base = OTHER; note = Thymine phosphorothioate
  modified_base 3: mod_base = OTHER; note = Thymine phosphorothioate
ttttattattattattatt

SEQ ID NO: 20 - DNA, 12 bp, synthetic construct
  source 1..12: mol_type = other DNA; note = Description of Artificial Sequence: Synthetic oligonucleotide
ttattattatta

SEQ ID NO: 21 - DNA, 30 bp, synthetic construct
  source 1..30: mol_type = other DNA; note = Description of Artificial Sequence: Synthetic oligonucleotide
tttttttttttttttttttttttttttttt

SEQ ID NO: 22 - DNA, 20 bp, synthetic construct
  source 1..20: mol_type = other DNA; note = Description of Artificial Sequence: Synthetic oligonucleotide
tttttttttttttttttttt

SEQ ID NO: 23 - DNA, 23 bp, synthetic construct
  source 1..23: mol_type = other DNA; note = Description of Artificial Sequence: Synthetic oligonucleotide
  modified_base 1: mod_base = OTHER; note = Thymine phosphorothioate
  modified_base 2: mod_base = OTHER; note = Thymine phosphorothioate
  modified_base 3: mod_base = OTHER; note = Thymine phosphorothioate
ttttttttttttttttttttttt

SEQ ID NO: 24 - DNA, 22 bp, synthetic construct
  source 1..22: mol_type = other DNA; note = Description of Artificial Sequence: Synthetic oligonucleotide
  modified_base 20: mod_base = OTHER; note = Thymine phosphorothioate
  modified_base 21: mod_base = OTHER; note = Thymine phosphorothioate
tttttttttttttttttttttt

SEQ ID NO: 25 - DNA, 23 bp, synthetic construct
  source 1..23: mol_type = other DNA; note = Description of Artificial Sequence: Synthetic oligonucleotide
  modified_base 1: mod_base = OTHER; note = Thymine phosphorothioate
  modified_base 2: mod_base = OTHER; note = Thymine phosphorothioate
  modified_base 3: mod_base = OTHER; note = Thymine phosphorothioate
ttttttttttttttttttttttt

SEQ ID NO: 26 - DNA, 21 bp, synthetic construct
  source 1..21: mol_type = other DNA; note = Description of Artificial Sequence: Synthetic oligonucleotide
ttttttttttttttttttttt

SEQ ID NO: 27 - DNA, 22 bp, synthetic construct
  source 1..22: mol_type = other DNA; note = Description of Artificial Sequence: Synthetic oligonucleotide
  modified_base 20: mod_base = OTHER; note = Thymine phosphorothioate
  modified_base 21: mod_base = OTHER; note = Thymine phosphorothioate
tttttttttttttttttttttt

SEQ ID NO: 28 - DNA, 21 bp, synthetic construct
  source 1..21: mol_type = other DNA; note = Description of Artificial Sequence: Synthetic oligonucleotide
ttttttttttttttttttttt

SEQ ID NO: 29 - DNA, 23 bp, synthetic construct
  source 1..23: mol_type = other DNA; note = Description of Artificial Sequence: Synthetic oligonucleotide; note = Description of Combined DNA/RNA Molecule: Synthetic oligonucleotide
  misc_feature 1..12: note = DNA
  misc_feature 13..17: note = RNA
  misc_feature 18..23: note = DNA
ttttttttttttttttttttttt

SEQ ID NO: 30 - DNA, 12 bp, synthetic construct
  source 1..12: mol_type = other DNA; note = Description of Artificial Sequence: Synthetic oligonucleotide
tttttttttttt

SEQ ID NO: 31 - DNA, 13 bp, synthetic construct
  source 1..13: mol_type = other DNA; note = Description of Artificial Sequence: Synthetic oligonucleotide
ttttttttttttt

SEQ ID NO: 32 - DNA, 12 bp, synthetic construct
  source 1..12: mol_type = other DNA; note = Description of Artificial Sequence: Synthetic oligonucleotide
tttttttttttt

SEQ ID NO: 33 - DNA, 21 bp, synthetic construct
  source 1..21: mol_type = other DNA; note = Description of Artificial Sequence: Synthetic oligonucleotide
ttttttttttttttttttttt

SEQ ID NO: 34 - DNA, 21 bp, synthetic construct
  source 1..21: mol_type = other DNA; note = Description of Artificial Sequence: Synthetic oligonucleotide
ttttttttttttttttttttt

SEQ ID NO: 35 - DNA, 20 bp, synthetic construct
  source 1..20: mol_type = other DNA; note = Description of Artificial Sequence: Synthetic oligonucleotide
tttttttttttttttttttt

SEQ ID NO: 36 - DNA, 20 bp, synthetic construct
  source 1..20: mol_type = other DNA; note = Description of Artificial Sequence: Synthetic oligonucleotide
tttttttttttttttttttt

SEQ ID NO: 37 - RNA, 41 nt, synthetic construct; identical to SEQ ID NO: 5
  source 1..41: mol_type = other RNA; note = Description of Artificial Sequence: Synthetic oligonucleotide

SEQ ID NO: 38 - RNA, 41 nt, synthetic construct; identical to SEQ ID NO: 5
  source 1..41: mol_type = other RNA; note = Description of Artificial Sequence: Synthetic oligonucleotide

SEQ ID NO: 39 - RNA, 41 nt, synthetic construct; identical to SEQ ID NO: 5
  source 1..41: mol_type = other RNA; note = Description of Artificial Sequence: Synthetic oligonucleotide

SEQ ID NO: 40 - RNA, 41 nt, synthetic construct; identical to SEQ ID NO: 5
  source 1..41: mol_type = other RNA; note = Description of Artificial Sequence: Synthetic oligonucleotide

SEQ ID NO: 41 - RNA, 41 nt, synthetic construct
  source 1..41: mol_type = other RNA; note = Description of Artificial Sequence: Synthetic oligonucleotide
taatttctactaagtgtagatttacatggctctggtccgag

SEQ ID NO: 42 - RNA, 41 nt, synthetic construct
  source 1..41: mol_type = other RNA; note = Description of Artificial Sequence: Synthetic oligonucleotide
taatttctactaagtgtagatgccgataatgatgtagggat

SEQ ID NO: 43 - RNA, 41 nt, synthetic construct; identical to SEQ ID NO: 5
  source 1..41: mol_type = other RNA; note = Description of Artificial Sequence: Synthetic oligonucleotide

SEQ ID NO: 44 - DNA, 23 bp, synthetic construct
  source 1..23: mol_type = other DNA; note = Description of Artificial Sequence: Synthetic oligonucleotide
  modified_base 1: mod_base = OTHER; note = Thymine phosphorothioate
  modified_base 2: mod_base = OTHER; note = Thymine phosphorothioate
  modified_base 3: mod_base = OTHER; note = Thymine phosphorothioate
ttttttttttttttttttttttt

SEQ ID NO: 45 - RNA, 41 nt, synthetic construct; identical to SEQ ID NO: 42
  source 1..41: mol_type = other RNA; note = Description of Artificial Sequence: Synthetic oligonucleotide

SEQ ID NO: 46 - RNA, 41 nt, synthetic construct; identical to SEQ ID NO: 41
  source 1..41: mol_type = other RNA; note = Description of Artificial Sequence: Synthetic oligonucleotide

SEQ ID NO: 47 - RNA, 41 nt, synthetic construct
  source 1..41: mol_type = other RNA; note = Description of Artificial Sequence: Synthetic oligonucleotide
taatttctactaagtgtagatctgccaattgcaggaatgat

SEQ ID NO: 48 - DNA, 13 bp, synthetic construct
  source 1..13: mol_type = other DNA; note = Description of Artificial Sequence: Synthetic oligonucleotide
ttttttttttttt

SEQ ID NO: 49 - DNA, 20 bp, synthetic construct
  source 1..20: mol_type = other DNA; note = Description of Artificial Sequence: Synthetic oligonucleotide
tttttttttttttttttttt

SEQ ID NO: 50 - protein, 1224 aa, synthetic construct
  source 1..1224: note = Description of Artificial Sequence: Synthetic polypeptide
MKKIDNFVGCYPVSKTLRFKAIPIGKTQENIEKKRLVEEDEVRAKDYKAVKKLIDRYHREFIEGVLDNVKLDGLEEYYMLFNKSDREESDNKKIEIMEERFRRVISKSFKNNEEYKKIFSKKIIEEILPNYIKDEEEKELVKGFKGFYTAFVGYAQNRENMYSDEKKSTAISYRIVNENMPRFITNIKVFEKAKSILDVDKINEINEYILNNDYYVDDFFNIDFFNYVLNQKGIDIYNAIIGGIVTGDGRKIQGLNECINLYNQENKKIRLPQFKPLYKQILSESESMSFYIDEIESDDMLIDMLKESLQIDSTINNAIDDLKVLFNNIFDYDLSGIFINNGLPITTISNDVYGQWSTISDGWNERYDVLSNAKDKESEKYFEKRRKEYKKVKSFSISDLQELGGKDLSICKKINEIISEMIDDYKSKIEEIQYLFDIKELEKPLVTDLNKIELIKNSLDGLKRIERYVIPFLGTGKEQNRDEVFYGYFIKCIDAIKEIDGVYNKTRNYLTKKPYSKDKFKLYFENPQLMGGWDRNKESDYRSTLLRKNGKYYVAIIDKSSSNCMMNIEEDENDNYEKINYKLLPGPNKMLPKVFFSKKNREYFAPSKEIERIYSTGTFKKDTNFVKKDCENLITFYKDSLDRHEDWSKSFDFSFKESSAYRDISEFYRDVEKQGYRVSFDLLSSNAVNTLVEEGKLYLFQLYNKDFSEKSHGIPNLHTMYFRSLFDDNNKGNIRLNGGAEMFMRRASLNKQDVTVHKANQPIKNKNLLNPKKTTTLPYDVYKDKRFTEDQYEVHIPITMNKVPNNPYKINHMVREQLVKDDNPYVIGIDRGERNLIYVVVVDGQGHIVEQLSLNEIINENNGISIRTDYHTLLDAKERERDESRKQWKQIENIKELKEGYISQVVHKICELVEKYDAVIALEDLNSGFKNSRVKVEKQVYQKFEKMLITKLNYMVDKKKDYNKPGGVLNGYQLTTQFESFSKMGTQNGIMFYIPAWLTSKMDPTTGFVDLLKPKYKNKADAQKFFSQFDSIRYDNQEDAFVFKVNYTKFPRTDADYNKEWEIYTNGERIRVFRNPKKNNEYDYETVNVSERMKELFDSYDLLYDKGELKETICEMEESKFFEELIKLFRLTLQMRNSISGRTDVDYLISPVKNSNGYFYNSNDYKKEGAKYPKDADANGAYNIARKVLWAIEQFKMADEDKLDKTKISIKNQEWLEYAQTHCE

SEQ ID NO: 51 - RNA, 140 nt, synthetic construct
  source 1..140: mol_type = other RNA; note = Description of Artificial Sequence: Synthetic polynucleotide
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcattt

SEQ ID NO: 52 - RNA, 57 nt, synthetic construct
  source 1..57: mol_type = other RNA; note = Description of Artificial Sequence: Synthetic oligonucleotide
gttgcagaacccgaatagacgaatgaaggaatgcaaccccccagcgcttcagcgttc

SEQ ID NO: 53 - RNA, 57 nt, synthetic construct
  source 1..57: mol_type = other RNA; note = Description of Artificial Sequence: Synthetic oligonucleotide
gttgcagaacccgaatagacgaatgaaggaatgcaacgccgataatgatgtagggat

SEQ ID NO: 54 - RNA, 57 nt, synthetic construct
  source 1..57: mol_type = other RNA; note = Description of Artificial Sequence: Synthetic oligonucleotide
gttgcagaacccgaatagacgaatgaaggaatgcaactggcactgagaatttgacta

SEQ ID NO: 55 - RNA, 57 nt, synthetic construct
  source 1..57: mol_type = other RNA; note = Description of Artificial Sequence: Synthetic oligonucleotide
gttgcagaacccgaatagacgaatgaaggaatgcaactacaagtgcctgtaggtata

SEQ ID NO: 56 - DNA, 52 bp, synthetic construct
  source 1..52: mol_type = other DNA; note = Description of Artificial Sequence: Synthetic oligonucleotide; note = Description of Combined DNA/RNA Molecule: Synthetic oligonucleotide
  misc_feature 1..9: note = DNA
  misc_feature 10..14: note = RNA
  misc_feature 15..38: note = DNA
  misc_feature 39..43: note = RNA
  misc_feature 44..52: note = DNA
gctcagtattttttatactgaccgcctcacctctcttttttttaaacagagg

SEQ ID NO: 57 - DNA, 20 bp, synthetic construct
  source 1..20: mol_type = other DNA; note = Description of Artificial Sequence: Synthetic primer
ttacaaacattggccgcaaa

SEQ ID NO: 58 - DNA, 18 bp, synthetic construct
  source 1..18: mol_type = other DNA; note = Description of Artificial Sequence: Synthetic primer
gcgcgacattccgaagaa

SEQ ID NO: 59 - RNA, 41 nt, synthetic construct; identical to SEQ ID NO: 5
  source 1..41: mol_type = other RNA; note = Description of Artificial Sequence: Synthetic oligonucleotide

SEQ ID NO: 60 - DNA, 20 bp, synthetic construct
  source 1..20: mol_type = other DNA; note = Description of Artificial Sequence: Synthetic primer
cctccgtgatatggctcttc

SEQ ID NO: 61 - DNA, 18 bp, synthetic construct
  source 1..18: mol_type = other DNA; note = Description of Artificial Sequence: Synthetic primer
agagtcctttgggcttcc

SEQ ID NO: 62 - RNA, 41 nt, synthetic construct; identical to SEQ ID NO: 41
  source 1..41: mol_type = other RNA; note = Description of Artificial Sequence: Synthetic oligonucleotide

SEQ ID NO: 63 - DNA, 25 bp, synthetic construct
  source 1..25: mol_type = other DNA; note = Description of Artificial Sequence: Synthetic primer
aggtgcctggaatattggtgaacag

SEQ ID NO: 64 - DNA, 26 bp, synthetic construct
  source 1..26: mol_type = other DNA; note = Description of Artificial Sequence: Synthetic primer
tcaagagtgcgggagaaaattgatcg

SEQ ID NO: 65 - RNA, 41 nt, synthetic construct
  source 1..41: mol_type = other RNA; note = Description of Artificial Sequence: Synthetic oligonucleotide
taatttctactaagtgtagatcatcagaggctgctcgtgtt

SEQ ID NO: 66 - DNA, 48 bp, synthetic construct
  source 1..48: mol_type = other DNA; note = Description of Artificial Sequence: Synthetic oligonucleotide
tttcttgctttcgtggtattcttgctagttacactagccatccttact

SEQ ID NO: 67 - DNA, 12 bp, synthetic construct
  source 1..12: mol_type = other DNA; note = Description of Artificial Sequence: Synthetic oligonucleotide
tttttttttttt

SEQ ID NO: 68 - DNA, 16 bp, synthetic construct
  source 1..16: mol_type = other DNA; note = Description of Artificial Sequence: Synthetic oligonucleotide
tttttttttttttttt

SEQ ID NO: 69 - DNA, 20 bp, synthetic construct
  source 1..20: mol_type = other DNA; note = Description of Artificial Sequence: Synthetic oligonucleotide
tttttttttttttttttttt

SEQ ID NO: 70 - DNA, 30 bp, synthetic construct; identical to SEQ ID NO: 21
  source 1..30: mol_type = other DNA; note = Description of Artificial Sequence: Synthetic oligonucleotide

SEQ ID NO: 71 - DNA, 20 bp, synthetic construct
  source 1..20: mol_type = other DNA; note = Description of Artificial Sequence: Synthetic oligonucleotide
tttttttttttttttttttt

SEQ ID NO: 72 - DNA, 20 bp, synthetic construct
  source 1..20: mol_type = other DNA; note = Description of Artificial Sequence: Synthetic oligonucleotide
tttttttttttttttttttt

SEQ ID NO: 73 - DNA, 20 bp, synthetic construct
  source 1..20: mol_type = other DNA; note = Description of Artificial Sequence: Synthetic oligonucleotide
tttttttttttttttttttt

SEQ ID NO: 74 - DNA, 10 bp, synthetic construct
  source 1..10: mol_type = other DNA; note = Description of Artificial Sequence: Synthetic oligonucleotide
tttttttttt

(19) United States
(12) Patent Application Publication
(10) Pub. No.: US 2024/0326048 A1
(43) Pub. Date: Oct. 3, 2024

(54) PROGRAMMABLE NUCLEASE DIAGNOSTIC DEVICE

(71) Applicant: MAMMOTH BIOSCIENCES, INC., Brisbane, CA (US)

(72) Inventors: Clare Louise FASCHING, Redwood City, CA (US); Matthew VEROSLOFF, South San Francisco, CA (US); Jesus CHING, Saratoga, CA (US); James Paul BROUGHTON, South San Francisco, CA (US); Maria-Nefeli TSALOGLOU, San Francisco, CA (US); Phillip You Fai LEE, South San Francisco, CA (US); Timothy James PATNO, Barrington, IL (US); Janice Sha CHEN, San Francisco, CA (US); Sarah Jane SHAPIRO, Pacifica, CA (US); James Raymond CORDOVA, Sunnyvale, CA (US); Brian J. EASTMAN, Napa, CA (US); Donald Allan LOWMILLER, Chicago, IL (US); Lior KREINDLER, Foster City, CA (US); Daniel Thomas DRZAL, Pacifica, CA (US); Devin SPRATT, Mountain View, CA (US); Jacob LESINSKI, Oakland, CA (US); Joshua BAIK, San Francisco, CA (US); Rachel Nouha DERANEY, San Francisco, CA (US); Sonal JAIN, San Mateo, CA (US); Sophia HUBBELL, San Francisco, CA (US); Carley Gelenter HENDRIKS, Burlingame, CA (US)

(21) Appl. No.: 18/058,122

(22) Filed: Nov. 22, 2022

Related U.S. Application Data
(63) Continuation of application No. PCT/US21/35031, filed on May 28, 2021.
(60) Provisional application No. 63/181,130, filed on Apr. 28, 2021.
(60) Provisional application No. 63/151,592, filed on Feb. 19, 2021.
(60) Provisional application No. 63/166,538, filed on Mar. 26, 2021.
(60) Provisional application No. 63/113,798, filed on Nov. 13, 2020.
(60) Provisional application No. 63/032,455, filed on May 29, 2020.

(51) Int. Cl. B01L 3/00; B01L 7/00; C12N 9/22; C12N 15/11; C12Q 1/48; C12Q 1/6806; C12Q 1/6823; C12Q 1/6834; C12Q 1/6844

(52) U.S. Cl. CPC B01L 3/502761; B01L 3/502715; B01L 7/52; C12N 9/22; C12N 15/111; C12Q 1/485; C12Q 1/6806; C12Q 1/6823; C12Q 1/6834; C12Q 1/6844; B01L 2200/16; B01L 2300/0681; B01L 2300/1877; B01L 2400/0487; C12N 2310/20

(57) ABSTRACT

The present disclosure provides various diagnostic devices. Devices can comprising a sample interface configured to receive a sample that may comprise at least one sequence of interest; a channel in fluid communication with the sample interface and a detection chamber, said channel comprising one or more movable mechanisms to separate the sample into a plurality of droplets, wherein said detection chamber is configured to receive and contact the plurality of droplets with at least one programmable nuclease probe disposed on a surface of said detection chamber, and wherein said at least one programmable nuclease probe may comprise a guide nucleic acid complexed with a programmable nuclease; and a plurality of sensors that determine a presence of said at least one sequence of interest by detecting a signal produced upon cleavage of a target nucleic acid region of said at least one sequence of interest by said at least one programmable nuclease probe.

### CROSS-REFERENCE

This application claims the benefit of U.S. Provisional Application No. 63/032,455, filed on May 29, 2020; U.S. Provisional Application No. 63/113,798, filed on Nov. 13, 2020; U.S. Provisional Application No. 63/151,592 filed on Feb. 19, 2021; U.S. Provisional Application No. 63/166,538 filed on Mar. 26, 2021; and U.S. Provisional Application No. 63/181,130, filed on Apr. 28, 2021, each of which is incorporated herein by reference in its entirety.

### STATEMENT AS TO FEDERALLY SPONSORED RESEARCH

This invention was made with government support under Contract No. N66001-21-C-4048 awarded by the Department of Defense, Defense Advanced Research Projects Agency (DARPA). The US government has certain rights in the invention.

### SEQUENCE LISTING

The instant application contains a Sequence Listing which has been submitted electronically in XML format and is hereby incorporated by reference in its entirety. Said XML copy, created on Nov. 29, 2022, is named 203477-732301_US_SL.xml and is 111,033 bytes in size.

### BACKGROUND

Detection of ailments, especially at the early stages of disease or infection, can provide guidance on treatment or intervention to reduce the progression or transmission of said ailments. Such ailments can be detected at the point of need by devices capable of running diagnostic assays. Various biological species associated with an organism, disease state, phenotype, or genotype can be detected by these devices. Challenges in deploying such devices include developing methods to immobilize diagnostic assay components on surfaces without compromising the performance of the assay, as well as performing amplification of samples without significant additional instrumentation.

### SUMMARY

In an aspect, the present disclosure provides a programmable nuclease diagnostic device that may comprise a sample interface configured to receive a sample that may comprise at least one sequence of interest; a channel in fluid communication with the sample interface and a detection chamber, said channel comprising one or more movable mechanisms to separate the sample into a plurality of droplets, wherein said detection chamber is configured to receive and contact the plurality of droplets with at least one programmable nuclease probe disposed on a surface of said detection chamber, wherein said at least one programmable nuclease probe may comprise a guide nucleic acid complexed with a programmable nuclease; and a plurality of sensors that determine a presence of said at least one sequence of interest by detecting a signal produced upon cleavage of a target nucleic acid region of said at least one sequence of interest by said at least one programmable nuclease probe. In some embodiments, the cleavage of said target nucleic acid region occurs after a complementary binding of said target nucleic acid region to said guide nucleic acid of said at least one programmable nuclease probe. In some embodiments, the programmable nuclease probe may comprise a CRISPR/Cas enzyme. In some embodiments, the guide nucleic acid may comprise a guide RNA. In some embodiments, the one or more movable mechanisms comprise one or more valves configured to restrict flow through one or more sections of the channel. In some embodiments, the one or more movable mechanisms comprise a plunger or a bristle that is configured to restrict flow through one or more sections of the channel. In some embodiments, the one or more movable mechanisms are operatively coupled to a power source that is integrated with or insertable into the device. In some embodiments, the power source may comprise a battery. In some embodiments, the device may comprise a physical filter to filter one or more particles from the sample that do not comprise the sequence of interest. In some embodiments, the device may comprise a sample preparation chamber. In some embodiments, the sample preparation chamber may comprise a lysing agent. In some embodiments, the sample preparation chamber may comprise a heating unit configured for heat inactivation. In some embodiments, the sample preparation chamber may comprise one or more reagents for nucleic acid purification. In some embodiments, the channel may comprise a plurality of heating elements and a plurality of heat sinks for amplifying the at least one sequence of interest or a portion thereof. In some embodiments, the plurality of heating elements and the plurality of heat sinks are configured to perform one or more thermocycling operations on the plurality of droplets. In some embodiments, the device may comprise a plurality of programmable nuclease probes comprising different guide RNAs. In some embodiments, the signal is associated with a physical, chemical, or electrochemical change or reaction. In some embodiments, the signal may comprise an optical signal. In some embodiments, the signal may comprise a fluorescent or colorimetric signal. In some embodiments, the signal may comprise a potentiometric or amperometric signal. In some embodiments, the signal may comprise a piezo-electric signal. In some embodiments, the signal is associated with a change in an index of refraction of a solid or gel volume in which said at least one programmable nuclease probe is disposed.

In another aspect, the present disclosure provides a device that may comprise a sample interface configured to receive a sample that may comprise one or more target sequences of interest; one or more channels comprising one or more movable mechanisms to separate the sample into partitioned samples, wherein the one or more channels are in fluid communication with the sample interface and a reaction chamber that is configured to receive and contact the partitioned samples with an enzyme, reagent, or programmable detection agent that is configured to cleave a nucleic acid of said one or more target sequences of interest; and a plurality of sensors for determining a presence of the one or more target sequences of interest by detecting one or more reporters released upon said cleavage of said nucleic acid. In some embodiments, the programmable detection agent may comprise a CRISPR/Cas enzyme. In some embodiments, the one or more target sequences of interest comprise a sequence of nucleic acids comprising said nucleic acid. In some embodiments, the one or more movable mechanisms comprise a plurality of valves configured to restrict flow in a first direction through the one or more channels towards the sample interface. In some embodiments, the plurality of valves are configured to selectively permit flow in a second direction through the one or more channels towards the reaction chamber. In some embodiments, a first valve and a second valve of the plurality of valves are configured to physically, fluidically, or thermally isolate a first portion of the sample from a second portion of the sample when the first valve and the second valve are in a closed state. In some embodiments, the one or more channels comprise a plurality of heating elements and a plurality of heat sinks to perform thermocycling on the partitioned samples. In some embodiments, a first heating element of the plurality of heating elements and a first heat sink of the plurality of heat sinks are positioned between a first movable mechanism and a second movable mechanism of the one or more movable mechanisms. In some embodiments, the reporter may comprise a nucleic acid and a detection moiety. In some embodiments, the reporter may comprise at least one ribonucleotide or at least one deoxyribonucleotide. In some embodiments, the reporter may comprise a DNA nucleic acid or an RNA nucleic acid. In some embodiments, the device may comprise a telemedicine unit configured to provide one or more detection results to a computing unit that is remote from the device, wherein the one or more detection results indicate a presence or an absence of a target nucleic acid of interest in the sample.

In another aspect, the present disclosure provides a method for target detection, comprising: contacting a sample with any of the devices described herein; and detecting a presence or an absence of one or more genes of interest in said sample. In some embodiments, the method may comprise generating one or more detection results indicating the presence or the absence of the one or more genes of interest in the sample. In some embodiments, the method may comprise transmitting the one or more detection results to a remote computing unit. In some embodiments, the remote computing unit may comprise a mobile device. In another aspect, the present disclosure provides a method for target detection, comprising: providing a sample comprising at least one gene of interest; separating the sample into a plurality of sub-samples using one or more movable mechanisms; receiving the plurality of sub-samples in a detection chamber and contacting the plurality of sub-samples with at least one programmable nuclease probe disposed on a surface of said detection chamber, wherein said at least one programmable nuclease probe may comprise a guide nucleic acid complexed with a programmable nuclease; and using a plurality of sensors to determine a presence or an absence of said at least one gene of interest by detecting a signal produced upon cleavage of a target nucleic acid region in said at least one gene of interest by said at least one programmable nuclease probe. In some embodiments, the method may comprise amplifying the at least one gene of interest after separating the sample into a plurality of sub-samples. In some embodiments, the method may comprise amplifying the at least one gene of interest before the plurality of sub-samples are received in the detection chamber. In some embodiments, amplifying the at least one gene of interest may comprise using a plurality of heating elements and a plurality of heat sinks to perform thermocycling on the plurality of sub-samples.

In some embodiments, the programmable nuclease probe may comprise a CRISPR/Cas enzyme. In some embodiments, the guide nucleic acid may comprise a guide RNA. In some embodiments, the one or more movable mechanisms comprise one or more valves configured to restrict flow through one or more sections of the channel. In some embodiments, the one or more movable mechanisms comprise a plunger or a bristle that is configured to restrict flow through one or more sections of the channel. In some embodiments, the method may comprise using a physical filter to filter one or more particles from the sample that do not comprise the at least one gene of interest. In some embodiments, the method may comprise lysing the sample before detecting the at least one gene of interest. In some embodiments, the method may comprise performing heat inactivation on the sample. In some embodiments, the method may comprise performing nucleic acid purification on the sample. In some embodiments, the method may comprise contacting the plurality of sub-samples with a plurality of programmable nuclease probes comprising different guide RNAs. In some embodiments, the signal is associated with a physical, chemical, or electrochemical change or reaction. In some embodiments, the signal is selected from the group consisting of an optical signal, a fluorescent signal, a colorimetric signal, a potentiometric signal, an amperometric signal, and a piezo-electric signal. In some embodiments, the signal is associated with a change in an index of refraction of a solid or gel volume in which said at least one programmable nuclease probe is disposed. In some embodiments, the method may comprise using the signal to detect pathogenic viruses, pathogenic bacteria, pathogenic worms, pathogenic fungi, or cancer cells. In some embodiments, the pathogenic viruses are selected from the group consisting of respiratory viruses, adenoviruses, parainfluenza viruses, severe acute respiratory syndrome (SARS), coronavirus, SARS-CoV, SARS-CoV-2, MERS, gastrointestinal viruses, noroviruses, rotaviruses, astroviruses, exanthematous viruses, hepatic viral diseases, cutaneous viral diseases, herpes, hemorrhagic viral diseases, Ebola, Lassa fever, dengue fever, yellow fever, Marburg hemorrhagic fever, Crimean-Congo hemorrhagic fever, neurologic viruses, polio, viral meningitis, viral encephalitis, rabies, sexually transmitted viruses, HIV, HPV, immunodeficiency viruses, influenza virus, dengue virus, West Nile virus, herpes virus, yellow fever virus, Hepatitis Virus C, Hepatitis Virus A, Hepatitis Virus B, and papillomavirus. In some embodiments, the method may comprise amplifying or modifying the signal using a physical or chemical interaction between a reporter that is released upon cleavage and another material, entity, or molecular species in the detection chamber. In some embodiments, the devices of the present disclosure are configured to detect pathogenic viruses, pathogenic bacteria, pathogenic worms, pathogenic fungi, or cancer cells based on the signal. In some embodiments, the pathogenic viruses are selected from the group consisting of respiratory viruses, adenoviruses, parainfluenza viruses, severe acute respiratory syndrome (SARS), coronavirus, SARS-CoV, SARS-CoV-2, MERS, gastrointestinal viruses, noroviruses, rotaviruses, astroviruses, exanthematous viruses, hepatic viral diseases, cutaneous viral diseases, herpes, hemorrhagic viral diseases, Ebola, Lassa fever, dengue fever, yellow fever, Marburg hemorrhagic fever, Crimean-Congo hemorrhagic fever, neurologic viruses, polio, viral meningitis, viral encephalitis, rabies, sexually transmitted viruses, HIV, HPV, immunodeficiency viruses, influenza virus, dengue virus, West Nile virus, herpes virus, yellow fever virus, Hepatitis Virus C, Hepatitis Virus A, Hepatitis Virus B, and papillomavirus. In some embodiments, the detection chamber or the reaction chamber of the device may comprise another material, entity, or molecular species that is configured to physically or chemically interact or react with a reporter that is released upon cleavage to amplify or modify the signal. In some embodiments, the sequence of interest may comprise a biological sequence. The biological sequence can comprise a nucleic acid sequence or an amino acid sequence. In some embodiments, the sequence of interest is associated with an organism of interest, a disease of interest, a disease state of interest, a phenotype of interest, a genotype of interest, or a gene of interest.

In another aspect, a reaction chamber may comprise a surface wherein a probe comprising at least one of said enzyme, said reagent, said programmable detection reagent, a programmable nuclease, a guide nucleic acid, a reporter or a combination thereof, and wherein said probe is immobilized to said surface by a linkage. In some embodiments, a linkage may comprise a surface functionality and a probe functionality. In some embodiments, a surface functionality is disposed on said surface. In some embodiments, a surface functionality is streptavidin. In some embodiments, an amino acid residue of said programmable nuclease is connected to said surface by said linkage. In some embodiments, an amino acid residue is modified with said probe functionality. In some embodiments, a probe functionality is biotin. In some embodiments, a guide nucleic acid is connected to said surface by said linkage. In some embodiments, a guide nucleic acid is modified at the 3′ end or 5′ end with said probe functionality. In some embodiments, a reporter is connected to said surface by said linkage. In some embodiments, a reporter may comprise at least one of said nucleic acid, said probe functionality, a detection moiety, a quencher or a combination thereof. In some embodiments, a reporter is configured for said detection moiety to remain immobilized to said surface and said quencher to be released into solution upon cleavage of said reporter. In some embodiments, a reporter is configured for said quencher to remain immobilized to said surface and for said detection moiety to be released into solution, upon cleavage of said reporter.

In various aspects described herein, a reporter is connected to said surface by a linkage. In some embodiments, a linkage may comprise a surface functionality and a probe functionality. In some embodiments, a surface functionality is disposed on said surface and said reporter may comprise said probe functionality.

In certain aspects, described herein are embodiments of a method for target detection, comprising: providing a sample comprising at least one sequence of interest; separating the sample into a plurality of sub-samples using one or more movable mechanisms; receiving the plurality of sub-samples in a detection chamber and contacting the plurality of sub-samples with at least one probe, wherein said at least one probe is connected to a surface of said detection chamber by a linkage, wherein said at least one probe may comprise a programmable nuclease, a guide nucleic acid, a reporter or a combination thereof; and using a plurality of sensors to determine a presence of said at least one sequence of interest by detecting a signal produced upon cleavage of said reporter by said programmable nuclease.

Described herein are various embodiments of a device comprising: a sample interface configured to receive a sample that may comprise one or more target sequences of interest; one or more channels comprising one or more movable mechanisms to separate said sample into partitioned samples, wherein said one or more channels are in fluid communication with said sample interface and a reaction chamber comprising a surface, wherein at least one probe may comprise a programmable nuclease, a guide nucleic acid, a reporter or a combination thereof, wherein said at least one probe is connected to said surface by a linkage; and a plurality of sensors for determining a presence of said one or more target sequences of interest by detecting a signal emitted upon cleavage of said reporter by said programmable nuclease.

Described herein are various devices for detecting a target nucleic acid, comprising: a sample interface configured to receive a sample comprising a target nucleic acid; a reaction chamber (e.g., a heating region) in fluid communication with the sample interface and configured to amplify the sample received via the sample interface; a detection region in fluid communication with the heating region; and a programmable nuclease probe disposed within the sample interface, the heating region, and/or the detection region, wherein a signal is produced via selective binding between the programmable nuclease probe and the target nucleic acid within the heating region, the sample interface, and/or the detection region, wherein the detection region is configured to detect the signal corresponding to a presence of the target nucleic acid, and wherein the presence or absence of the target nucleic acid is determined within a time of less than 30 minutes after the sample is received at the sample interface. In some embodiments, a reagent mix comprising amplification reagents. In some embodiments, the reagent mix is lyophilized. In some embodiments, the reagent mix is located in a region of the device that is in fluid communication with both the sample interface and the heating region. In some embodiments, the reagent mix is located within the sample interface, the heating region, the detection region, and/or a region between the sample interface and the heating region. In some embodiments, the heating region comprises amplification reagents. In some embodiments, the sample is amplified via Loop-Mediated Isothermal Amplification (LAMP). In some embodiments, the heating region is configured to maintain an isothermal, or non-cycled temperature profile. In some embodiments, the isothermal, or non-cycled temperature profile is between about 30° C. to about 60° C. In some embodiments, the isothermal, or non-cycled temperature profile is about 55° C. to about 60° C. In some embodiments, the sample interface comprises a compartment configured to receive a swab containing the sample. In some embodiments, the compartment comprises a scraper configured to transfer the sample from the swab to the device. In some embodiments, the compartment contains a interface solution configured to extract the sample from the swab. In some embodiments, the interface solution comprises a buffer solution or a lysis buffer solution. In some embodiments, the sample interface is configured to receive the sample from a swab via pipetting. In some embodiments, the sample interface comprises a compartment configured to receive the sample from a container containing the sample. In some embodiments, the container comprises a syringe. In some embodiments, the syringe interface comprises an opening for receiving the sample therethrough. In some embodiments, the syringe interface opening is in fluid communication with i) the heating region, and/or ii) another compartment that is in fluid communication with the heating region. In some embodiments, the sample interface is configured to receive the sample as a fluid. In some embodiments, the heating region and detection region are disposed on the same location on the device. In some embodiments, the heating region and the detection region are disposed within a same compartment of the device. In some embodiments, the heating region comprises a channel for fluid movement therethrough. In some embodiments, the channel is in fluid communication with the sample interface and the detection region, either directly or indirectly, thereby enabling the sample to move from the sample interface to the detection region. In some embodiments, the channel comprises a spiral configuration or a serpentine configuration. In some embodiments, two or more channels for fluid movement therethrough, wherein at least one channel of the two or more channels configured to move the sample from the sample interface to the detection region. In some embodiments, each channel of the heating region comprises one or more movable mechanisms. In some embodiments, the one or more movable mechanisms comprises i) a first movable mechanism between the sample interface and heating region for controlling transfer of the sample therebetween, and/or ii) a second movable mechanism between the heating region and the detecting region for controlling transfer of the sample therebetween. In some embodiments, at least one channel of the heating region comprises two or more heating compartments configured to separate the sample into two or more sub-samples, wherein the two or more compartments are separated from each other via a movable mechanism of the one or more movable mechanisms. In some embodiments, each heating compartment is configured to be heated by a corresponding heating element. In some embodiments, at least one heating element of the device comprises a chemical heating element. In some embodiments, at least one chemical heating element is sodium acetate. In some embodiments, the heating region comprises a chamber in fluid communication with the sample interface and the detection region. In some embodiments, the heating region comprises a reporter immobilized therein, wherein the report is configured to release a detection moiety via the selective binding between the activated programmable nuclease and the target nucleic acid, thereby enabling the signal to be produced. In some embodiments, the reporter is immobilized in the heating region via a support that is immobilized on a surface of the heating region. In some embodiments, the support comprises a bead, a coating, and an interspersed polymer. In some embodiments, the support comprises a solid support. In some embodiments, the surface of the heating region comprises a well that is a recessed portion of the surface, wherein the support is disposed within the well. In some embodiments, one or more channels to move the sample from the sample interface to the detection region. In some embodiments, the one or more channels are located within the sample interface, between the sample interface and the heating region, within the heating region, between the heating region and the detection region, and/or within the detection region. In some embodiments, one or more channels comprises a plurality of channels, wherein the plurality of channels comprises at least one set of channels arranged in series. In some embodiments, the one or more channels comprises a plurality of channels, wherein the plurality of channels comprises at least one set of channels arranged in parallel (parallel channels), thereby enabling the sample to be split into sub-samples within each channel of the at least one set of parallel channels. In some embodiments, the one or more channels comprises a plurality of channels, wherein the plurality of channels comprises at least one set of channels configured to move the sample from a first location within the device to a second location within the device, thereby enabling the sample to be split into sub-samples within each channel of the at least one set of channels. In some embodiments, the at least one set of channels comprises two or more channels having a different length and/or different configuration, thereby enabling specific conditions to be specified for two or more corresponding sub-samples. In some embodiments, the specific conditions comprise a specified heating temperature range, a specified heating duration, a specified residence time within any region or location on the device, a specific incubation time, contact with specific reagents, or any combination thereof. In some embodiments, the at least one set of channels comprises two or more channels having a same length and/or configuration, thereby enabling specific conditions to be specified for two or more corresponding sub-samples. In some embodiments, a channel of the one or more channels comprises a radial configuration, a spiral configuration, a serpentine configuration, a linear configuration, or any combination thereof. In some embodiments, at least one actuator. In some embodiments, the at least one actuator comprises a plunger, a spring-actuated plunger, or a spring mechanism. In some embodiments, the at least one actuator is manually actuated. In some embodiments, a first actuator of the at least one actuator is configured to move the sample from the sample interface to the heating region via manual actuation of the first actuator. In some embodiments, a second actuator of the at least one actuator is configured to move the detection moiety from the heating region to the detection region via manual actuation of the second actuator. In some embodiments, the device is configured to be operated manually without electrical power. In some embodiments, a power source. In some embodiments, the power source comprises one or more batteries. In some embodiments, the heating region is configured to heat the sample via a heating element. In some embodiments, the heating element comprises a chemical heating element. In some embodiments, the chemical heating element is sodium acetate. In some embodiments, the signal is visually detectable. In some embodiments, the programmable nuclease comprises a guide nucleic acid. In some embodiments, the guide nucleic acid is modified. In some embodiments, the guide nucleic acid is modified with at least one methyl group. In some embodiments, the programmable nuclease further comprises a Cas enzyme. In some embodiments, the Cas enzyme is selected from the group consisting Cas12, Cas13, Cas14, Cas14a, Cas14a1, and CasPhi. In some embodiments, the target nucleic acid is indicative of a respiratory disorder or respiratory pathogen. In some embodiments, the respiratory disorder or respiratory pathogen selected from the group consisting of SARS-CoV-2 and corresponding variants, 29E), NL63, OC43, HKU1, MERS-CoV, (MERS), SARS-CoV (SARS, Flu A, Flu B, RSV, Rhinovirus, Strep A, and TB. In some embodiments, the device is configured to differentiate between a viral infection and a bacterial infection. In some embodiments, the target nucleic acid is indicative of a sexually transmitted infection (STI) or infection related to a woman's health. In some embodiments, the STI or infection related to a woman's health is selected from the group consisting of CT, NG, MG, TV, HPV, Candida, B. Vaginosis Syphilis and UTI. In some embodiments, the target nucleic acid comprises a single nucleotide polymorphism (SNP). In some embodiments, the SNP is indicative of NASH disorder or Alpha-1 disorder. In some embodiments, the target nucleic acid is a blood borne pathogen selected from the group consisted of HIV, HBV, HCV and Zika. In some embodiments, the target nucleic acid is indicative of H. Pylori, C. Difficile, Norovirus, HSV and Meningitis. In some embodiments, a physical filter configured to filter one or more particles from the sample that do not comprise the target nucleic acid. In some embodiments, the physical filter is located between and in fluid communication with the sample interface and heating region. In some embodiments, the programmable nuclease, guide nucleic acid, or the reporter are immobilized to a device surface by a linkage. In some embodiments, the linkage comprises a covalent bond, a non-covalent bond, an electrostatic bond, a bond between streptavidin and biotin, an amide bond or any combination thereof. In some embodiments, the linkage comprises non-specific absorption. In some embodiments, the programmable nuclease is immobilized to the device surface by the linkage, wherein the linkage is between the programmable nuclease and the surface. In some embodiments, the reporter is immobilized to the device surface by the linkage, wherein the linkage is between the reporter and the surface. In some embodiments, the guide nucleic acid is immobilized to the surface by the linkage, wherein the linkage is between the 5′ end of the guide nucleic acid and the surface. In some embodiments, the guide nucleic acid is immobilized to the surface by the linkage, wherein the linkage is between the 3′ end of the guide nucleic acid and the surface. In some embodiments, a plurality of guide nucleic acids, wherein each guide nucleic acid of the plurality of guide nucleic acids is complementary, or partially complementary to a different segment of the target nucleic acid. In some embodiments, the sample comprises the sample containing the target nucleic acid(s), the sample containing the amplification reagents, the amplified sample, and/or the sample containing the detection moiety.

Described herein are various devices for detecting a target nucleic acid in a sample, comprising: a sample interface for receiving the sample; a reaction chamber (e.g., a heating region) in fluid communication with the sample interface, the heating region comprising: a programmable nuclease comprising a guide nucleic acid, and a reporter, wherein the programmable nuclease is activated by selective binding between the guide nucleic acid and a target nucleic acid, wherein the reporter is configured to release a detection moiety upon cleavage by the activated programmable nuclease; a chemical heating element configured to heat to the heating region; a detection region in fluid communication with the heating region, wherein the detection region is configured to detect a signal produced by the released detection moiety; a first manual actuator configured to transfer the sample from the heating region to the detection region; and a reagent mix comprising amplification reagents, wherein the reagent mix is disposed within the sample interface, the heating region, the detection region, and/or between the sample interface and the heating region, wherein the device is configured to determine the presence or absence of the target nucleic acid within a time of less than 30 minutes via the produced signal. In some embodiments, the reagent mix is lyophilized. In some embodiments, the heating region is configured to amplify the target nucleic acid. In some embodiments, the heating region comprises the amplification reagents. In some embodiments, the target nucleic acid is amplified via Loop-Mediated Isothermal Amplification (LAMP). In some embodiments, the heating region is configured to maintain an isothermal, or non-cycled temperature profile. In some embodiments, the isothermal, or non-cycled temperature profile is between about 30° C. to about 60° C. In some embodiments, the isothermal, or non-cycled temperature profile is about 55° C. to about 60° C. In some embodiments, the sample interface comprises a compartment configured to receive a swab containing the sample. In some embodiments, the compartment comprises a scraper configured to transfer the sample from the swab to the device. In some embodiments, the compartment contains an interface solution configured to extract the sample from the swab. In some embodiments, the interface solution comprises a buffer solution or a lysis buffer solution. In some embodiments, the sample interface is configured to receive the sample from a swab via pipetting. In some embodiments, the sample interface comprises a compartment configured to receive the sample from a container containing the sample. In some embodiments, the container comprises a syringe. In some embodiments, the syringe interface comprises an opening for receiving the sample therethrough. In some embodiments, the syringe interface opening is in fluid communication with the heating region, or another compartment that is in fluid communication with the heating region. In some embodiments, the sample interface is configured to receive the sample as a fluid. In some embodiments, the heating region and detection region are disposed on the same location on the device. In some embodiments, the heating region and the detection region are disposed within a same compartment of the device. In some embodiments, the heating region comprises a channel for fluid movement therethrough. In some embodiments, the channel is in fluid communication with the sample interface and the detection region, either directly or indirectly, thereby enabling the sample to move from the sample interface to the detection region. In some embodiments, the channel comprises a spiral configuration or a serpentine configuration. In some embodiments, two or more channels for fluid movement therethrough, wherein at least one channel of the two or more channels is configured to move the sample from the sample interface to the detection region. In some embodiments, each channel of the heating region comprises one or more movable mechanisms. In some embodiments, the one or more movable mechanisms comprises i) a first movable mechanism between the sample interface and heating region for controlling transfer of the sample therebetween, and/or ii) a second movable mechanism between the heating region and the detecting region for controlling transfer of the sample therebetween. In some embodiments, at least one channel of the heating region comprises two or more heating compartments configured to separate the sample into two or more sub-samples, wherein the two or more compartments are separated from each other via a movable mechanism of the one or more movable mechanism. In some embodiments, each heating compartment is configured to be heated by a corresponding heating element of at least one heating element. In some embodiments, the at least one heating element of the device comprises a chemical heating element. In some embodiments, the at least one chemical heating element is sodium acetate. In some embodiments, the heating region comprises a chamber. In some embodiments, the heating region comprises the reporter immobilized therein. In some embodiments, the reporter is immobilized in the heating region via a support that is immobilized on a surface of the heating region. In some embodiments, the support comprises a bead, a coating, and an interspersed polymer. In some embodiments, the support comprises a solid support. In some embodiments, the surface of the heating region comprises a well that is recessed portion of the surface, wherein the support is disposed within the well. In some embodiments, one or more channels to move the sample from the sample interface to the detection region. In some embodiments, the one or more channels are located within the sample interface, between the sample interface and the heating region, within the heating region, between the heating region and the detection region, and/or within the detection region. In some embodiments, the one or more channels comprises a plurality of channels, wherein the plurality of channels comprises at least one set of channels arranged in series. In some embodiments, the one or more channels comprises a plurality of channels, wherein the plurality of channels comprises at least one set of parallel channels arranged in parallel (parallel channels), thereby enabling the sample to be split into sub-samples within each channel of the at least one set of parallel channels. In some embodiments, the one or more channels comprises a plurality of channels, wherein the plurality of channels comprises at least one set of channels configured to move the sample from a first location within the device to a second location within the device, thereby enabling the sample to be split into sub-samples within each channel of the at least one set of channels. In some embodiments, the at least one set of channels comprises two or more channels having a different length and/or different configuration, thereby enabling specific conditions to be specified for two or more corresponding sub-samples. In some embodiments, the specific conditions comprise a specified heating temperature range, a specified heating duration, a specified residence time within any region or location on the device, a specific incubation time, contact with specific reagents, or any combination thereof. In some embodiments, the at least one set of channels comprises two or more channels having a same length and/or configuration, thereby enabling specific conditions to be specified for two or more corresponding sub-samples. In some embodiments, a channel of the one or more channels comprises a radial configuration, a spiral configuration, a serpentine configuration, a linear configuration, or any combination thereof. In some embodiments, the at least one actuator comprises a plunger, a spring-actuated plunger, or a spring mechanism. In some embodiments, the device is configured to be operated manually without electrical power. In some embodiments, the device may comprise a power source. In some embodiments, the power source comprises one or more batteries. In some embodiments, the heating region is configured to heat the sample via a heating element. In some embodiments, the heating element comprises a chemical heating element. In some embodiments, the chemical heating element is sodium acetate. In some embodiments, the signal is visually detectable. In some embodiments, the guide nucleic acid is modified. In some embodiments, the guide nucleic acid is modified with at least one methyl group. In some embodiments, the programmable nuclease further comprises a Cas enzyme. In some embodiments, the Cas enzyme is selected from the group consisting Cas12, Cas13, Cas14, Cas14a, Cas14a1, and CasPhi. In some embodiments, the target nucleic acid is indicative of a respiratory disorder or respiratory pathogen. In some embodiments, the respiratory disorder or respiratory pathogen selected from the group consisting of SARS-CoV-2 and corresponding variants, 29E), NL63, OC43, HKU1, MERS-CoV, (MERS), SARS-CoV (SARS, Flu A, Flu B, RSV, Rhinovirus, Strep A, and TB. In some embodiments, the device is configured to differentiate between a viral infection and a bacterial infection. In some embodiments, the target nucleic acid is indicative of a sexually transmitted infection (STI) or infection related to a woman's health. In some embodiments, the STI or infection related to a woman's health is selected from the group consisting of CT, NG, MG, TV, HPV, Candida, B. Vaginosis Syphilis, and UTI. In some embodiments, the target nucleic acid comprises a single nucleotide polymorphism (SNP). In some embodiments, the SNP is indicative of NASH disorder or Alpha-1 disorder. In some embodiments, the target nucleic acid is a blood borne pathogen selected from the group consisted of HIV, HBV, HCV, and Zika. In some embodiments, the target nucleic acid is indicative of H. Pylori, C. Difficile, Norovirus, HSV, and Meningitis. In some embodiments, a physical filter configured to filter one or more particles from the sample that do not comprise the target nucleic acid. In some embodiments, the physical filter is located between and in fluid communication with the sample interface and heating region. In some embodiments, the programmable nuclease, guide nucleic acid, or the reporter are immobilized to a device surface by a linkage. In some embodiments, the linkage comprises a covalent bond, a non-covalent bond, an electrostatic bond, a bond between streptavidin and biotin, an amide bond or any combination thereof. In some embodiments, the linkage comprises non-specific absorption. In some embodiments, the programmable nuclease is immobilized to the device surface by the linkage, wherein the linkage is between the programmable nuclease and the surface. In some embodiments, the reporter is immobilized to the device surface by the linkage, wherein the linkage is between the reporter and the surface. In some embodiments, the guide nucleic acid is immobilized to the surface by the linkage, wherein the linkage is between the 5′ end of the guide nucleic acid and the surface. In some embodiments, the guide nucleic acid is immobilized to the surface by the linkage, wherein the linkage is between the 3′ end of the guide nucleic acid and the surface. In some embodiments, a plurality of guide nucleic acids, wherein each guide nucleic acid of the plurality of guide nucleic acids is complementary, or partially complementary to a different segment of the target nucleic acid. In some embodiments, the sample comprises the sample containing the target nucleic acid(s), the sample containing the amplification reagents, the amplified sample, and/or the sample containing the detection moiety.

Described herein are various embodiments for a microarray device for multiplexed detection of a plurality of target nucleic acids in a sample, comprising: a sample interface for receiving the sample; a reagent mix comprising amplification reagents; a reaction chamber (e.g., a heating region) in fluid communication with the sample interface; and a detection region comprising a surface comprising a plurality of detection spots in a microarray format, wherein each of the plurality of detection spots comprise a reporter probe, wherein each reporter probe is configured to release a detection moiety via cleavage by an activated programmable nuclease, wherein each of the plurality of detection spots comprise a different programmable nuclease probe, and wherein the device is configured to determine the presence or absence of each of the plurality of target nucleic acids within a time of less than 30 minutes via the release of each detection moiety of each reporter at each of the plurality of detection spots. In some embodiments, at least one programmable nuclease probe comprises a Cas enzyme. In some embodiments, each Cas enzyme of the at least one programmable nuclease is selected from the group comprising of Cas12, Cas13, Cas14, Cas14a, and Cas14a1. In some embodiments, the microarray device may comprise all the various embodiments of devices as described herein.

Described herein are various embodiment of a kit for the detection of a target nucleic acid in a sample, the kit comprising: a swab; elution reagents; lysis reagents; a device comprising a sample interface for receiving the sample; a reaction chamber (e.g., a heating region) in fluidic communication with the sample interface and configured to receive the sample, the heating region comprising a programmable nuclease comprising a guide nucleic acid and a reporter disposed within the heating region, wherein the programmable nuclease is activated by selective binding between the guide nucleic acid and the target nucleic acid, wherein the reporter is configured to release a detection moiety via the activated programmable nuclease; a chemical heating element configured to provide heat to the heating region; a detection region in fluid communication with the heating region and the sample interface, wherein the detection region is configured to detect a signal produced by the released detection moiety, thereby detecting the presence of the target nucleic acid; and a reagent mix comprising amplification reagents, wherein the reagent mix is disposed within the sample interface, the heating region, the detection region, and/or between the sample interface and the heating region, and wherein the device is configured to determine the presence or absence of the target nucleic acid within a time of less than 30 minutes via the released detection moiety. In some embodiments, the sample interface is configured to receive the sample from the swab. In some embodiments, a collection tube, wherein the collection tube is configured to accept the swab, wherein the sample contained in the swab is transferred to the collection tube, and wherein the collection tube is separate from the device. In some embodiments, the collection tube is configured to be inserted into the sample interface to transfer the sample to the device. In some embodiments, the collection tube is a syringe. In some embodiments, the kit comprises a first container containing the elution reagents and/or the lysis reagents. In some embodiments, the kit comprises dilution reagents. In some embodiments, the kit comprises a second container containing the dilution reagents. In some embodiments, the programmable nuclease comprises a Cas enzyme. In some embodiments, the Cas enzyme is selected from the group comprising of Cas12, Cas13, Cas14, Cas14a, and Cas14a1. In some embodiments, the kit may comprise all the various embodiments of devices, as described herein.

Described herein are various embodiments of a method for the detection of a target nucleic acid in a sample, the method comprising: providing a device configured to determine a presence or absence of a target nucleic acid in less than 30 minutes after a sample is introduced into the device, the device comprising a sample interface, a heating region in fluid communication with the sample interface, and a detection region in fluid communication with the heating region; introducing the sample into the sample interface of the device; mixing the sample with a reagent mix comprising amplification reagents to generate a mixed sample solution; transferring the mixed sample solution from the sample interface to the heating region; amplifying the sample by heating the mixed sample solution in the heating region; performing a programmable nuclease-based assay, wherein selective binding between a guide nucleic acid and the target nucleic acid activates a programmable nuclease probe configured to cleave a reporter probe, thereby releasing a detection moiety into the sample solution when the target nucleic acid is present; transferring the mixed sample solution with the amplified sample from the heating region to the detection region; and determining the presence or absence of the target nucleic acid in the sample via capture of the released detection moiety in the detection region. In some embodiments, the amplifying the target nucleic acid and the performing the programmable nuclease assay are performed as a one-pot reaction in the heating region. In some embodiments, the one-pot reaction is performed between about 30° C. to 60° C. In some embodiments, the one-pot reaction is performed at about 55° C. In some embodiments, the programmable nuclease of the one-pot reaction comprises a Cas enzyme, the Cas enzyme selected from the group comprising of Cas12, Cas13, Cas14, Cas14a, and Cas14a1. In some embodiments, the method further comprises filtering the sample with the reagent mix prior to entering the heating region. In some embodiments, the method further comprises filtering the sample comprises filtering one or more particles from the sample that do not comprise the target nucleic acid. In some embodiments, the filter is located between and in fluid communication with the sample interface and heating region. In some embodiments, the method further comprises a plurality of guide nucleic acids, each guide nucleic acid of the plurality of guide nucleic acids is complementary, or partially complementary to a different segment of the target nucleic acid. In some embodiments, the method further comprises, prior to step (a): providing a collection tube comprising a sample solution comprising the target nucleic acid; and transferring the sample solution to the device via inserting the collection tube into a sample interface of the device, wherein the sample solution dissolves and mixes with a lyophilized reaction mix comprising amplification reagents. In some embodiments, the method may comprise all of the embodiments the various devices, as described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a process flow chart for a programmable nuclease-based detection device, whereby a sample comprising one or more target sequences is collected and prepared before the one or more target sequences are detected. Sample preparation includes compartmentalized thermocycling.

FIGS. 2A-2B show top down and cross-section views of a programmable nuclease-based detection device, as described herein.

FIGS. 3A-3B illustrate a cross sectional view of a programmable nuclease-based detection device comprising a plurality of thermocycling compartments with movable mechanisms, as described herein.

FIGS. 4A-4B illustrate a programmable nuclease probe comprising a programmable nuclease and a guide nucleic acid complexed with the programmable nuclease before and after a complementary binding event, as described herein.

FIGS. 5A-5B show a programmable nuclease probe before and after a complementary binding event and the generation of a signal indicating a presence of a target sequence or target nucleic acid, as described herein.

FIG. 6 illustrates a large molecular weight reporter that can undergo a reaction to generate an amplified signal that is detectable by a sensor, as described herein.

FIGS. 7A-7C illustrate various multiplexing embodiments of the programmable nuclease-based detection device, as described herein.

FIG. 8 illustrates an assay design for a point-of-need (PON) 5-plex respiratory panel. FIG. 8 as originally filed discloses viral detection by CRISPR-Cas complexes pooled in discrete regions, and potential for broad spectrum application. This text was located below the “Pan-CoV” and “Endogenous human control” panel columns.

FIG. 9 illustrates a PON disposable device, according to an embodiment, as described herein. FIG. 9 as originally filed discloses directions at the left of the “PON Device Front Cover.” The directions recite: 1. Insert Swab; 2. Close lid; 3. Spring-loaded agitation activated; 4. Chemical heating activated; 5. Wait 15 minutes; 6. Read results.

FIGS. 10A-10C illustrate a consumable and embodiments of components, as described herein.

FIGS. 11A-11D further illustrate various embodiments of components of a consumable, as described herein.

FIG. 12 illustrates valve positioning of the consumable device, as described herein.

FIG. 13 presents an oxidation curve for HERC2 gene DETECTR reaction with electrochemical reporters, as described herein.

FIG. 14 presents a reduction curve for HERC2 DETECTR reaction with electrochemical reporters, as described herein.

FIG. 15 presents a cyclic voltammogram taken before and after HERC2 DETECTR reaction, as described herein.

FIG. 16 presents an oxidation curve for SARS-CoV-2 DETECTR reaction with electrochemical reporters, as described herein.

FIG. 17 presents full data set for square wave voltammetry measurements of SARS-CoV-2 DETECTR reaction with electrochemical reporters and controls, as described herein.

FIG. 18 presents complexing master mix with R1763 (N-gene), as described herein.

FIG. 19 presents experimental conditions for square wave voltammetry measurements, as described herein.

FIG. 20 illustrates immobilization strategies for CRISPR-Cas diagnostic assay components, as described herein

FIG. 21 illustrates an embodiment where immobilization strategies are combined to enable CRISPR diagnostic readouts, as described herein.

FIG. 22 presents results for the evaluation of the compatibility of various chemical modifications to gRNAs, as described herein.

FIG. 23 presents results for the immobilization of gRNAs to a streptavidin coated surface, as described herein.

FIG. 24 presents results for immobilization of Cas protein-RNA complexes, as described herein.

FIGS. 25A-25B present results for immobilization of reporters, as described herein.

FIG. 26 presents results for functional testing of combined immobilized ribonucleoprotein (RNP) and reporter system, as described herein.

FIGS. 27A-27E present results for evaluation of different reporters for immobilization in combination with Cas complex immobilization, as described herein.

FIGS. 28A-28C present results for Cy5 reporter is functional for DETECTR, as described herein.

FIGS. 29A-29F present results for immobilization optimization involving the complex formation step, as described herein.

FIGS. 30A-30B present results for immobilization optimization involving a gRNA/reporter binding time and reporter concentration.

FIGS. 31A-31C present results showing target discrimination of modified gRNAs.ly

FIGS. 32A-32E present results showing biotin-modified Cas13a gRNA is functional.

FIG. 33 presents results of a streptavidin coated microscope slide with biotinylated reporter.

FIGS. 34A-34B present results of DETECR reaction on glass slide.

FIG. 35 present experiment conditions, as described herein.

FIG. 36 present experiment conditions, as described herein. FIG. 36 discloses SEQ ID NOS 21-24, respectively, in order of appearance.

FIGS. 37A-37B present experiment conditions, as described herein.

FIGS. 38A-38B present experiment conditions, as described herein.

FIGS. 39A-39B present experiment conditions, as described herein.

FIGS. 40A-40B present experiment conditions, as described herein.

FIGS. 41A-41B present experiment conditions, as described herein.

FIG. 42 presents experiment conditions, as described herein.

FIG. 43 shows a layout for a DETECTR assay device.

FIG. 44 shows a schematic of a sliding valve device.

FIG. 45 shows a diagram of sample movement through the sliding valve device as shown in FIG. 44.

FIG. 46 presents results for sample preparation optimization for an initial lysis and concentration buffer screen.

FIG. 47 presents results for sample preparation optimization involving Hotpot with Cas 14a.1. FIG. 47 as originally filed discloses additional details under the first trio of graphs, including: (1) Crude lysis: 25 uL sample+25 uL lysis buffer, incubated at 25 C for 1 minute; (2) HotPot: 55 C, IB1 buffer, Bsm polymerase, 0.08 dNTP conc., 1× primers; (3) Sample=250 copies/rxn SeraCare; (4) Rxn=5 ul sample+2.5 ul Cas 14a.1 complex+17.5 uL master mix.

FIG. 48 presents results for sample preparation optimization involving a LANCR (Cas12MO8 DETECTR) control run. FIG. 48 as originally filed discloses additional details to the left of the “VTE5” graphs, including: (1) Crude lysis: 25 uL sample+25 uL lysis buffer, incubated at 25 C for 1 minute; (2) LANCR: 5 uL Sample in a 25 uL reaction volume (standard conditions); (3) DETECTR: 2 uL LANCR product in 20 uL reaction volume (standard conditions); (4) Sample: 250 copies/rxn SeraCare.

FIGS. 49A-49B present lyophilization optimization results with Group 1: Trehalose using RT-LAMP on the left and DETECTR on the right.

FIGS. 50A-50B present lyophilization optimization results for Group 2: PVP 40, sorbitol, Mannitol, Mannosse, using RT-LAMP MM with 3-8% of the candidate excipient as shown in FIG. 50A and results for DETECTR as shown in FIG. 50B.

FIGS. 51A-51B presents lyophilization optimization results for Group 2: PVP 40, Sorbitol, Mannitol, Mannose, using DETECTR MM with 3-5% of candidate excipient.

FIG. 52 presents lyophilization optimization results for RT-LAMP Mastermix in Trehalose Functionality Screening.

FIGS. 53A-53B presents lyophilization optimization results for DETECTR Mastermix in Trehalose, as described herein.

FIGS. 54A-54B present results for HotPot involving LAMP amplification with Cas14a DETECTR in single reaction volume (one-pot).

FIG. 55 presents results for RT-LAMP amplification with Cas14a DETECTR in single reaction volume (one-pot).

FIGS. 56A-56B presents results for identifying buffers that are compatible with Cas14a and low temperature RT-LAMP (LowLAMP).

FIG. 57 presents results involving the impact of individual components on the performance of Cas14 at low temperature RT-LAMP conditions.

FIG. 58 presents results for LAMP amplification with Cas14a DETECTR in single reaction volume (one-pot).

FIG. 59A-59B presents results for one-pot Cas14 with LowLAMP at 50 C.

FIG. 60 presents results for one-pot Cas14 with Bsm DNA polymerase at 55 C.

FIG. 61 presents results for a limit of detection study involving one-pot DETECTR (HotPot).

FIG. 62 presents results for a limit of detection study involving one-pot DETECTR (HotPot), where two different DNA polymerases at 55 C were tested.

FIG. 63 presents results for a study involving replacing Bst polymerase in the NEAR assay, showing enablement for SARS-CoV-2 detection at lower temperatures.

FIG. 64 presents results for NEAR assay amplification functions in Cas14a optimal buffers.

FIG. 65 presents results for Cas14a functions in a range of KOAc salt concentrations.

FIG. 66 presents results for a study involving increasing concentrations of KOAc to improve NEAR performance in Cas14a optimal buffers.

FIG. 67 presents results for a study involving increasing concentrations of KOAc to improve NEAR performance in Cas14a optimal buffers.

FIGS. 68A-68B present sequences and results for performance of Cas14a.1 crRNAs on SARS-CoV-2 E-gene amplicon, respectively. FIG. 68A discloses SEQ ID NO: 66.

FIG. 69 presents results for the evaluation of the performance of Klenow(exo-) NEAR assay in IB13 buffer at decreasing salt concentrations.

FIG. 70 presents an overview of sRCA.

FIG. 71 presents results from screening dumbbell DNA templates for sRCA.

FIG. 72 presents results from a study involving the ability of Cas14a to detect product of RCA reaction across increasing temperatures.

FIG. 73 presents results from a study involving the effects of trigger oligos.

FIG. 74 presents results from a study involving a titration of trigger oligos for Cas14 one-pot sRCA.

FIG. 75 presents results from evaluating of Cas12M08 in one-pot sRCA.

FIG. 76 presents an overview of RCA positive feedback for Cas13.

FIG. 77 presents results from evaluating Cas13-compatible DNA templates for RCA.

FIG. 78 presents results from a study evaluating whether a Cas13-compatible DNA template is functional in RCA.

FIG. 79 presents results from a study involving Cas13 functionality in a one-pot sRCA reaction across increasing temperatures.

FIG. 80 presents an overview of CasPin.

FIG. 81 presents potential hairpin structures for CasPin.

FIG. 82 presents results for an initial design using two hairpins. FIG. 82 discloses SEQ ID NO: 56.

FIG. 83 presents a schematic of combined gRNA and reporter immobilization on the left and results for immobilization of DETECTR components using NHS-Amine chemistries on the right.

FIG. 84 presents results from optimizing the conjugation buffer to reduce non-specific binding.

FIG. 85 presents results from a study involving immobilizing different combinations of reporter+guide+Cas12M08.

FIG. 86 presents results from a study optimizing gRNA and target concentrations to improve signal-to-noise ratio for immobilized DETECTR.

FIGS. 87A-87B present modifications and results from evaluating various amino modifications for DETECTR immobilization, respectively.

FIG. 88 presents results for the FASTR assay, involving detection of SARS-CoV-2 with rapid thermocycling+CRISPR Dx.

FIG. 89 presents results from a study to determine top performing polymerases and buffers for the FASTR assay.

FIG. 90 presents results for single copy detection of SARS-CoV-2 with FASTR.

FIG. 91 presents results for variations on rapid cycling times for denaturation and annealing/extension in FASTR.

FIG. 92 presents results for minimizing RT time for FASTR.

FIG. 93 presents results for higher pH buffers that improve FASTR performance.

FIG. 94 presents results for FASTR compatibility with crude lysis buffers.

FIG. 95 presents results for non-optimized multiplexing of FASTR.

FIG. 96 presents results for multiplex FASTR.

FIG. 97 presents results for the limit of detection of multiplex FASTR.

FIG. 98 presents key primers and gRNAs. FIG. 98 discloses SEQ ID NOS 57-65, respectively, in order of appearance.

FIGS. 99A-99B present results for a one-pot master mix of both RT-LAMP and DETECTR assay reagents pooled together. Aliquots of the same reaction mixture containing Cas12M08 protein were run separately for each assay. FIG. 99A shows results for the RT-LAMP assay and FIG. 99B shows results for the DETECTR assay.

FIG. 100 presents results for the reconstituted Cas12M08-based DETECTR master mix after lyophilization.

FIG. 101 presents results for a Cas14a1-based DETECTR assay.

FIGS. 102A-102B present results for RT-LAMP and Cas12M08-based DETECTR assays where one sample containing the reagent master mix was stored for two weeks prior to lyophilization.

FIG. 103 presents results for small volume, lyophilized reaction master mixes.

FIG. 104 presents dynamic scanning calorimetry results for a pooled, lyophilized master mix of DETECTR and RT-LAMP reagents.

FIG. 105 presents a list of excipients.

FIG. 106 presents results for a one-pot DETECTR assay ran on a handheld microfluidic device.

FIG. 107 illustrates an embodiment of a multiplex lateral flow strip, as described herein.

FIG. 108 illustrates an embodiment of a workflow with multiplex “HotPot” as described herein. FIG. 108 as originally filed recites “HotPot reaction well immobilized Cas, sgRNA, and reporters with distinct functional groups” between the dashed ovals of reporters (10805) and (10806).

FIG. 109 illustrates an embodiment for HRP paper-based detection, as described herein.

FIG. 110 illustrates an embodiment for an HRP-based multiplex lateral flow assay, as described herein.

FIG. 111 illustrates an embodiment for Multiplexed Cas13 immobilization approach to an HRP-based multiplex lateral flow assay, as described herein. FIG. 111 as originally filed recites “Cas13 reaction with immobilized gRNA (opt. pooled in spot)” under the circle indicating the surface of the well (11101).

FIG. 112 shows results for both DNAse and DETECTR based assays for two replicate runs a week apart.

FIG. 113 illustrates the use of multiple Cas-complex probes guide pooling enhanced signal detection to a lateral flow assay, as described herein.

FIG. 114 depicts results of a DETECTR assay showing enhanced Cas12a-based detection of the GF184 target using a pooled-guide (pooled-gRNA) format compared to DETECTR Cas12a-based assay using an individual gRNA format.

FIG. 115 depicts results of a DETECTR assay showing enhanced sensitivity of the Cas13a-based detection of the SC2 target using a pooled-guide format compared to the Cas13a-based assays using an individual guide format.

FIG. 116 shows images corresponding to each chamber, used to count the number of positive droplets, showing that the Cas13a-DETECTR assay samples containing the pooled guide RNAs generated more crystals containing the amplified products per starting copy of the target RNA than the Cas13a-DETECTR assay samples containing the guide RNAs in individual format.

FIG. 117 shows that measurement of signal intensity following amplification showed that the Cas13a-DETECTR assay samples containing the pooled guide RNAs generated more signal intensity per starting copy of the target template RNA than the Cas13a-DETECTR assay samples containing the guide RNAs in individual format.

FIG. 118 shows that measurement of signal intensity following amplification showed that the Cas13a-DETECTR assay samples containing the pooled guide RNAs generated more signal intensity per starting copy of the target template RNA than the Cas13a-DETECTR assay samples containing the guide RNAs in individual format. FIG. 118 also shows that relative quantification performed by counting the number of positive droplets showed that the Cas13a-DETECTR assay samples containing the pooled guide RNAs generated more crystals containing the amplified products per starting copy of the target template RNA than the Cas13a-DETECTR assay samples containing the guide RNAs in individual format.

FIG. 119 shows that Cas13a DETECTR assay samples containing the pooled guides (R4637, R4638, R4667, R4676, R4684, R4689, R4691) did not exhibit higher target detection sensitivity per starting copy of the target than the Cas13a DETECTR samples containing the single guides R4684, R4667, or R4785 (RNAseP guide) in individual format.

FIG. 120 illustrates an embodiment of a handheld device comprising a lateral flow assay, as described herein.

FIGS. 121A-121B illustrate an embodiment of a point of care device comprising a lateral flow assay, as described herein.

FIG. 122 illustrates an embodiment of a point of care device comprising a lateral flow assay, as described herein.

FIG. 123 illustrates an embodiment of a point of care device comprising a lateral flow assay and a spiral reaction chamber as described herein.

FIG. 124 illustrates an embodiment of a point of care device comprising switch triggers for triggering device actuators as described herein.

FIG. 125 illustrates an embodiment of a reaction chamber that is coupled to an input port and is substantially spiral in shape as described herein.

FIG. 126 illustrates an embodiment of a housing structure for lateral flow assays comprising channel structures of substantially the same contour length for each assay as described herein.

FIGS. 127A-127B illustrates an embodiment of a point of care device before actuation and after partial actuation of device actuators as described herein.

FIGS. 128A-128B illustrates an embodiment of a chemical heating element and its time-dependent temperature profile as described herein.

FIGS. 129A-129B illustrates embodiments of lateral flow strips configurations as described herein.

FIG. 130 illustrates an embodiment of a point of care device comprising a chemical heating element and an electrical heating element.

FIGS. 131A-131B illustrates results of DETECTR assays showing successful nucleic acids amplification utilizing one of the point of care device embodiments described herein.

FIG. 132 illustrates results of DETECTR assays showing successful detection of nucleic acids utilizing one of the flow strip assay embodiments described herein.

FIG. 133 illustrates a flow diagram of a process used to evaluate, characterize, and optimize proteins for diagnostic applications.

FIG. 134 illustrates a schematic showing a workflow for the process using Labcyte Echo.

FIG. 135 shows experimental results for fluorescence of three candidate Cas enzymes.

FIG. 136 shows the performance of three candidate Cas enzymes at different temperatures and buffers.

FIG. 137 shows the results of testing conducted with CasM. 1740 with three additional buffers at 35° C.

FIG. 138 shows the results of experiments investigating the limits of detection on single-strand oligo or synthetic dsDNA target at 35° C.

FIG. 139 shows the results of experiments evaluating the limit of detection at both 35° C. and the highest temperature that the protein was demonstrated to function at for CasM. 1740.

FIG. 140 shows the results of experiments investigating the effects of additives and assay formulations on the performance of the proteins.

FIG. 141 shows the results for single nucleotide mutation sensitivity experiments with CasM. 124070.

FIG. 142 shows the results for single nucleotide mutation sensitivity experiments with CasM. 08.

FIG. 143 shows the results for single nucleotide mutation sensitivity experiments with CasM. 124070 and CasM. 08 with the same target site.

FIGS. 144A-144B show the results of experiments investigating the kinetics of trans-cleavage for proteins that have the best performance in terms of sensitivity, specificity, or thermostability.

FIG. 145 summarizes the performance results for various Cas enzymes.

### DETAILED DESCRIPTION

The present disclosure provides systems and methods for nucleic acid target detection. The systems and methods of the present disclosure can be implemented using devices that are configured for programmable nuclease-based detection. In some embodiments, the devices can be configured for single reaction detection. In some embodiments, the devices can be disposable devices. The devices disclosed herein can be particularly well suited for carrying out highly efficient, rapid, and accurate reactions for detecting whether a target is present in a sample. The target can comprise a target sequence or target nucleic acid. As used herein, a target can be referred to interchangeably as a target nucleic acid. Further, a target can be referred to as a target amplicon or a target nucleic acid amplicon if such target undergoes amplification (e.g., through a thermocycling process as described elsewhere herein). The target nucleic acid can be a portion of a nucleic acid of interest, e.g., a target nucleic acid from any plant, animal, virus, or microbe of interest. The devices provided herein can be used to perform rapid tests in a single integrated system.

The target nucleic acid can be a nucleic acid or a portion of a nucleic acid from a pathogen, virus, bacterium, fungi, protozoa, worm, or other agent(s) or organism(s) responsible for and/or related to a disease or condition in living organisms (e.g., humans, animals, plants, crops, and the like). The target nucleic acid can be a nucleic acid, or a portion thereof. The target nucleic acid can be a portion of a nucleic acid from a gene expressed in a cancer or genetic disorder in the sample. The target nucleic acid can be a portion of an RNA or DNA from any organism in the sample. In some embodiments, one or more programmable nucleases as disclosed herein can be activated to initiate trans cleavage activity of a reporter (also referred to herein as a reporter molecule). A programmable nuclease as disclosed herein can, in some cases, bind to a target sequence or target nucleic acid to initiate trans cleavage of a reporter. The programmable nuclease can be referred to as an RNA-activated programmable RNA nuclease. In some instances, the programmable nuclease as disclosed herein can bind to a target DNA to initiate trans cleavage of an RNA reporter. Such a programmable nuclease can be referred to herein as a DNA-activated programmable RNA nuclease. In some cases, a programmable nuclease as described herein can be activated by a target RNA or a target DNA. For example, a programmable nuclease, e.g., a Cas enzyme, can be activated by a target RNA nucleic acid or a target DNA nucleic acid to cleave RNA reporters. In some embodiments, the Cas enzyme can bind to a target ssDNA which initiates trans cleavage of RNA reporters. In some instances, a programmable nuclease as disclosed herein can bind to a target DNA to initiate trans cleavage of a DNA reporter, and this programmable nuclease can be referred to as a DNA-activated programmable DNA nuclease.

The nucleic acids described and referred to herein can comprise a plurality of base pairs. A base pair can be a biological unit comprising two nucleobases bound to each other by hydrogen bonds. Nucleobases can comprise adenine, guanine, cytosine, thymine, and/or uracil. In some cases, the nucleic acids described and referred to herein can comprise different base pairs. In some cases, the nucleic acids described and referred to herein can comprise one or more modified base pairs. The one or more modified base pairs can be produced when one or more base pairs undergo a chemical modification leading to new bases. The one or more modified base pairs can be, for example, Hypoxanthine, Inosine, Xanthine, Xanthosine, 7-Methylguanine, 7-Methylguanosine, 5,6-Dihydrouracil, Dihydrouridine, 5-Methylcytosine, 5-Methylcytidine, 5-hydroxymethylcytosine (5hmC), 5-formylcytosine (5fC), or 5-carboxylcytosine (5caC).

The programmable nuclease can become activated after binding of a guide nucleic acid that is complexed with the programmable nuclease with a target nucleic acid, and the activated programmable nuclease can cleave the target nucleic acid, which can result in a trans cleavage activity. Trans cleavage activity can be non-specific cleavage of nearby single-stranded nucleic acids by the activated programmable nuclease, such as trans cleavage of detector nucleic acids with a detection moiety. Once the target nucleic acid is cleaved by the activated programmable nuclease, the detection moiety can be released or separated from the reporter and can directly or indirectly generate a detectable signal. The reporter and/or the detection moiety can be immobilized on a support medium. Often the detection moiety is at least one of a fluorophore, a dye, a polypeptide, or a nucleic acid. Sometimes the detection moiety binds to a capture molecule on the support medium to be immobilized. The detectable signal can be visualized on the support medium to assess the presence or concentration of one or more target nucleic acids associated with an ailment, such as a disease, cancer, or genetic disorder.

The systems and methods of the present disclosure can be implemented using a device that is compatible with any type of programmable nuclease that is human-engineered or naturally occurring. The programmable nuclease can comprise a nuclease that is capable of being activated when complexed with a guide nucleic acid and a target nucleic acid segment or a portion thereof. A programmable nuclease can become activated when complexed with a guide nucleic acid and a target sequence of a target gene of interest. The programmable nuclease can be activated upon binding of a guide nucleic acid to a target nucleic acid and can exhibit or enable trans cleavage activity once activated. In any instances or embodiments where a CRISPR-based programmable nuclease is described or used, it is recognized herein that any other type of programmable nuclease can be used in addition to or in substitution of such CRISPR-based programmable nuclease.

The systems and methods of the present disclosure can be implemented using a device that is compatible with a plurality of programmable nucleases. The device can comprise a plurality of programmable nuclease probes comprising the plurality of programmable nucleases and one or more corresponding guide nucleic acids. The plurality of programmable nuclease probes can be the same. Alternatively, the plurality of programmable nuclease probes can be different. For example, the plurality of programmable nuclease probes can comprise different programmable nucleases and/or different guide nucleic acids associated with the programmable nucleases.

As used herein, a programmable nuclease generally refers to any enzyme that can cleave nucleic acid. The programmable nuclease can be any enzyme that can be or has been designed, modified, or engineered by human contribution so that the enzyme targets or cleaves the nucleic acid in a sequence-specific manner. Programmable nucleases can include, for example, zinc-finger nucleases (ZFNs), transcription activator-like effector nucleases (TALENs), and/or RNA-guided nucleases such as the bacterial clustered regularly interspaced short palindromic repeat (CRISPR)-Cas (CRISPR-associated) nucleases or Cpf1. Programmable nucleases can also include, for example, PfAgo and/or NgAgo.

ZFNs can cut genetic material in a sequence-specific matter and can be designed, or programmed, to target specific viral targets. A ZFN is composed of two domains: a DNA-binding zinc-finger protein linked to the Fokl nuclease domain. The DNA-binding zinc-finger protein is fused with the non-specific Fokl cleave domain to create ZFNs. The protein will typically dimerize for activity. Two ZFN monomers form an active nuclease; each monomer binds to adjacent half-sites on the target. The sequence specificity of ZFNs is determined by ZFPs. Each zinc-finger recognizes a 3-bp DNA sequence, and 3-6 zinc-fingers are used to generate a single ZFN subunit that binds to DNA sequences of 9-18 bp. The DNA-binding specificities of zinc-fingers is altered by mutagenesis. New ZFPs are programmed by modular assembly of pre-characterized zinc fingers.

Transcription activator-like effector nucleases (TALENs) can cut genetic material in a sequence-specific matter and can be designed, or programmed, to target specific viral targets. TALENs contain the Fokl nuclease domain at their carboxyl termini and a class of DNA binding domains known as transcription activator-like effectors (TALEs). TALENs are composed of tandem arrays of 33-35 amino acid repeats, each of which recognizes a single base-pair in the major groove of target viral DNA. The nucleotide specificity of a domain comes from the two amino acids at positions 12 and 13 where Asn-Asn, Asn-Ile, His-Asp and Asn-Gly recognize guanine, adenine, cytosine and thymine, respectively. That pattern allows one to program TALENs to target various nucleic acids.

The programmable nuclease can comprise any type of human engineered enzymes. Alternatively, the programmable nuclease can comprise CRISPR enzymes derived from naturally occurring bacteria or phage. A programmable nuclease can be a Cas protein (also referred to, interchangeably, as a Cas nuclease). A crRNA and Cas protein can form a CRISPR enzyme. The programmable nuclease can be a CRISPR-Cas (clustered regularly interspaced short palindromic repeats—CRISPR associated) nucleoprotein complex with trans cleavage activity, which can be activated by binding of a guide nucleic acid with a target nucleic acid. The programmable nuclease can comprise one or more amino acid modifications. The programmable nuclease be a nuclease derived from a CRISPR-Cas system. The programmable nuclease can be a nuclease derived from recombineering.

### Programmable Nucleases

Disclosed herein are programmable nucleases and uses thereof, e.g., detection and editing of target nucleic acids. In some instances, programmable nucleases comprise a Type V CRISPR/Cas protein. In some instances, Type V CRISPR/Cas proteins comprise nucleic acid cleavage activity. In some instances, Type V CRISPR/Cas proteins cleave or nick single-stranded nucleic acids, double, stranded nucleic acids, or a combination thereof. In some cases, Type V CRISPR/Cas proteins cleave single-stranded nucleic acids. In some cases, Type V CRISPR/Cas proteins cleave double-stranded nucleic acids. In some cases, Type V CRISPR/Cas proteins nick double-stranded nucleic acids. Typically, guide RNAs of Type V CRISPR/Cas proteins hybridize to ssDNA or dsDNA. However, the trans cleavage activity of Type V CRISPR/Cas protein is typically directed towards ssDNA. [41] In some cases, the Type V CRISPR/Cas protein comprises a catalytically inactive nuclease domain. In some cases, the Type V CRISPR/Cas protein comprises a catalytically inactive nuclease domain. A catalytically inactive domain of a Type V CRISPR/Cas protein may comprise at least 1, at least 2, at least 3, at least 4, or at least 5 mutations relative to a wild type nuclease domain of the Type V CRISPR/Cas protein. Said mutations may be present within a cleaving or active site of the nuclease. [42] The Type V CRISPR/Cas protein may be a Cas14 protein. The Cas 14 protein may be a Cas14a.1 protein. The Cas14a.1 protein may be represented by SEQ ID NO: 1, presented in Table 1. The Cas14 protein may comprise an amino acid sequence that is at least 70%, at least 75%, at least 80%, at least 85%, at least 90%, at least 95%, at least 98%, at least 99%, or 100% identical to SEQ ID NO: 1. The Cas14 protein may consist of an amino acid sequence that is at least 70%, at least 75%, at least 80%, at least 85%, at least 90%, at least 95%, at least 98%, at least 99%, or 100% identical to SEQ ID NO: 1. The Cas14 protein may comprise at least about 50, at least about 100, at least about 150, at least about 200, at least about 250, at least about 300, at least about 350, at least about 400, at least about 450, at least about 500 consecutive amino acids of SEQ ID NO: 1.

TABLE 1
Cas14a.1 Protein Sequence

| SEQ ID | |
|---|---|
| NO: | SEQUENCE |
| SEQ ID | MAKNTITKTLKLRIVRPYNSAEVEKIVADEK |
| NO: 1 | NNREKIALEKNKDKVKEACSKHLKVAAYCT |
| | TQVERNACLFCKARKLDDKFYQKLRGQFPD |
| | AVFWQEISEIFRQLQKQAAEIYNQSLIELY |
| | YEIFIKGKGIANASSVEHYLSDVCYTRAAE |
| | LFKNAAIASGLRSKIKSNFRLKELKNMKSG |
| | LPTTKSDNFPIPLVKQKGGQYTGFEISNHN |
| | SDFIIKIPFGRWQVKKEIDKYRPWEKFDFE |
| | QVQKSPKPISLLLSTQRRKRNKGWSKDEGT |
| | EAEIKKVMNGDYQTSYIEVKRGSKIGEKSA |
| | WMLNLSIDVPKIDKGVDPSIIGGIDVGVKS |
| | PLVCAINNAFSRYSISDNDLFHFNKKMFAR |
| | RRILLKKNRHKRAGHGAKNKLKPITILTEK |
| | SERFRKKLIERWACEIADFFIKNKVGTVQM |
| | ENLESMKRKEDSYFNIRLRGFWPYAEMQNK |
| | IEFKLKQYGIEIRKVAPNNTSKTCSKCGHL |
| | NNYFNFEYRKKNKFPHFKCEKCNFKENADY |
| | NAALNISNPKLKSTKEEP |

In some instances, the Type V CRISPR/Cas protein has been modified (also referred to as an engineered protein). For example, a Type V CRISPR/Cas protein disclosed herein or a variant thereof may comprise a nuclear localization signal (NLS). In some cases, the NLS may comprise a sequence of KRPAATKKAGQAKKKKEF (SEQ ID NO: 2).Type V CRISPR/Cas proteins may be codon optimized for expression in a specific cell, for example, a bacterial cell, a plant cell, a eukaryotic cell, an animal cell, a mammalian cell, or a human cell. In some embodiments, the Type V CRISPR/Cas protein is codon optimized for a human cell.

In some instances, the Type V CRISPR/Cas protein has been modified (also referred to as an engineered protein). For example, a Type V CRISPR/Cas protein disclosed herein or a variant thereof may comprise a nuclear localization signal (NLS). In some cases, the NLS may comprise a sequence of KRPAATKKAGQAKKKKEF (SEQ ID NO: 2). Type V CRISPR/Cas proteins may be codon optimized for expression in a specific cell, for example, a bacterial cell, a plant cell, a eukaryotic cell, an animal cell, a mammalian cell, or a human cell. In some embodiments, the Type V CRISPR/Cas protein is codon optimized for a human cell.

### Cas14 Proteins

In some instances, the TypeV CRISPR/Cas protein comprises a Cas14 protein. Cas14 proteins may comprise a bilobed structure with distinct amino-terminal and carboxy-terminal domains. The amino- and carboxy-terminal domains may be connected by a flexible linker. The flexible linker may affect the relative conformations of the amino- and carboxyl-terminal domains. The flexible linker may be short, for example less than 10 amino acids, less than 8 amino acids, less than 6 amino acids, less than 5 amino acids, or less than 4 amino acids in length. The flexible linker may be sufficiently long to enable different conformations of the amino- and carboxy-terminal domains among two Cas14 proteins of a Cas14 dimer complex (e.g., the relative orientations of the amino- and carboxy-terminal domains differ between two Cas14 proteins of a Cas14 homodimer complex). The linker domain may comprise a mutation which affects the relative conformations of the amino- and carboxyl-terminal domains. The linker may comprise a mutation which affects Cas14 dimerization. For example, a linker mutation may enhance the stability of a Cas14 dimer.

In some instances, the amino-terminal domain of a Cas14 protein comprises a wedge domain, a recognition domain, a zinc finger domain, or any combination thereof. The wedge domain may comprise a multi-strand β-barrel structure. A multi-strand β-barrel structure may comprise an oligonucleotide/oligosaccharide-binding fold that is structurally comparable to those of some Cas12 proteins. The recognition domain and the zinc finger domain may each (individually or collectively) be inserted between O-barrel strands of the wedge domain. The recognition domain may comprise a 4-α-helix structure, structurally comparable but shorter than those found in some Cas12 proteins. The recognition domain may comprise a binding affinity for a guide nucleic acid or for a guide nucleic acid-target nucleic acid heteroduplex. In some cases, a REC lobe may comprise a binding affinity for a PAM sequence in the target nucleic acid. The amino-terminal may comprise a wedge domain, a recognition domain, and a zinc finger domain. The carboxy-terminal may comprise a RuvC domain, a zinc finger domain, or any combination thereof. The carboxy-terminal may comprise one RuvC and one zinc finger domain.

Cas14 proteins may comprise a RuvC domain or a partial RuvC domain. The RuvC domain may be defined by a single, contiguous sequence, or a set of partial RuvC domains that are not contiguous with respect to the primary amino acid sequence of the Cas14 protein. In some instances, a partial RuvC domain does not have any substrate binding activity or catalytic activity on its own. A Cas14 protein of the present disclosure may include multiple partial RuvC domains, which may combine to generate a RuvC domain with substrate binding or catalytic activity. For example, a Cas14 may include 3 partial RuvC domains (RuvC-I, RuvC-II, and RuvC-III, also referred to herein as subdomains) that are not contiguous with respect to the primary amino acid sequence of the Cas14 protein, but form a RuvC domain once the protein is produced and folds. A Cas14 protein may comprise a linker loop connecting a carboxy terminal domain of the Cas14 protein with the amino terminal domain of the Cas 14 protein, and wherein the carboxy terminal domain comprises one or more RuvC domains and the amino terminal domain comprises a recognition domain.

Cas14 proteins may comprise a zinc finger domain. In some instances, a carboxy terminal domain of a Cas14 protein comprises a zinc finger domain. In some instances, an amino terminal domain of a Cas14 protein comprises a zinc finger domain. In some instances, the amino terminal domain comprises a wedge domain (e.g., a multi-β-barrel wedge structure), a zinc finger domain, or any combination thereof. In some cases, the carboxy terminal domain comprises the RuvC domains and a zinc finger domain, and the amino terminal domain comprises a recognition domain, a wedge domain, and a zinc finger domain.

Cas14 proteins may be relatively small compared to many other Cas proteins, making them suitable for nucleic acid detection or gene editing. For instance, a Cas14 protein may be less likely to adsorb to a surface or another biological species due to its small size. The smaller nature of these proteins also allows for them to be more easily packaged as a reagent in a system or assay, and delivered with higher efficiency as compared to other larger Cas proteins. In some cases, a Cas14 protein is 400 to 800 amino acid residues long, 400 to 600 amino acid residues long, 440 to 580 amino acid residues long, 460 to 560 amino acid residues long, 460 to 540 amino acid residues long, 460 to 500 amino acid residues long, 400 to 500 amino acid residues long, or 500 to 600 amino acid residues long. In some cases, a Cas14 protein is less than about 550 amino acid residues long. In some cases, a Cas14 protein is less than about 500 amino acid residues long.

In some instances, a Cas14 protein may function as an endonuclease that catalyzes cleavage at a specific position within a target nucleic acid. In some instances, a Cas14 protein is capable of catalyzing non-sequence-specific cleavage of a single stranded nucleic acid. In some cases, a Cas14 protein is activated to perform trans cleavage activity after binding of a guide nucleic acid with a target nucleic acid. This trans cleavage activity is also referred to as “collateral” or “transcollateral” cleavage. Trans cleavage activity may be non-specific cleavage of nearby single-stranded nucleic acid by the activated programmable nuclease, such as trans cleavage of detector nucleic acids with a detection moiety.

### Engineered Programmable Nuclease Probes

Disclosed herein are non-naturally occurring compositions and systems comprising at least one of an engineered Cas protein and an engineered guide nucleic acid, which may simply be referred to herein as a Cas protein and a guide nucleic acid, respectively. In general, an engineered Cas protein and an engineered guide nucleic acid refer to a Cas protein and a guide nucleic acid, respectively, that are not found in nature. In some instances, systems and compositions comprise at least one non-naturally occurring component. For example, compositions and systems may comprise a guide nucleic acid, wherein the sequence of the guide nucleic acid is different or modified from that of a naturally-occurring guide nucleic acid. In some instances, compositions and systems comprise at least two components that do not naturally occur together. For example, compositions and systems may comprise a guide nucleic acid comprising a repeat region and a spacer region which do not naturally occur together. Also, by way of example, composition and systems may comprise a guide nucleic acid and a Cas protein that do not naturally occur together. Conversely, and for clarity, a Cas protein or guide nucleic acid that is “natural,” “naturally-occurring,” or “found in nature” includes Cas proteins and guide nucleic acids from cells or organisms that have not been genetically modified by a human or machine.

In some instances, the guide nucleic acid may comprise a non-natural nucleobase sequence. In some instances, the non-natural sequence is a nucleobase sequence that is not found in nature. The non-natural sequence may comprise a portion of a naturally occurring sequence, wherein the portion of the naturally occurring sequence is not present in nature absent the remainder of the naturally-occurring sequence. In some instances, the guide nucleic acid may comprise two naturally occurring sequences arranged in an order or proximity that is not observed in nature. In some instances, compositions and systems comprise a ribonucleotide complex comprising a CRISPR/Cas effector protein and a guide nucleic acid that do not occur together in nature. Engineered guide nucleic acids may comprise a first sequence and a second sequence that do not occur naturally together. For example, an engineered guide nucleic acid may comprise a sequence of a naturally occurring repeat region and a spacer region that is complementary to a naturally occurring eukaryotic sequence. The engineered guide nucleic acid may comprise a sequence of a repeat region that occurs naturally in an organism and a spacer region that does not occur naturally in that organism. An engineered guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. For example, an engineered guide nucleic acid may comprise a naturally occurring crRNA and tracrRNA coupled by a linker sequence.

In some instances, compositions and systems described herein comprise an engineered Cas protein that is similar to a naturally occurring Cas protein. The engineered Cas protein may lack a portion of the naturally occurring Cas protein. The Cas protein may comprise a mutation relative to the naturally-occurring Cas protein, wherein the mutation is not found in nature. The Cas protein may also comprise at least one additional amino acid relative to the naturally-occurring Cas protein. For example, the Cas protein may comprise an addition of a nuclear localization signal relative to the natural occurring Cas protein. In certain embodiments, the nucleotide sequence encoding the Cas protein is codon optimized (e.g., for expression in a eukaryotic cell) relative to the naturally occurring sequence.

In some instances, compositions and systems provided herein comprise a multi-vector system encoding a Cas protein and a guide nucleic acid described herein, wherein the guide nucleic acid and the Cas protein are encoded by the same or different vectors. In some embodiments, the engineered guide and the engineered Cas protein are encoded by different vectors of the system.

### Thermostable Programmable Nuclease

Described herein are various embodiments of thermostable programmable nucleases. In some embodiments, a programmable nuclease is referred to as an effector protein. An effector protein may be thermostable. In some instances, known effector proteins (e.g., Cas12 nucleases) are relatively thermo-sensitive and only exhibit activity (e.g., cis and/or trans cleavage) sufficient to produce a detectable signal in a diagnostic assay at temperatures less than 40° C., and optimally at about 37° C. A thermostable protein may have enzymatic activity, stability, or folding comparable to those at 37° C. In some instances, the trans cleavage activity (e.g., the maximum trans cleavage rate as measured by fluorescent signal generation) of an effector protein in a trans cleavage assay at 40° C. may be at least 50% of that at 37° C. In some instances, the trans cleavage activity of an effector protein in a trans cleavage assay at 40° C. may be at least 55% of that at 37° C. In some instances, the trans cleavage activity of an effector protein in a trans cleavage assay at 40° C. may be at least 60% of that at 37° C. In some instances, the trans cleavage activity of an effector protein in a trans cleavage assay at 40° C. may be at least 65% of that at 37° C. In some instances, the trans cleavage activity of an effector protein in a trans cleavage assay at 40° C. may be at least 70% of that at 37° C. In some instances, the trans cleavage activity of an effector protein in a trans cleavage assay at 40° C. may be at least 75% of that at 37° C. In some instances, the trans cleavage activity of an effector protein in a trans cleavage assay at 40° C. may be at least 80% of that at 37° C. In some instances, the trans cleavage activity of an effector protein in a trans cleavage assay at 40° C. may be at least 85% of that at 37° C. In some instances, the trans cleavage activity of an effector protein in a trans cleavage assay at 40° C. may be at least 90% of that at 37° C. In some instances, the trans cleavage activity of an effector protein in a trans cleavage assay at 40° C. may be at least 95% of that at 37° C. In some instances, the trans cleavage activity of an effector protein in a trans cleavage assay at 40° C. may be at least 100% of that at 37° C. In some instances, the trans cleavage activity of an effector protein in a trans cleavage assay at 40° C. may be at least 1-fold of that at 37° C. In some instances, the trans cleavage activity of an effector protein in a trans cleavage assay at 40° C. may be at least 2-fold of that at 37° C. In some instances, the trans cleavage activity of an effector protein in a trans cleavage assay at 40° C. may be at least 3-fold of that at 37° C. In some instances, the trans cleavage activity of an effector protein in a trans cleavage assay at 40° C. may be at least 4-fold of that at 37° C. In some instances, the trans cleavage activity of an effector protein in a trans cleavage assay at 40° C. may be at least 5-fold of that at 37° C. In some instances, the trans cleavage activity of an effector protein in a trans cleavage assay at 40° C. may be at least 6-fold of that at 37° C. In some instances, the trans cleavage activity of an effector protein in a trans cleavage assay at 40° C. may be at least 7-fold of that at 37° C. In some instances, the trans cleavage activity of an effector protein in a trans cleavage assay at 40° C. may be at least 8-fold of that at 37° C. In some instances, the trans cleavage activity of an effector protein in a trans cleavage assay at 40° C. may be at least 9-fold of that at 37° C. In some instances, the trans cleavage activity of an effector protein in a trans cleavage assay at 40° C. may be at least 10-fold of that at 37° C. In some instances, the trans cleavage activity of an effector protein in a trans cleavage assay at 40° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of an effector protein in a trans cleavage assay at 45° C. may be at least 50% of that at 37° C. In some instances, the trans cleavage activity of an effector protein in a trans cleavage assay at 45° C. may be at least 55% of that at 37° C. In some instances, the trans cleavage activity of an effector protein in a trans cleavage assay at 45° C. may be at least 60% of that at 37° C. In some instances, the trans cleavage activity of an effector protein in a trans cleavage assay at 45° C. may be at least 65% of that at 37° C. In some instances, the trans cleavage activity of an effector protein in a trans cleavage assay at 45° C. may be at least 70% of that at 37° C. In some instances, the trans cleavage activity of an effector protein in a trans cleavage assay at 45° C. may be at least 75% of that at 37° C. In some instances, the trans cleavage activity of an effector protein in a trans cleavage assay at 45° C. may be at least 80% of that at 37° C. In some instances, the trans cleavage activity of an effector protein in a trans cleavage assay at 45° C. may be at least 85% of that at 37° C. In some instances, the trans cleavage activity of an effector protein in a trans cleavage assay at 45° C. may be at least 90% of that at 37° C. In some instances, the trans cleavage activity of an effector protein in a trans cleavage assay at 45° C. may be at least 95% of that at 37° C. In some instances, the trans cleavage activity of an effector protein in a trans cleavage assay at 45° C. may be at least 100% of that at 37° C. In some instances, the trans cleavage activity of an effector protein in a trans cleavage assay at 45° C. may be at least 1-fold of that at 37° C. In some instances, the trans cleavage activity of an effector protein in a trans cleavage assay at 45° C. may be at least 2-fold of that at 37° C. In some instances, the trans cleavage activity of an effector protein in a trans cleavage assay at 45° C. may be at least 3-fold of that at 37° C. In some instances, the trans cleavage activity of an effector protein in a trans cleavage assay at 45° C. may be at least 4-fold of that at 37° C. In some instances, the trans cleavage activity of an effector protein in a trans cleavage assay at 45° C. may be at least 5-fold of that at 37° C. In some instances, the trans cleavage activity of an effector protein in a trans cleavage assay at 45° C. may be at least 6-fold of that at 37° C. In some instances, the trans cleavage activity of an effector protein in a trans cleavage assay at 45° C. may be at least 7-fold of that at 37° C. In some instances, the trans cleavage activity of an effector protein in a trans cleavage assay at 45° C. may be at least 8-fold of that at 37° C. In some instances, the trans cleavage activity of an effector protein in a trans cleavage assay at 45° C. may be at least 9-fold of that at 37° C. In some instances, the trans cleavage activity of an effector protein in a trans cleavage assay at 45° C. may be at least 10-fold of that at 37° C. In some instances, the trans cleavage activity of an effector protein in a trans cleavage assay at 45° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of an effector protein in a trans cleavage assay at 50° C. may be at least 50% of that at 37° C. In some instances, the trans cleavage activity of an effector protein in a trans cleavage assay at 50° C. may be at least 55% of that at 37° C. In some instances, the trans cleavage activity of an effector protein in a trans cleavage assay at 50° C. may be at least 60% of that at 37° C. In some instances, the trans cleavage activity of an effector protein in a trans cleavage assay at 50° C. may be at least 65% of that at 37° C. In some instances, the trans cleavage activity of an effector protein in a trans cleavage assay at 50° C. may be at least 70% of that at 37° C. In some instances, the trans cleavage activity of an effector protein in a trans cleavage assay at 50° C. may be at least 75% of that at 37° C. In some instances, the trans cleavage activity of an effector protein in a trans cleavage assay at 50° C. may be at least 80% of that at 37° C. In some instances, the trans cleavage activity of an effector protein in a trans cleavage assay at 50° C. may be at least 85% of that at 37° C. In some instances, the trans cleavage activity of an effector protein in a trans cleavage assay at 50° C. may be at least 90% of that at 37° C. In some instances, the trans cleavage activity of an effector protein in a trans cleavage assay at 50° C. may be at least 95% of that at 37° C. In some instances, the trans cleavage activity of an effector protein in a trans cleavage assay at 50° C. may be at least 100% of that at 37° C. In some instances, the trans cleavage activity of an effector protein in a trans cleavage assay at 50° C. may be at least 1-fold of that at 37° C. In some instances, the trans cleavage activity of an effector protein in a trans cleavage assay at 50° C. may be at least 2-fold of that at 37° C. In some instances, the trans cleavage activity of an effector protein in a trans cleavage assay at 50° C. may be at least 3-fold of that at 37° C. In some instances, the trans cleavage activity of an effector protein in a trans cleavage assay at 50° C. may be at least 4-fold of that at 37° C. In some instances, the trans cleavage activity of an effector protein in a trans cleavage assay at 50° C. may be at least 5-fold of that at 37° C. In some instances, the trans cleavage activity of an effector protein in a trans cleavage assay at 50° C. may be at least 6-fold of that at 37° C. In some instances, the trans cleavage activity of an effector protein in a trans cleavage assay at 50° C. may be at least 7-fold of that at 37° C. In some instances, the trans cleavage activity of an effector protein in a trans cleavage assay at 50° C. may be at least 8-fold of that at 37° C. In some instances, the trans cleavage activity of an effector protein in a trans cleavage assay at 50° C. may be at least 9-fold of that at 37° C. In some instances, the trans cleavage activity of an effector protein in a trans cleavage assay at 50° C. may be at least 10-fold of that at 37° C. In some instances, the trans cleavage activity of an effector protein in a trans cleavage assay at 50° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of an effector protein in a trans cleavage assay at 55° C. may be at least 50% of that at 37° C. In some instances, the trans cleavage activity of an effector protein in a trans cleavage assay at 55° C. may be at least 55% of that at 37° C. In some instances, the trans cleavage activity of an effector protein in a trans cleavage assay at 55° C. may be at least 60% of that at 37° C. In some instances, the trans cleavage activity of an effector protein in a trans cleavage assay at 55° C. may be at least 65% of that at 37° C. In some instances, the trans cleavage activity of an effector protein in a trans cleavage assay at 55° C. may be at least 70% of that at 37° C. In some instances, the trans cleavage activity of an effector protein in a trans cleavage assay at 55° C. may be at least 75% of that at 37° C. In some instances, the trans cleavage activity of an effector protein in a trans cleavage assay at 55° C. may be at least 80% of that at 37° C. In some instances, the trans cleavage activity of an effector protein in a trans cleavage assay at 55° C. may be at least 85% of that at 37° C. In some instances, the trans cleavage activity of an effector protein in a trans cleavage assay at 55° C. may be at least 90% of that at 37° C. In some instances, the trans cleavage activity of an effector protein in a trans cleavage assay at 55° C. may be at least 95% of that at 37° C. In some instances, the trans cleavage activity of an effector protein in a trans cleavage assay at 55° C. may be at least 100% of that at 37° C. In some instances, the trans cleavage activity of an effector protein in a trans cleavage assay at 55° C. may be at least 1-fold of that at 37° C. In some instances, the trans cleavage activity of an effector protein in a trans cleavage assay at 55° C. may be at least 2-fold of that at 37° C. In some instances, the trans cleavage activity of an effector protein in a trans cleavage assay at 55° C. may be at least 3-fold of that at 37° C. In some instances, the trans cleavage activity of an effector protein in a trans cleavage assay at 55° C. may be at least 4-fold of that at 37° C. In some instances, the trans cleavage activity of an effector protein in a trans cleavage assay at 55° C. may be at least 5-fold of that at 37° C. In some instances, the trans cleavage activity of an effector protein in a trans cleavage assay at 55° C. may be at least 6-fold of that at 37° C. In some instances, the trans cleavage activity of an effector protein in a trans cleavage assay at 55° C. may be at least 7-fold of that at 37° C. In some instances, the trans cleavage activity of an effector protein in a trans cleavage assay at 55° C. may be at least 8-fold of that at 37° C. In some instances, the trans cleavage activity of an effector protein in a trans cleavage assay at 55° C. may be at least 9-fold of that at 37° C. In some instances, the trans cleavage activity of an effector protein in a trans cleavage assay at 55° C. may be at least 10-fold of that at 37° C. In some instances, the trans cleavage activity of an effector protein in a trans cleavage assay at 55° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of an effector protein in a trans cleavage assay at 60° C. may be at least 50% of that at 37° C. In some instances, the trans cleavage activity of an effector protein in a trans cleavage assay at 60° C. may be at least 55% of that at 37° C. In some instances, the trans cleavage activity of an effector protein in a trans cleavage assay at 60° C. may be at least 60% of that at 37° C. In some instances, the trans cleavage activity of an effector protein in a trans cleavage assay at 60° C. may be at least 65% of that at 37° C. In some instances, the trans cleavage activity of an effector protein in a trans cleavage assay at 60° C. may be at least 70% of that at 37° C. In some instances, the trans cleavage activity of an effector protein in a trans cleavage assay at 60° C. may be at least 75% of that at 37° C. In some instances, the trans cleavage activity of an effector protein in a trans cleavage assay at 60° C. may be at least 80% of that at 37° C. In some instances, the trans cleavage activity of an effector protein in a trans cleavage assay at 60° C. may be at least 85% of that at 37° C. In some instances, the trans cleavage activity of an effector protein in a trans cleavage assay at 60° C. may be at least 90% of that at 37° C. In some instances, the trans cleavage activity of an effector protein in a trans cleavage assay at 60° C. may be at least 95% of that at 37° C. In some instances, the trans cleavage activity of an effector protein in a trans cleavage assay at 60° C. may be at least 100% of that at 37° C. In some instances, the trans cleavage activity of an effector protein in a trans cleavage assay at 60° C. may be at least 1-fold of that at 37° C. In some instances, the trans cleavage activity of an effector protein in a trans cleavage assay at 60° C. may be at least 2-fold of that at 37° C. In some instances, the trans cleavage activity of an effector protein in a trans cleavage assay at 60° C. may be at least 3-fold of that at 37° C. In some instances, the trans cleavage activity of an effector protein in a trans cleavage assay at 60° C. may be at least 4-fold of that at 37° C. In some instances, the trans cleavage activity of an effector protein in a trans cleavage assay at 60° C. may be at least 5-fold of that at 37° C. In some instances, the trans cleavage activity of an effector protein in a trans cleavage assay at 60° C. may be at least 6-fold of that at 37° C. In some instances, the trans cleavage activity of an effector protein in a trans cleavage assay at 60° C. may be at least 7-fold of that at 37° C. In some instances, the trans cleavage activity of an effector protein in a trans cleavage assay at 60° C. may be at least 8-fold of that at 37° C. In some instances, the trans cleavage activity of an effector protein in a trans cleavage assay at 60° C. may be at least 9-fold of that at 37° C. In some instances, the trans cleavage activity of an effector protein in a trans cleavage assay at 60° C. may be at least 10-fold of that at 37° C. In some instances, the trans cleavage activity of an effector protein in a trans cleavage assay at 60° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of an effector protein in a trans cleavage assay at 65° C. may be at least 50% of that at 37° C. In some instances, the trans cleavage activity of an effector protein in a trans cleavage assay at 65° C. may be at least 55% of that at 37° C. In some instances, the trans cleavage activity of an effector protein in a trans cleavage assay at 65° C. may be at least 60% of that at 37° C. In some instances, the trans cleavage activity of an effector protein in a trans cleavage assay at 65° C. may be at least 65% of that at 37° C. In some instances, the trans cleavage activity of an effector protein in a trans cleavage assay at 65° C. may be at least 70% of that at 37° C. In some instances, the trans cleavage activity of an effector protein in a trans cleavage assay at 65° C. may be at least 75% of that at 37° C. In some instances, the trans cleavage activity of an effector protein in a trans cleavage assay at 65° C. may be at least 80% of that at 37° C. In some instances, the trans cleavage activity of an effector protein in a trans cleavage assay at 65° C. may be at least 85% of that at 37° C. In some instances, the trans cleavage activity of an effector protein in a trans cleavage assay at 65° C. may be at least 90% of that at 37° C. In some instances, the trans cleavage activity of an effector protein in a trans cleavage assay at 65° C. may be at least 95% of that at 37° C. In some instances, the trans cleavage activity of an effector protein in a trans cleavage assay at 65° C. may be at least 100% of that at 37° C. In some instances, the trans cleavage activity of an effector protein in a trans cleavage assay at 65° C. may be at least 1-fold of that at 37° C. In some instances, the trans cleavage activity of an effector protein in a trans cleavage assay at 65° C. may be at least 2-fold of that at 37° C. In some instances, the trans cleavage activity of an effector protein in a trans cleavage assay at 65° C. may be at least 3-fold of that at 37° C. In some instances, the trans cleavage activity of an effector protein in a trans cleavage assay at 65° C. may be at least 4-fold of that at 37° C. In some instances, the trans cleavage activity of an effector protein in a trans cleavage assay at 65° C. may be at least 5-fold of that at 37° C. In some instances, the trans cleavage activity of an effector protein in a trans cleavage assay at 65° C. may be at least 6-fold of that at 37° C. In some instances, the trans cleavage activity of an effector protein in a trans cleavage assay at 65° C. may be at least 7-fold of that at 37° C. In some instances, the trans cleavage activity of an effector protein in a trans cleavage assay at 65° C. may be at least 8-fold of that at 37° C. In some instances, the trans cleavage activity of an effector protein in a trans cleavage assay at 65° C. may be at least 9-fold of that at 37° C. In some instances, the trans cleavage activity of an effector protein in a trans cleavage assay at 65° C. may be at least 10-fold of that at 37° C. In some instances, the trans cleavage activity of an effector protein in a trans cleavage assay at 65° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C. In some instances, the trans cleavage activity of an effector protein in a trans cleavage assay at 70° C., 75° C. 80° C., or more may be at least 50, at least 60%, at least 65%, at least 70%, at least 75%, at least 80%, at least 85%, at least 95%, at least 100%, at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity may be measured against a negative control in a trans cleavage assay. In some instances, the trans cleavage activity of an effector protein against a nucleic acid in a trans cleavage assay at 37° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 37° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 40° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 40° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 45° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 45° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 50° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 50° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 55° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 55° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 60° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 60° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 65° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 65° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 70° C., 75° C., 80° C., or more may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 70° C., 75° C., 80° C., or more may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid.

The reporters described herein can be RNA reporters. The RNA reporters can comprise at least one ribonucleic acid and a detectable moiety. In some embodiments, a programmable nuclease probe or a CRISPR probe comprising a Cas enzyme can recognize and detect ssDNA and, further, can specifically trans-cleave RNA reporters. The detection of the target nucleic acid in the sample can indicate the presence of the disease (or disease-causing agent) in the sample and can provide information for taking action to reduce the transmission of the disease to individuals in the disease-affected environment or near the disease-carrying individual.

Cleavage of a reporter (e.g., a protein-nucleic acid) can produce a signal. The signal can indicate a presence of the target nucleic acid in the sample, and an absence of the signal can indicate an absence of the target nucleic acid in the sample. In some cases, cleavage of the protein-nucleic acid can produce a calorimetric signal, a potentiometric signal, an amperometric signal, an optical signal, or a piezo-electric signal. Various devices and/or sensors can be used to detect these different types of signals, which indicate whether a target nucleic acid is present in the sample. The sensors usable to detect such signals can include, for example, optical sensors (e.g., imaging devices for detecting fluorescence or optical signals with various wavelengths and frequencies), electric potential sensors, surface plasmon resonance (SPR) sensors, interferometric sensors, or any other type of sensor suitable for detecting calorimetric signals, potentiometric signals, amperometric signals, optical signals, or piezo-electric signals.

In an aspect, the present disclosure provides a method for target detection. The method can comprise sample collection. The method can further comprise sample preparation. The method can further comprise detection of one or more target molecules in the collected and prepared sample.

In another aspect, the present disclosure provides a detection device for target detection. The detection device can be configured for multiplexed target detection. The detection device can be used to collect one or more samples, prepare or process the one or more samples for detection, and optionally divide the one or more samples into a plurality of droplets, aliquots, or subsamples for amplification of one or more target sequences or target nucleic acids. The target sequences may comprise, for example, a biological sequence. The biological sequence can comprise a nucleic acid sequence or an amino acid sequence. In some embodiments, the target sequences are associated with an organism of interest, a disease of interest, a disease state of interest, a phenotype of interest, a genotype of interest, or a gene of interest.

The detection device can be configured to amplify target nucleic acids contained within the plurality of droplets, aliquots, or subsamples. The detection device can be configured to amplify the target sequences or target nucleic acids contained within the plurality of droplets by individually processing each of the plurality of droplets (e.g., by using a thermocycling process or any other suitable amplification process as described in greater detail below). In some cases, the plurality of droplets can undergo separate thermocycling processes. In some cases, the thermocycling processes can occur simultaneously. In other cases, the thermocycling processes can occur at different times for each droplet.

The detection device can be further configured to remix the droplets, aliquots, or subsamples after the target nucleic acids in each of the droplets undergo amplification. The detection device can be configured to provide the remixed sample comprising the droplets, aliquots, or subsamples to a detection chamber of the device. The detection chamber can be configured to direct the remixed droplets, aliquots, or subsamples to a plurality of programmable nuclease probes. The detection chamber can be configured to direct the remixed droplets, aliquots, or subsamples along one or more fluid flow paths such that the remixed droplets, aliquots, or subsamples are positioned adjacent to and/or in contact with the one or more programmable nuclease probes. In some cases, the detection chamber can be configured to recirculate or recycle the remixed droplets, aliquots, or subsamples such that the remixed droplets, aliquots, or subsamples are repeatedly placed in contact with one or more programmable nuclease probes over a predetermined period of time.

The detection device can comprise one or more sensors. The one or more sensors of the detection device can be configured to detect one or more signals that are generated after one or more programmable nucleases of the one or more programmable nuclease probes become activated due to a binding of a guide nucleic acid of the programmable nuclease probes with a target nucleic acid present in the sample. As described elsewhere herein, the activated programmable nuclease can cleave the target nucleic acid, which can result in a trans cleavage activity. Trans cleavage activity can be a non-specific cleavage of nearby single-stranded nucleic acids by the activated programmable nuclease, such as trans cleavage of target nucleic acids with a detection moiety. Once the target nucleic acids are cleaved by the activated programmable nucleases, the detection moiety can be released or separated from the reporter, thereby generating one or more detectable signals. The one or more sensors of the detection device can be configured to register and/or process the one or more detectable signals to confirm a presence and/or an absence of a particular target (e.g., a target nucleic acid).

The one or more programmable nuclease probes of the detection device can be configured for multiplexed detection. In some cases, each programmable nuclease probe can be configured to detect a particular target. In other cases, each programmable nuclease probe can be configured to detect a plurality of targets. In some cases, a first programmable nuclease probe can be configured to detect a first target or a first set of targets, and a second programmable nuclease probe can be configured to detect a second target or a second set of targets. In other cases, a first programmable nuclease probe can be configured to detect a first set of targets, and a second programmable nuclease probe can be configured to detect a second set of targets. The programmable nuclease probes of the present disclosure can be used to detect a plurality of different target sequences or target nucleic acids. In any of the embodiments described herein, the sample provided to the detection device can comprise a plurality of target sequences or target nucleic acids. In any of the embodiments described herein, the sample provided to the detection device can comprise multiple classes of target sequences or target nucleic acids. Each class of target sequences or class of target nucleic acids can comprise a plurality of target sequences or target nucleic acids associated with a particular organism, disease state, phenotype, or genotype present within the sample. In some cases, each programmable nuclease probe can be used to detect a particular class of target sequences or a particular class of target nucleic acids associated with a particular organism, disease state, phenotype, or genotype present within the sample. In some cases, two or more programmable nuclease probes can be used to detect different classes of target sequences or different classes of target nucleic acids. In such cases, the two or more programmable nuclease probes can comprise different sets or classes of guide nucleic acids complexed to the programmable nucleases of the probes.

In any of the embodiments described herein, the detection device can comprise a single integrated system that is configured to perform sample collection, sample processing, droplet generation, droplet processing (e.g., amplification of target nucleic acids in droplets), droplet remixing, and/or circulation of the remixed droplets within a detection chamber so that at least a portion of the remixed droplets is placed in contact with one or more programmable nuclease probes coupled to the detection chamber. The detection devices of the present disclosure can be disposable devices configured to perform one or more rapid single reaction or multi-reaction tests to detect a presence and/or an absence of one or more target sequences or target nucleic acids.

The systems and methods of the present disclosure can be used to detect one or more target sequences or nucleic acids in one or more samples. The one or more samples can comprise one or more target sequences or nucleic acids for detection of an ailment, such as a disease, cancer, or genetic disorder, or genetic information, such as for phenotyping, genotyping, or determining ancestry and are compatible with the reagents and support mediums as described herein. Generally, a sample can be taken from any place where a nucleic acid can be found. Samples can be taken from an individual/human, a non-human animal, or a crop, or an environmental sample can be obtained to test for presence of a disease, virus, pathogen, cancer, genetic disorder, or any mutation or pathogen of interest. A biological sample can be blood, serum, plasma, lung fluid, exhaled breath condensate, saliva, spit, urine, stool, feces, mucus, lymph fluid, peritoneal, cerebrospinal fluid, amniotic fluid, breast milk, gastric secretions, bodily discharges, secretions from ulcers, pus, nasal secretions, sputum, pharyngeal exudates, urethral secretions/mucus, vaginal secretions/mucus, anal secretion/mucus, semen, tears, an exudate, an effusion, tissue fluid, interstitial fluid (e.g., tumor interstitial fluid), cyst fluid, tissue, or, in some instances, any combination thereof. A sample can be an aspirate of a bodily fluid from an animal (e.g., human, animals, livestock, pet, etc.) or plant. A tissue sample can be from any tissue that can be infected or affected by a pathogen (e.g., a wart, lung tissue, skin tissue, and the like). A tissue sample (e.g., from animals, plants, or humans) can be dissociated or liquified prior to application to detection system of the present disclosure. A sample can be from a plant (e.g., a crop, a hydroponically grown crop or plant, and/or house plant). Plant samples can include extracellular fluid, from tissue (e.g., root, leaves, stem, trunk etc.). A sample can be taken from the environment immediately surrounding a plant, such as hydroponic fluid/water, or soil. A sample from an environment can be from soil, air, or water. In some instances, the environmental sample is taken as a swab from a surface of interest or taken directly from the surface of interest. In some instances, the raw sample is applied to the detection system. In some instances, the sample is diluted with a buffer or a fluid or concentrated prior to application to the detection system. In some cases, the sample is contained in no more than about 200 nanoliters (nL). In some cases, the sample is contained in about 200 nL. In some cases, the sample is contained in a volume that is greater than about 200 nL and less than about 20 microliters (μL). In some cases, the sample is contained in no more than 20 μl. In some cases, the sample is contained in no more than 1, 5, 10, 15, 20, 25, 30, 35 40, 45, 50, 55, 60, 65, 70, 75, 80, 90, 100, 200, 300, 400, 500 μl, or any of value from 1 μl to 500 μl. In some cases, the sample is contained in from 1 μL to 500 μL, from 10 μL to 500 μL, from 50 μL to 500 μL, from 100 μL to 500 μL, from 200 μL to 500 μL, from 300 μL to 500 μL, from 400 μL to 500 μL, from 1 μL to 200 μL, from 10 μL to 200 μL, from 50 μL to 200 μL, from 100 μL to 200 μL, from 1 μL to 100 μL, from 10 μL to 100 μL, from 50 μL to 100 μL, from 1 μL to 50 μL, from 10 μL to 50 μL, from 1 μL to 20 μL, from 10 μL to 20 μL, or from 1 μL to 10 μL. Sometimes, the sample is contained in more than 500 μl.

In some instances, the sample is taken from a single-cell eukaryotic organism; a plant or a plant cell; an algal cell; a fungal cell; an animal or an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample may comprise nucleic acids from a cell lysate from a eukaryotic cell, a mammalian cell, a human cell, a prokaryotic cell, or a plant cell. In some cases, the sample may comprise nucleic acids expressed from a cell.

The sample used for disease testing can comprise at least one target sequence that can bind to a guide nucleic acid of the reagents described herein. In some cases, the target sequence is a portion of a nucleic acid. A nucleic acid can be from a genomic locus, a transcribed mRNA, or a reverse transcribed cDNA. A nucleic acid can be from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. A nucleic acid can be from 10 to 90, from 20 to 80, from 30 to 70, or from 40 to 60 nucleotides in length. A nucleic acid sequence can be from 10 to 95, from 20 to 95, from 30 to 95, from 40 to 95, from 50 to 95, from 60 to 95, from 10 to 75, from 20 to 75, from 30 to 75, from 40 to 75, from 50 to 75, from 5 to 50, from 15 to 50, from 25 to 50, from 35 to 50, or from 45 to 50 nucleotides in length. A nucleic acid can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The target nucleic acid can be reverse complementary to a guide nucleic acid. In some cases, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides of a guide nucleic acid can be reverse complementary to a target nucleic acid.

In some cases, the target sequence is a portion of a nucleic acid from a virus or a bacterium or other agents responsible for a disease in the sample. The target sequence, in some cases, is a portion of a nucleic acid from a sexually transmitted infection or a contagious disease, in the sample. The target sequence, in some cases, is a portion of a nucleic acid from an upper respiratory tract infection, a lower respiratory tract infection, or a contagious disease, in the sample. The target sequence, in some cases, is a portion of a nucleic acid from a hospital acquired infection or a contagious disease, in the sample. The target sequence, in some cases, is a portion of a nucleic acid from sepsis, in the sample. These diseases can include but are not limited to respiratory viruses (e.g., SARS-CoV-2 (i.e., a virus that causes COVID-19), SARS, MERS, influenza, Adenovirus, Coronavirus HKU1, Coronavirus NL63, Coronavirus 229E, Coronavirus OC43, Severe Acute Respiratory Syndrome Coronavirus 2 (SARS-CoV-2), Human Metapneumovirus (hMPV), Human Rhinovirus/Enterovirus, Influenza A, Influenza A/H1, Influenza A/H3, Influenza A/H1-2009, Influenza B, Influenza C, Parainfluenza Virus 1, Parainfluenza Virus 2, Parainfluenza Virus 3, Parainfluenza Virus 4, Respiratory Syncytial Virus) and respiratory bacteria (e.g. Bordetella parapertussis, Bordetella pertussis, Chlamydia pneumoniae, Mycoplasma pneumoniae). Other viruses include human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, Chlamydia pneumoniae, Chlamydia psittaci, and Candida albicans. Pathogenic viruses include but are not limited to: respiratory viruses (e.g., adenoviruses, parainfluenza viruses, severe acute respiratory syndrome (SARS), coronavirus, MERS), gastrointestinal viruses (e.g., noroviruses, rotaviruses, some adenoviruses, astroviruses), exanthematous viruses (e.g., the virus that causes measles, the virus that causes rubella, the virus that causes chickenpox/shingles, the virus that causes roseola, the virus that causes smallpox, the virus that causes fifth disease, chikungunya virus infection); hepatic viral diseases (e.g., hepatitis A, B, C, D, E); cutaneous viral diseases (e.g., warts (including genital, anal), herpes (including oral, genital, anal), molluscum contagiosum); hemmorhagic viral diseases (e.g. Ebola, Lassa fever, dengue fever, yellow fever, Marburg hemorrhagic fever, Crimean-Congo hemorrhagic fever); neurologic viruses (e.g., polio, viral meningitis, viral encephalitis, rabies), sexually transmitted viruses (e.g., HIV, HPV, and the like), immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Klebsiella pneumoniae, Acinetobacter baumannii, Bacillus anthracis, Bortadella pertussis, Burkholderia cepacia, Corynebacterium diphtheriae, Coxiella burnetii, Streptococcus agalactiae, methicillin-resistant Staphylococcus aureus, Legionella longbeachae, Legionella pneumophila, Leptospira interrogans, Moraxella catarrhalis, Streptococcus pyogenes, Escherichia coli, Neisseria gonorrhoeae, Neisseria meningitidis, Neisseria elongate, Neisseria gonorrhoeae, Parechovirus, Pneumococcus, Pneumocystis jirovecii, Cryptococcus neoformans, Histoplasma capsulatum, Haemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M genitalium, T. Vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Enterobacter cloacae, Kiebsiella aerogenes, Proteus vulgaris, Serratia macesens, Enterococcus faecalis, Enterococcus faecium, Streptococcus intermdius, Streptococcus pneumoniae, and Streptococcus pyogenes. Often the target nucleic acid may comprise a sequence from a virus or a bacterium or other agents responsible for a disease that can be found in the sample. In some cases, the target nucleic acid is a portion of a nucleic acid from a genomic locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Staphylococcus epidermidis, Legionella pneumophila, Streptococcus pyogenes, Streptococcus salivarius, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

The sample used for cancer testing or cancer risk testing can comprise at least one target sequence or target nucleic acid segment that can bind to a guide nucleic acid of the reagents described herein. The target nucleic acid segment, in some cases, is a portion of a nucleic acid from a gene with a mutation associated with cancer, from a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes for a cancer biomarker, such as a prostate cancer biomarker or non-small cell lung cancer. In some cases, the assay can be used to detect “hotspots” in target nucleic acids that can be predictive of cancer, such as lung cancer, cervical cancer, in some cases, the cancer can be a cancer that is caused by a virus. Some non-limiting examples of viruses that cause cancers in humans include Epstein-Barr virus (e.g., Burkitt's lymphoma, Hodgkin's Disease, and nasopharyngeal carcinoma); papillomavirus (e.g., cervical carcinoma, anal carcinoma, oropharyngeal carcinoma, penile carcinoma); hepatitis B and C viruses (e.g., hepatocellular carcinoma); human adult T-cell leukemia virus type 1 (HTLV-1) (e.g., T-cell leukemia); and Merkel cell polyomavirus (e.g., Merkel cell carcinoma). One skilled in the art will recognize that viruses can cause or contribute to other types of cancers. In some cases, the target nucleic acid is a portion of a nucleic acid that is associated with a blood fever. In some cases, the target nucleic acid segment is a portion of a nucleic acid from a genomic locus, a transcribed mRNA, or a reverse transcribed cDNA from a locus of at least one of: ALK, APC, ATM, AXIN2, BAP1, BARDi, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, KIT, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RBT, RECQL4, RET, RUNX1, SDHA, SDHAF2, SDHB, SDHC, SDHD, SMAD4, SMARCA4, SMARCB1, SMARCE1, STKTT, SUFU, TERC, TERT, TMEM127, TP53, TSC1, TSC2, VHL, WRN, and WT1.

The sample used for genetic disorder testing can comprise at least one target sequence or target nucleic acid segment that can bind to a guide nucleic acid of the reagents described herein. In some embodiments, the genetic disorder is hemophilia, sickle cell anemia, β-thalassemia, Duchene muscular dystrophy, severe combined immunodeficiency, or cystic fibrosis. The target nucleic acid segment, in some cases, is a portion of a nucleic acid from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid segment is a portion of a nucleic acid from a genomic locus, a transcribed mRNA, or a reverse transcribed cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARGI, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRElC, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHEl, EVC, EVC2, EYS, F9, FAH, FAM161A, FANCA, FANCC, FANCG, FH, FKRP, FKTN, G6PC, GAA, GALC, GALKI, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEXI, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

The sample used for phenotyping testing can comprise at least one target nucleic acid segment that can bind to a guide nucleic acid of the reagents described herein. The target nucleic acid segment, in some cases, is a portion of a nucleic acid from a gene associated with a phenotypic trait.

The sample used for genotyping testing can comprise at least one target nucleic acid segment that can bind to a guide nucleic acid of the reagents described herein. The target nucleic acid segment, in some cases, is a portion of a nucleic acid from a gene associated with a genotype.

The sample used for ancestral testing can comprise at least one target nucleic acid segment that can bind to a guide nucleic acid of the reagents described herein. The target nucleic acid segment, in some cases, is a portion of a nucleic acid from a gene associated with a geographic region of origin or ethnic group.

The sample can be used for identifying a disease status. For example, a sample is any sample described herein, and is obtained from a subject for use in identifying a disease status of a subject. The disease can be a cancer or genetic disorder. Sometimes, a method may comprise obtaining a serum sample from a subject; and identifying a disease status of the subject. Often, the disease status is prostate disease status. In any of the embodiments described herein, the device can be configured for asymptomatic, pre-symptomatic, and/or symptomatic diagnostic applications, irrespective of immunity. In any of the embodiments described herein, the device can be configured to perform one or more serological assays on a sample (e.g., a sample comprising blood).

In some embodiments, the sample can be used to identify a mutation in a target nucleic acid of a plant or of a bacteria, virus, or microbe associated with a plant or soil. The devices and methods of the present disclosure can be used to identify a mutation of a target nucleic acid that affects the expression of a gene. A mutation that affects the expression of gene can be a mutation of a target nucleic acid within the gene, a mutation of a target nucleic acid comprising RNA associated with the expression of a gene, or a target nucleic acid comprising a mutation of a nucleic acid associated with regulation of expression of a gene, such as an RNA or a promoter, enhancer, or repressor of the gene. Often, the mutation is a single nucleotide mutation

In some instances, the target nucleic acid is a single stranded nucleic acid. Alternatively, or in combination, the target nucleic acid is a double stranded nucleic acid and is prepared into single stranded nucleic acids before or upon contacting the reagents. The target nucleic acid can be a RNA, DNA, synthetic nucleic acids, or nucleic acids found in biological or environmental samples. The target nucleic acids include but are not limited to mRNA, rRNA, tRNA, non-coding RNA, long non-coding RNA, and microRNA (miRNA). In some cases, the target nucleic acid is mRNA. In some cases, the target nucleic acid is from a virus, a parasite, or a bacterium described herein. In some cases, the target nucleic acid is transcribed from a gene as described herein.

A number of target nucleic acids are consistent with the systems and methods disclosed herein. Some methods described herein can detect a target nucleic acid present in the sample in various concentrations or amounts as a target nucleic acid population. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the sample has from 1 to 10,000, from 100 to 8000, from 400 to 6000, from 500 to 5000, from 1000 to 4000, or from 2000 to 3000 target nucleic acids. In some cases, the sample has from 100 to 9500, from 100 to 9000, from 100 to 8500, from 100 to 8000, from 100 to 7500, from 100 to 7000, from 100 to 6500, from 100 to 6000, from 100 to 5500, from 100 to 5000, from 250 to 9500, from 250 to 9000, from 250 to 8500, from 250 to 8000, from 250 to 7500, from 250 to 7000, from 250 to 6500, from 250 to 6000, from 250 to 5500, from 250 to 5000, from 2500 to 9500, from 2500 to 9000, from 2500 to 8500, from 2500 to 8000, from 2500 to 7500, from 2500 to 7000, from 2500 to 6500, from 2500 to 6000, from 2500 to 5500, or from 2500 to 5000 target nucleic acids. In some cases, the method detects target nucleic acid present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

A number of target nucleic acid populations are consistent with the systems and methods disclosed herein. Some methods described herein can be implemented to detect two or more target nucleic acid populations present in the sample in various concentrations or amounts. In some cases, the sample has at least 2 target nucleic acid populations. In some cases, the sample has at least 3, 4, 5, 6, 7, 8, 9, 10, 20, 30, 40, or 50 target nucleic acid populations. In some cases, the sample has from 3 to 50, from 5 to 40, or from 10 to 25 target nucleic acid populations. In some cases, the sample has from 2 to 50, from 5 to 50, from 10 to 50, from 2 to 25, from 3 to 25, from 4 to 25, from 5 to 25, from 10 to 25, from 2 to 20, from 3 to 20, from 4 to 20, from 5 to 20, from 10 to 20, from 2 to 10, from 3 to 10, from 4 to 10, from 5 to 10, from 6 to 10, from 7 to 10, from 8 to 10, or from 9 to 10 target nucleic acid populations. In some cases, the methods of the present disclosure can be implemented to detect target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations can be present at different concentrations or amounts in the sample.

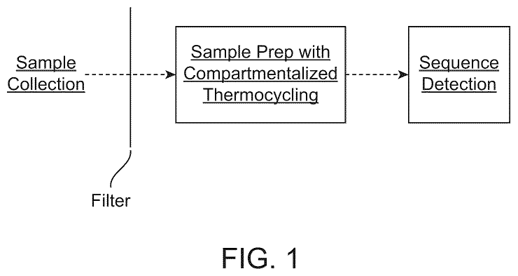

FIG. 1 illustrates an exemplary method for programmable nuclease-based detection. The method can comprise collecting a sample. The sample can comprise any type of sample as described herein. The method can comprise preparing the sample. Sample preparation can comprise one or more sample preparation steps. The one or more sample preparation steps can be performed in any suitable order. The one or more sample preparation steps can comprise physical filtration of non-target materials using a macro filter. The one or more sample preparation steps can comprise nucleic acid purification. The one or more sample preparation steps can comprise lysis. The one or more sample preparation steps can comprise heat inactivation. The one or more sample preparation steps can comprise adding one or more enzymes or reagents to prepare the sample for target detection.

The method can comprise generating one or more droplets, aliquots, or subsamples from the sample. The one or more droplets, aliquots, or subsamples can correspond to a volumetric portion of the sample. The sample can be divided into 1, 2, 3, 4, 5, 6, 7, 8, 9, 10, 20, 30, 40, 50, 60, 70, 80, 90, 100, 200, 300, 400, 500, 600, 700, 800, 900, 1000, or more droplets, aliquots, or subsamples. In some embodiments, the sample is not divided into subsamples.

The method can comprise amplifying one or more targets within each droplet, aliquot, or subsample. Amplification of the one or more targets within each droplet can be performed in parallel and/or simultaneously for each droplet. Dividing the sample into a plurality of droplets can enhance a speed and/or an efficiency of the amplification process (e.g., a thermocycling process) since the droplets comprise a smaller volume of material than the bulk sample introduced. Amplifying the one or more targets within each individual droplet can also permit effective amplification of various target nucleic acids that cannot be amplified as efficiently in a bulk sample containing the various target nucleic acids if the bulk sample were to undergo a singular amplification process. In some embodiments, amplification is performed on the bulk sample without first dividing the sample into subsamples.

The method can further comprise using a CRISPR-based or programmable nuclease-based detection module to detect one or more targets (e.g., target sequences or target nucleic acids) in the sample. In some cases, the sample can be divided into a plurality of droplets, aliquots, or subsamples to facilitate sample preparation and to enhance the detection capabilities of the devices of the present disclosure. In some cases, the sample is not divided into subsamples.

In some embodiments, the sample can be provided manually to the detection device of the present disclosure. For example, a swab sample can be dipped into a solution and the sample/solution can be pipetted into the device. In other embodiments, the sample can be provided via an automated syringe. The automated syringe can be configured to control a flow rate at which the sample is provided to the detection device. The automated syringe can be configured to control a volume of the sample that is provided to the detection device over a predetermined period.

In some embodiments, the sample can be provided directly to the detection device of the present disclosure. For example, a swab sample can be inserted into a sample chamber on the detection device.

The sample can be prepared before one or more targets are detected within the sample. The sample preparation steps described herein can process a crude sample to generate a pure or purer sample. Sample preparation can one or more physical or chemical processes, including, for example, nucleic acid purification, lysis, binding, washing, and/or eluting. In certain instances, sample preparation can comprise the following steps, in any order, including sample collection, nucleic acid purification, heat inactivation, and/or base/acid lysis.

In some embodiments, nucleic acid purification can be performed on the sample. Purification can comprise disrupting a biological matrix of a cell to release nucleic acids, denaturing structural proteins associated with the nucleic acids (nucleoproteins), inactivating nucleases that can degrade the isolated product (RNase and/or DNase), and/or removing contaminants (e.g., proteins, carbohydrates, lipids, biological or environmental elements, unwanted nucleic acids, and/or other cellular debris).

In some embodiments, lysis of a collected sample can be performed. Lysis can be performed using a protease (e.g., a Proteinase K or PK enzyme). In some cases, a solution of reagents can be used to lyse the cells in the sample and release the nucleic acids so that they are accessible to the programmable nuclease. Active ingredients of the solution can be chaotropic agents, detergents, salts, and can be of high osmolality, ionic strength, and pH. Chaotropic agents or chaotropes are substances that disrupt the three-dimensional structure in macromolecules such as proteins, DNA, or RNA. One example protocol may comprise a 4 M guanidinium isothiocyanate, 25 mM sodium citrate.2H20, 0.5% (w/v) sodium lauryl sarcosinate, and 0.1 M (3-mercaptoethanol), but numerous commercial buffers for different cellular targets can also be used. Alkaline buffers can also be used for cells with hard shells, particularly for environmental samples. Detergents such as sodium dodecyl sulphate (SDS) and cetyl trimethylammonium bromide (CTAB) can also be implemented to chemical lysis buffers. Cell lysis can also be performed by physical, mechanical, thermal or enzymatic means, in addition to chemically-induced cell lysis mentioned previously. In some cases, depending on the type of sample, nanoscale barbs, nanowires, acoustic generators, integrated lasers, integrated heaters, and/or microcapillary probes can be used to perform lysis.

In certain instances, heat inactivation can be performed on the sample. In some embodiments, a processed/lysed sample can undergo heat inactivation to inactivate, in the lysed sample, the proteins used during lysing (e.g., a PK enzyme or a lysing reagent). In some cases, a heating element integrated into the detection device can be used for heat-inactivation. The heating element can be powered by a battery or another source of thermal or electric energy that is integrated with the detection device.

In some cases, a target nucleic acid within the sample can undergo amplification before binding to a guide nucleic acid, for example a crRNA of a CRISPR enzyme. The target nucleic acid within a purified sample can be amplified. In some instances, amplification can be accomplished using loop mediated amplification (LAMP), isothermal recombinase polymerase amplification (RPA), and/or polymerase chain reaction (PCR). In some instances, digital droplet amplification can used. Such nucleic acid amplification of the sample can improve at least one of a sensitivity, specificity, or accuracy of the detection of the target RNA. The reagents for nucleic acid amplification can comprise a recombinase, an oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence-based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification is performed for from 1 to 60, from 5 to 55, from 10 to 50, from 15 to 45, from 20 to 40, or from 25 to 35 minutes. Sometimes, the nucleic acid amplification is performed for from 5 to 60, from 10 to 60, from 15 to 60, from 30 to 60, from 45 to 60, from 1 to 45, from 5 to 45, from 10 to 45, from 30 to 45, from 1 to 30, from 5 to 30, from 10 to 30, from 15 to 30, from 1 to 15, from 5 to 15, or from 10 to 15 minutes.

In some embodiments, amplification can comprise thermocycling of the sample. Thermocycling can be carried out for one or more droplets of the sample in parallel and/or independently in separate locations. This can be accomplished by methods such as (1) by holding droplets stationary in locations where a heating element is in close proximity to the droplet on one of the droplet sides and a heat sink element is in close proximity to the other side of the droplet, or (2) flowing the droplet through zones in a fluid channel where heat flows across it from a heating source to a heat sink. In some cases, one or more resistive heating elements can be used to perform thermocycling. Sometimes, the nucleic acid amplification reaction is performed at a temperature of around 20-45° C. The nucleic acid amplification reaction can be performed at a temperature no greater than 20° C., 25° C., 30° C., 35° C., 37° C., 40° C., 45° C., 50° C., 55° C., 60° C., or 65° C. The nucleic acid amplification reaction can be performed at a temperature of at least 20° C., 25° C., 30° C., 35° C., 37° C., 40° C., 45° C., 50° C., 55° C., 60° C., or 65° C. In some cases, the nucleic acid amplification reaction is performed at a temperature of from 20° C. to 45° C., from 25° C. to 40° C., from 30° C. to 40° C., or from 35° C. to 40° C. In some cases, the nucleic acid amplification reaction is performed at a temperature of from 45° C. to 65° C., from 50° C. to 65° C., from 55° C. to 65° C., or from 60° C. to 65° C. In some cases, the nucleic acid amplification reaction can be performed at a temperature that ranges from about 20° C. to 45° C., from 25° C. to 45° C., from 30° C. to 45° C., from 35° C. to 45° C., from 40° C. to 45° C., from 20° C. to 37° C., from 25° C. to 37° C., from 30° C. to 37° C., from 35° C. to 37° C., from 20° C. to 30° C., from 25° C. to 30° C., from 20° C. to 25° C., or from about 22° C. to 25° C. In some cases, the nucleic acid amplification reaction can be performed at a temperature that ranges from about 40° C. to 65° C., from 45° C. to 65° C., from 50° C. to 65° C., from 55° C. to 65° C., from 60° C. to 65° C., from 40° C. to 60° C., from 45° C. to 60° C., from 50° C. to 60° C., from 55° C. to 60° C., from 40° C. to 55° C., from 45° C. to 55° C., from 50° C. to 55° C., from 40° C. to 50° C., or from about 45° C. to 50° C.

Additionally, target nucleic acid can optionally be amplified before binding to the guide nucleic acid (e.g., crRNA) of the programmable nuclease (e.g., CRISPR enzyme). This amplification can be PCR amplification or isothermal amplification. This nucleic acid amplification of the sample can improve at least one of sensitivity, specificity, or accuracy of the detection the target RNA. The reagents for nucleic acid amplification can comprise a recombinase, a oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. Sometimes, the nucleic acid amplification reaction is performed at a temperature of around 45-65° C. The nucleic acid amplification reaction can be performed at a temperature no greater than 20° C., 25° C., 30° C., 35° C., 37° C., 40° C., 45° C., 50° C., 55° C., 60° C., or 65° C. The nucleic acid amplification reaction can be performed at a temperature of at least 20° C., 25° C., 30° C., 35° C., 37° C., 40° C., 45° C., 50° C., 55° C., 60° C., or 65° C.

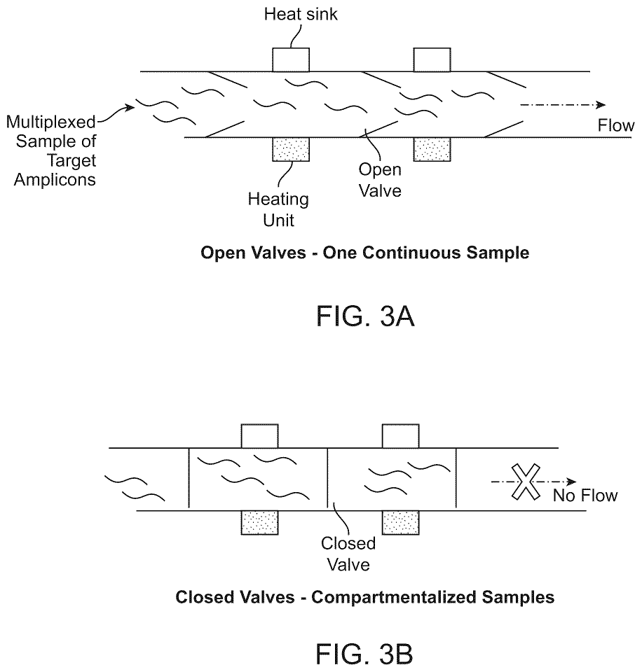

FIG. 3A illustrates an example of a channel through which a continuous flow of a sample can travel to undergo a thermocycling procedure. The device can comprise one or more movable mechanisms that are integrated into the device. The one or more movable mechanisms can be powered using a battery that is integrated with the device. The one or more movable mechanisms can be configured to stop and start the continuous flow of the sample through the channel at one or more predetermined time intervals. The one or more movable mechanisms can be configured to chop or divide the continuous flow of the sample into a plurality of smaller volumes, which can be referred to herein as “droplets.” The one or more movable mechanisms can have an open configuration and a closed configuration. The open configuration can permit a continuous flow of the sample through one or more sections of the channel, as seen in FIG. 3A. The closed configuration can restrict or completely inhibit a flow of the sample through one or more sections of the channel, as seen in FIG. 3B. The closed configuration can permit a physical and/or thermal separation of one or more volumes or portions of the sample flowing through the channel. When the movable mechanisms are in the closed position, the movable mechanisms can provide a physical barrier between different volumes or portions of the sample flowing through the channel. The droplet volume can range from 0.01 to 1 microliter, 1 to 5 microliters and 5 to 50 microliters. The different volumes or partitions can correspond to the droplets described elsewhere herein. The movable mechanisms can switch from the open position to the closed position, or from the closed position to the open position, depending on an operation of a syringe that is providing the sample or another flow regulator that is controlling a flow of the sample through the channel. The movable mechanisms can be powered using a battery that is integrated with the device.

In some cases, the movable mechanism can comprise a plurality of valves. The plurality of valves can comprise, for example, a check valve. In some cases, the movable mechanism can comprise a plunger or a bristle. The plunger or bristle can have an open configuration and a closed configuration. As described above, the open configuration can permit a continuous flow of the sample through one or more sections of the channel, and the closed configuration can restrict or completely inhibit a flow of the sample through one or more sections of the channel. The closed configuration can permit a physical and/or thermal separation of one or more volumes or portions of the sample flowing through the channel. When in the open configuration, the plunger or the bristle can be positioned flush to a bottom of the channel so that the sample can flow through the channel. When in the closed configuration, the plunger or the bristle can be configured to extend from a bottom portion of the channel to a top portion of the channel so that the sample flow is restricted and the sample is divided into a plurality of different droplets, or partitions, that are physically and/or thermally isolated from each other. In some cases, the movable mechanism can comprise any physical object (e.g., a plate) that can be configured to restrict flow through the channel at one or more sections of the channel. In some cases, the movable mechanism can comprise a hinge or spring mechanism to move the movable mechanism between an open configuration and a closed configuration.

The movable mechanisms can be used to generate one or more droplets, aliquots, or subsamples. Each of the one or more droplets, aliquots, or subsamples generated using the movable mechanism can be physically and/or thermally isolated within a plurality of different portions within the channel. The droplets, aliquots, or subsamples can be physically constrained within different portions within the channel. The droplets, aliquots, or subsamples can be constrained between a first movable mechanism that is in a closed position and a second movable mechanism that is in a closed position. The first movable mechanism can be located at a first distance from an inlet of the channel, and the second movable mechanism can be located at a second distance from the inlet of the channel. The channel can be part of a closed system through which the sample can flow. In some cases, when the sample flow through an inlet of the channel is stopped (e.g., a plunger of a syringe containing the sample is pulled back), the one or more movable mechanisms can be placed in a closed configuration, thereby separating the sample already within the channel into a plurality of thermally and physically isolated droplets. Generating droplets, aliquots, or subsamples can simplify the solution, reduce a complexity of the solution, and enhance an accessibility of targets for amplification.

The one or more droplets, aliquots, or subsamples generated using the movable mechanism can undergo an amplification step or a thermocycling step as described elsewhere herein. In some cases, the one or more droplets generated using the movable mechanisms can come into contact with separate heating units and heat sinks while constrained between two movable mechanisms. Different sections of the channel can comprise a plurality of heating units and heat sinks configured to perform thermocycling for different droplets. Individual thermocycling of the droplets, aliquots, or subsamples can permit more efficient thermocycling of smaller volumes of fluid, and can require less energy usage (e.g., from a battery). One or more valves can control a flow or a movement of the sample through the channel. The one or more valves can comprise a check valve that is configured to restrict a movement of the sample or the one or more droplets such that the sample or the one or more droplets do not travel backwards towards an inlet portion of the channel. The one or more valves can control when the sample or the droplets come into thermal contact with the heating unit and/or the heat sink. The timing of such thermal contact can correspond to a timing of one or more thermocycling steps. In some cases, a first droplet of the sample can be in thermal contact with a first heating unit and a first heat sink, a second droplet of the sample can be in thermal contact with a second heating unit and a second heat sink, and so on.

As described above, the devices of the present disclosure can be configured to perform droplet digitization or droplet generation. Droplet digitization or generation can comprise splitting a volume of the sample into multiple droplets, aliquots, or subsamples. The sample can have a volume that ranges from about 10 microliters to about 500 microliters. The plurality of droplets, aliquots, or subsamples can have a volume that ranges from about 0.01 microliters to about 100 microliters. The plurality of droplets, aliquots, or subsamples can have a same or substantially similar volume. In some cases, the plurality of droplets, aliquots, or subsamples can have different volumes. In some cases, the droplets, aliquots, or subsamples can be generated using a physical filter or the one or more movable mechanisms described above. In some cases, each droplet of the sample can undergo one or more sample preparation steps (e.g., nucleic acid purification, lysis, heat inactivation, amplification, etc.) independently and/or in parallel while the droplets are physically constrained or thermally isolated between two movable mechanisms.

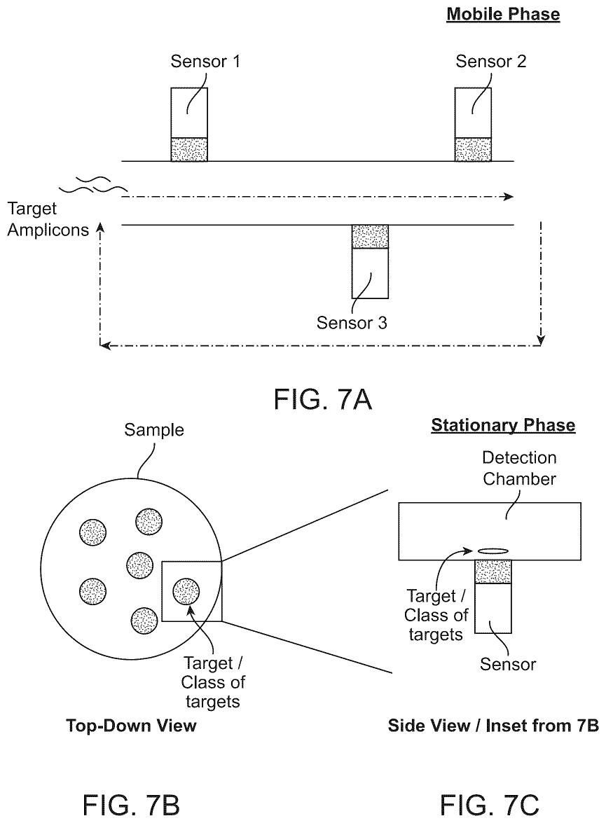

After amplification, the sample can be remixed. The sample can be circulated through the detection chamber using a bulk circulation mechanism that is configured to stir the remixed sample around such that the remixed sample comes into contact with one or more programmable nuclease probes, as shown in FIG. 7A. In some cases, the sample can be provided on a portion of a surface of the detection chamber that is proximal to one or more programmable nuclease probes, as shown in FIG. 7C. In some cases, the detection chamber can be configured to direct the sample along one or more fluid flow paths that position the remixed sample adjacent and/or proximal to one or more programmable nuclease probes. The one or more fluid flow paths can be used to target delivery of at least a portion of the remixed droplets to one or more detection regions associated with the one or more programmable nuclease probes. The remixed droplets can be circulated through the detection chamber along one or more desired fluid flow paths with aid of a piezoelectric device.

In some embodiments, electrowetting can be used by the device for sample transport. In some cases, the device can be configured for electrowetting-on-dielectric (EWOD) applications. The devices of the present disclosure can comprise an array of independently addressable electrodes integrated into the device.

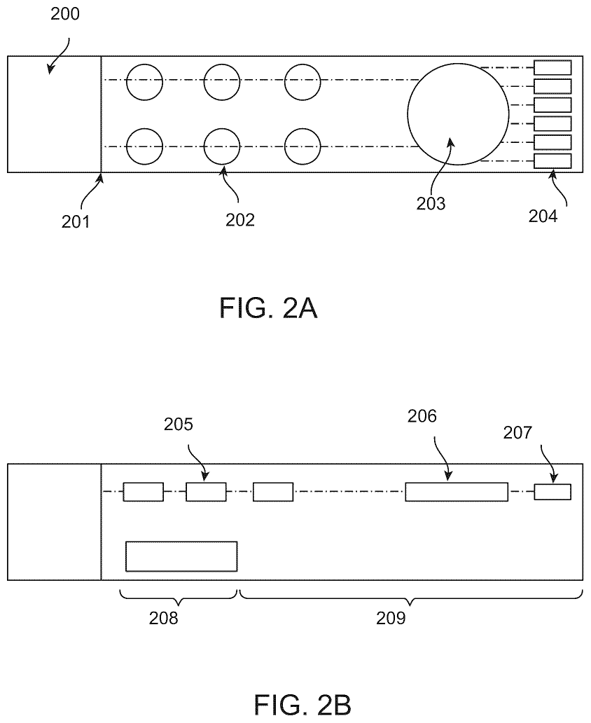

Described herein are various embodiments of a device for programmable nuclease-based (e.g., CRISPR-based) assays. FIG. 2A illustrates a top-down view of an exemplary device for CRISPR-based detection. The device can comprise a sample interface (200) that is configured to receive a sample. The sample can undergo one or more processing steps as described elsewhere herein. Any of the devices described herein may comprise a physical filter (201) to filter one or more particles from the sample that do not comprise the one or more targets (e.g., a gene of interest). In some embodiments, the device may comprise thermocycling components (202). In some cases, the sample can be divided into a plurality of digitized droplets. The plurality of digitized droplets can be provided in a plurality of different chambers of the device. The plurality of digitized droplets can undergo separate processing steps (e.g., thermocycling). In some embodiments, the independent digital reactions can be conducted in parallel with no cross-talk therebetween. In other embodiments, the independent digital reactions can be conducted in parallel with a minimal level or amount of cross-talk. The plurality of droplets can be mixed together after the separate processing steps upon completion of the processing steps. A plurality of programmable nuclease probes comprising one or more programmable nucleases (e.g., Cas enzymes) can be operatively coupled to the detection chamber (203) to detect one or more targets in the sample or the plurality of digitized droplets that are mixed together in the detection chamber. One or more detectors (204) may be configured to detect a signal from the detection chamber as described herein. A side profile of an embodiment of an exemplary programmable nuclease-based device is shown in FIG. 2B. In some embodiments, the device may comprise one or more thermocycling compartments (205) as seen in sideview. In some embodiments, the device may comprise a detection chamber (206). In some embodiments, the device may comprise one or more detectors (207). In some embodiments, the device may comprise a battery (208). In some embodiments, the device may comprise telemedicine components.

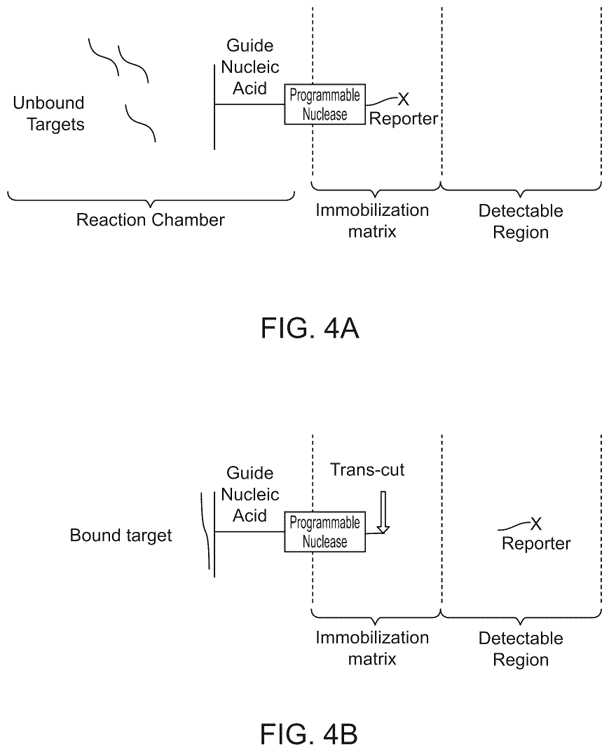

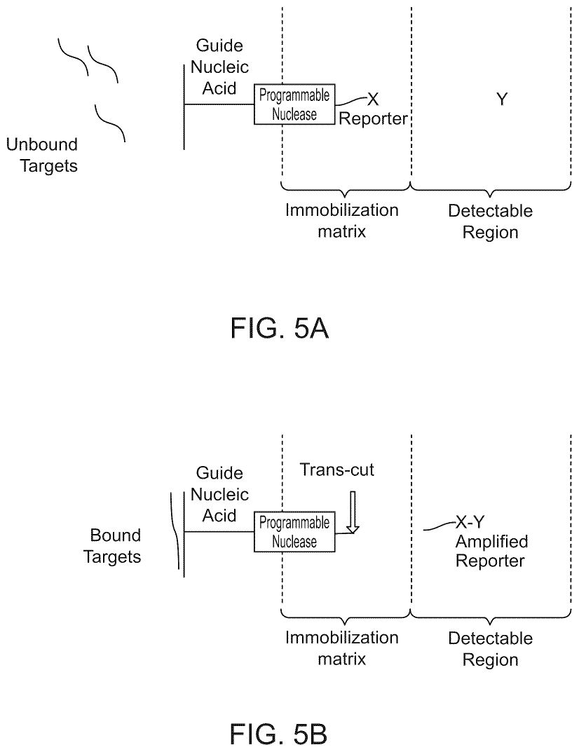

FIGS. 4A, 4B, 5A, and 5B illustrate an exemplary programmable nuclease probe that can be used in a compatible manner with the devices of the present disclosure. The programmable nuclease probe can comprise a guide nucleic acid complexed with a programmable nuclease. The programmable nuclease can comprise any type of programmable nuclease as described herein. In some cases, the programmable nuclease probe may comprise a guide nucleic acid complexed with a CRISPR enzyme. For example, FIG. 4A shows unbound target amplicons in the circulation chamber prior to binding to a guide RNA, which in turn is contacted to a programmable nuclease (e.g., a CRISPR enzyme). The guide RNA-CRISPR enzyme complex also includes a reporter. The programmable nuclease probe (e.g., a CRISPR probe) is immobilized to an immobilization matrix, where the interior side of the immobilization matrix is exposed to the inside wall of the circulation chamber. The guide nucleic acid or guide RNA is exposed to the target amplicons inside the circulation chamber. The reporter is in proximity to the “exterior” side of the immobilization matrix, where the exterior side of the immobilization matrix be in proximity to a detection region. FIG. 4B illustrates a programmable nuclease probe (e.g., a CRISPR probe) after binding with a complementary target amplicon. The binding event triggers a trans-cut that releases the reporter into a detectable region or changes the reporter. Detection mechanisms can involve interferometry, surface plasmon resonance, electrochemical detection such as potentiometry, or other detection mechanisms.

In certain instances, as seen in FIGS. 5A and 5B, the reporter of the programmable nuclease probe can initiate a signal amplification reaction with another molecular species after the complementary binding induced trans-cutting. Such species can be a reactive solid or gel matrix, or other reactive entity to generate an amplified signal during detection. The signal amplification reaction can be physical or chemical in nature. In certain instances, as seen in FIGS. 5A and 5B, after a complementary binding induced trans-cut, the released reporter, X, can initiate an interaction and/or a reaction with another entity, Y to produce an amplified or modified signal. Such entities can comprise a molecular species, a solid, a gel, or other entities. The signal amplification interaction can be a physical or chemical reaction. In some embodiments, the interaction involves free-radical, anionic, cationic or coordination polymerization reactions. In other embodiments the cut reporter can trigger aggregation, or agglutination, of molecules, cells, or nanoparticles. In some instances, the cut reporter can interact with a semiconductor material. In some embodiments, the chemical or physical change caused by the interaction is detected by optical detection means such as interferometry, surface plasmon resonance, reflectivity or other. In other embodiments, the chemical or physical change caused by the interaction is detected by potentiometric, amperometric, field effect transistor, or other electronic means

The programmable nuclease probe can comprise a programmable nuclease and/or a guide nucleic acid. The guide nucleic acid can bind to a target nucleic acid, as described in greater detail below. In some case, to minimize off-target binding (which can slow down detection or inhibit accurate detection), the device can be configured to generate an electro-potential gradient or to provide heat energy to one or more regions proximal to the programmable nuclease probe, to enhance targeting.

In some embodiments, one or more guide nucleic acids can be used to carry out highly efficient, rapid, and accurate reactions for detecting whether a target nucleic acid is present in a sample. The guide nucleic acid binds to the single stranded target nucleic acid comprising a portion of a nucleic acid from a virus or a bacterium or other agents responsible for a disease as described herein. The guide nucleic acid can bind to the single stranded target nucleic acid comprising a portion of a nucleic acid from a bacterium or other agents responsible for a disease as described herein and further comprising a mutation, such as a single nucleotide polymorphism (SNP), which can confer resistance to a treatment, such as antibiotic treatment. The guide nucleic acid binds to the single stranded target nucleic acid comprising a portion of a nucleic acid from a cancer gene or gene associated with a genetic disorder as described herein. The guide nucleic acid is complementary to the target nucleic acid. Often the guide nucleic acid binds specifically to the target nucleic acid. The target nucleic acid can be a RNA, DNA, or synthetic nucleic acids. A guide nucleic acid can comprise a sequence that is reverse complementary to the sequence of a target nucleic acid. A guide nucleic acid can be a crRNA. Sometimes, a guide nucleic acid may comprise a crRNA and tracrRNA. The guide nucleic acid can bind specifically to the target nucleic acid. In some cases, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. The target nucleic acid can be designed and made to provide desired functions. In some cases, the targeting region of a guide nucleic acid is 20 nucleotides in length. The targeting region of the guide nucleic acid can have a length of at least 10, 11, 12, 13, 14, 15, 16, 17, 18, 19, 20, 21, 22, 23, 24, 25, 26, 27, 28, 29, or 30 nucleotides in length. In some instances, the targeting region of the guide nucleic acid is 10, 11, 12, 13, 14, 15, 16, 17, 18, 19, 20, 21, 22, 23, 24, 25, 26, 27, 28, 29, or 30 nucleotides in length. In some cases, the targeting region of a guide nucleic acid has a length from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some cases, the targeting region of a guide nucleic acid has a length of from about 10 nt to about 60 nt, from about 20 nt to about 50 nt, or from about 30 nt to about 40 nt. In some cases, the targeting region of a guide nucleic acid has a length of from 15 nt to 55 nt, from 25 nt to 55 nt, from 35 nt to 55 nt, from 45 nt to 55 nt, from 15 nt to 45 nt, from 25 nt to 45 nt, from 35 nt to 45 nt, from 15 nt to 35 nt, from 25 nt to 35 nt, or from 15 nt to 25 nt. It is understood that the sequence of a polynucleotide need not be 100% complementary to that of its target nucleic acid to be specifically hybridizable or bind specifically. The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20 that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid.

The guide nucleic acid can be selected from a group of guide nucleic acids that have been tiled against the nucleic acid of a strain of an infection or genomic locus of interest. The guide nucleic acid can be selected from a group of guide nucleic acids that have been tiled against the nucleic acid of a strain of HPV 16 or HPV 18. Often, guide nucleic acids that are tiled against the nucleic acid of a strain of an infection or genomic locus of interest can be pooled for use in a method described herein. Often, these guide nucleic acids are pooled for detecting a target nucleic acid in a single assay. The pooling of guide nucleic acids that are tiled against a single target nucleic acid can enhance the detection of the target nucleic using the methods described herein. The pooling of guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling may comprise gaps between the tiled guide nucleic acids along the target nucleic acid. In some instances, the tiling of the guide nucleic acids is non-sequential. Often, a method for detecting a target nucleic acid may comprise contacting a target nucleic acid to a pool of guide nucleic acids and a programmable nuclease, wherein a guide nucleic acid of the pool of guide nucleic acids has a sequence selected from a group of tiled guide nucleic acid that is reverse complementary to a sequence of a target nucleic acid; and assaying for a signal produce by cleavage of at least some detector nucleic acids of a population of detector nucleic acids. Pooling of guide nucleic acids can ensure broad spectrum identification, or broad coverage, of a target species within a single reaction. This can be particularly helpful in diseases or indications, like sepsis, that can be caused by multiple organisms.

In some embodiments, programmable nucleases can be used to carry out highly efficient, rapid, and accurate reactions for detecting whether a target nucleic acid is present in a sample. A programmable nuclease can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby single-stranded nucleic acids by the activated programmable nuclease, such as trans cleavage of detector nucleic acids with a detection moiety. Once the detector nucleic acid is cleaved by the activated programmable nuclease, the detection moiety can be released from the detector nucleic acid and can generate a signal. A signal can be a calorimetric, potentiometric, amperometric, optical (e.g., fluorescent, colorimetric, etc.), or piezo-electric signal. Often, the signal is present prior to detector nucleic acid cleavage and changes upon detector nucleic acid cleavage. Sometimes, the signal is absent prior to detector nucleic acid cleavage and is present upon detector nucleic acid cleavage. The detectable signal can be immobilized on a support medium for detection. The programmable nuclease can be a CRISPR-Cas (clustered regularly interspaced short palindromic repeats—CRISPR associated) nucleoprotein complex with trans cleavage activity, which can be activated by binding of a guide nucleic acid with a target nucleic acid. The CRISPR-Cas nucleoprotein complex can comprise a Cas protein (also referred to as a Cas nuclease) complexed with a guide nucleic acid, which can also be referred to as CRISPR enzyme. A guide nucleic acid can be a CRISPR RNA (crRNA). Sometimes, a guide nucleic acid may comprise a crRNA and a trans-activating crRNA (tracrRNA).

The programmable nuclease system used to detect modified target nucleic acids can comprise CRISPR RNAs (crRNAs), trans-activating crRNAs (tracrRNAs), Cas proteins, and detector nucleic acids.

Described herein are reagents comprising a programmable nuclease capable of being activated when complexed with the guide nucleic acid and the target nucleic acid segment or portion. A programmable nuclease can be capable of being activated when complexed with a guide nucleic acid and the target sequence. The programmable nuclease can be activated upon binding of the guide nucleic acid to its target nucleic acid and degrades non-specifically nucleic acid in its environment. The programmable nuclease has trans cleavage activity once activated. A programmable nuclease can be a Cas protein (also referred to, interchangeably, as a Cas nuclease). A crRNA and Cas protein can form a CRISPR enzyme.

Several programmable nucleases are consistent with the methods and devices of the present disclosure. For example, CRISPR/Cas enzymes are programmable nucleases used in the methods and systems disclosed herein. CRISPR/Cas enzymes can include any of the known Classes and Types of CRISPR/Cas enzymes. Programmable nucleases disclosed herein include Class 1 CRISPR/Cas enzymes, such as the Type I, Type IV, or Type III CRISPR/Cas enzymes. Programmable nucleases disclosed herein also include the Class 2 CRISPR/Cas enzymes, such as the Type II, Type V, and Type VI CRISPR/Cas enzymes. Preferable programmable nucleases included in the several devices disclosed herein (e.g., a microfluidic device such as a pneumatic valve device or a sliding valve device or a lateral flow assay) and methods of use thereof include a Type V or Type VI CRISPR/Cas enzyme.

In some embodiments, the Type V CRISPR/Cas enzyme is a programmable Cas12 nuclease. Type V CRISPR/Cas enzymes (e.g., Cas12 or Cas14) lack an HNH domain. A Cas12 nuclease of the present disclosure cleaves a nucleic acid via a single catalytic RuvC domain. The RuvC domain is within a nuclease, or “NUC” lobe of the protein, and the Cas12 nucleases further comprise a recognition, or “REC” lobe. The REC and NUC lobes are connected by a bridge helix and the Cas12 proteins additionally include two domains for PAM recognition termed the PAM interacting (PI) domain and the wedge (WED) domain. In some instances, a programmable Cas12 nuclease can be a Cas12a (also referred to as Cpf1) protein, a Cas12b protein, Cas12c protein, Cas12d protein, or a Cas12e protein.

In some embodiments, the programmable nuclease can be Cas13. Sometimes the Cas13 can be Cas13a, Cas13b, Cas13c, Cas13d, or Cas13e. In some cases, the programmable nuclease can be Mad7 or Mad2. In some cases, the programmable nuclease can be Cas12. Sometimes the Cas12 can be Cas12a, Cas12b, Cas12c, Cas12d, or Cas12e. In some cases, the Cas12 can be Cas12M08, which is a specific protein variant within the Cas12 protein family/classification). In some cases, the programmable nuclease can be Csm1, Cas9, C2c4, C2c8, C2c5, C2c10, C2c9, or CasZ. Sometimes, the Csm1 can also be also called smCms1, miCms1, obCms1, or suCms1. Sometimes Cas13a can also be also called C2c2. Sometimes CasZ can also be called Cas14a, Cas14b, Cas14c, Cas14d, Cas14e, Cas14f, Cas14g, or Cas14h. Sometimes, the programmable nuclease can be a type V CRISPR-Cas system. In some cases, the programmable nuclease can be a type VI CRISPR-Cas system. Sometimes the programmable nuclease can be a type III CRISPR-Cas system. Sometimes the programmable nuclease can be an engineered nuclease that is not from a naturally occurring CRISPR-Cas system. In some cases, the programmable nuclease can be from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a. The trans cleavage activity of the CRISPR enzyme can be activated when the crRNA is complexed with the target nucleic acid. The trans cleavage activity of the CRISPR enzyme can be activated when the guide nucleic acid comprising a tracrRNA and crRNA are complexed with the target nucleic acid. The target nucleic acid can be RNA or DNA.

In some embodiments, a programmable nuclease as disclosed herein is an RNA-activated programmable RNA nuclease. In some embodiments, a programmable nuclease as disclosed herein is a DNA-activated programmable RNA nuclease. In some embodiments, a programmable nuclease is capable of being activated by a target RNA to initiate trans cleavage of an RNA reporter and is capable of being activated by a target DNA to initiate trans cleavage of an RNA reporter, such as a Type VI CRISPR/Cas enzyme (e.g., a Cas13 nuclease). For example, Cas13a of the present disclosure can be activated by a target RNA to initiate trans cleavage activity of the Cas13a for the cleavage of an RNA reporter and can be activated by a target DNA to initiate trans cleavage activity of the Cas13a for trans cleavage of an RNA reporter. An RNA reporter can be an RNA-based reporter. In some embodiments, the Cas13a recognizes and detects ssDNA to initiate transcleavage of RNA reporters. Multiple Cas13a isolates can recognize, be activated by, and detect target DNA, including ssDNA, upon hybridization of a guide nucleic acid with the target DNA. For example, Lbu-Cas13a and Lwa-Cas13a can both be activated to transcollaterally cleave RNA reporters by target DNA. Thus, Type VI CRISPR/Cas enzyme (e.g., a Cas13 nuclease, such as Cas13a) can be DNA-activated programmable RNA nucleases, and therefore can be used to detect a target DNA using the methods as described herein. DNA-activated programmable RNA nuclease detection of ssDNA can be robust at multiple pH values. For example, target ssDNA detection by Cas13 can exhibit consistent cleavage across a wide range of pH conditions, such as from a pH of 6.8 to a pH of 8.2. In contrast, target RNA detection by Cas13 can exhibit high cleavage activity of pH values from 7.9 to 8.2. In some embodiments, a DNA-activated programmable RNA nuclease that also is capable of being an RNA-activated programmable RNA nuclease, can have DNA targeting preferences that are distinct from its RNA targeting preferences. For example, the optimal ssDNA targets for Cas13a have different properties than optimal RNA targets for Cas13a. As one example, gRNA performance on ssDNA can not necessarily correlate with the performance of the same gRNAs on RNA. As another example, gRNAs can perform at a high level regardless of target nucleotide identity at a 3′ position on a target RNA sequence. In some embodiments, gRNAs can perform at a high level in the absence of a G at a 3′ position on a target ssDNA sequence. Furthermore, target DNA detected by Cas13 disclosed herein can be directly taken from organisms or can be indirectly generated by nucleic acid amplification methods, such as PCR and LAMP or any amplification method described herein. Key steps for the sensitive detection of a target DNA, such as a target ssDNA, by a DNA-activated programmable RNA nuclease, such as Cas13a, can include: (1) production or isolation of DNA to concentrations above about 0.1 nM per reaction for in vitro diagnostics, (2) selection of a target sequence with the appropriate sequence features to enable DNA detection as these features are distinct from those required for RNA detection, and (3) buffer composition that enhances DNA detection.

The detection of a target DNA by a DNA-activated programmable RNA nuclease can be connected to a variety of readouts including fluorescence, lateral flow, electrochemistry, or any other readouts described herein. Multiplexing of programmable DNA nuclease, such as a Type V CRISPR-Cas protein, with a DNA-activated programmable RNA nuclease, such as a Type VI protein, with a DNA reporter and an RNA reporter, can enable multiplexed detection of target ssDNAs or a combination of a target dsDNA and a target ssDNA, respectively. Multiplexing of different RNA-activated programmable RNA nucleases that have distinct RNA reporter cleavage preferences can enable additional multiplexing. Methods for the generation of ssDNA for DNA-activated programmable RNA nuclease-based diagnostics can include (1) asymmetric PCR, (2) asymmetric isothermal amplification, such as RPA, LAMP, SDA, etc. (3) NEAR for the production of short ssDNA molecules, and (4) conversion of RNA targets into ssDNA by a reverse transcriptase followed by RNase H digestion. Thus, DNA-activated programmable RNA nuclease detection of target DNA is compatible with the various systems, kits, compositions, reagents, and methods disclosed herein. For example, target ssDNA detection by Cas13a can be employed in a detection device as disclosed herein.

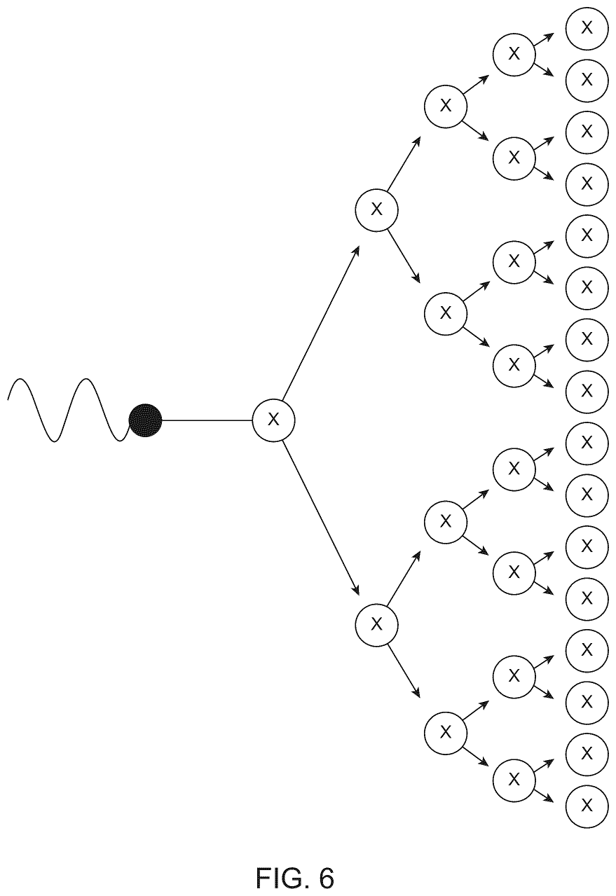

In any of the embodiments described herein, the programmable nuclease can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid, which can initiate trans cleavage activity. In some cases, the trans cut or trans cleavage can cut and/or release a reporter. In other cases, the trans cut or trans cleavage can produce an analog of a target, which can be directly detected. Trans cleavage activity can be non-specific cleavage of nearby nucleic acids by the activated programmable nuclease, such as trans cleavage of detector nucleic acids with a detection moiety. Once the detector nucleic acid is cleaved by the activated programmable nuclease, the detection moiety can be released from the detector nucleic acid and can generate a signal. For example, the detection moiety can correspond to the element, or moiety, (X) shown in FIG. 4A, 4B, 5A, 5B, and FIG. 6. The signal can be immobilized on a support medium for detection. The signal can be visualized to assess whether a target nucleic acid is present or absent.

Reporters, which can be referred to interchangeably as reporters or detector nucleic acids, can be used in conjunction with the compositions disclosed herein (e.g., programmable nucleases, guide nucleic acids, etc.) to carry out highly efficient, rapid, and accurate reactions for detecting whether a target nucleic acid is present in a sample. The reporter can be suspended in solution or immobilized on a surface. For example, the reporter can be immobilized on the surface of a chamber in a device as disclosed herein. In some cases, the reporter can be immobilized on beads, such as magnetic beads, in a chamber of a device as disclosed herein where they are held in position by a magnet placed below the chamber. The reporter can be capable of being cleaved by the activated programmable nuclease, thereby generating a detectable signal. The detectable signal can correspond to a release of one or more elements (X) as illustrated in FIGS. 5A and 5B. The release of the one or more elements (X) can initiate a reaction with another element (Y) when the element (Y) is in the presence of the element (X). The reaction between the element (Y) and the element (X) can initiate a chemical chain reaction in a solid phase material. Such a chemical chain reaction can produce one or more physical or chemical changes. In some cases, the physical or chemical changes can be optically detected. In some embodiments, one or more cascade amplification reactions can occur to further amplify the signal before sensing or detection. There can be a single point of attachment between the reporter and the element (X). Cutting the single point of attachment can release a macro molecule (X), which can undergo a series of reactions based on the macro molecule (X) itself. In any of the embodiments described herein, the reporter can comprise a single stranded detector nucleic acid comprising a detection moiety.

As used herein, a detector nucleic acid is used interchangeably with reporter or reporter. In some cases, the detector nucleic acid is a single-stranded nucleic acid comprising deoxyribonucleotides. In other cases, the detector nucleic acid is a single-stranded nucleic acid comprising ribonucleotides. The detector nucleic acid can be a single-stranded nucleic acid comprising at least one deoxyribonucleotide and at least one ribonucleotide. In some cases, the detector nucleic acid is a single-stranded nucleic acid comprising at least one ribonucleotide residue at an internal position that functions as a cleavage site. In some cases, the detector nucleic acid may comprise at least 2, 3, 4, 5, 6, 7, 8, 9, or 10 ribonucleotide residues at an internal position. In some cases, the detector nucleic acid may comprise from 2 to 10, from 3 to 9, from 4 to 8, or from 5 to 7 ribonucleotide residues at an internal position. In some cases, the detector nucleic acid may comprise from 3 to 10, from 4 to 10, from 5 to 10, from 6 to 10, from 7 to 10, from 8 to 10, from 9 to 10, from 2 to 8, from 3 to 8, from 5 to 8, from 6 to 8, from 7 to 8, from 2 to 5, from 3 to 5, or from 4 to 5 ribonucleotide residues at an internal position. Sometimes the ribonucleotide residues are continuous. Alternatively, the ribonucleotide residues are interspersed in between non-ribonucleotide residues. In some cases, the detector nucleic acid has only ribonucleotide residues. In some cases, the detector nucleic acid has only deoxyribonucleotide residues. In some cases, the detector nucleic acid may comprise nucleotides resistant to cleavage by the programmable nuclease described herein. In some cases, the detector nucleic acid may comprise synthetic nucleotides. In some cases, the detector nucleic acid may comprise at least one ribonucleotide residue and at least one non-ribonucleotide residue. In some cases, the detector nucleic acid is 5-20, 5-15, 5-10, 7-20, 7-15, or 7-10 nucleotides in length. In some cases, the detector nucleic acid is from 3 to 20, from 4 to 20, from 5 to 20, from 6 to 20, from 7 to 20, from 8 to 20, from 9 to 20, from 10 to 20, from 15 to 20, from 3 to 15, from 4 to 15, from 5 to 15, from 6 to 15, from 7 to 15, from 8 to 15, from 9 to 15, from 10 to 15, from 3 to 10, from 4 to 10, from 5 to 10, from 6 to 10, from 7 to 10, from 8 to 10, from 9 to 10, from 3 to 8, from 4 to 8, from 5 to 8, from 6 to 8, or from 7 to 8 nucleotides in length. In some cases, the detector nucleic acid may comprise at least one uracil ribonucleotide. In some cases, the detector nucleic acid may comprise at least two uracil ribonucleotides. Sometimes the detector nucleic acid has only uracil ribonucleotides. In some cases, the detector nucleic acid may comprise at least one adenine ribonucleotide. In some cases, the detector nucleic acid may comprise at least two adenine ribonucleotides. In some cases, the detector nucleic acid has only adenine ribonucleotides. In some cases, the detector nucleic acid may comprise at least one cytosine ribonucleotide. In some cases, the detector nucleic acid may comprise at least two cytosine ribonucleotides. In some cases, the detector nucleic acid may comprise at least one guanine ribonucleotide. In some cases, the detector nucleic acid may comprise at least two guanine ribonucleotides. A detector nucleic acid can comprise only unmodified ribonucleotides, only unmodified deoxyribonucleotides, or a combination thereof. In some cases, the detector nucleic acid is from 5 to 12 nucleotides in length. In some cases, the detector nucleic acid is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some cases, the detector nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by a programmable nuclease comprising Cas13, a detector nucleic acid can be 5, 8, or 10 nucleotides in length. For cleavage by a programmable nuclease comprising Cas12, a detector nucleic acid can be 10 nucleotides in length.

The single stranded detector nucleic acid can comprise a detection moiety capable of generating a first detectable signal. Sometimes the detector nucleic acid may comprise a protein capable of generating a signal. A signal can be a calorimetric, potentiometric, amperometric, optical (e.g., fluorescent, colorometric, etc.), or piezo-electric signal. In some cases, a detection moiety is on one side of the cleavage site. Optionally, a quenching moiety is on the other side of the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the detector nucleic acid. Sometimes the detection moiety is at the 3′ terminus of the detector nucleic acid. In some cases, the detection moiety is at the 5′ terminus of the detector nucleic acid. In some cases, the quenching moiety is at the 3′ terminus of the detector nucleic acid. In some cases, the single-stranded detector nucleic acid is at least one population of the single-stranded nucleic acid capable of generating a first detectable signal. In some cases, the single-stranded detector nucleic acid is a population of the single stranded nucleic acid capable of generating a first detectable signal. Optionally, there are more than one population of single-stranded detector nucleic acid. In some cases, there are 2, 3, 4, 5, 6, 7, 8, 9, 10, 11, 12, 13, 14, 15, 20, 30, 40, 50, or greater than 50, or any number spanned by the range of this list of different populations of single-stranded detector nucleic acids capable of generating a detectable signal. In some cases, there are from 2 to 50, from 3 to 40, from 4 to 30, from 5 to 20, or from 6 to 10 different populations of single-stranded detector nucleic acids capable of generating a detectable signal. In some cases there are from 2 to 50, from 5 to 50, from 10 to 50, from 15 to 50, from 20 to 50, from 25 to 50, from 30 to 50, from 35 to 50, from 40 to 50, from 2 to 40, from 5 to 40, from 10 to 40, from 15 to 40, from 20 to 40, from 25 to 40, from 30 to 40, from 35 to 40, from 2 to 30, from 5 to 30, from 10 to 30, from 15 to 30, from 20 to 30, from 25 to 30, from 2 to 20, from 5 to 20, from 10 to 20, from 15 to 20, from 2 to 10, or from 5 to 10 different populations of single-stranded detector nucleic acids capable of generating a detectable signal.

In some embodiments, target nucleic acid amplicons are detected by immobilized programmable nuclease probes, such as, for example, CRISPR CAS guide RNA probes (referred to as CRISPR probe). Upon a complementary binding event between a target nucleic acid amplicon and a programmable nuclease probe (e.g., an immobilized CRISPR CAS/guide RNA complex) a cutting event will occur that release a reporter that is then detected by a sensor. There are two main schemes for detection of the binding events between the target and the programmable nuclease probe, a mobile phase scheme as illustrated in FIG. 7A and a stationary phase scheme as illustrated in FIG. 7C. In any of the embodiments described herein, each programmable nuclease probe can have one or more programmable nucleases (e.g., CRISPR enzymes) with one or more specific guide RNAs for detecting different target nucleic acids or different classes of target nucleic acids.

In certain circumstances, a mobile phase detection scheme (FIG. 7A) is used. In these circumstances, the remixed sample is flown through a channel. The walls of the channel can comprise one or more regions on which one or more programmable nuclease probes or CRISPR probes are disposed. The programmable nuclease probes or CRISPR probes can comprise a guide nucleic acid (e.g., a guide RNA) complexed with a programmable nuclease as described elsewhere herein. The guide nucleic acid and/or the programmable nuclease can be immobilized relative to the walls of the channel. The guide nucleic acid and/or the programmable nuclease can be exposed to the remixed sample flowing through the channel. Reporter compounds can be cut and released upon a complementary binding event of a target nucleic acid amplicon and a specific guide nucleic acid. The reporter compounds can freely participate in one or more cascading amplification reactions that generate an amplified signal, as illustrated in FIG. 6. The amplified signal can be detected using one or more sensors as described herein.

In some embodiments, a stationary phase detection scheme is used. In these embodiments the remixed sample can contain one or more target nucleic acid amplicon sequence types and copies thereof. FIG. 7B shows a top-down view of a plurality of target amplicons that can interact with programmable nuclease probes positioned at known locations on the interior walls of the reaction chamber. FIG. 7C shows a cross-sectional side view of the detection chamber with a target amplicon interacting with a programmable nuclease probe. The guide RNA of the programmable nuclease or CRISPR probe can be immobilized adjacent to a bottom surface of the chamber. When a complementary interaction between the probe and the target occurs, the CRISPR enzyme will cut and release a reporter which will then be sensed. Since the specific guide RNA of the immobilized programmable nuclease or CRISPR probe can be spatially registered, multiplexed detection can be achieved. In some cases, where one sensor corresponds to one immobilized probe, electrical detection can be used. Other methods of detection can also be used, such as optical imaging, surface plasmon resonance (SPR), and/or interferometric sensing.

The plurality of programmable nuclease probes shown in FIG. 2, FIG. 7A, and FIG. 7C and described herein can be arranged in various configurations. For example, the plurality of programmable nuclease probes can be arranged in a lateral configuration. Alternatively, the plurality of programmable nuclease probes can be arranged in a circular configuration such that each programmable nuclease probe is equidistant from a common center point. In some cases, the plurality of programmable nuclease probes can be distributed with a same separation distance or spacing between the programmable nuclease probes. In other cases, a first programmable nuclease probe and a second programmable nuclease probe can be separated by a first separation distance or spacing, and a third programmable nuclease probe and a fourth programmable nuclease probe can be separated by a second separation distance or spacing that is different than the first separation distance or spacing.

As described above, the single stranded detector nucleic acid can comprise a detection moiety capable of generating a first detectable signal. Sometimes the detector nucleic acid may comprise a protein capable of generating a signal. A signal can be a colorimetric, potentiometric, amperometric, optical (e.g., fluorescent, colorometric, etc.), or piezo-electric signal. The generation of the detectable signal from the release of the detection moiety indicates that cleavage by the programmable nuclease has occurred and that the sample contains the target nucleic acid. A detection moiety can be any moiety capable of generating a colorimetric, potentiometric, amperometric, optical (e.g., fluorescent, colorometric, etc.), or piezo-electric signal. A detector nucleic acid, sometimes, is protein-nucleic acid that can generate a calorimetric, potentiometric, amperometric, optical (e.g., fluorescent, colorometric, etc.), or piezo-electric signal upon cleavage of the nucleic acid. Often a calorimetric signal is heat produced after cleavage of the detector nucleic acids. Sometimes, a calorimetric signal is heat absorbed after cleavage of the detector nucleic acids. A potentiometric signal, for example, is electrical potential produced after cleavage of the detector nucleic acids. An amperometric signal can be movement of electrons produced after the cleavage of detector nucleic acid. Often, the signal is an optical signal, such as a colorometric signal or a fluorescence signal. An optical signal is, for example, a light output produced after the cleavage of the detector nucleic acids. Sometimes, an optical signal is a change in light absorbance between before and after the cleavage of detector nucleic acids. Often, a piezo-electric signal is a change in mass between before and after the cleavage of the detector nucleic acid.

Detecting the presence or absence of a target nucleic acid of interest can involve measuring a signal emitted from a detection moiety present in a reporter, after cleavage of the reporter by an activated programmable nuclease. The signal can be measured using one or more sensors integrated with the device or operatively coupled to the device. Thus, the detecting steps disclosed herein can involve measuring the presence of a target nucleic acid, quantifying how much of the target nucleic acid is present, or, measuring a signal indicating that the target nucleic acid is absent in a sample. In some embodiments, a signal is generated upon cleavage of the detector nucleic acid by the programmable nuclease. In other embodiments, the signal changes upon cleavage of the detector nucleic acid by the programmable nuclease. In other embodiments, a signal can be present in the absence of detector nucleic acid cleavage and disappear upon cleavage of the target nucleic acid by the programmable nuclease. For example, a signal can be produced in a microfluidic device or lateral flow device after contacting a sample with a composition comprising a programmable nuclease.

In some cases, the signal can comprise a colorimetric signal or a signal visible by eye. In some instances, the signal is fluorescent, electrical, chemical, electrochemical, or magnetic. A signal can be a calorimetric, potentiometric, amperometric, optical (e.g., fluorescent, colorometric, etc.), or piezo-electric signal. In some cases, the detectable signal is a colorimetric signal or a signal visible by eye. In some instances, the detectable signal is fluorescent, electrical, chemical, electrochemical, or magnetic. In some cases, the first detection signal is generated by binding of the detection moiety to the capture molecule in the detection region, where the first detection signal indicates that the sample contained the target nucleic acid. Sometimes the system can detect more than one type of target nucleic acid, wherein the system may comprise more than one type of guide nucleic acid and more than one type of detector nucleic acid. In some cases, the detectable signal is generated directly by the cleavage event. Alternatively, or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on its spatial location on the detection region of the support medium.

In some cases, the one or more detectable signals generated after cleavage can produce an index of refraction change or one or more electrochemical changes. In some cases, real-time detection of the Cas reaction can be achieved using fluorescence, electrochemical detection, and/or electrochemiluminescence.

In some cases, the detectable signals can be detected and analyzed in various ways. For example, the detectable signals can be detected using an imaging device. The imaging device can a digital camera, such a digital camera on a mobile device. The mobile device can have a software program or a mobile application that can capture fluorescence, ultraviolet (UV), infrared (IR), or visible wavelength signals. Any suitable detection or measurement device can be used to detect and/or analyze the colorimetric, fluorescence, amperometric, potentiometric, or electrochemical signals described herein. In some embodiments, the colorimetric, fluorescence, amperometric, potentiometric, or another electrochemical sign can be detected using a measurement device connected to a detection chamber of the device (e.g., a fluorescence measurement device, a spectrophotometer, and/or an oscilloscope).

In certain aspects of this disclosure, multiplexing refers to parallel sensing of multiple target nucleic acid sequences in one sample by multiple probes.

FIGS. 7A, 7B, and 7C illustrate two multiplexing embodiments of a CRISPR detection device, as described herein. FIGS. 7A, 7B, and 7C illustrate two multiplexing embodiments of the CRISPR detection device. FIG. 7A illustrates a capillary flow or mobile sample phase embodiment and FIGS. 7B and 7C illustrate a stationary sample phase embodiment. FIG. 7A illustrates, in certain embodiments, a reaction chamber that is in the form of a capillary circuit. Functionalized programmable nuclease probes, e.g., CRISPR probes, can be disposed on the capillary walls, and one or more guide nucleic acids associated with the programmable nuclease probes, e.g., CRISPR probes can be exposed to the sample for binding. Upon binding to a complementary target nucleic acid amplicon (or a target nucleic acid sequence) the programmable nuclease probe or CRISPR probe then cuts and releases at least a portion of a reporter that generates a signal indicating the presence of the particular target nucleic acid amplicon. This identification process is repeated in parallel across multiple programmable nuclease probes or CRISPR probes, where each programmable nuclease or CRISPR probe is configured to detect a particular target sequence, nucleic acid amplicon, set of target sequences, or set of target nucleic acid amplicons. In certain aspects, as seen in FIGS. 7B and 7C, multiplexed detection can also be achieved in a stationary phase, or microarray format. In some embodiments, programmable nuclease probes or CRISPR probes, each designed to detect certain target nucleic acid sequences, are immobilized in known locations. When the remixed sample containing multiple types of target amplicons is exposed to the array of programmable nuclease or CRISPR probes, the specific probe-target pairs will bind and trigger signal events. These signal events can be associated with a particular target nucleic acid amplicon or set of target nucleic acid amplicons either by its location when imaging is used, or by a signal received by a particular sensor when sensors are individually linked to each probe. In some instances, one or more target nucleic acid amplicons can be detected by a programmable nuclease probe. In some instances, the programmable nuclease probe can interact with and/or detect a class of sequences or a class of target nucleic acid amplicons, which can indicate a presence or an existence of a particular organism, disease state, or phenotype present within the sample.

The devices of the present disclosure can be used to implement for detection of one or more target nucleic acids within the sample. The devices of the present disclosure can comprise one or multiple pumps, valves, reservoirs, and chambers for sample preparation, optional amplification of a target nucleic acid within the sample, mixing with a programmable nuclease, and detection of a detectable signal arising from cleavage of detector nucleic acids by a programmable nuclease.

Methods consistent with the present disclosure include a multiplexing method of assaying for a plurality of target nucleic acids in a sample. A multiplexing method may comprise contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some reporters (e.g., protein-nucleic acids) of a population of reporter molecules (e.g., protein-nucleic acids), wherein the signal indicates a presence of the target nucleic acid in the sample and wherein absence of the signal indicates an absence of the target nucleic acid in the sample.

Multiplexing can comprise spatial multiplexing wherein multiple different target nucleic acids are detected at the same time, but the reactions are spatially separated. In some cases, the multiple target nucleic acids are detected using the same programmable nuclease, but different guide nucleic acids. The multiple target nucleic acids sometimes are detected using the different programmable nucleases. In the case wherein multiple target nucleic acids are detected using the different programmable nucleases, the method involves using a first programmable nuclease, which upon activation (e.g., after binding of a first guide nucleic acid to a first target), cleaves a nucleic acid of a first reporter and using a second programmable nuclease, which upon activation (e.g., after binding of a second guide nucleic acid to a second target), cleaves a nucleic acid of a second reporter.

Sometimes, multiplexing can be single reaction multiplexing wherein multiple different target acids are detected in a single reaction volume. Often, at least two different programmable nucleases are used in single reaction multiplexing. For example, multiplexing can be enabled by immobilization of multiple categories of detector nucleic acids within a fluidic system, to enable detection of multiple target nucleic acids within a single fluidic system. Multiplexing allows for detection of multiple target nucleic acids in one kit or system. In some cases, the multiple target nucleic acids comprise different target nucleic acids to a virus, a bacterium, or a pathogen responsible for one disease. In some cases, the multiple target nucleic acids comprise different target nucleic acids associated with a cancer or genetic disorder. Multiplexing for one disease, cancer, or genetic disorder increases at least one of sensitivity, specificity, or accuracy of the assay to detect the presence of the disease in the sample. In some cases, the multiple target nucleic acids comprise target nucleic acids directed to different viruses, bacteria, or pathogens responsible for more than one disease. In some cases, multiplexing allows for discrimination between multiple target nucleic acids, such as target nucleic acids that comprise different genotypes of the same bacteria or pathogen responsible for a disease, for example, for a wild-type genotype of a bacteria or pathogen and for genotype of a bacteria or pathogen comprising a mutation, such as a single nucleotide polymorphism (SNP) that can confer resistance to a treatment, such as antibiotic treatment. Multiplexing, thus, allows for multiplexed detection of multiple genomic alleles. For example, multiplexing may comprise method of assaying comprising a single assay for a microorganism species using a first programmable nuclease and an antibiotic resistance pattern in a microorganism using a second programmable nuclease. Sometimes, multiplexing allows for discrimination between multiple target nucleic acids of different HPV strains, for example, HPV16 and HPV18. In some cases, the multiple target nucleic acids comprise target nucleic acids directed to different cancers or genetic disorders. Often, multiplexing allows for discrimination between multiple target nucleic acids, such as target nucleic acids that comprise different genotypes, for example, for a wild-type genotype and for SNP genotype. Multiplexing for multiple diseases, cancers, or genetic disorders provides the capability to test a panel of diseases from a single sample. For example, multiplexing for multiple diseases can be valuable in a broad panel testing of a new patient or in epidemiological surveys. Often multiplexing is used for identifying bacterial pathogens in sepsis or other diseases associated with multiple pathogens.

Furthermore, signals from multiplexing can be quantified. For example, a method of quantification for a disease panel may comprise assaying for a plurality of unique target nucleic acids in a plurality of aliquots from a sample, assaying for a control nucleic acid control in a second aliquot of the sample, and quantifying a plurality of signals of the plurality of unique target nucleic acids by measuring signals produced by cleavage of detector nucleic acids compared to the signal produced in the second aliquot. In this context, a unique target nucleic acid refers to the sequence of a nucleic acid that has an at least one nucleotide difference from the sequences of the other nucleic acids in the plurality. Multiple copies of each target nucleic acid can be present. For example, a unique target nucleic population can comprise multiple copies of the unique target nucleic acid. Often the plurality of unique target nucleic acids is from a plurality of bacterial pathogens in the sample.

In some instances, the multiplexed devices, systems, fluidic devices, kits, and methods detect at least 2 different target nucleic acids in a single reaction. In some instances, the multiplexed devices, systems, fluidic devices, kits, and methods detect at least 3 different target nucleic acids in a single reaction. In some instances, the multiplexed devices, systems, fluidic devices, kits, and methods detect at least 4 different target nucleic acids in a single reaction. In some instances, the multiplexed devices, systems, fluidic devices, kits, and methods detect at least 5 different target nucleic acids in a single reaction. In some cases, the multiplexed devices, systems, fluidic devices, kits, and methods detect at least 6, 7, 8, 9, or 10 different target nucleic acids in a single reaction. In some instances, the multiplexed kits detect at least 2 different target nucleic acids in a single kit. In some instances, the multiplexed kits detect at least 3 different target nucleic acids in a single kit. In some instances, the multiplexed kits detect at least 4 different target nucleic acids in a single kit. In some instances, the multiplexed kits detect at least 5 different target nucleic acids in a single kit. In some instances, the multiplexed kits detect at least 6, 7, 8, 9, or 10 different target nucleic acids in a single kit. In some instances, the multiplexed kits detect from 2 to 10, from 3 to 10, from 4 to 10, from 5 to 10, from 6 to 10, from 7 to 10, from 8 to 10, from 9 to 10, from 2 to 9, from 3 to 9, from 4 to 9, from 5 to 9, from 6 to 9, from 7 to 9, from 8 to 9, from 2 to 8, from 3 to 8, from 4 to 8, from 5 to 8, from 6 to 8, from 7 to 8, from 2 to 7, from 3 to 7, from 4 to 7, from 5 to 7, from 6 to 7, from 2 to 6, from 3 to 6, from 4 to 6, from 5 to 6, from 2 to 5, from 3 to 5, from 4 to 5, from 2 to 4, from 3 to 4, or from 2 to 3 different target nucleic acids in a single kit. Multiplexing can be carried out in a single-pot or “one-pot” reaction, where reverse transcription, amplification, in vitro transcription, or any combination thereof, and detection are carried out in a single volume. Multiplexing can be carried out in a “two-pot reaction”, where reverse transcription, amplification, in vitro transcription, or any combination thereof, are carried out in a first volume and detection is carried out in a second volume.

In some cases, multiplexing can comprise detecting multiple targets with a single probe. Alternatively, multiplexing can comprise detecting multiple targets with multiple probes. The multiple probes can be configured to detect a presence of a particular sequence, target nucleic acid, or a plurality of different target sequences or nucleic acids.

The devices of the present disclosure can be manufactured from a variety of different materials. Exemplary materials that can be used include plastic polymers, such as poly-methacrylate (PMMA), cyclic olefin polymer (COP), cyclic olefin copolymer (COC), polyethylene (PE), high-density polyethylene (HDPE), polypropylene (PP); glass; and silicon. Features of the device (e.g., chambers, channels, etc.) can be manufactured by various processes. For example, the features can be (1) embossed using injection molding, (2) micro-milled or micro-engraved using computer numerical control (CNC) micromachining or non-contact laser drilling (by means of a CO2 laser source); (3) generated using additive manufacturing, and/or (4) generated using one or more photolithographic or stereolithographic methods.

In some embodiments, any of the devices of the present disclosure can comprise a sample interface configured to receive a sample that may comprise at least one gene of interest. The device can further comprise a channel in fluid communication with the sample interface and a detection chamber. In some cases, the channel may comprise one or more movable mechanisms to separate the sample into a plurality of droplets. As used herein, a droplet can refer to a volumetric portion of the sample, a partitioned sub-sample of the sample, and/or an aliquot of the sample. In some cases, the detection chamber is configured to receive and contact the plurality of droplets with at least one programmable nuclease probe disposed on a surface of said detection chamber. The at least one programmable nuclease probe can comprise a guide nucleic acid complexed with a programmable nuclease. In some cases, the programmable nuclease probe may comprise a CRISPR/Cas enzyme. In some cases, the guide nucleic acid may comprise a guide RNA. In some embodiments, the device may comprise a plurality of programmable nuclease probes comprising different guide RNAs.

The device can further comprise a plurality of sensors that determine a presence of said at least one gene of interest by detecting a signal produced upon cleavage of a target nucleic acid region in said at least one gene of interest by said at least one programmable nuclease probe. The cleavage of the target nucleic acid region can occur after a complementary binding of said target nucleic acid region to said guide nucleic acid of said at least one programmable nuclease probe.

As described elsewhere herein, the one or more movable mechanisms can comprise one or more valves configured to restrict flow through one or more sections of the channel. The one or more movable mechanisms can comprise a plunger or a bristle that is configured to restrict flow through one or more sections of the channel. The one or more movable mechanisms can be operatively coupled to a power source that is integrated with or insertable into the device. The power source can comprise a battery.

In some cases, any of the devices described herein may comprise a physical filter to filter one or more particles from the sample that do not comprise the one or more targets (e.g., a gene of interest). In some cases, the device may comprise a sample preparation chamber. The sample preparation chamber can comprise a lysing agent. The sample preparation chamber can comprise a heating unit configured for heat inactivation. The sample preparation chamber can comprise one or more reagents for nucleic acid purification.

In some cases, the channel between the sample interface and the detection chamber may comprise a plurality of heating elements and a plurality of heat sinks for amplifying the at least one gene of interest or a portion thereof. The plurality of heating elements and the plurality of heat sinks can be configured to perform one or more thermocycling operations on the sample or at least a portion of the sample (e.g., the plurality of droplets).

As described elsewhere herein, the signal produced upon cleavage of a target nucleic acid can be associated with a physical, chemical, or electrochemical change or reaction. The signal can comprise an optical signal, a fluorescent or colorimetric signal, a potentiometric or amperometric signal, and/or a piezo-electric signal. In some cases, the signal is associated with a change in an index of refraction of a solid or gel volume in which the at least one programmable nuclease probe is disposed.

In some embodiments, the device may comprise a sample interface configured to receive a sample that may comprise one or more genomic targets of interest. In some cases, the one or more genomic targets of interest comprise a sequence of nucleic acids comprising the nucleic acid.

The device can further comprise one or more channels comprising one or more movable mechanisms to separate the sample into partitioned samples. The one or more channels can be in fluid communication with the sample interface and a reaction chamber that is configured to receive and contact the partitioned samples with an enzyme, reagent, or programmable detection agent that is configured to cleave a nucleic acid of said one or more genomic targets of interest.

The device can further comprise a plurality of sensors for determining a presence of the one or more genomic targets of interest by detecting one or more reporters released by said cleavage of said nucleic acid. The programmable detection agent can be a CRISPR/Cas enzyme. In some cases, the reporter may comprise a nucleic acid and a detection moiety. In some cases, the reporter may comprise at least one ribonucleotide or at least one deoxyribonucleotide. In some cases, the reporter may comprise a DNA nucleic acid or an RNA nucleic acid. The reported molecule can be immobilized on a surface of the detection chamber (i.e., a movement of the reporter can be physically or chemically constrained).

In some cases, the one or more movable mechanisms comprise a plurality of valves configured to restrict flow in a first direction through the one or more channels towards the sample interface. The plurality of valves can be configured to selectively permit flow in a second direction through the one or more channels towards the reaction chamber. A first valve and a second valve of the plurality of valves can be configured to physically, fluidically, or thermally isolate a first portion of the sample from a second portion of the sample when the first valve and the second valve are in a closed state.

The one or more channels can comprise a plurality of heating elements and a plurality of heat sinks to perform thermocycling on the partitioned samples. A first heating element of the plurality of heating elements and a first heat sink of the plurality of heat sinks can be positioned between a first movable mechanism and a second movable mechanism of the one or more movable mechanisms.

In any of the embodiments described herein, the device can further comprise a telemedicine unit configured to provide one or more detection results to a computing unit that is remote from the device. In some embodiments, the telemedicine unit provides one or more detection results to a computing unit that is remote to the device through a cloud-based connection. In some embodiments, the telemedicine unit is HIPAA compliant. In some embodiments, the telemedicine unit transmits encrypted data. The computing unit can comprise a mobile device or a computer. The one or more detection results can indicate a presence or an absence of a target nucleic acid of interest in the sample.

In another aspect, the present disclosure provides a method for target detection. The method can comprise contacting a sample with the device of any of the preceding claims and detecting a presence or an absence of one or more genes of interest in said sample. In some cases, the method can comprise generating one or more detection results indicating the presence or the absence of the one or more genes of interest in the sample. In some cases, the method can comprise transmitting the one or more detection results to a remote computing unit. The remote computing unit can comprise, for example, a mobile device.

In another aspect, the present disclosure provides a method for target detection. The method can comprise providing a sample comprising at least one gene of interest. The method can comprise separating the sample into a plurality of sub-samples using one or more movable mechanisms described herein. The method can comprise receiving the plurality of sub-samples in a detection chamber and contacting the plurality of sub-samples with at least one programmable nuclease probe disposed on a surface of said detection chamber. The at least one programmable nuclease probe can comprise a guide nucleic acid complexed with a programmable nuclease. In some cases, the method can comprise contacting the plurality of sub-samples with a plurality of programmable nuclease probes comprising different guide RNAs. The method can comprise using a plurality of sensors to determine a presence or an absence of said at least one gene of interest by detecting a signal produced upon cleavage of a target nucleic acid region in said at least one gene of interest by said at least one programmable nuclease probe.

In some cases, the method can further comprise amplifying the at least one gene of interest after separating the sample into a plurality of sub-samples. In some cases, the method can further comprise amplifying the at least one gene of interest before mixing the plurality of sub-samples in the detection chamber. Amplifying the at least one gene of interest can comprise using a plurality of heating elements and a plurality of heat sinks to perform thermocycling on the plurality of sub-samples.

In some cases, the method can comprise using a physical filter to filter one or more particles from the sample that do not comprise the one or more target genes of interest. In some cases, the method can comprise lysing the sample before detecting the one or more target genes of interest. In some cases, the method can comprise performing heat inactivation on the sample. In some cases, the method can comprise performing nucleic acid purification on the sample.

In some cases, the detection devices described herein can be configured to implement process control procedures to ensure that the sample preparation, target amplification, and target detection processes are performed accurately and as intended.

### Disposable Fluidic Workflow

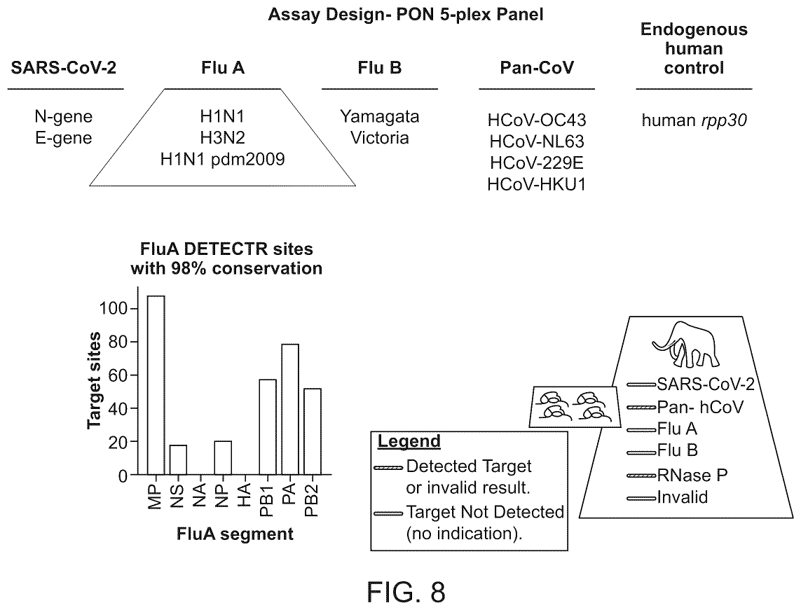

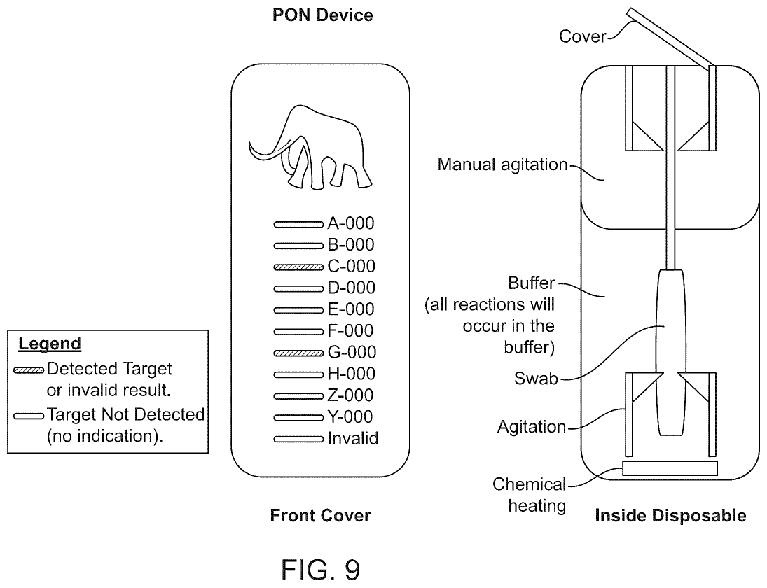

Described herein are various embodiments, for point of need (PON) programmable nuclease-based devices. In some embodiments, the PON device is configured for a 5-plex respiratory panel as shown in FIG. 8. FIG. 8 shows an exemplary assay design for a PON 5-plex panel comprising pooled CRISPR-Cas complexes in discrete regions for viral detection. The discrete regions are for detection of: (1) SARS-CoV-2, (2) Flu A, (3) Flu B, (4) Pan-CoV, and (5) Endogenous human control. The (1) SARS-CoV-2 region may comprise gRNA for detecting N-gene targets and E-gene targets, the (2) Flu A region may comprise gRNA for detecting H1N1 targets, H3N2 targets, and H1N1 pdm2009 targets, the (3) Flu B region may comprise gRNA for detecting Yamagata targets and Victoria targets, the (4) Pan-CoV region may comprise gRNA for detecting HCoV-OC43 targets, HCoV-NL63 targets, HCoV-229E targets, and HCoV-HKU1 targets, and the (5) Endogenous human control region may comprise gRNA for human rpp30 targets. Each region can comprise pooled gRNA. For example, the gRNAs for the Flu A region bind to target sites that are 98% conserved among H1N1, H3N2, and H1N1 pdm2009, such as Matrix Protein 1 (MP), Nonstructural Protein 1 (NS), Neuraminidase (NA), Nucleoprotein (NP), Hemagglutinin (HA), PB1, Polymerase Acidic Protein (PA), and Polymerase Basic Protein 2 (PB2). Detected signal from each region can indicate the detection of a target within that region. In some embodiments, PON programmable nuclease-based devices are disposable, as shown in FIG. 9.

Described herein are various Point-of-need (PON) diagnostics that can rapidly identify causes of ailments on location where needed by patients. In some embodiments, disposable PON devices can be manufactured and delivered to the user in a sterile condition to help fill this need. However, in some embodiments, transferring assays from the lab to a point-of-need device can be a challenge. In some embodiments, integrating a fluidic system capable of delivering rapid, reliable, and easy to interpret results while still being disposable is especially challenging.

Disclosed herein are various embodiments of methods, devices, and compositions for a disposable fluidic workflow. In some embodiments, the workflow method may comprise: (1) sample collection from the patient and delivery to the device, (2) lysis, (3) amplification, and (4) detection/readout. In some embodiments, any of the disposable fluidic devices described herein, may comprise an exterior housing and an interior control printed circuit board (PCB) on which to mount sub-components. In some embodiments, such subcomponents may comprise a swab to collect a sample from patient, a mechanism for extracting sample from the swab (e.g., a scraper), fluidics configured to move the sample within the device, one or more sample chambers, one or more reagent storage bags or chambers, amplification mechanisms, detectors, valves and heating elements. In some embodiments, described herein, valves may be rotary valves capable of diverting sample to and from one chamber or channel to multiple other chambers or channels.

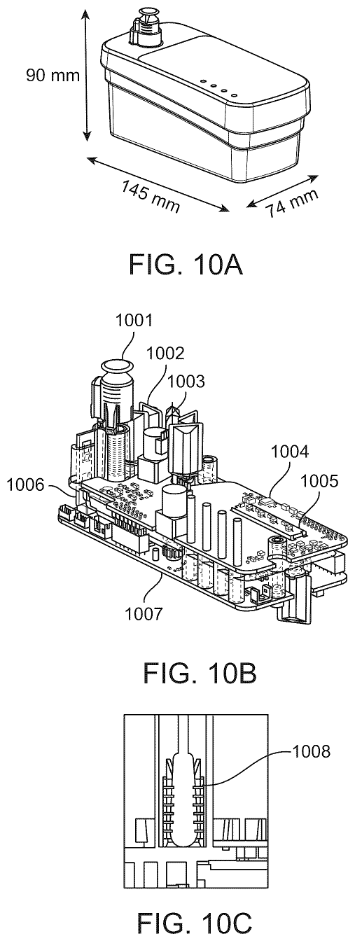

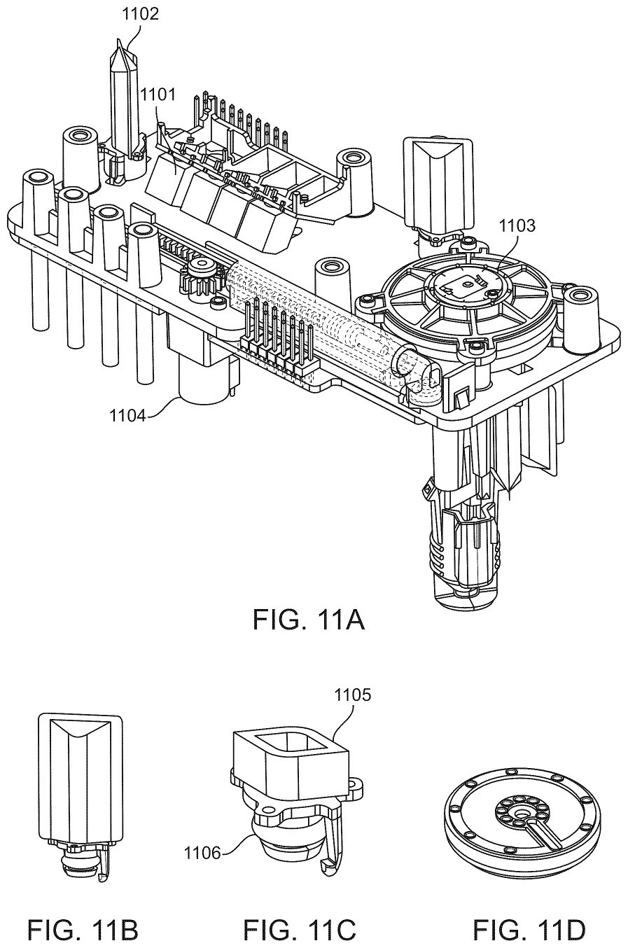

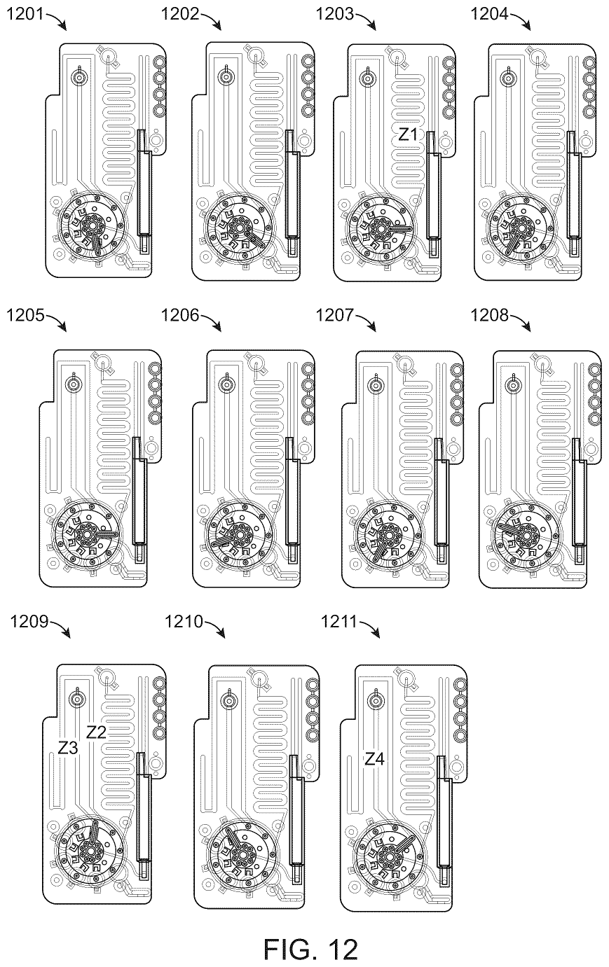

FIG. 10A illustrates an example of a consumable exterior housing. FIG. 10B illustrates an example of components mounted on a printed circuit board (PCB) 1007 and housed within the consumable device housing of FIG. 10A. In some embodiments, a swab cap 1001 may be configured (e.g., rotated, depressed, etc.) to allow user to activate the device. In some embodiments, reagent storage bags (or chambers, etc.) 1002 may be mounted vertically on the PCB 1007. In some embodiments, a motor 1003 to drive the valve, seen in FIG. 11D, may be integrated onto the PCB 1007. In some embodiments, heat traces 1004 may be integrated onto the PCB 1007 to facilitate heating of the sample or a portion thereof. In some embodiments, a light emitting diode (LED) 1005 for excitation of a reporter (e.g., one or more fluorescent labels) may be integrated onto the PCB. In some embodiments, a start button may be activated by pushing on the swab cap 1006. FIG. 10C illustrates an example swab housing, containing a scraper configured to facilitate removal of a sample from the swab 1008. In some embodiments, the sample interface may comprise a scraper. FIG. 9 shows an embodiment of a device comprising a scraper that agitates a swab carrying the sample when the swab is inserted into the inside disposable. FIG. 10C shows another embodiment of a device comprising a scraper (1008). Those skilled in the art will recognize that the scraper may comprise a variety of forms, shapes, and sizes while retaining its function of agitating an input sample. In some embodiments, the scraper facilitates the transfer of sample from the swab to the device.

FIGS. 11A, 11B, 11C, 11D further illustrate various example components of a consumable, as described herein. FIG. 11A illustrates an example of photodiodes for emission detection 1101, expansion chamber bags 1102, encoder PCB 1103, and a syringe pump, which may be disposed in a layer on top of the PCB. In some embodiments, the syringe pump may be driven by a motor 1004. In other embodiments, the syringe pump is driven by a shape memory alloy (SMA) (e.g., a compressed spring) for mass production. FIG. 11B illustrates an exemplary expansion bag 1102. FIG. 11C illustrates an exemplary injection molded bag core of the expansion bag 1102, where a filter (not shown) can be heat sealed on a proximal end thereof 1105. In some embodiments, an O-Ring seal may be made with the injection molded bag core 1106 at a distal end thereof to facilitate coupling of the expansion bag (1102) to the device during manufacture. In other embodiments, an overmold component 1106, or soft polymer such as TPE, may be integrated with the injection molded bag core for mass production. FIG. 11D illustrates an embodiment of a rotatory valve overmold configured to regulate fluid flow within the device as described herein.

FIG. 12 illustrates various valve position examples of the consumable device in use during the workflow method as follows: extract lysis (1201); transfer lysis swab to extract sample (1202); move lysis to heat zone (Z1) (1203); move lysis to beads for binding (1204); move lysis to waste (1205); extract elution (1206); move elution to beads (1207) move elution to MM (1208); move to PCR cycling (1209); move to dilution (1210); move to DETECTR (1211). FIG. 12 additionally illustrates an embodiment of the consumable with heat zones (Z) for lysis heating at 95 C (Z1); PCR cycling at 95 C (Z2); PCR cycling at 65 C (Z3) and DETECTR heating at 37 C (Z4). In some embodiments, a sample may be exposed to multiple heat zones within one chamber or channel, thus enabling constant sample fluid flow.

### Electrochemical DETECTR Reaction

The present disclosure provides various devices, systems, fluidic devices, and kits for rapid tests, which may quickly assess whether a target nucleic acid is present in a sample by using a programmable nuclease that can interact with functionalized surfaces of the fluidic systems to generate a detectable signal. For example, disclosed herein are particular microfluidic devices, lateral flow devices, sample preparation devices, and compositions (e.g., programmable nucleases, guide RNAs, reagents for in vitro transcription, amplification, reverse transcription, and reporters, or any combination thereof) for use in said devices that are particularly well suited to carry out a highly efficient, rapid, and accurate reactions for detecting the presence of a target nucleic acid (e.g., a DETECTR reaction). The systems and programmable nucleases disclosed herein can be used as a companion diagnostic with any of the diseases disclosed herein (e.g., RSV, sepsis, flu, COVID-19), or can be used in reagent kits, point-of-care diagnostics, or over-the-counter diagnostics. The systems may be used as a point of care diagnostic or as a lab test for detection of a target nucleic acid and, thereby, detection of a condition, for example, in a subject from which the sample was taken. The systems may be used in various sites or locations, such as in laboratories, in hospitals, in physician offices/laboratories (POLs), in clinics, at remotes sites, or at home. Sometimes, the present disclosure provides various devices, systems, fluidic devices, and kits for consumer genetic use or for over-the-counter use.

### Guide Nucleic Acids

Guide nucleic acids are compatible for use in the devices described herein (e.g., pneumatic valve devices, sliding valve devices, rotating valve devices, and lateral flow devices) and may be used in conjunction with compositions disclosed herein (e.g., programmable nucleases, reagents for in vitro transcription, reagents for amplification, reagents for reverse transcription, and reporters, or any combination thereof) to carry out highly efficient, rapid, and accurate reactions for detecting whether a target nucleic acid is present in a sample (e.g., DETECTR reactions). The guide nucleic acid binds to the single stranded target nucleic acid comprising a portion of a nucleic acid from a virus or a bacterium or other agents responsible for a disease as described herein. The guide nucleic acid can bind to the single stranded target nucleic acid comprising a portion of a nucleic acid from a bacterium or other agents responsible for a disease as described herein and further comprising a mutation, such as a single nucleotide polymorphism (SNP), which can confer resistance to a treatment, such as antibiotic treatment. The guide nucleic acid binds to the single stranded target nucleic acid comprising a portion of a nucleic acid from a cancer gene or gene associated with a genetic disorder as described herein. The guide nucleic acid is complementary to the target nucleic acid. Often the guide nucleic acid binds specifically to the target nucleic acid. The target nucleic acid may be a RNA, DNA, or synthetic nucleic acids. A guide nucleic acid can comprise a sequence that is reverse complementary to the sequence of a target nucleic acid. A guide nucleic acid can be a crRNA. Sometimes, a guide nucleic acid may comprise a crRNA and tracrRNA. The guide nucleic acid can bind specifically to the target nucleic acid. In some cases, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. The target nucleic acid can be designed and made to provide desired functions. In some cases, the targeting region of a guide nucleic acid is 20 nucleotides in length. The targeting region of the guide nucleic acid may have a length of at least 10, 11, 12, 13, 14, 15, 16, 17, 18, 19, 20, 21, 22, 23, 24, 25, 26, 27, 28, 29, or 30 nucleotides in length. In some instances, the targeting region of the guide nucleic acid is 10, 11, 12, 13, 14, 15, 16, 17, 18, 19, 20, 21, 22, 23, 24, 25, 26, 27, 28, 29, or 30 nucleotides in length. In some cases, the targeting region of a guide nucleic acid has a length from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some cases, the targeting region of a guide nucleic acid has a length of from about 10 nt to about 60 nt, from about 20 nt to about 50 nt, or from about 30 nt to about 40 nt. In some cases, the targeting region of a guide nucleic acid has a length of from 15 nt to 55 nt, from 25 nt to 55 nt, from 35 nt to 55 nt, from 45 nt to 55 nt, from 15 nt to 45 nt, from 25 nt to 45 nt, from 35 nt to 45 nt, from 15 nt to 35 nt, from 25 nt to 35 nt, or from 15 nt to 25 nt. It is understood that the sequence of a polynucleotide need not be 100% complementary to that of its target nucleic acid to be specifically hybridizable or hybridizable or bind specifically. The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20 that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid.

The guide nucleic acid can be selected from a group of guide nucleic acids that have been tiled against the nucleic acid of a strain of an infection or genomic locus of interest. The guide nucleic acid can be selected from a group of guide nucleic acids that have been tiled against the nucleic acid of a strain of HPV 16 or HPV 18. Often, guide nucleic acids that are tiled against the nucleic acid of a strain of an infection or genomic locus of interest can be pooled for use in a method described herein. Often, these guide nucleic acids are pooled for detecting a target nucleic acid in a single assay. The pooling of guide nucleic acids that are tiled against a single target nucleic acid can enhance the detection of the target nucleic using the methods described herein. The pooling of guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling may comprise gaps between the tiled guide nucleic acids along the target nucleic acid. In some instances, the tiling of the guide nucleic acids is non-sequential. Often, a method for detecting a target nucleic acid may comprise contacting a target nucleic acid to a pool of guide nucleic acids and a programmable nuclease, wherein a guide nucleic acid of the pool of guide nucleic acids has a sequence selected from a group of tiled guide nucleic acid that is reverse complementary to a sequence of a target nucleic acid; and assaying for a signal produce by cleavage of at least some detector nucleic acids of a population of detector nucleic acids. Pooling of guide nucleic acids can ensure broad spectrum identification, or broad coverage, of a target species within a single reaction. This can be particularly helpful in diseases or indications, like sepsis, that may be caused by multiple organisms.

### Reporter

Reporters, which can be referred to interchangeably reporters, or detector nucleic acids, described herein are compatible for use in the devices described herein (e.g., pneumatic valve devices, sliding valve devices, rotating valve devices, and lateral flow devices) and may be used in conjunction with compositions disclosed herein (e.g., programmable nucleases, guide nucleic acids, reagents for in vitro transcription, reagents for amplification, reagents for reverse transcription, reporters, or any combination thereof) to carry out highly efficient, rapid, and accurate reactions for detecting whether a target nucleic acid is present in a sample (e.g., DETECTR reactions). Described herein is a reporter comprising a single stranded detector nucleic acid comprising a detection moiety, wherein the reporter is capable of being cleaved by the activated programmable nuclease, thereby generating a first detectable signal. As used herein, a detector nucleic acid is used interchangeably with reporter or reporter. In some cases, the detector nucleic acid is a single-stranded nucleic acid comprising deoxyribonucleotides. In other cases, the detector nucleic acid is a single-stranded nucleic acid comprising ribonucleotides. The detector nucleic acid can be a single-stranded nucleic acid comprising at least one deoxyribonucleotide and at least one ribonucleotide. In some cases, the detector nucleic acid is a single-stranded nucleic acid comprising at least one ribonucleotide residue at an internal position that functions as a cleavage site. In some cases, the detector nucleic acid may comprise at least 2, 3, 4, 5, 6, 7, 8, 9, or 10 ribonucleotide residues at an internal position. In some cases, the detector nucleic acid may comprise from 2 to 10, from 3 to 9, from 4 to 8, or from 5 to 7 ribonucleotide residues at an internal position. In some cases, the detector nucleic acid may comprise from 3 to 10, from 4 to 10, from 5 to 10, from 6 to 10, from 7 to 10, from 8 to 10, from 9 to 10, from 2 to 8, from 3 to 8, from 5 to 8, from 6 to 8, from 7 to 8, from 2 to 5, from 3 to 5, or from 4 to 5 ribonucleotide residues at an internal position. Sometimes the ribonucleotide residues are continuous. Alternatively, the ribonucleotide residues are interspersed in between non-ribonucleotide residues. In some cases, the detector nucleic acid has only ribonucleotide residues. In some cases, the detector nucleic acid has only deoxyribonucleotide residues. In some cases, the detector nucleic acid may comprise nucleotides resistant to cleavage by the programmable nuclease described herein. In some cases, the detector nucleic acid may comprise synthetic nucleotides. In some cases, the detector nucleic acid may comprise at least one ribonucleotide residue and at least one non-ribonucleotide residue. In some cases, the detector nucleic acid is 5-20, 5-15, 5-10, 7-20, 7-15, or 7-10 nucleotides in length. In some cases, the detector nucleic acid is from 3 to 20, from 4 to 20, from 5 to 20, from 6 to 20, from 7 to 20, from 8 to 20, from 9 to 20, from 10 to 20, from 15 to 20, from 3 to 15, from 4 to 15, from 5 to 15, from 6 to 15, from 7 to 15, from 8 to 15, from 9 to 15, from 10 to 15, from 3 to 10, from 4 to 10, from 5 to 10, from 6 to 10, from 7 to 10, from 8 to 10, from 9 to 10, from 3 to 8, from 4 to 8, from 5 to 8, from 6 to 8, or from 7 to 8 nucleotides in length. In some cases, the detector nucleic acid may comprise at least one uracil ribonucleotide. In some cases, the detector nucleic acid may comprise at least two uracil ribonucleotides. Sometimes the detector nucleic acid has only uracil ribonucleotides. In some cases, the detector nucleic acid may comprise at least one adenine ribonucleotide. In some cases, the detector nucleic acid may comprise at least two adenine ribonucleotide. In some cases, the detector nucleic acid has only adenine ribonucleotides. In some cases, the detector nucleic acid may comprise at least one cytosine ribonucleotide. In some cases, the detector nucleic acid may comprise at least two cytosine ribonucleotide. In some cases, the detector nucleic acid may comprise at least one guanine ribonucleotide. In some cases, the detector nucleic acid may comprise at least two guanine ribonucleotide. A detector nucleic acid can comprise only unmodified ribonucleotides, only unmodified deoxyribonucleotides, or a combination thereof. In some cases, the detector nucleic acid is from 5 to 12 nucleotides in length. In some cases, the detector nucleic acid is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some cases, the detector nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by a programmable nuclease comprising Cas13, a detector nucleic acid can be 5, 8, or 10 nucleotides in length. For cleavage by a programmable nuclease comprising Cas12, a detector nucleic acid can be 10 nucleotides in length.

### Signals

The devices, systems, fluidic devices, kits, and methods for detecting the presence of a target nucleic acid in a sample described herein may comprise a generation of a signal indicative of the presence or absence of the target nucleic acid in the sample. The generation of a signal indicative of the presence or absence of the target nucleic acid in the sample as described herein is compatible with the methods and devices described herein (e.g., pneumatic valve devices, sliding valve devices, rotating valve devices, and lateral flow devices) and may result from the use of compositions disclosed herein (e.g. programmable nucleases, guide nucleic acids, reagents for in vitro transcription, reagents for amplification, reagents for reverse transcription, reporters, or any combination thereof) to carry out highly efficient, rapid, and accurate reactions for detecting whether a target nucleic acid is present in a sample (e.g., DETECTR reactions). As disclosed herein, in some embodiments, detecting the presence or absence of a target nucleic acid of interest involves measuring a signal emitted from a detection moiety present in a reporter, after cleavage of the reporter by an activated programmable nuclease. Alternatively, or in combination, in some embodiments, detecting the presence or absence of a target nucleic acid of interest involves measuring a signal emitted from a conjugate bound to a detection moiety present in a reporter, after cleavage of the reporter by an activated programmable nuclease. The conjugates may comprise a nanoparticle, a gold nanoparticle, a latex nanoparticle, a quantum dot, a chemiluminescent nanoparticle, a carbon nanoparticle, a selenium nanoparticle, a fluorescent nanoparticle, a liposome, or a dendrimer. The surface of the conjugate may be coated by a conjugate binding molecule that binds to the detection moiety or another affinity molecule of the cleaved detector molecule as described herein. Thus, the detecting steps disclosed herein involve indirectly (e.g., via a reporter) measuring the presence of a target nucleic acid, quantifying how much of the target nucleic acid is present, or, measuring a signal indicating that the target nucleic acid is absent in a sample. In some embodiments, a signal is generated upon cleavage of the detector nucleic acid by the programmable nuclease. In other embodiments, the signal changes upon cleavage of the detector nucleic acid by the programmable nuclease. In other embodiments, a signal may be present in the absence of detector nucleic acid cleavage and disappear upon cleavage of the target nucleic acid by the programmable nuclease. For example, a signal may be produced in a microfluidic device or lateral flow device after contacting a sample with a composition comprising a programmable nuclease.

### Buffers

The reagents described herein can also include buffers, which are compatible with the devices, systems, fluidic devices, kits, and methods disclosed herein. The buffers described herein are compatible for use in the devices described herein (e.g., pneumatic valve devices, sliding valve devices, rotating valve devices, and lateral flow devices) and may be used in conjunction with compositions disclosed herein (e.g., programmable nucleases, guide nucleic acids, reagents for in vitro transcription, reagents for amplification, reagents for reverse transcription, reporters, or any combination thereof) to carry out highly efficient, rapid, and accurate reactions for detecting whether the target nucleic acid is in the sample (e.g., DETECTR reactions). These buffers are compatible with the other reagents, samples, and support mediums as described herein for detection of an ailment, such as a disease, cancer, or genetic disorder, or genetic information, such as for phenotyping, genotyping, or determining ancestry. The methods described herein can also include the use of buffers, which are compatible with the methods disclosed herein. For example, a buffer may comprise HEPES, MES, TCEP, EGTA, Tween 20, KCl, MgCl², glycerol, or any combination thereof. In some instances, a buffer may comprise Tris-HCl pH 8.8, VLB, EGTA, CH3COOH, TCEP, IsoAmp, (NH4)2SO4, KCl, MgSO4, Tween20, KOAc, MgOAc, BSA, TCEP, or any combination thereof. In some instances the buffer may comprise from 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM HEPES pH 6.8. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KCl. In other instances the buffer may comprise 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl². The buffer can comprise 0 to 25, 0 to 20, 0 to 10, 0 to 5, 5 to 10, 5 to 15, 5 to 20, 5 to 25, 5 to 30% glycerol. The buffer can comprise from 0% to 30%, from 5% to 30%, from 10% to 30%, from 15% to 30%, from 20% to 30%, from 25% to 30%, from 0% to 25%, from 2% to 25%, from 5% to 25%, from 10% to 25%, from 15% to 25%, from 20% to 25%, from 0% to 20%, from 5% to 20%, from 10% to 20%, from 15% to 20%, from 0% to 15%, from 5% to 15%, from 10% to 15%, from 0% to 10%, from 5% to 10%, or from 0% to 5% glycerol. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM Tris-HCl pH 8.8. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KOAc. The buffer can comprise to 0 to 500, 0 to 400, 0 to 300, 0 to 250, 0 to 200, 0 to 150, Oto 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM MgOAc. In some instances the buffer may comprise from 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM EGTA. The buffer can comprise from 0% to 30%, from 5% to 30%, from 10% to 30%, from 15% to 30%, from 20% to 30%, from 25% to 30%, from 0% to 25%, from 2% to 25%, from 5% to 25%, from 10% to 25%, from 15% to 25%, from 20% to 25%, from 0% to 20%, from 5% to 20%, from 10% to 20%, from 15% to 20%, from 0% to 15%, from 5% to 15%, from 10% to 15%, from 0% to 10%, from 5% to 10%, or from 0% to 5% Tween 20.

### DETECTR Device Layout

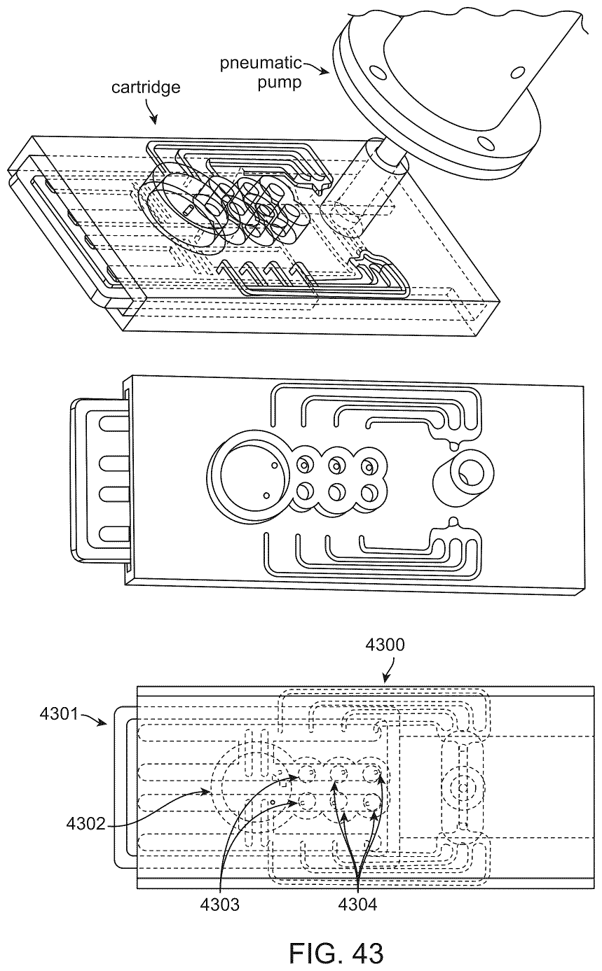

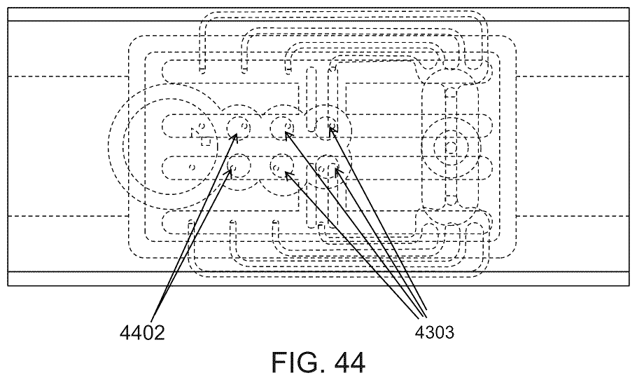

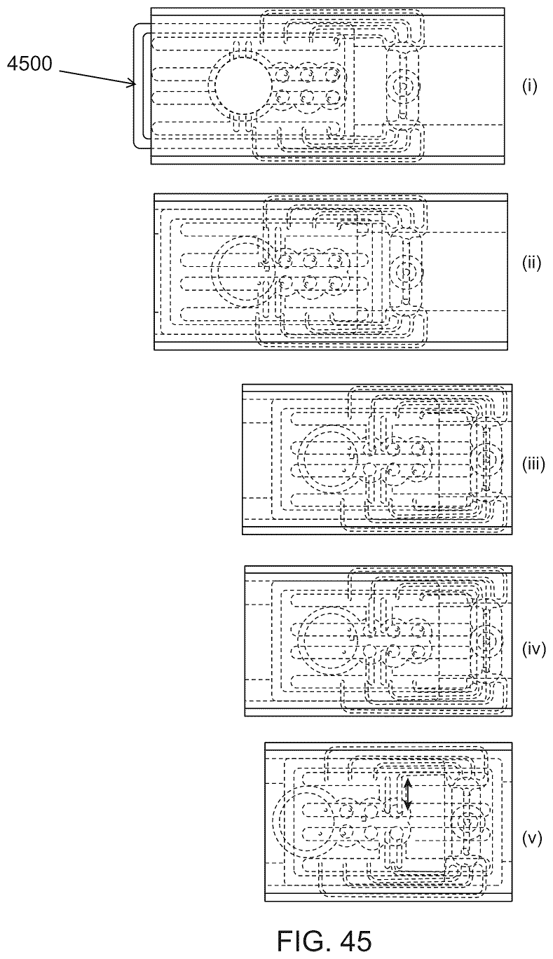

FIG. 43 shows a layout for a device cartridge configured to run a programmable nuclease assay (e.g., a DETECTR assay) as described herein. Shown at top is a pneumatic pump configured to interface with the cartridge. Shown at middle is a top view of the cartridge showing a top layer with reservoirs. Shown at bottom is a sliding valve (4301) containing the sample and arrows pointing to the lysis chamber (4302) at left, followed by amplification chambers (4303) to the right, and detection chambers (4304) further to the right. FIG. 44 shows a schematic of a sliding valve device. The offset pitch of the channels allows aspirating and dispensing into each well separately and helps to mitigate cross talk between the amplification chambers (4402) and corresponding detection chambers (4303). FIG. 45 shows a diagram of sample movement through the sliding valve (4500) device shown in FIG. 44. In the initial closed position (i.), the sample is loaded into the sample well and lysed. The sliding valve (4500) is then actuated by the instrument, and samples are loaded into each of the channels using the pipette pump, which dispenses the appropriate volume into the channel (ii.). The sample is delivered to the amplification chambers by actuating the sliding valve (4500) and mixed with the pipette pump (iii.). Samples from the amplification chamber are aspirated into each channel (iv.) and then dispensed and mixed into each DETECTR chamber (v.) by actuating the sliding valve (4500) and pipette pump. In some embodiments the sliding valve device has a surface area of 5 cm by 8 cm, 5 by 6 cm, 6 by 7 cm, 7 by 8 cm, 8 by 9 cm, 9 by 10 cm, 10 by 11 cm, 11 by 12 cm, 6 by 9 cm, 7 by 10 cm, 8 by 11 cm, 9 by 12 cm, 10 by 13 cm, 11 by 14 cm, 12 by 11 cm, about 30 sq cm, about 35 sq cm, about 40 sq cm, about 45 sq cm, about 50 sq cm, about 55 sq cm, about 60 sq cm, about 65 sq cm, about 70 sq cm, about 75 sq cm, about 25 sq cm, about 20 sq cm, about 15 sq cm, about 10 sq cm, about 5 sq cm, from 1 to 100 sq cm, from 5 to 10 sq cm, from 10 to 15 sq cm, from 15 to 20 sq cm, from 20 to 25 sq cm, from 25 to 30 sq cm, from 30 to 35 sq cm, from 35 to 40 sq cm, from 40 to 45 sq cm, from 45 to 50 sq cm, from 5 to 90 sq cm, from 10 to 0 sq cm, from 15 to 5 sq cm, from 20 to 10 sq cm, or from 25 to 15 sq cm.

In some embodiments, a sliding valve device may comprise a first chamber for sample lysis, a second chamber for detection, and a third chamber for amplification. Another way of referring to these chambers is a sample chamber (e.g., the first chamber), a detection chamber (e.g., the second chamber), and an amplification chamber (e.g., the third chamber). In this layout, the present disclosure provides a device for measuring a signal which may comprise: a sliding layer comprising a channel with an opening at a first end of the channel and an opening at a second end of the channel; and a fixed layer comprising: i) a first chamber having an opening; ii) a second chamber having an opening, wherein the second chamber may comprise a programmable nuclease and a reporter comprising a nucleic acid and a detection moiety; iii) a first side channel having an opening aligned with the opening of the first chamber; and iv) a second side channel having an opening aligned with the opening of the second chamber, wherein the sliding layer and the fixed layer move relative to each other to fluidically connect the first chamber and the first side channel via the opening at the first end of the channel, the opening at the second end of the channel, the opening of the first chamber, and the opening of the first side channel, and wherein the sliding layer and the fixed layer move relative to each other to fluidically connect the second chamber and the second side channel via the opening at the first end of the channel, the opening at the second end of the channel, the opening of the second chamber, and the opening of the second side channel. The fixed layer further may comprise i) a third chamber having an opening; and ii) a third side channel having an opening aligned with the opening of the third chamber, wherein the sliding layer and the fixed layer move relative to each other to fluidically connect the third chamber and the third side channel via the opening at the first end of the channel, the opening at the second end of the channel, the opening of the third chamber, and the opening of the third side channel. The second chamber is coupled to a measurement device for measuring the signal from the detection moiety produced by cleavage of the nucleic acid of the reporter. Additionally, the opening of the first end of the channel overlaps with the opening of the first chamber and the opening of the second end of the channel overlaps with the opening of the first side channel. The opening of the first end of the channel overlaps with the opening of the second chamber and the opening of the second end of the channel overlaps with the opening of the second side channel. The opening of the first end of the channel overlaps with the opening of the third chamber and the opening of the second end of the channel overlaps with the opening of the third channel. Additionally, the first side channel, the second side channel, and the third side channel are fluidically connected to a mixing chamber. In this embodiment, the second chamber additionally includes a guide nucleic acid.

In another embodiment, a sliding valve device may comprise a first chamber for sample lysis and a second chamber for detection. Another way of referring to these chambers is a sample chamber (e.g., the first chamber) and a detection chamber. In this layout, the present disclosure provides a device for measuring a signal may comprise: a sliding layer comprising a channel with an opening at a first end of the channel and an opening at a second end of the channel; and a fixed layer comprising: i) a first chamber having an opening; ii) a second chamber having an opening, wherein the second chamber may comprise a programmable nuclease and a reporter comprising a nucleic acid and a detection moiety; iii) a first side channel having an opening aligned with the opening of the first chamber; and iv) a second side channel having an opening aligned with the opening of the second chamber, wherein the sliding layer and the fixed layer move relative to each other to fluidically connect the first chamber and the first side channel via the opening at the first end of the channel, the opening at the second end of the channel, the opening of the first chamber, and the opening of the first side channel, and wherein the sliding layer and the fixed layer move relative to each other to fluidically connect the second chamber and the second side channel via the opening at the first end of the channel, the opening at the second end of the channel, the opening of the second chamber, and the opening of the second side channel. The second chamber is coupled to a measurement device for measuring the signal from the detection moiety produced by cleavage of the nucleic acid of the reporter. Additionally, the opening of the first end of the channel overlaps with the opening of the first chamber and the opening of the second end of the channel overlaps with the opening of the first side channel. The opening of the first end of the channel overlaps with the opening of the second chamber and the opening of the second end of the channel overlaps with the opening of the second side channel. Additionally, the first side channel and the second side channel are fluidically connected to a mixing chamber. In this embodiment, the second chamber additionally includes a guide nucleic acid.

In some embodiments, the DETECTR assay relies on fluorescence-based detection. In certain embodiments, the DETECTR assay relies on electrochemical-based detection. Electrochemical-based assays have been found to have a lower limit of detection than fluorescence-based assays by roughly two orders of magnitude (Lou et al., 2015).

In some embodiments, electrochemical probes are incorporated into the DETECTR assay to achieve a lower limit of detection. For example, the following electrochemical probe may comprise: 5′-2XXTTATTXX-3′, where 2=5′ 6-FAM; X=ferrocene dT; and 3′=3′ Biotin TEG, where TEG is a 15 atom triethylene glycol spacer. In some embodiments, electrochemical probes are tested with cyclic voltammetry. In some embodiments, electrochemical probes are tested with square wave voltammetry. In some embodiments, a DropSens pSTAT ECL instrument is used for electrochemical measurements. In some embodiments, DropSens screen-printed carbon electrodes are used for electrochemical measurements.

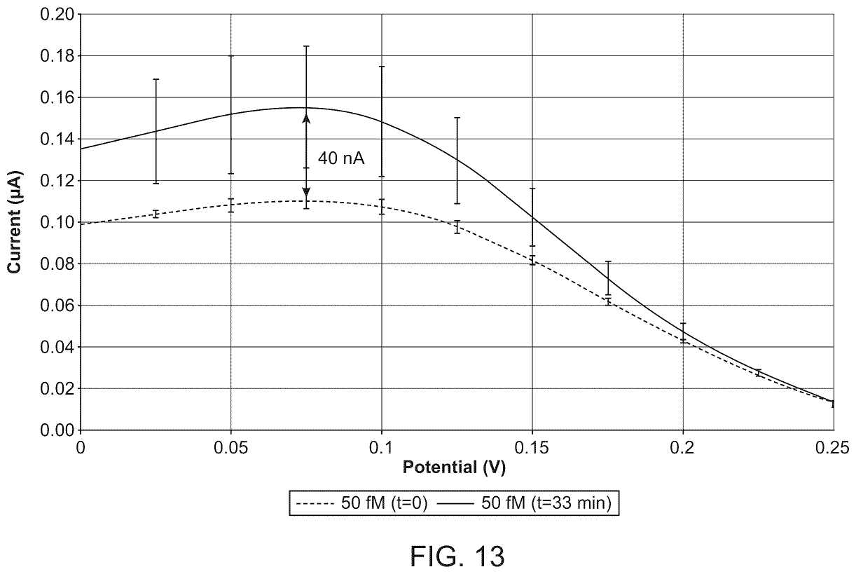

FIG. 13 shows a line graph depicting current as a function of potential. Potential (V) is shown on the x-axis from 0 V to 0.25 V in increments of 0.05 V. Current (μA) is shown on the y-axis from 0 μA to 0.20 μA in increments of 0.02 μA. The graph depicts two lines. The dashed line depicts an oxidation curve for 50 nM HERC2 DETECTR at time=0 of the reaction. The solid line depicts an oxidation curve for 50 nM HERC2 DETECTR at 33 minutes after initiation of the reaction. In this example, error bars represent standard deviation of two measurements of the same solution, using three traces from each measurement. The 40 nA difference in signal indicates detection of the 50 nM HERC2 DETECTR.

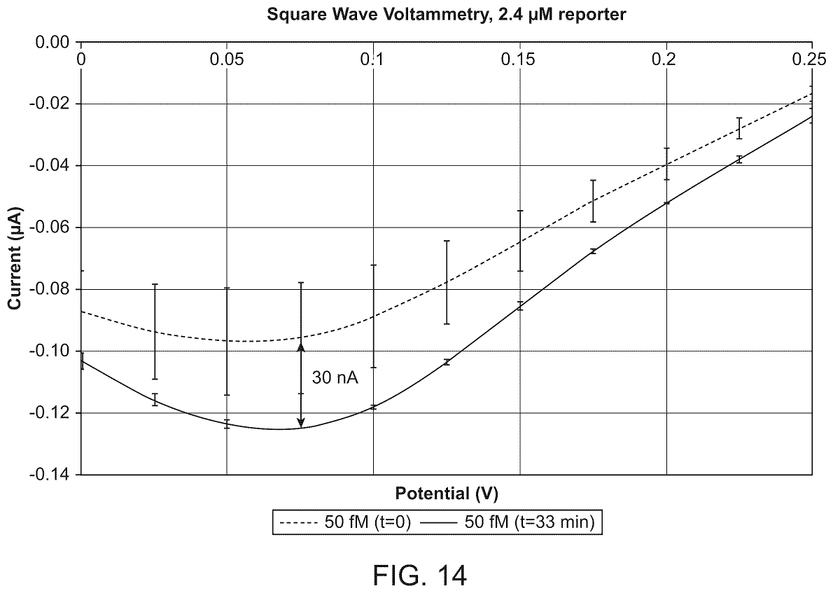

FIG. 14 shows a line graph depicting current as a function of potential. Potential (V) is shown on the x-axis from 0 V to 0.25 V in increments of 0.05 V. Current (μA) is shown on the y-axis from 0 μA to −0.14 μA in increments of 0.02 μA. The graph depicts two lines. The dashed line depicts a reduction curve for HERC2 DETECTR at 50 fM at time=0 for the reaction. The solid line depicts a reduction curve for HERC2 DETECTR at 50 fM at 33 minutes after initiation of the reaction. In this example, error bars represent standard deviation of two measurements of the same solution, using three traces from each measurement.

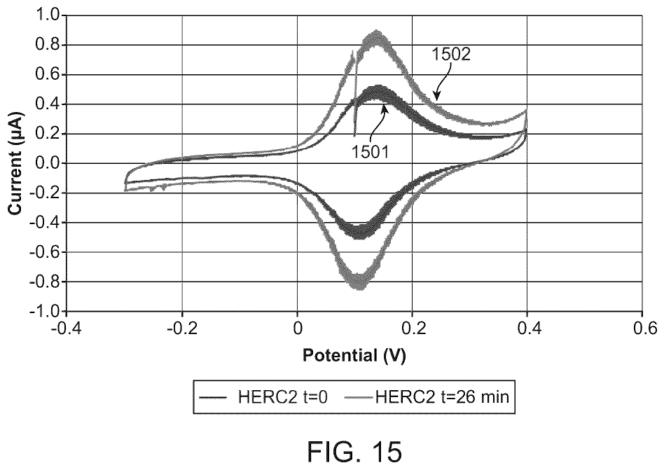

FIG. 15 shows a line graph depicting the current as a function of potential. Potential (V) is shown on the x-axis from −0.4 V to 0.6 V in increments of 0.2 V. Current (μA) is shown on the y-axis from −1 μA to 1 μA in increments of 0.2 μA. The dark green line (1501) and light green line (1502) depict cyclic voltammograms taken before and after HERC2 DETECTR reaction using 24 μM electrochemical reporter, respectively. In this example, each trace is the average of three scans of the same solution and error bars represent standard deviations. The difference in current between the voltammograms indicates detection of HERC2 DETECTR at 24 μM.

### SARS-CoV-2 Electrochemical DETECTR Reaction

In certain embodiments, described herein DETECTR has been demonstrated to be a powerful technology for detection of pathogens such as SARS-CoV-2 (Broughton et al., 2020). In some embodiments, electrochemical probes are utilized in the DETECTR assay for detection of pathogens. In some embodiments, the electrochemical probe-based DETECTR assay is configured for detection of the pathogen SARS-CoV-2. In some embodiments, the electrochemical probe is 5′-2XXTTATTXX-3′, where 2=5′ 6-FAM; X=ferrocene dT; and 3=3′ BiotinTEG.

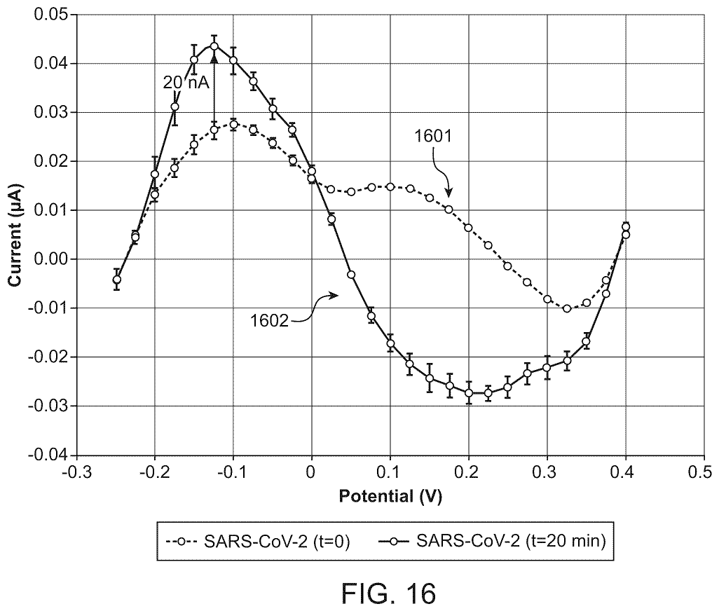

FIG. 16 shows an example line graph depicting current as a function of potential. Potential (V) is shown on the x-axis from −0.3 V to 0.5 V in increments of 0.1 V. Current (μA) is shown on the y-axis from −0.04 μA to 0.05 μA in increments of 0.01 μA. The green line (1601) depicts an oxidation curve for SARS-CoV2 at 0 seconds into the reaction and the yellow line (1602) depicts an oxidation curve for SARS-CoV2 at 20 minutes after initiation of the reaction. In some embodiments, error bars represent standard deviation of three traces from each measurement. The oxidation curve at 20 minutes is observed to be 20 nA higher than the oxidation curve at time 0. The 20 nA difference indicates the presence of SARS-CoV2.

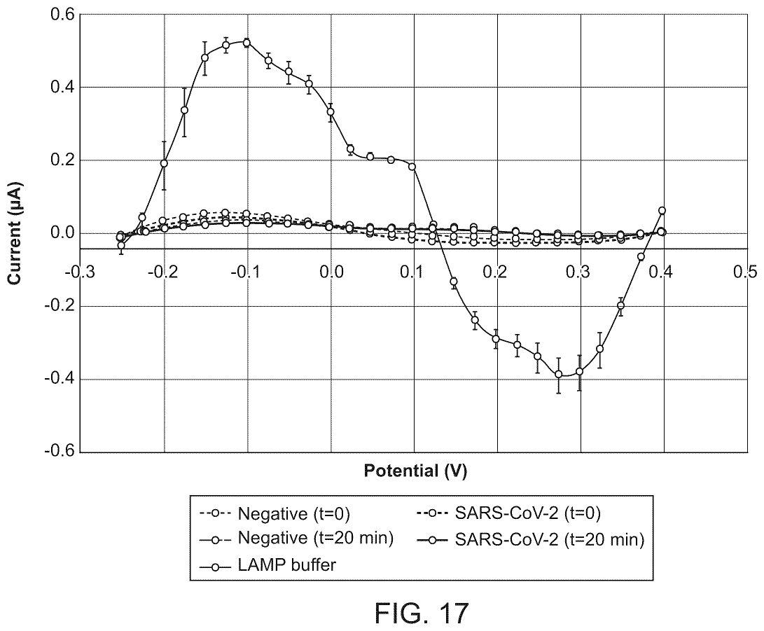

FIG. 17 shows an example line graph depicting current as a function of potential. Potential (V) is shown on the x-axis from −0.3 V to 0.5 V in increments of 0.1 V. Current (μA) is shown on the y-axis from −0.6 μA to 0.6 μA in increments of 0.2 μA. The lines depict square wave voltammetry measurements for SARS-COV-2 DETECTR reaction with electrochemical reporters and controls.

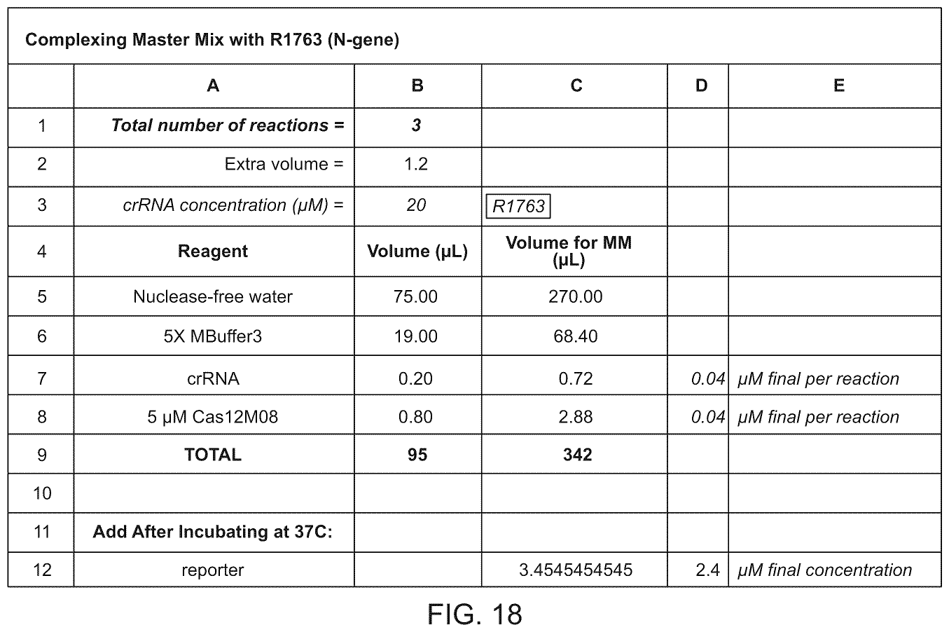

FIG. 18 shows an example of a complexing master mix with R1763 (N-gene).

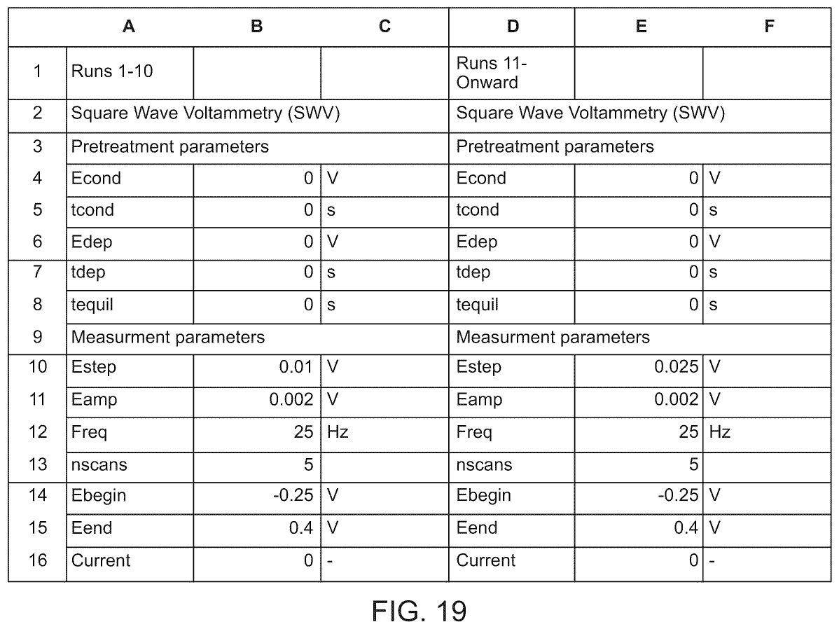

FIG. 19 presents an example of experimental conditions described herein, for square wave voltammetry.

### DETECTR Assay Immobilization

CRISPR diagnostic reactions are generally performed in solution where the Cas protein-RNA complexes can freely bind target molecules and reporters. However, reactions where all components are in solution limit the designs of CRISPR diagnostic assays, especially in microfluidic devices. A system where various components of the CRISPR diagnostic reaction are immobilized on a surface enables designs where multiple readouts can be accomplished within a single reaction chamber.

Described herein are various methods to immobilize CRISPR diagnostic reaction components to the surface of a reaction chamber or other surface (e.g., a surface of a bead). Any of the devices described herein may comprise one or more immobilized detection reagent components (e.g., programmable nuclease, guide nucleic acid, and/or reporter). In certain instances, methods include immobilization of programmable nucleases (e.g., Cas proteins or Cas enzymes), reporters, and guide nucleic acids (e.g., gRNAs). In some embodiments, various CRISPR diagnostic reaction components are modified with biotin. In some embodiments, these biotinylated CRISPR diagnostic reaction components are tested for immobilization on surfaces coated with streptavidin. In some embodiments, the biotin-streptavidin interaction is used as a model system for other immobilization chemistries.

TABLE 1
presents gRNA and reporter immobilization sequences

| | | | SEQ ID |
|---|---|---|---|
| Name | Sequence | Description | NO: |
| R003 | rGrGrCrCrArCrCrCrCrArArArArArUrGrArAr | unmodified Cas13 crRNA | 3 |
| | GrGrGrGrArCrUrArArArArCrArGrUrGrArUr | | |
| | ArArGrUrGrGrArArUrGrCrCrArUrG | | |
| mod023 | /5BiotinTEG/rGrGrCrCrArCrCrCrCrArArArAr | biotin modified Cas13 | 4 |
| | ArUrGrArArGrGrGrGrArCrUrArArArArCrAr | crRNA | |
| | CrGrArCrCrUrArCrUrCrUrCrCrCrArUrArCrU | | |
| | rC | | |
| R1763 | rUrArArUrUrUrCrUrArCrUrArArGrUrGrUrAr | Unmodified gRNA | 5 |
| | GrArUrCrCrCrCrCrArGrCrGrCrUrUrCrArGrC | targeting a sequence in | |
| | rGrUrUrC | SARS-CoV-2 | |
| mod011 | /5Biosg/rUrA rArUrU rUrCrU rArCrUrArArG | R1763 with a 5′ biotin | 6 |
| | rUrGrU rArGrArUrCrCrCrCrCrArGrCrGrCrU | | |
| | rUrCrA rGrCrG rUrUrC | | |
| mod012 | /5Biosg/T*T*T*T*T*rUrArArUrUrUrCrUrAr | R1763 with 5 | 7 |
| | CrUrArArGrUrGrUrArGrArUrCrCrCrCrCrAr | phosphorothioated | |
| | GrCrGrCrUrUrCrArGrCrGrUrUrC | nucleotides on 5′ end | |
| mod013 | /5Biosg/T*T*T*T*T*T*T*T*T*T*rUrArArUr | R1763 with 10 | 8 |
| | UrUrCrUrArCrUrArArGrUrGrUrArGrArUrCr | phosphorothioated | |
| | CrCrCrCrArGrCrGrCrUrUrCrArGrCrGrUrUrC | nucleotides on 5′ end | |
| mod014 | rUrArArUrUrUrCrUrA/iBiodUK/rCrUrArArGr | R1763 with an internal | 9 |
| | UrGrUrArGrArUrCrCrCrCrCrArGrCrGrCrUr | biotin modification | |
| | UrCrArGrCrGrUrUrC | | |
| mod015 | rUrArArUrUrUrCrUrArCrUrArArGrUrGrUrAr | R1763 with an internal | 10 |
| | GrArUrC/iBiodUK/rCrCrCrCrArGrCrGrCrUr | biotin modification | |
| | UrCrArGrCrGrUrUrC | | |
| mod016 | rUrArArUrUrUrC/iBiodUK/rArCrUrArArGrUr | R1763 with an internal | 11 |
| | GrUrArGrArUrCrCrCrCrCrArGrCrGrCrUrUr | biotin modification | |
| | CrArGrCrGrUrUrC | | |
| mod017 | rUrArArUrUrUrCrUrArCrUrArArGrUrGrUrAr | R1763 with an internal | 12 |
| | GrA/iBiodUK/rCrCrCrCrCrArGrCrGrCrUrUr | biotin modification | |
| | CrArGrCrGrUrUrC | | |
| mod018 | /5BiotinTEG/rUrArArUrUrUrCrUrArCrUrArA | R1763 with a 5′ biotin- | 13 |
| | rGrUrGrUrArGrArUrCrCrCrCrCrArGrCrGrCr | TEG modification | |
| | UrUrCrArGrCrGrUrUrC | | |
| mod019 | rUrA rArUrU rUrCrU rArCrU rArArGrUrGrU | R1763 with a 3′ biotin | 14 |
| | rArGrA rUrCrC rCrCrC rArGrCrGrCrU | modification | |
| | rUrCrA rGrCrG rUrUrC/3Bio/ | | |
| mod020 | rUrA rArUrU rUrCrU rArCrU rArArGrUrGrU | R1763 with a 3′ biotin- | 15 |
| | rArGrA rUrCrC rCrCrCrArGrCrGrCrU | TEG modification | |
| | rUrCrA rGrCrGrUrUrC/3BioTEG/ | | |
| rep072 | /56-FAM/TTATTATT/3Bio/ | FAM reporter with 3′ | 16 |
| | | biotin | |
| rep103 | /5Alex488N/TT ATT ATT /3Bio/ | 5′ modified with | 17 |
| | | Alexa488 reporter and 3′ | |
| | | modified with biotin | |
| rep104 | /5 Alex488N/TT ATT ATT | 5′ modified with | 18 |
| | AT/iBiodT/ATT/3IABKFQ/ | Alexa488 reporter with | |
| | | internal biotin and 3′ | |
| | | modified with a quencher | |
| rep105 | /5BiotinTEG/*T*T* /16-FAMK/*TATTA TTA | 5′ modified with biotin- | 19 |
| | TTA TTA TT/3IABKFQ/ | TEG followed by two | |
| | | phosphorothioated | |
| | | nucleotides (*) should not | |
| | | be cleavable and an | |
| | | internal FAM and 3′ | |
| | | modified with a quencher | |
| | | *T* could also be some | |
| | | other non-cleavable su | |
| rep106 | /56-FAM/TT ATT ATT ATT A/3Dig_N/ | 5′ FAM reporter with a 3′ | 20 |
| | | DIG | |
| rep115 | [biotinTEG]TTTTTTTTTTTTTTTTTTTTTTT | biotinTEG modified 5′ | 21 |
| | TTTTTTT[Phycoerythrin] | and Phycoetrythrin | |
| | | modified 3′, where 5′ and | |
| | | 3′ linked by T30 (SEQ ID | |
| | | NO: 70) | |
| rep116 | [FAM]n[branch]TTTTTTTTTTTTTTTTTTTT | 5′ modified with 9 FAMs | 22 |
| | [biotin TEG] | and 3′ modified with | |
| | | biotinTEG, where 5′ and | |
| | | 3′ linked by T20 (SEQ ID | |
| | | NO: 69) | |
| rep117 | [BiotinTEG]*T*T*[internalFAM]* | 5′ modified with | 23 |
| | TTTTTTTTTTTTTTTTTTTT[IABKFQ] | BiotinTEG and 3′ | |
| | | modified with FQ, where | |
| | | 5′ and 3′ linked by T20 | |
| | | (SEQ ID NO: 69) | |
| rep118 | [FAM]TTTTTTTTTTTTTTTTTTTT* | 5′ modified with FAM | 24 |
| | [internalBiotin]*T[IABKFQ] | and 3′ modified with | |
| | | Biotin-FQ, where 5′ and | |
| | | 3′ are linked by T20 (SEQ | |
| | | ID NO: 69) | |
| rep119 | [5BiotinTEG]*T*T*[internalCy5]* | 5′ modified with | 25 |
| | TTTTTTTTTTTTTTTTTTTT[RQ] | BiotinTEG and 3′ | |
| | | modified with BHQ-2, | |
| | | contains internal Cy5 dye | |
| | | linked to quencher by T20 | |
| | | (SEQ ID NO: 69) | |
| rep121 | /5BiotinTEG//iSp18//iCy5/ TTT TTT | 5′ modified with | 26 |
| | TTT TTT | BiotinTEG, an internal 18 | |
| | TTTTTT TT/3IAbRQSp/ | atom spacer, and an | |
| | | internal Cy5 followed by | |
| | | T20 (SEQ ID NO: 69) and | |
| | | a IAbRQSp quencher | |
| rep120 | [Cy5]TTTTTTTTTTTTTTTTTTTT* | 5′ modified with Cy5 | 27 |
| | [internalBiotin]*T[RQ] | followed by T20 (SEQ ID | |
| | | NO: 69) linker to an | |
| | | internal biotin and an RQ | |
| | | quencher. * indicates | |
| | | phosphorothioated | |
| | | nucleotides | |
| rep125 | /5 Alex647N//iBiodT/TTT TTT | 5′ modified with | 28 |
| | TTT TTT TTT | Alexa647 followed by an | |
| | TTT TT/3IAbRQSp/ | internal biotindT T20 | |
| | | (SEQ ID NO: 69) and a 3′ | |
| | | IAbRQSp quencher | |
| rep126 | /5BiotinTEG/TT/16- | 5′ biotinTEG modified | 29 |
| | FAMK/TTTTTTTTTrUrUrUrUrUTTTTTT | followed by two T, an | |
| | /3IABKFQ/ | internal FAM and, 10T | |
| | | (SEQ ID NO: 74), 5 RNA | |
| | | U, 6T, and a quencher. | |
| | | Acts as an RNA cleavage | |
| | | reporter. | |
| rep110 | /5AmMC6T/TTTTTTTTTTTT/3AlexF488N/ | 5′ amino with 6 carbons | 30 |
| | | followed by T12 (SEQ ID | |
| | | NO: 67) and a Alexa488 | |
| | | modification on 3′ end | |
| rep111 | /5AmMC6T//16- | 5′ amino with 6 carbons | 31 |
| | FAMK/TTTTTTTTTTTT/3IABKFQ/ | followed by internal | |
| | | FAM, T12 (SEQ ID NO: | |
| | | 67), and a quencher on 3′ | |
| rep112 | /5AmMC6T/TT TTT TTT TTT T/36-FAM/ | 5′ amino with 6 carbons | 32 |
| | | followed by T12 (SEQ ID | |
| | | NO: 67) and a 3′ FAM | |
| | | modification | |
| rep122 | /5AmMC12//iSp18//iCy5/ | 5′ amino with 12 carbons | 33 |
| | TTT TTT TTT TTT | followed by 18 atom | |
| | TTTTTT TT/3IAbRQSp/ | linker followed by Cy5 | |
| | | followed by T20 (SEQ ID | |
| | | NO: 69) and a 3′ quencher | |
| rep123 | /5AmMC12//iCy5/ TTTTTT TTTTTT TTT | 5′ amino with 12 carbons | 34 |
| | TTT TT/3IAbRQSp/ | followed by internal Cy5 | |
| | | and a T20 (SEQ ID NO: | |
| | | 69) linker to a 3′ quencher | |
| rep135 | /5AmMC12//16- | 5′ amino with 12 carbons | 35 |
| | FAMK/TTTTTTTTTTTTTTTTTTT/3IABKFQ/ | followed by internal FAM | |
| | | and a T20 (SEQ ID NO: | |
| | | 69) linker to a 3′ quencher | |
| rep136 | /5AmMC6//16- | 5′ amino with 6 carbons | 36 |
| | FAMK/TTTTTTTTTTTTTTTTTTT/3IABKFQ/ | followed by internal FAM | |
| | | and a T20 (SEQ ID NO: | |
| | | 69) linker to a 3′ quencher | |
| mod026 | /5AmMC6/rUrArArUrUrUrCrUrArCrUrArAr | 5′ amino with 6 carbons | 37 |
| | GrUrGrUrArGrArUrCrCrCrCrCrArGrCrGrCr | linked to gRNA for | |
| | UrUrCrArGrCrGrUrUrC | SARS-CoV-2 N-gene | |
| mod027 | /5AmMC12/rUrArArUrUrUrCrUrArCrUrArAr | 5′ amino with 12 carbons | 38 |
| | GrUrGrUrArGrArUrCrCrCrCrCrArGrCrGrCr | linked to gRNA for | |
| | UrUrCrArGrCrGrUrUrC | SARS-CoV-2 N-gene | |
| mod028 | /5AmMC6T/rUrArArUrUrUrCrUrArCrUrArAr | 5′ amino with 6 carbons | 39 |
| | GrUrGrUrArGrArUrCrCrCrCrCrArGrCrGrCr | on dT linked to gRNA for | |
| | UrUrCrArGrCrGrUrUrC | SARS-CoV-2 N-gene | |
| mod029 | rUrArArUrUrUrCrUrArCrUrArArGrUrGrUrAr | gRNA for SARS-CoV-2 | 40 |
| | GrArUrCrCrCrCrCrArGrCrGrCrUrUrCrArGrC | N-gene with 5′ amino | |
| | rGrUrUrC/3AmMC6T/ | with 6 carbons on 3′ end | |
| mod030 | /5AmMC12/rUrArArUrUrUrCrUrArCrUrArAr | 5′ amino with 12 carbons | 41 |
| | GrUrGrUrArGrArUrUrUrArCrArUrGrGrCrUr | linked to gRNA for | |
| | CrUrGrGrUrCrCrGrArG | human RNase P POP7 | |
| mod031 | /5AmMC12/rUrArArUrUrUrCrUrArCrUrArAr | 5′ amino with 12 carbons | 42 |
| | GrUrGrUrArGrArUrGrCrCrGrArUrArArUrGr | linked to gRNA for | |
| | ArUrGrUrArGrGrGrArU | Mammuthus primigenius | |
| | | control sequence | |
| mod059 | /5ThioMC6-D/rUrA rArUrU rUrCrU rArCrU | 5′ thiol modification with | 43 |
| | rArArG rUrGrU rArGrA rUrCrC rCrCrC | 6 carbons linked to gRNA | |
| | rArGrC rGrCrU rUrCrA rGrCrG rUrUrC | for SARS-CoV-2 N-gene | |
| rep130 | /5ThioMC6-D/*T*T*/16- | 5′ modified with | 44 |
| | FAMK/*TTTTTTTTTTTTTTTTTTTT/3IABkFQ | BiotinTEG and 3′ | |
| | | modified with FQ, where | |
| | | 5′ and 3′ linked by T20 | |
| | | (SEQ ID NO: 69); two | |
| | | phosphorothioated | |
| | | nucleotides between thiol | |
| | | and internal FAM | |
| mod024 | /5BiotinTEG/rUrArArUrUrUrCrUrArCrUrArA | 5′ modified with | 45 |
| | rGrUrGrUrArGrArUrGrCrCrGrArUrArArUrGr | BiotinTEG linked to a | |
| | ArUrGrUrArGrGrGrArU | gRNA that targets | |
| | | Mammuthus primigenius | |
| | | sequence | |
| mod025 | /5BiotinTEG/rUrArArUrUrUrCrUrArCrUrArA | 5′ modified with | 46 |
| | rGrUrGrUrArGrArUrUrUrArCrArUrGrGrCrUr | BiotinTEG linked to a | |
| | CrUrGrGrUrCrCrGrArG | gRNA that targets human | |
| | | RNase P POP7 | |
| mod058 | /5biotinTEG/rUrArArUrUrUrCrUrArCrUrArA | 5′ modified with | 47 |
| | rGrUrGrUrArGrArUrCrUrGrCrCrArArUrUrGr | BiotinTEG linked to a | |
| | CrArGrGrArArUrGrArU | gRNA that targets | |
| | | Mammuthus primigenius | |
| | | sequence | |

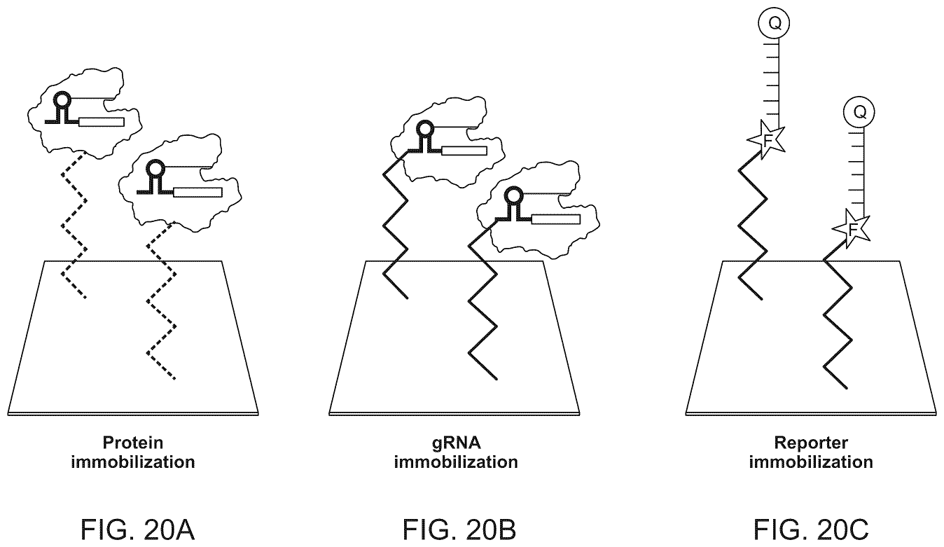

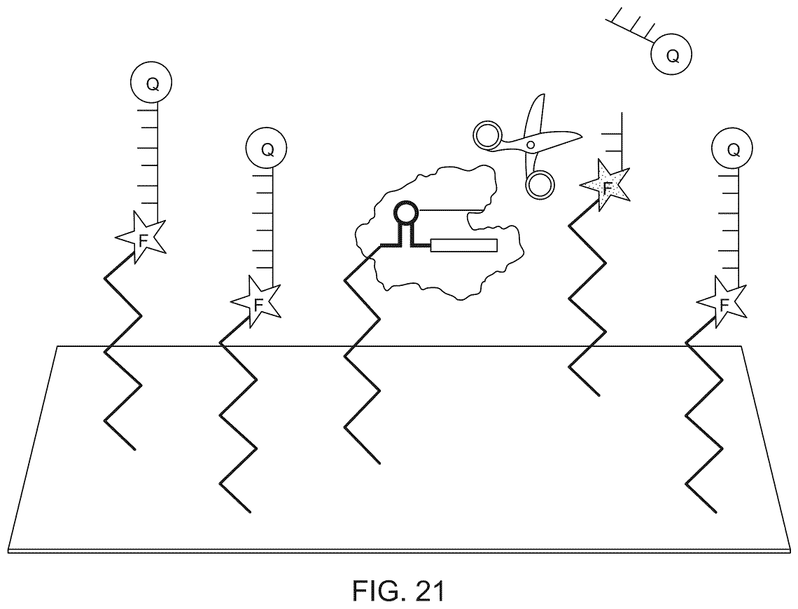

FIGS. 20A-20C illustrate three exemplary immobilization strategies for CRISPR-Cas diagnostic assay components. In some embodiments, as seen in FIG. 20A, chemical modifications of amino acid residues in the Cas protein enable attachment to a surface. In some embodiments, as seen in FIG. 20B, gRNAs are immobilized by adding various chemical modifications at the 5′ or 3′ end of the gRNA that are compatible with a selected surface chemistry. In some embodiments, as seen in FIG. 20C fluorescence-quenching (FQ), or other reporter chemistries, are attached to surfaces using similar chemical modifications as gRNAs. In some embodiments, these attached reporters are activated by a Cas protein, which leads to either activated molecules that remain attached to the surface or activated molecules that are released into solution.

For some embodiments, described herein, FIG. 21 provides an illustrative example of immobilization strategies for use with methods and compositions described herein where the RNP complex is immobilized by a gRNA and cleaves surrounding FQ reporters that are also immobilized to a surface. Here, the quencher is released into solution, leaving a localized fluorescent signal.

In some embodiments, the programmable nuclease, guide nucleic acid, or the reporter are immobilized to a device surface by a linkage or linker. In some embodiments, the linkage comprises a covalent bond, a non-covalent bond, an electrostatic bond, a bond between streptavidin and biotin, an amide bond or any combination thereof. In some embodiments, the linkage comprises non-specific absorption. In some embodiments, the programmable nuclease is immobilized to the device surface by the linkage, wherein the linkage is between the programmable nuclease and the surface. In some embodiments, the reporter is immobilized to the device surface by the linkage, wherein the linkage is between the reporter and the surface. In some embodiments, the guide nucleic acid is immobilized to the surface by the linkage, wherein the linkage is between the 5′ end of the guide nucleic acid and the surface. In some embodiments, the guide nucleic acid is immobilized to the surface by the linkage, wherein the linkage is between the 3′ end of the guide nucleic acid and the surface.

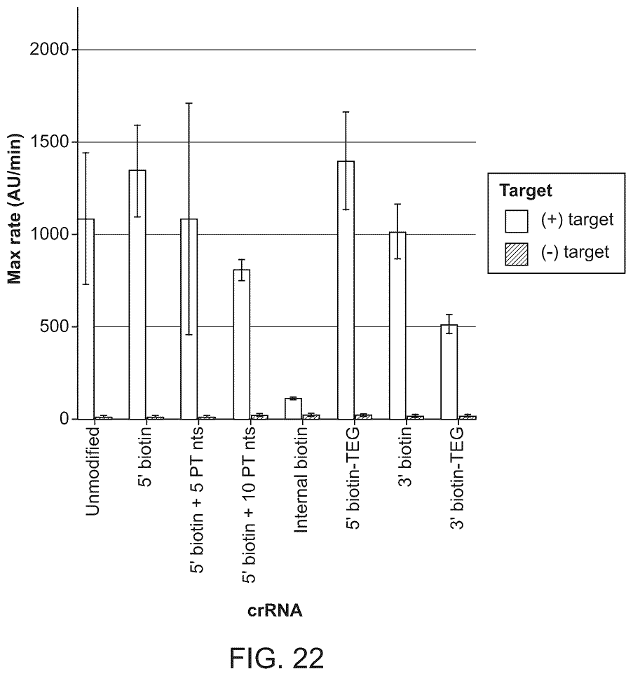

In some embodiments, various chemical modifications to gRNAs are described as shown in FIG. 22. The y-axis shows reaction rate in terms of fluorescence intensity over time and the x-axis presents various modifications of crRNA. In some embodiments, unmodified biotin and variations of biotin modifications are placed at various positions along a Cas12M08 gRNA. The modified gRNAs are then complexed with the protein and dsDNA target is added. In some embodiments, higher average fluorescence over the same period of time indicates that modifications are tolerated on the 5′ and 3′ ends of the gRNA, but not internally in the gRNA. 5′ modified gRNAs appear to be more robust than 3′ modifications.

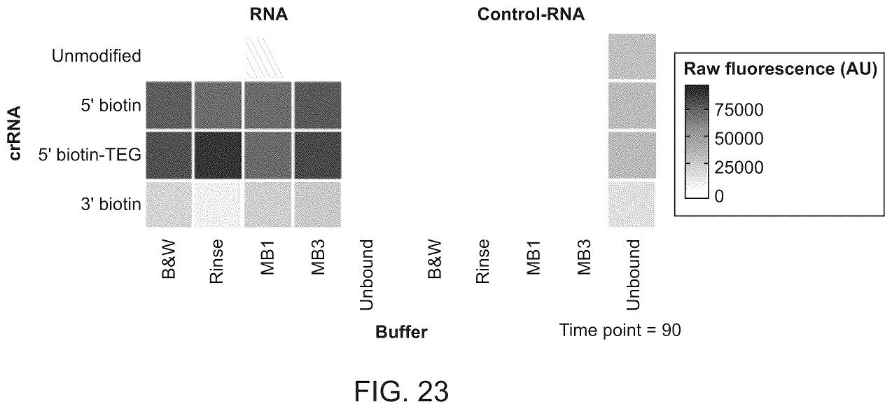

In some embodiments, the immobilization of gRNAs to a streptavidin surface are described as shown in FIG. 23. Two plots are shown, where the left-hand plot depicts RNA bound to a streptavidin coated surface and the right-hand plot depicts unbound control-RNA, in solution, mixed with target, reporter and protein solution. In each table, the y-axis depicts, in some embodiments, various modifications of crRNA and the x-axis depicts various buffer conditions that the crRNA is subjected to. The left-hand, experimental plot shows gRNA on 5′ or 3′ side are both functional approaches, but 5′ biotin modified gRNAs show increased signal in comparison to 3′ modified gRNAs. In some embodiments, unmodified gRNAs show no signal when bound to the plate. Conversely, the right-hand control plot depicts an unbound control where, in some embodiments, free gRNA is mixed with target, reporter, and protein solution. In this embodiment, sufficient signal is observed, indicating functionality in unmodified gRNAs.

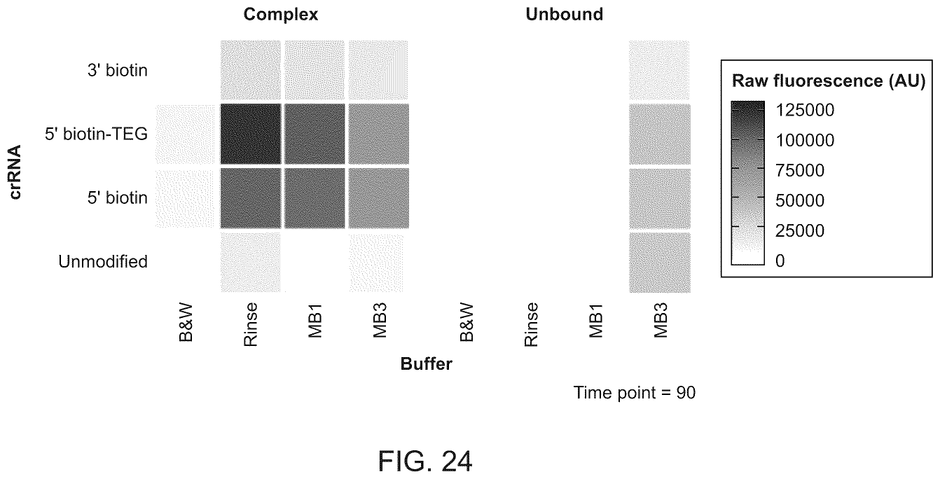

In some embodiments, Cas proteins are complexed with RNA complexes as described herein and shown in FIG. 5. FIG. 24 shows RNP complexes bound by 5′ biotin modified gRNAs exhibit higher signal, indicating functional attachment to the surface of the streptavidin coated plate. Samples exposed to unbound, high salt “B&W” buffer conditions show less fluorescence signal indicating inhibited protein activity or disruption of binding of functional RNP complexes to the surface of the plate. Unmodified gRNAs also exhibit lower fluorescence indicating failed binding to the RNP to the plate surface. Control assays, shown in the right-hand plot, indicate unbound gRNAs are still functional.

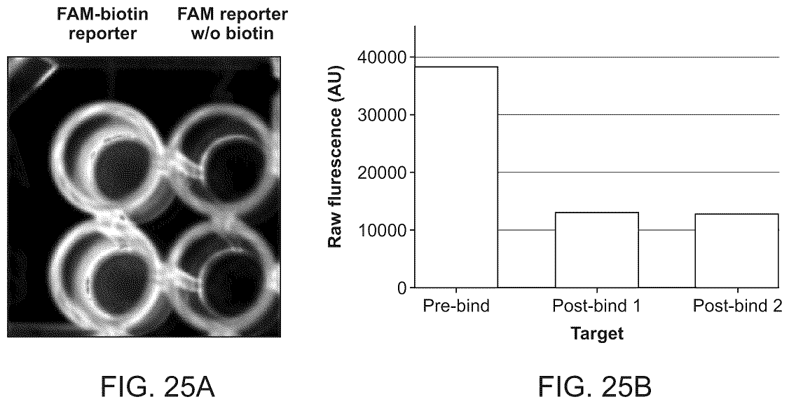

In some embodiments, reporters are immobilized to the surface as shown in FIGS. 25A-25B. FIG. 25A shows a fluorescence image of four wells with streptavidin coated surfaces, where the left-hand column of wells contains FAM-biotin reporters immobilized to a streptavidin coated surface. The right-hand column of wells contains FAM reporter without biotin functionalization. The left-hand column exhibits a higher signal. FIG. 25B shows a comparison of fluorescence intensity of the FAM-biotin pre-binding solution to the solution after incubating on the streptavidin plate. A decrease in signal for both wells containing FAM-biotin is observed.

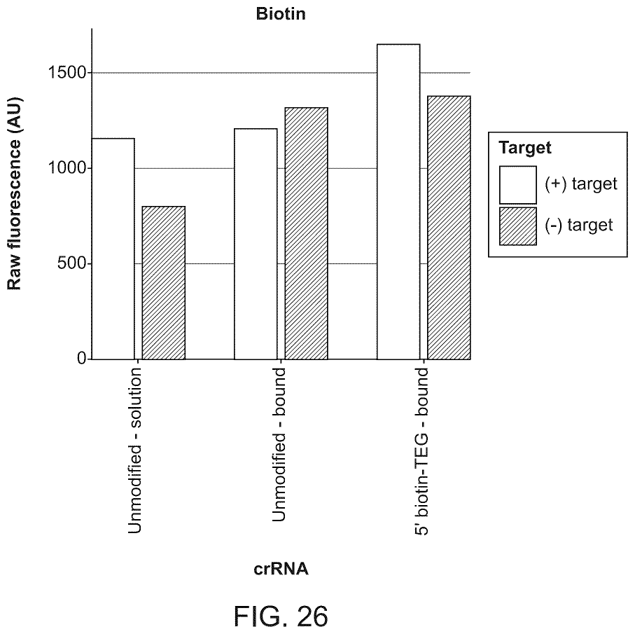

In some embodiments, combined RNP and a reporter system are immobilized for functional testing as shown in FIG. 26. Raw fluorescence (AU) is plotted against three conditions: (1) unmodified crRNA in solution, (2) unmodified crRNA bound to the surface and (3) 5′ biotin-TEG modified crRNA bound to the surface. The combined binding of the reporter and RNP to the plate shows a similar signal to RNP in solution with bound reporter.

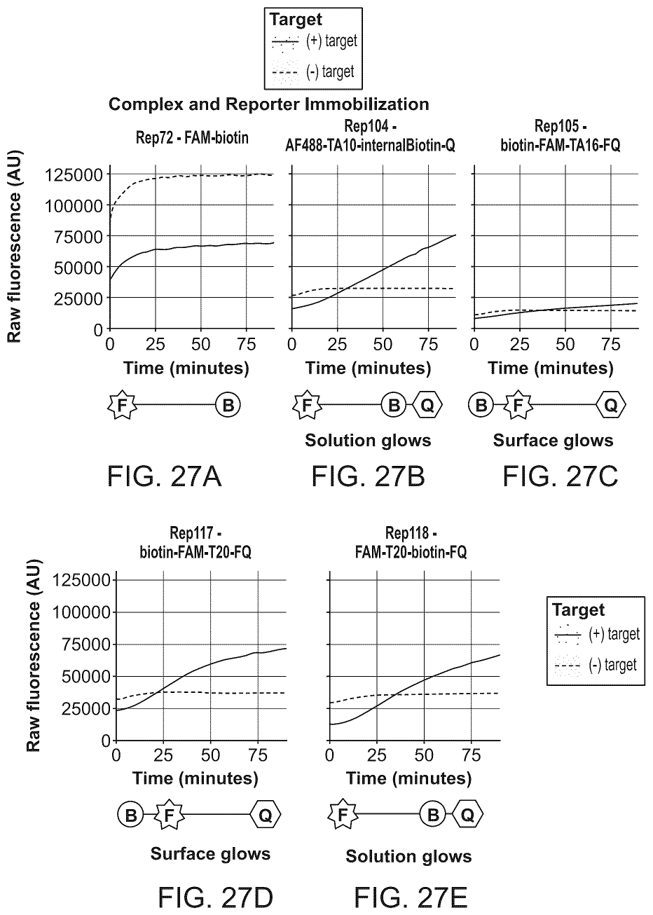

In some embodiments, different reporters are immobilized in combination with Cas complexes on a streptavidin surface for evaluation of the DETECTR assay. FIGS. 27A-27E present results for evaluation of different reporters for immobilization in combination with Cas complex immobilization on a streptavidin surface. In each figure, raw fluorescence is plotted against time in minutes representing kinetic binding curves for each type of reporter while binding with a positive control (+) and negative control (−) target. FIG. 27A presents the binding results for a FAM-biotin reporter, “rep” composed of the fluorophore FAM and biotin and is listed as rep72. The FIG. 27B plots the raw fluorescence for a reporter composed of the fluorophore AlexaFluor488, “AF488,” and TA10-internalBiotinQ. As predicted the positive control shows a positive slope indicating increased binding over the course of the reaction. This is due to the release of FAM dye into solution upon binding and transcleavage. In rep104 the cleavage point is between the FAM and the biotin, while the biotin in all reporters is the attachment point to the streptavidin surface. FIG. 27C plots the control, target binding kinetic plot for rep105. Rep105 is composed of biotin-FAM-T16-FQ (SEQ ID NO: 68). In this case the streptavidin coated surface emits fluorescence because the region between the FAM dye and the quencher is cleaved upon binding and the quencher is released. FIG. 27D plots the control for rep117. Rep117 is composed of biotin-FAM-T20-FQ (SEQ ID NO: 71). In this embodiment, the reporter is cleaved between the FAM dye and the quencher, thus allowing for release of the quencher in the solution upon binding and transcleavage. This in turn, causes the surface to emit fluorescence. FIG. 27E plots the control for rep118. Rep118 is composed of FAM-T20-biotin-FQ (SEQ ID NO: 72). In this embodiment, the solution emits fluorescence because upon binding the nucleic acid region between the biotin and the FAM is transcleaved, thus releasing the FAM into solution.

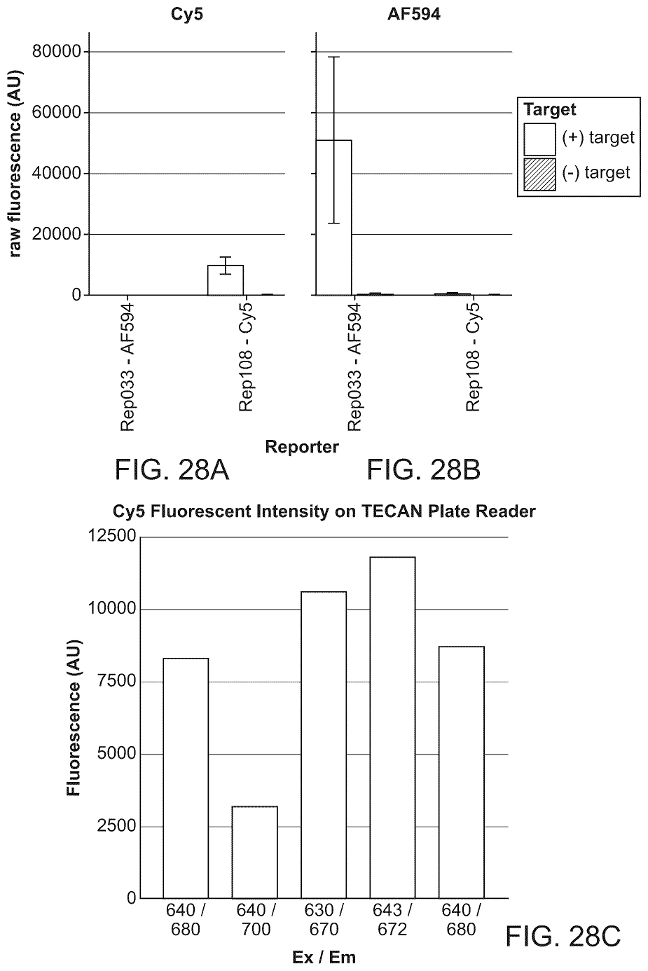

In some embodiments, Cy5 dye may be used as a reporter or a component of a reporter. FIGS. 28A-28C present results for the Cy5 reporter (rep108) showing that it is functional for DETECTR but produces a weaker signal (might be gain related). FIGS. 28A and 28B plot raw fluorescence versus reporter type for channels configured to read Cy5 dye and Alexa Fluor 594 “AF594” dye, respectively. In these plots the average raw fluorescence is shown for each reporter. Reporter, rep033, readout in the “AF594” channel had the most significant fluorescence signal. FIG. 28C plots raw fluorescence for various combinations of excitation and emission wavelengths on a plate reader for Cy5 dye. Under similar assay conditions AF594 exhibits stronger signal than Cy5, but Cy5 is functional. Optimum excitation and emission wavelengths for Cy5 are shown to be 643 nm and 672 nm, respectively.

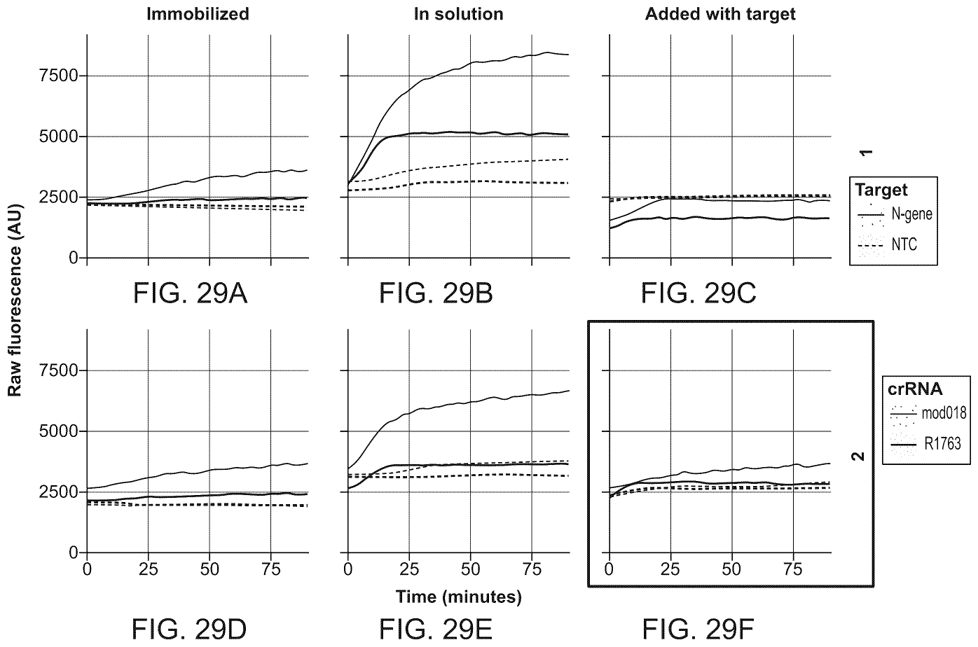

FIGS. 29A-29F present results for optimization of the complex formation step where certain components are immobilized, as described herein. In each figure raw fluorescence is plotted against time in minutes. FIGS. 29A-29C show results for replicate 1. FIGS. 29D-29F show results for replicate 2. In FIGS. 29A and 29D the reporter and gRNA are immobilized. In FIGS. 29B and 29E all components are in solution. In FIGS. 29C and 29F the reporter and gRNA are immobilized and Cas12 and target are added at the same time.

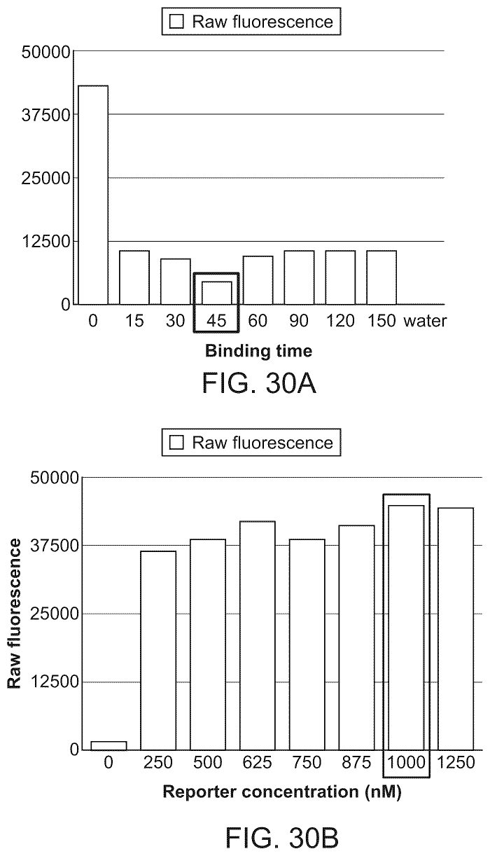

FIGS. 30A-30B present results for immobilization optimization involving a gRNA/reporter binding time and reporter concentration. FIG. 30A is a measurement of supernatant of the surface reaction over time showing the fluorescence dropping and thus indicating uptake of biotinylated dye reporter by the streptavidin surface. In this embodiment a 15 min binding time is found to be sufficient.

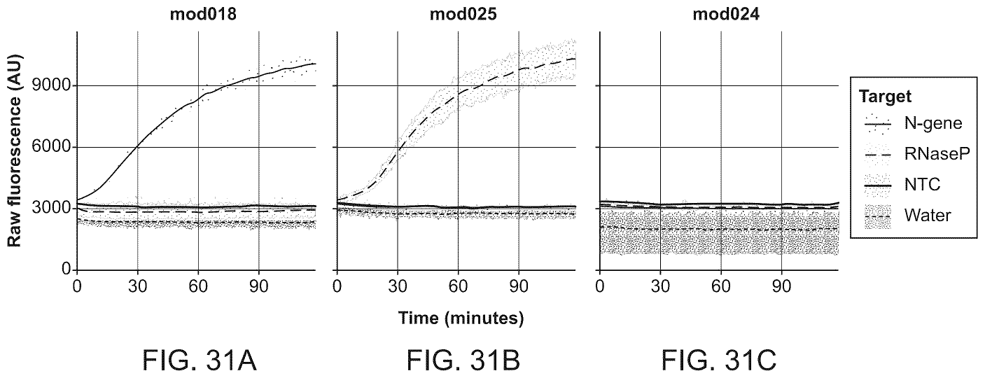

In some embodiments, gRNAs are modified. In some embodiments, the modified gRNAs are modified with linker molecules for immobilization onto a surface. FIGS. 31A-31C present results showing target discrimination of modified gRNAs.

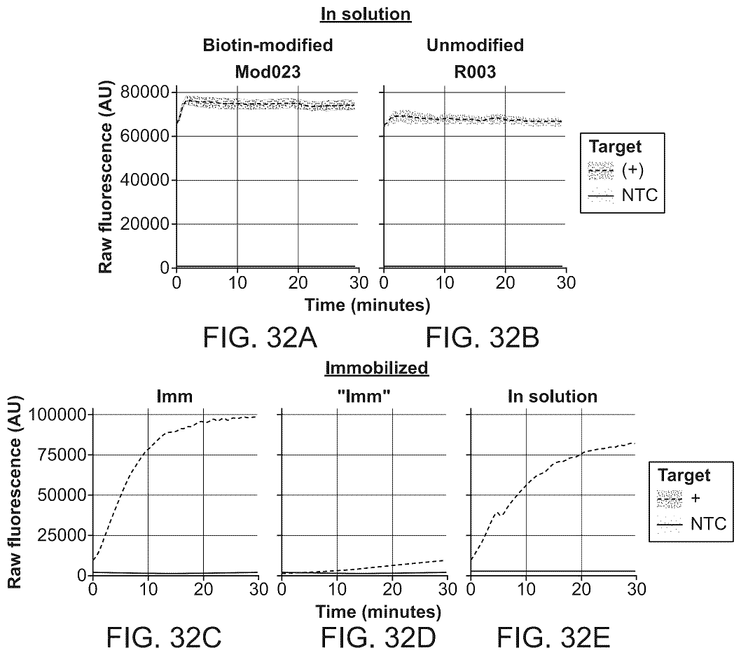

In some embodiments, guide RNAs are modified for surface modification. In some embodiments, reporters are modified for surface immobilization. In these embodiments, an immobilized gRNA, or immobilized reporter or a combination thereof participate in a diagnostic assay including a programmable nuclease. FIGS. 32A-32E present results demonstrating functionality of biotin-modified Cas13a gRNA. In each figure, raw fluorescence is plotted against time in minutes, where the dashed line series represents data for when the target is present, and the thin solid line with low amount of speckle for boundaries series represents when target is not (no target control or NTC). FIGS. 32A and 32B show results, in solution, for mod023, the biotin modified reporter and R003 the non-biotin-modified reporter, respectively. In some embodiments the biotin-modified gRNA has similar performance to the non-biotin-modified gRNA in solution. FIG. 32C shows results for gRNA that was modified with biotin and immobilized to the surface. FIG. 32D shows results for gRNA that was not modified with biotin but was deposited on the surface in the same manner as FIG. 32C. FIG. 32E, similar to FIGS. 32A and 32B, shows results for gRNA that was unmodified and in solution. Together these results showed that with biotin modification and surface immobilization functionality was maintained and DETECTR assay performance was not adversely affected.

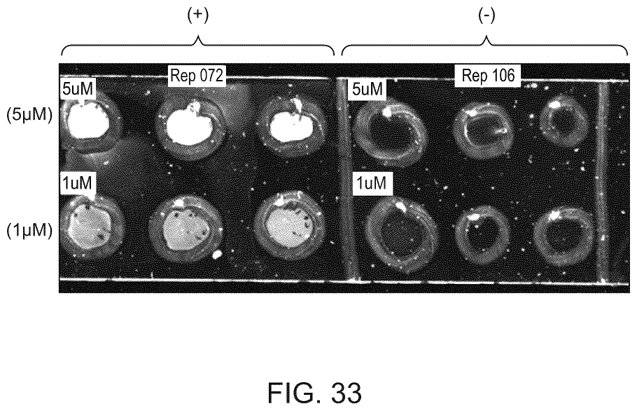

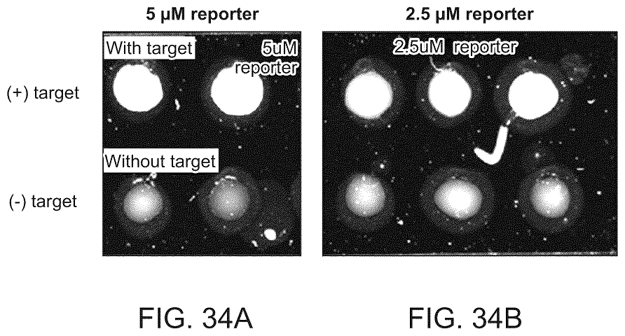

In some embodiments, biomolecules are immobilized to surfaces. In some embodiments, the surfaces were glass. FIG. 33 shows results for the test reporter, Rep072, and the negative control, Rep106. The replicates of Rep072 at 5 μM show the strongest signal and the three replicates of Rep072 at 1 μM concentration show the next strongest signal. The negative control reporter, rep106 shows the same low signal (on none at all) for both 5 μM and 1 μM concentrations. This result shows specific binding of a FAM-biotinylated reporter with a 30 minute incubation time at both 5 μM and 1 μM concentrations. FIGS. 34A-34B show similar results with reporters at 5 mM concentrations in FIG. 34A and 2.5 mM concentrations in FIG. 34B. The top row of FIGS. 34A and 34B show spots exhibiting bright fluorescence and the bottom row of FIGS. 34A and 34B show spots exhibiting similarly low fluorescence.

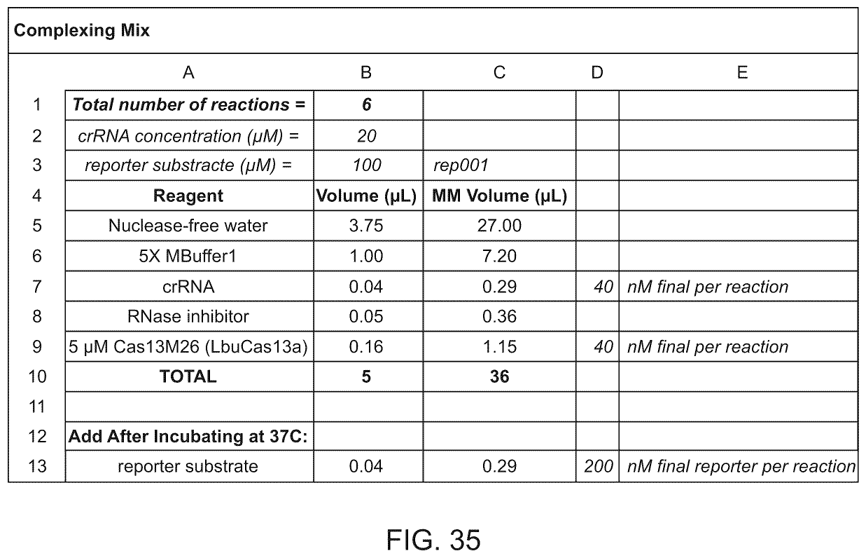

Experimental parameters for the preparation of an embodiment of a complexing mix are seen in FIG. 35.

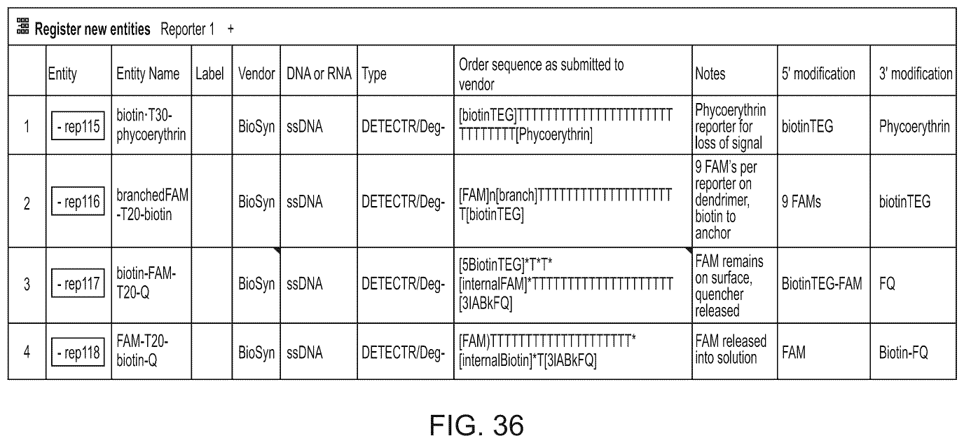

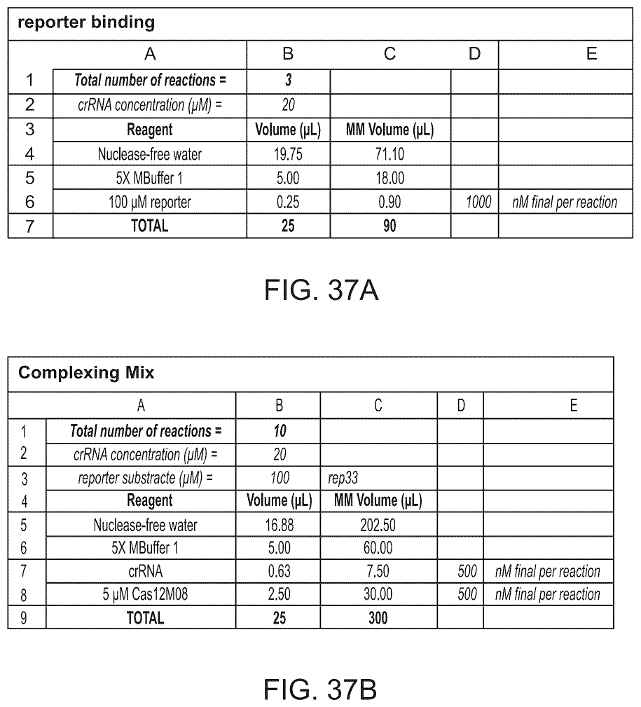

In some embodiments, fluorescent quencher-based reporters are used in the immobilized DETECTR assay. FIG. 36 shows sequence and other details for reporters used in some embodiments. In some embodiments, reporters rep072, rep104, rep105, rep117 and rep118 are used for binding to a reader plate. Reporter binding details and complexing mix parameters as seen in FIGS. 37A and 37B, respectively for some embodiments.

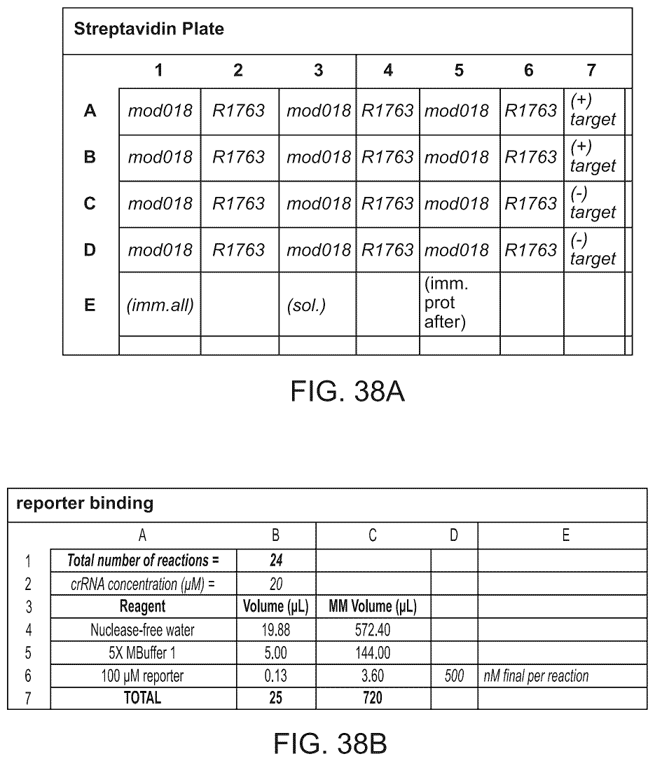

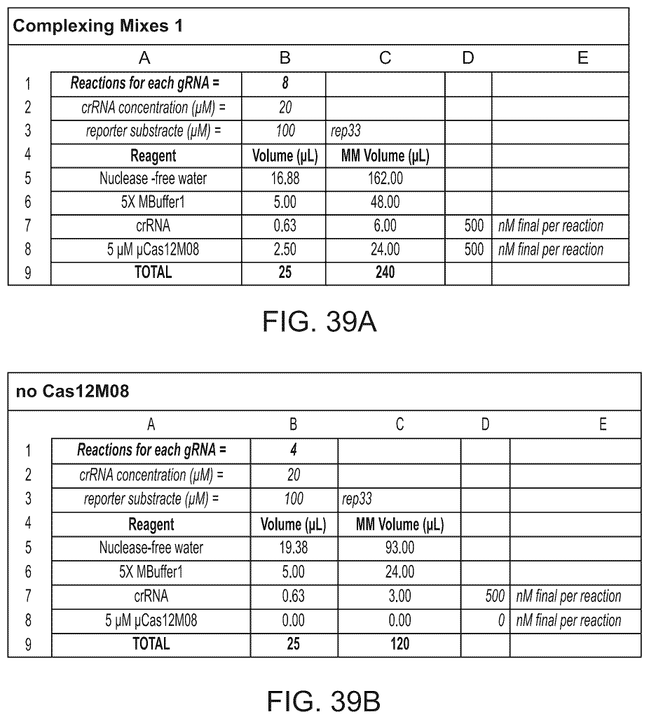

Described herein are various embodiments, where both the gRNA and reporter are bound to a plate as opposed to the gRNA, reporter and CAS protein. This removes the need to functionalize the surface with the pre-complex of gRNA and CAS protein, allowing for an easier manufacturing process. Additionally, greater specificity can be achieved by allowing for more stringent washes. An experimental design of this embodiment and conditions for binding a reporter to a plate in this embodiment are seen in FIGS. 38A and 38B, respectively. Complexing reactions for mod018 (5′ biotin-TEG R1763 SARS-CoV-2 N-gene) and R1763 CDC-N2-Wuhan prepared for a particular embodiment according to the conditions presented in FIG. 39A. Two sets of full complexing mix for an embodiment are seen in FIG. 39B.

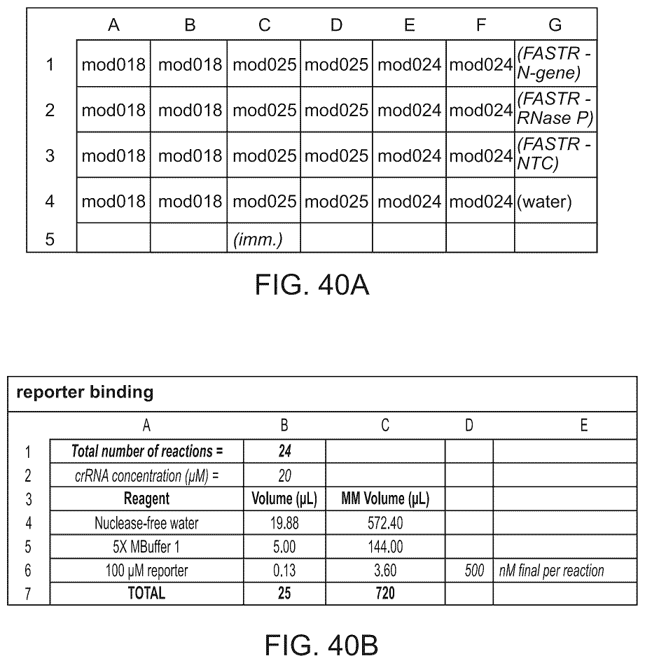

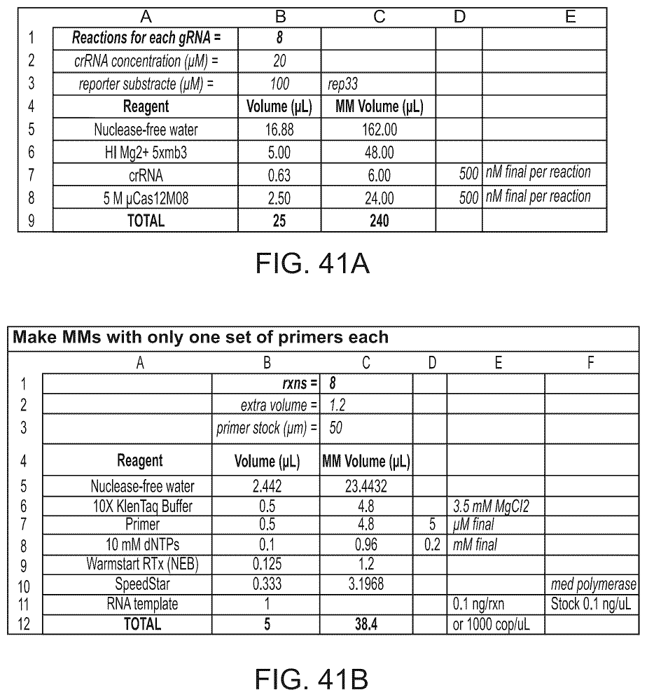

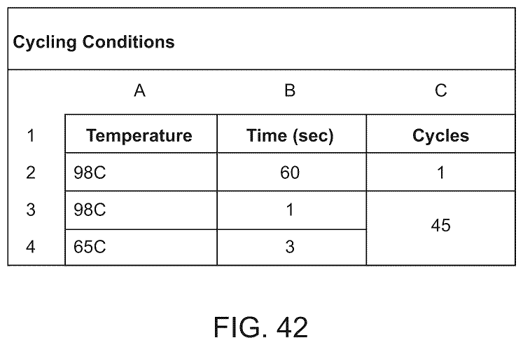

Described herein are various embodiments, that demonstrate target discrimination for immobilized reporters for the DETECTR reaction. An experiment design for such an embodiment is shown in FIG. 40A and reporter binding conditions shown in FIG. 40B. Reaction conditions are shown FIGS. 41A and 41B. PCR conditions are shown in FIG. 42.

In some embodiments, a pneumatic pump interfaces with the cartridge. In some embodiments, as shown in a top down view, in the middle of FIG. 43 of the device may comprise a cartridge with a top layer which may comprise reservoirs. Shown at the bottom of FIG. 43 the device may comprise a sliding valve containing the sample. In some embodiments, the device may comprise a lysis chamber shown at left, followed by amplification chambers to the right, and detection chambers further to the right.

In some embodiments, the DETECTR assay device may comprise a sliding valve as seen in FIG. 44. In some embodiments offset pitch of the channels allows aspirating and dispensing into each well separately and helps to mitigate cross talk between the amplification chambers and corresponding chambers. In some embodiments, a sample moves through the sliding valve device as seen in FIG. 45.

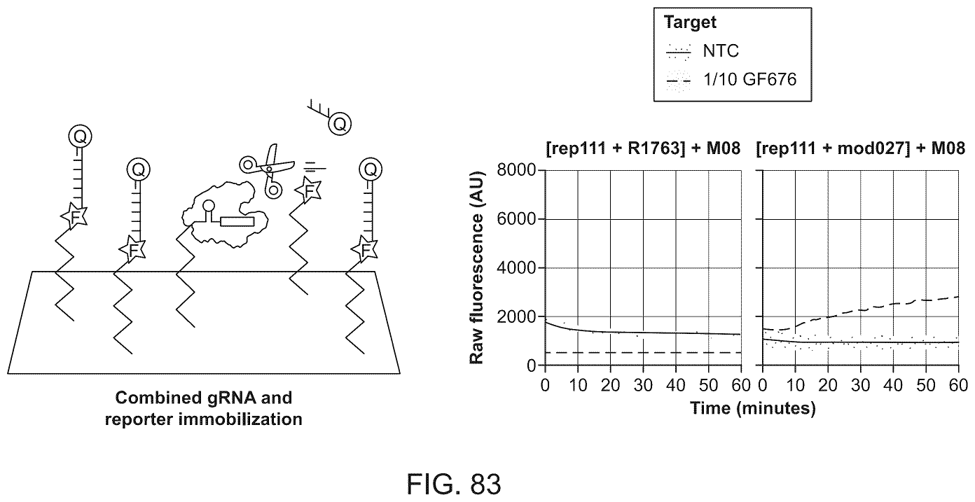

In some embodiments, NHS-Amine chemistry is used for immobilization of DETECTR components. FIG. 83 presents a schematic of combined gRNA and reporter immobilization and results for such embodiments. In this embodiment, a functional DETECTR reaction was immobilized to solid substrate (NHS plate) using primary amine modified reporters and gRNAs. In the example shown, a modified reporter (rep111) was bound to the surface in combination with either an unmodified crRNA (R1763) or a modified crRNA (mod027). After incubating these nucleic acids on the surface, the surface was washed 3 times, and then Cas12M08 was added with the target dsDNA. The immobilized DETECTR reaction was then incubated in a plate reader at 37 C for 60 minutes with continuous monitoring of the fluorescence.

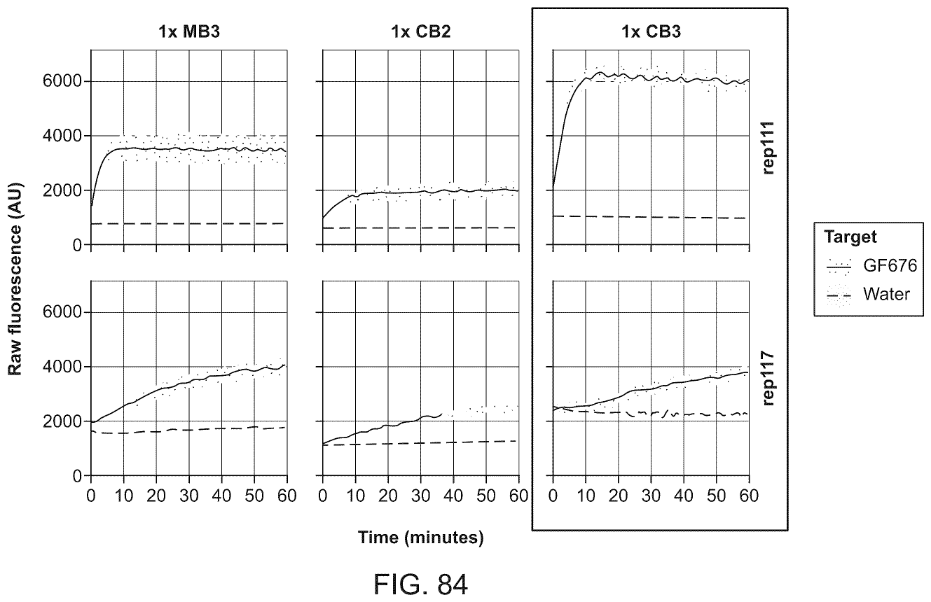

FIG. 84 presents results for various embodiments involving the optimizing conjugation buffer to reduce non-specific binding. For some embodiments, 1× Conjugation Buffer 3 (CB3) was selected as the buffer to perform binding studies. I was found that CB3 improved the binding of the amine-modified reporter (rep111) and reduced the binding of a biotinylated reporter (rep117) which should not bind to NHS covalently. In some embodiments, the wash buffer used was 1× MB3. In some embodiments, 1× MB3: 20 mM HEPES, pH 7.2, 2 mM KOAc, 5 mM MgAc, 1% Glycerol, 0.0016% Triiton X-100 was used. In some embodiments, 1× CB2: 20 mM HEPES, pH 8.0, 2 mM KOAc, 5 mM MgAc, 1% Glycerol, 0.0016% Triiton X-100 was used. In some embodiments, 1× CB3: 100 mM HEPES, pH 8.0, 10 mM KOAc, 25 mM MgAc was used.

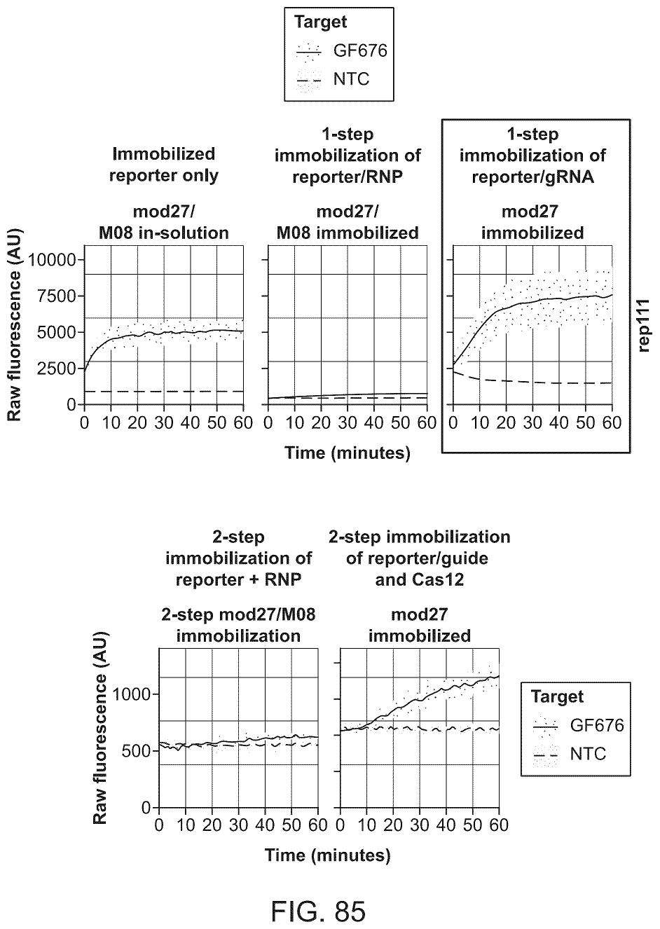

In some embodiments, different combinations of reporter+guide+Cas12M08 are immobilized. FIG. 85 presents results of such embodiments, involving the optimization the assay. For such embodiments, it was found that immobilizing guide and reporter first followed by the addition of Cas12M08 and target at the same time gave sufficient signal.

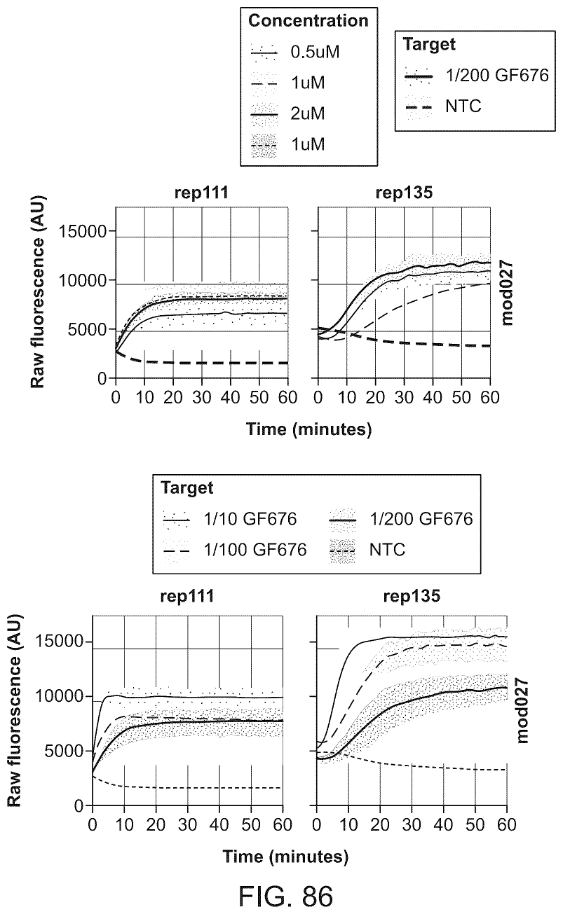

The results for optimizing gRNA and target concentrations to improve single-to-noise ratio for immobilized DETECTR assay are shown in FIG. 86. In some embodiments, guide concentration is increased while keeping reporter concentration constant at 0.5 μM, as seen on the left of FIG. 86, In such embodiments, the signal is not changed very much. In some embodiments, as seen on the right of FIG. 86, increasing target concentration 2-fold helped improve the overall signal with rep135. Additionally, for such embodiments, rep135 gave a better signal strength compared to rep111. The sequences for the two reporters are: rep111: 5AmMC6T//i6-FAMK/TTTTTTTTTTTT/3IABkFQ/(SEQ ID NO: 48) and rep135: 5AmMC12//i6-FAMK/TTTTTTTTTTTTTTTTTTT/3IABkFQ/(SEQ ID NO: 49).

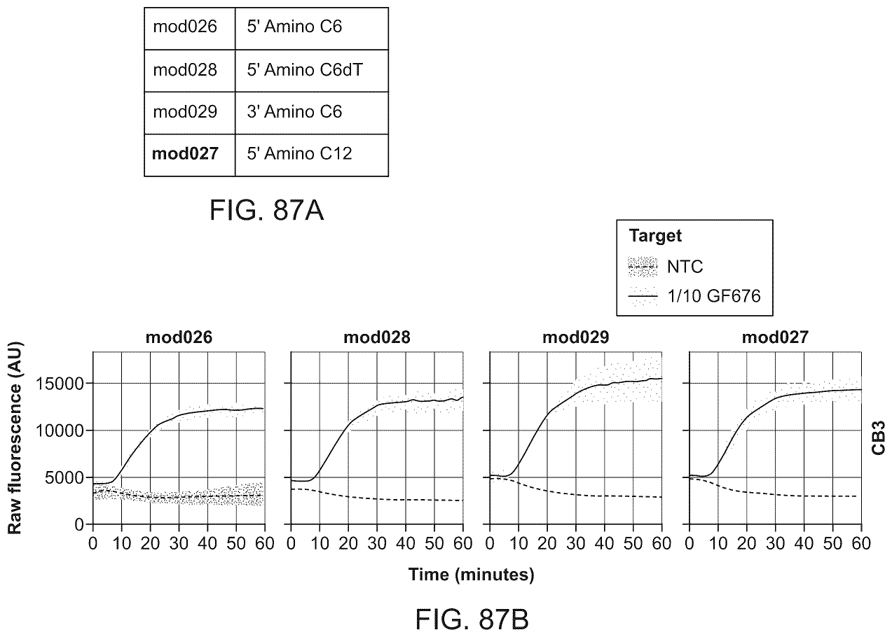

In some embodiments, amino modifications are used for DETECTR immobilization. FIG. 87 presents results for such embodiments. For each subplot raw fluorescence is plotted against time in minutes. Each of the four subplots represent different modifications. The solid line trace represents the no target control (NTC) and the dashed line trace represents a 1:10 dilution of target—GF676.

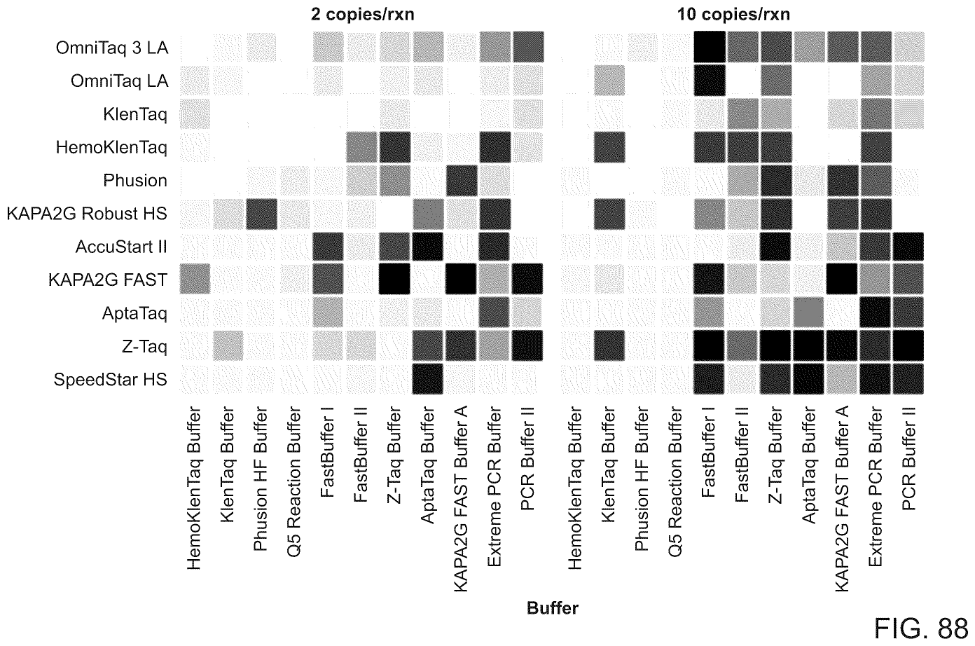

In some embodiments, rapid thermocycling and CRISPR diagnostics are used to detect SARS-CoV-2. Results are shown in FIG. 88. For some embodiments, polymerase and buffer combinations were identified that enabled the rapid amplification of SARS-CoV-2 using the N2 primers from the CDC assay. The assay of such embodiments was performed at two target concentrations: 2 copies/rxn and 10 copies/rxn. In some embodiments, reaction conditions are as follows: initial denaturation at 98 C for 30 seconds, followed by 45 cycles consisting of 1 second at 98 C and 3 seconds at 65 C. Following thermocycling, amplicon was transferred to a Cas12M08 detection reaction for 30 minutes at 37 C. Best performing enzyme/buffer pairs shown in FIG. 88 were those that gave strong signal in both tested concentrations.

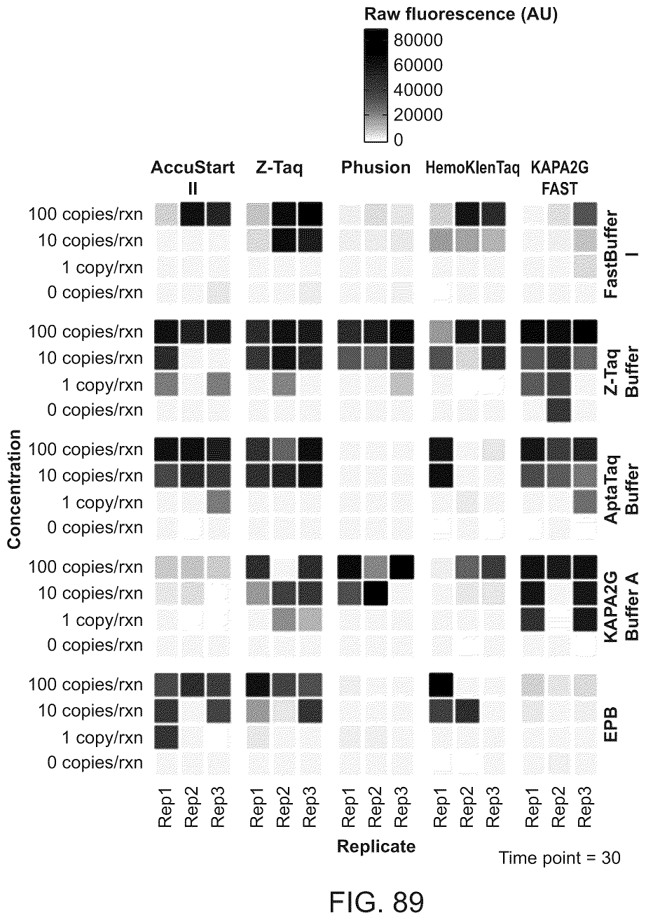

The top enzymes and buffers identified previously at various concentrations and with multiple replicates were tested for the FASTR assay. In some embodiments, the best performing enzymes and buffers as identified in the previously disclosed screening studies were used. Results of such embodiments are shown in FIG. 89. Reaction conditions of such embodiments are as follows: initial denaturation at 98 C for 30 seconds, followed by 45 cycles consisting of 1 second at 98 C and 3 seconds at 65 C. Primers used were from the CDC N2 assay for SARS-CoV-2. Following thermocycling, amplicon was transferred to a Cas12M08 detection reaction for 30 minutes at 37 C. The data present is the signal from the CRISPR reaction. Best performing enzyme/buffer pairs were those that gave strong signal at the lowest tested concentrations and with detection across replicates.

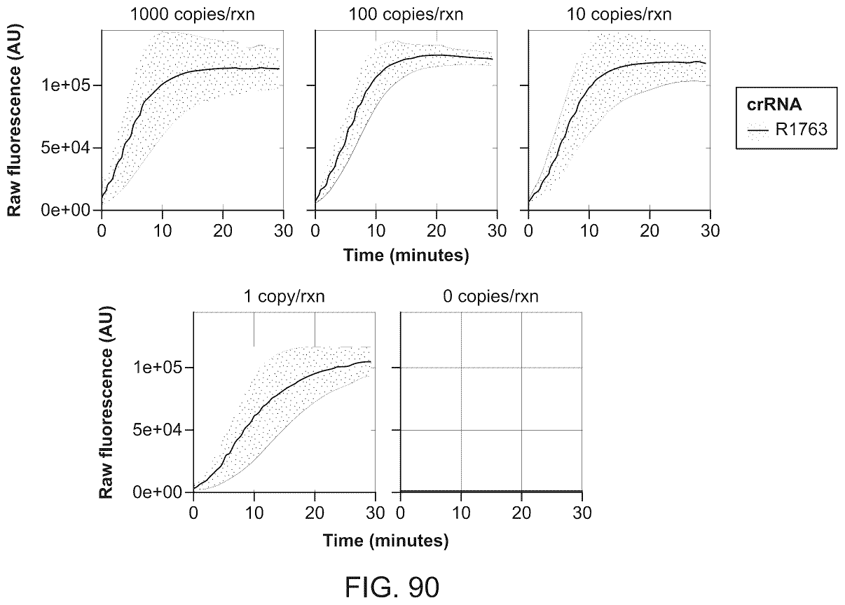

For some embodiments, single copy detection of SARS-CoV-2 with FASTR assay has been demonstrated as shown in FIG. 90. In some embodiments, the limit of detection of the FASTR assay was evaluated using solutions composed of 1000 copies of SARS-CoV-2 per reaction to 1 copy per reaction. For some embodiments, reaction conditions are as follows: reverse transcription at 55 C for 60 seconds, initial denaturation at 98 C for 30 seconds, followed by 45 cycles consisting of 1 second at 98 C and 3 seconds at 65 C. Primers used were from the CDC N2 assay for SARS-CoV-2. Following thermocycling, amplicon was transferred to a Cas12M08 detection reaction for 30 minutes at 37 C. The data presented in FIG. 90 is the signal from the CRISPR reaction. It was found that the limit of detection of the CRISPR assay was 1 copy of SARS-CoV-2 per reaction.

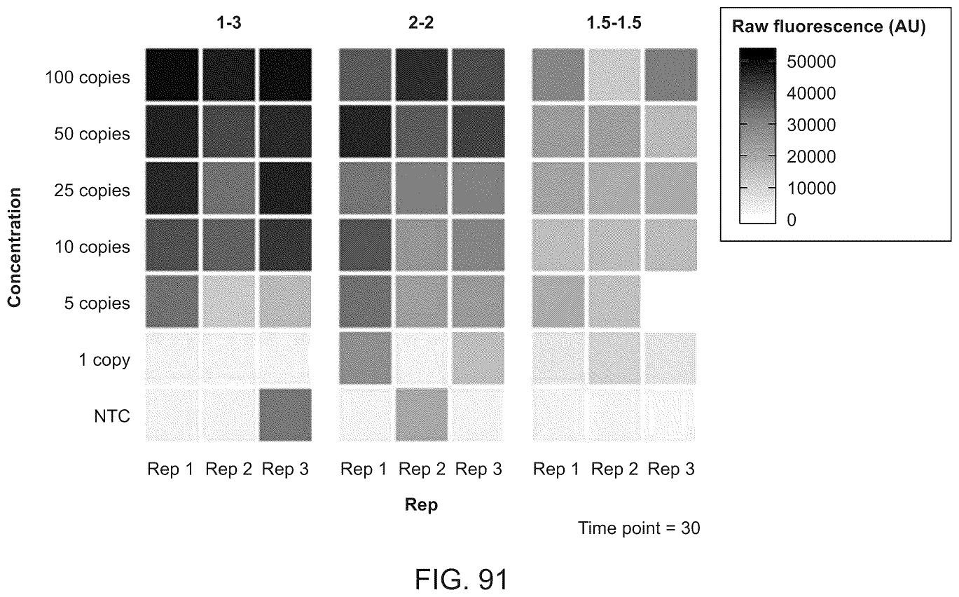

Variations on Across some embodiments, rapid cycling times are varied to evaluate denaturation and annealing/extension for the FASTR assay. Results for such embodiments are shown in FIG. 91. In some embodiments, reverse transcription is run at 55 C for 60 seconds and initial denaturation at 98 C for 30 seconds. In some embodiments, the tested cycling conditions were: 98 C for 1 second, 65 C for 3 seconds; 98 C for 2 seconds, 65 C for 2 seconds; or 98 C for 1.5 seconds, 65 C for 1.5 seconds. In some embodiments, primers used were from the CDC N2 assay for SARS-CoV-2. Following thermocycling, amplicon was transferred to a Cas12M08 detection reaction for 30 minutes at 37 C. The results shown in FIG. 91 indicate that >2 seconds of annealing/extension time at 65 C are necessary for robust sensitivity.

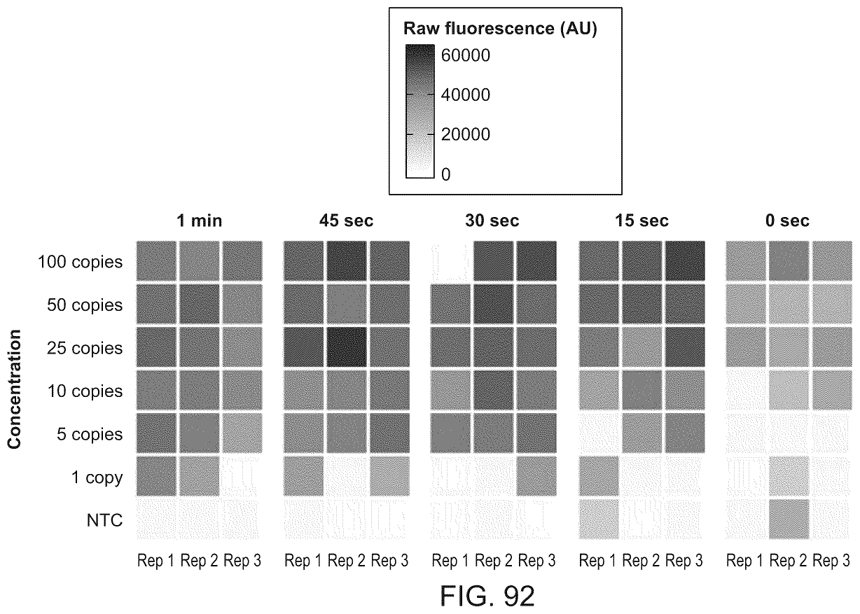

FIG. 92 presents results for Minimizing RT time for FASTR. The performance of the FASTR assay was evaluated, for various embodiments, where the reverse transcription incubation times were varied holding temperature at 55 C. The results shown in FIG. 92 indicate the assay is most robust above 30 seconds of reverse transcription.

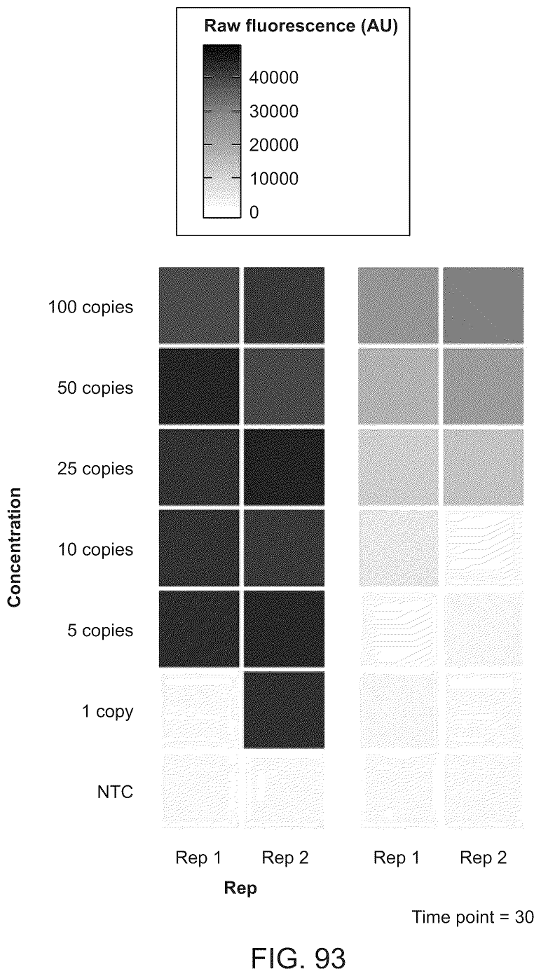

FIG. 93 presents results for Higher pH buffers improve FASTR performance. In some embodiments, the FASTR assay utilizes buffers with pH of either 9.2 or pH 7.8. The FASTR assay was evaluated using these buffer pH values. The results as shown in FIG. 93 indicate that the higher pH buffer produced superior results in terms of amplicon yield and sensitivity.

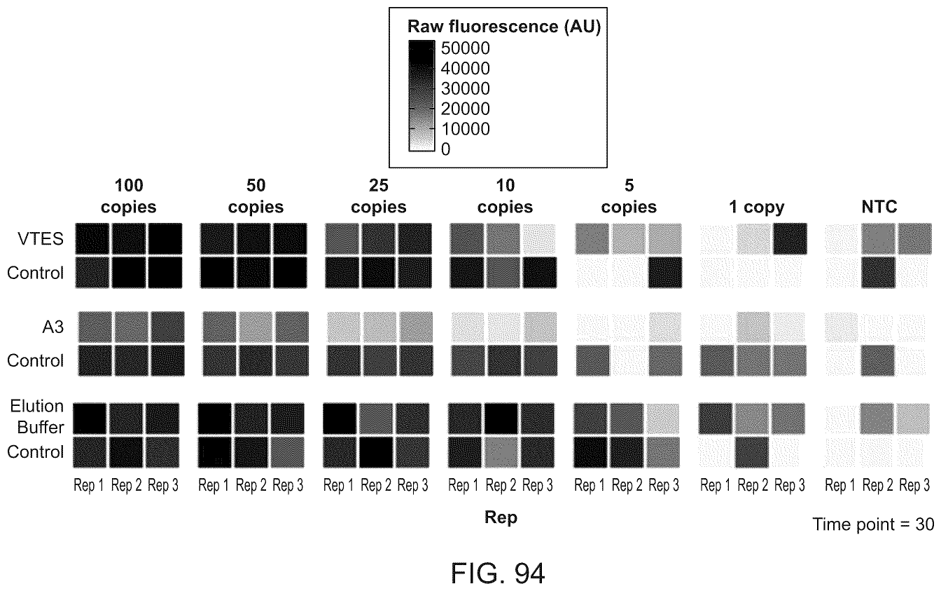

For some embodiments, the FASTR assay compatibility with crude lysis buffers was investigated. Results are shown in FIG. 94 where there are three row groups, each consisting of two sub rows representing a buffer and control from top to bottom respectively. The buffers, VTES, A3 and Elution buffer are plotted against a control, from top to bottom, respectively. In FIG. 94 there are also 7 subgroups showing the number of copies decreasing from left to right. For certain embodiments, the performance of the FASTR assay when combined with various crude lysis buffers was evaluated, where crude lysis buffers VTE5, A3, and the Elution Buffer from the ChargeSwitch kit (Thermo) were tested. For certain embodiments, the FASTR assay performed best for VTE5, but was performed slightly less robustly in the A3 buffer and the Elution Buffer from the ChargeSwitch kit performed similarly to the control reactions (water).

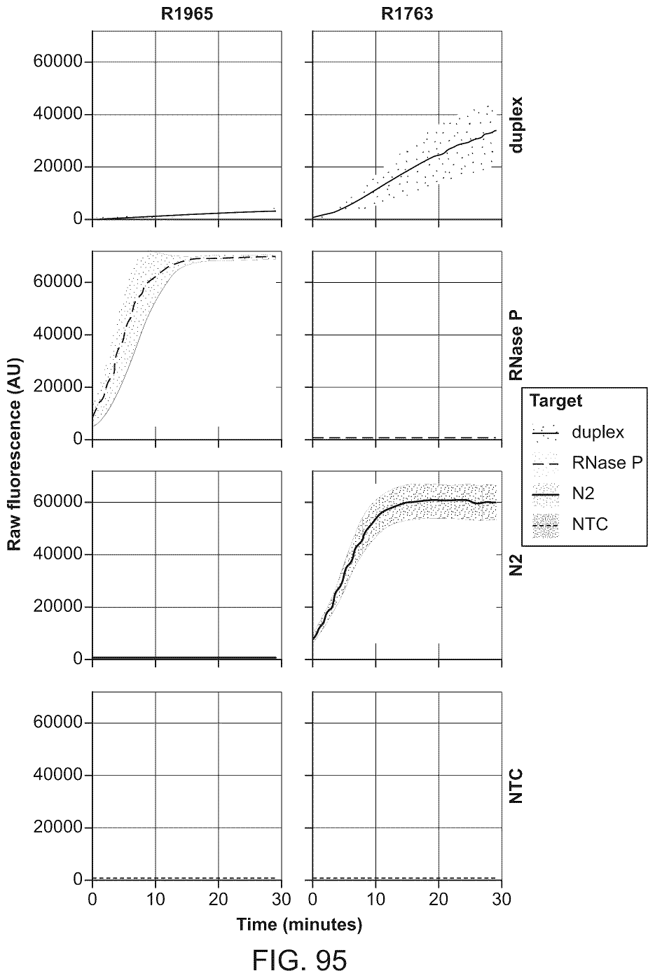

For some embodiments non-optimized multiplexing of FASTR was demonstrated as shown in FIG. 95. In FIG. 95, raw fluorescence is plotted in the y-axis and time is plotted in the x-axis for each sub-plot. Each column illustrates a particular sequence: R1965 and R1763, respectively Each row represents duplex, RNase P, N2 and the no target control, from top to bottom respectively. For some embodiments, initial testing of multiplexed FASTR for SARS-CoV-2 and RNase P POP7 (endogenous control) showed that while the single-plex assays generated a robust signal in DETECTR, the duplex assay tended to generate a weak signal for SARS-CoV-2 (R1763) and almost no signal for RNase P (R1965). In some embodiments, reaction conditions were as follows: reverse transcription at 55 C for 60 seconds, initial denaturation at 98 C for 30 seconds, followed by 45 cycles consisting of 1 second at 98 C and 3 seconds at 65 C. In some embodiments, primers from the CDC N2 assay for SARS-CoV-2, and M3637/M3638 were used.

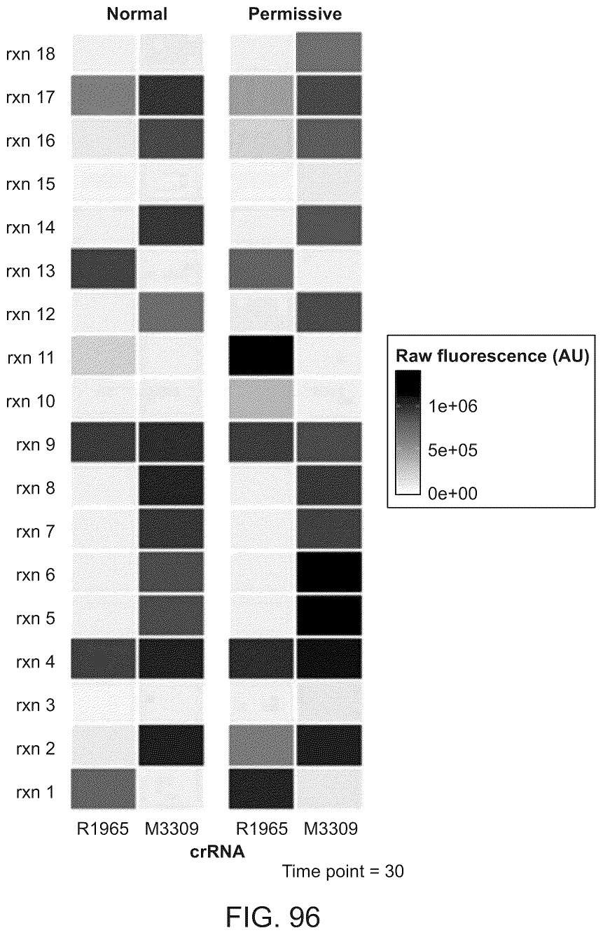

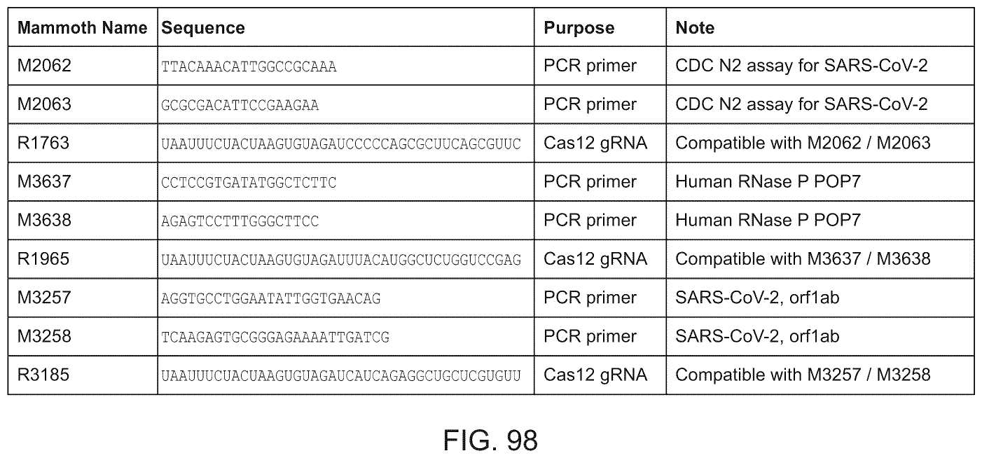

In various aspects described herein, FASTR can be used for multiplexed detection, as shown in FIG. 96. The components of a FASTR reaction, such as primer concentration, dNTP concentration, presence/absence of DMSO, and other factors, can impact the performance of a multiplex FASTR reaction. FIG. 98 shows 18 different experimental conditions for a multiplex FASTR reaction targeting human RNase P POP7 and SARS-CoV-2. In FIG. 96 each row of the y-axis represents experimental runs 1-18 and each column represents the detection signal from a particular crRNA at a time point of 30 minutes in the reaction. The color represents the value of the raw fluorescence. In some embodiments, the multiplexed FASTR assay for SARS-CoV-2 and RNase P, comprise a set of SARS-CoV-2 primers (M3257/M3258). A series of experiments of such embodiments was performed with varied reaction conditions containing different combinations of buffers, primer concentrations, dNTPs, and DMSO. Results identified two reaction conditions that performed robustly for the multiplex reaction. In one of these embodiments, reaction 4, conditions consisted of: 1× FastBuffer 2; 1 uM RNase P primers; 0.5 uM CoV primers; 0.2 mM dNTPs; and 2% DMSO. In another embodiment, reaction 9, conditions consisted of: 1× Klentaql buffer; 1 uM RNase P primers; 0.5 uM CoV primers; 0.4 mM dNTPs; and 0% DMSO. In some embodiments, normal reaction conditions consisted of reverse transcription at 55 C for 60 seconds, initial denaturation at 98 C for 30 seconds, followed by 45 cycles consisting of 1 second at 98 C and 3 seconds at 65 C. In various aspects, permissive reaction conditions consisted of reverse transcription at 55 C for 60 seconds, initial denaturation at 98 C for 30 seconds, followed by 45 cycles consisting of 3 seconds at 98 C and 5 seconds at 65 C.

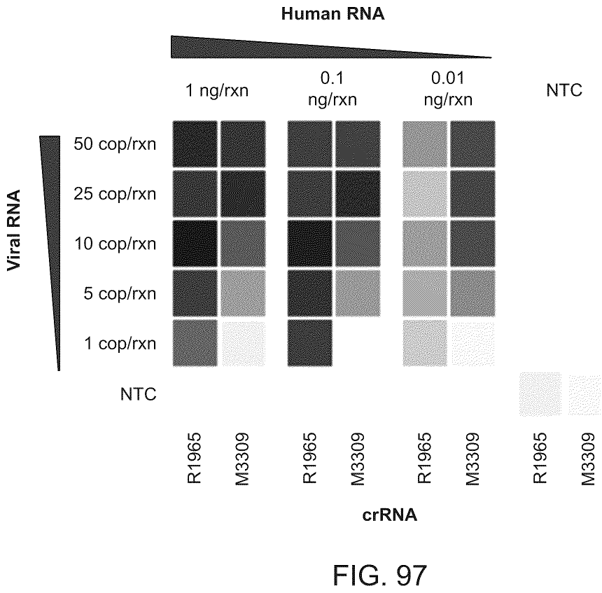

In some embodiments, the FASTR assay enables multiplexed detection. Results of a limit of detection (LOD) study of such embodiments are shown in FIG. 97. In FIG. 97, the x-axis shows the number of copies per reaction for viral RNA and the y-axis of each subplot identifies the particular crRNA. Each subplot shows nanograms of human RNA per reaction, decreasing in concentration from left to right. The 4^{th} subplot contains no human RNA, labeled as the no target control (NTC). For some embodiments, an optimized multiplexed FASTR assay was ran at various concentrations of human RNA and viral RNA. In some embodiments, results indicated that the assay performed at a range of human RNA concentrations, while maintaining a sensitivity of ˜5 copies per reaction. In certain aspects, results shown are from DETECTR reactions using either R1965 to detect the human RNase P, or R3185 (labeled M3309) to detect SARS-CoV-2. In various aspects, reaction conditions are as follows: reverse transcription at 55 C for 60 seconds, initial denaturation at 98 C for 30 seconds, followed by 45 cycles consisting of 1 second at 98 C and 3 seconds at 65 C. In some embodiments, primers used were M3257/M3258 (SARS-CoV-2) and M3637/M3638 (RNase P).

In some embodiments, key primers and gRNAs have the sequences as listed in FIG. 98.

### Sample Preparation and Lyophilization

Described herein are various methods of sample preparation and reagent storage. Any of the devices described herein may comprise one or more sample preparation reagents Any of the devices described herein may comprise sample preparation reagents as dried reagents. Dried reagents may comprise solids and/or semi-solids. In certain instances, dried reagents may comprise lyophilized reagents. Any of the devices described herein may comprise one or more lyophilized reagents (e.g., amplification reagents, programmable nucleases, buffers, excipients, etc.). In certain instances, methods include sample lysis, concentration, and/or filtration. In certain instances, methods include reconstitution of one or more lyophilized reagents. In some embodiments, lyophilized reagents may be in the form of lyophilized beads, spheres, and/or particulates. In some embodiments, the lyophilized bead, sphere, and/or particulate may comprise either single or multiple compounds. In some embodiments, the lyophilized bead, sphere, and/or particulate may be adjusted to various moisture levels or hygroscopy. In some embodiments, the lyophilized bead, sphere, and/or particulate may comprise assay internal standards. In some embodiments, the lyophilized bead, sphere, and/or particulate may have diameters between about 0.5 millimeters to about 5 millimeters in diameter.

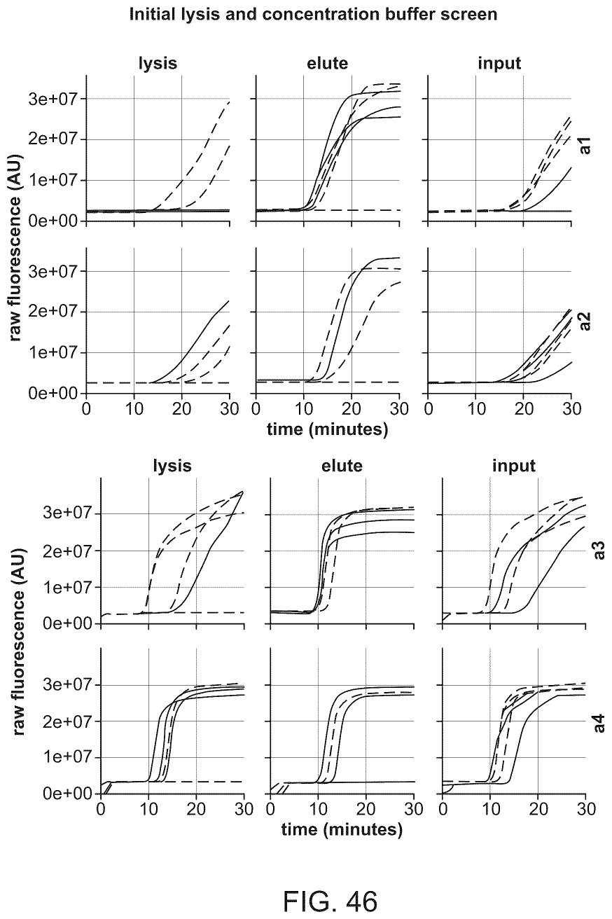

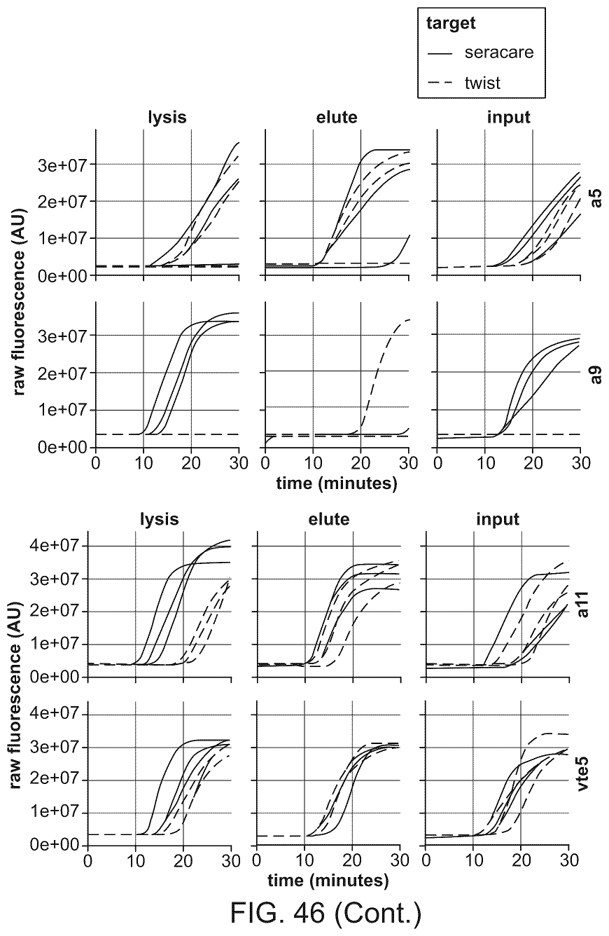

Described herein are various embodiments of the DETECTR reaction involving optimization of sample preparation and lyophilization. Such embodiments allow for adapting the buffer for binding a substrate to perform a concentration step. In some embodiments, experiments may be performed to evaluate the lysis (sample is evaluated directly in the assay) and binding (the sample is eluted from magnetic beads) characteristics of buffers with different components. In such embodiments, the input sample is the same concentration as the eluted sample. Results showing strong lysis activity, but minimal binding/concentration potential are shown in FIG. 46. Buffers al, a3 and potentially all of such embodiments show acceptable binding activity. Buffer a5, of such embodiments, shows modest activity for both lysis and binding and buffer a2 is not suitable for either activity.

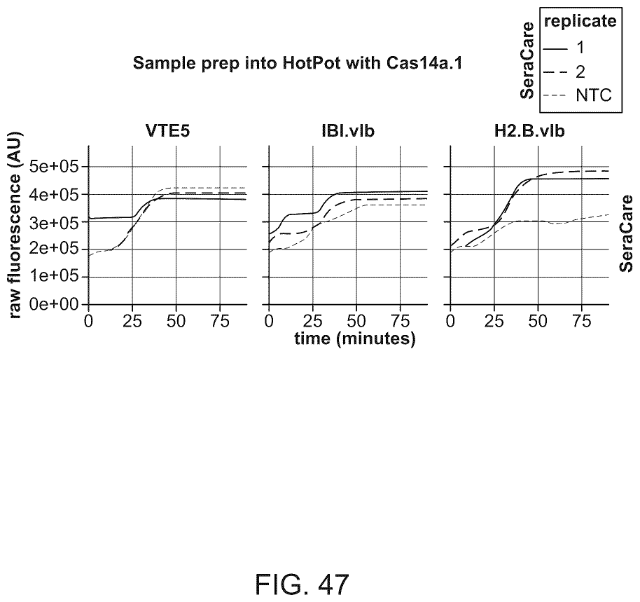

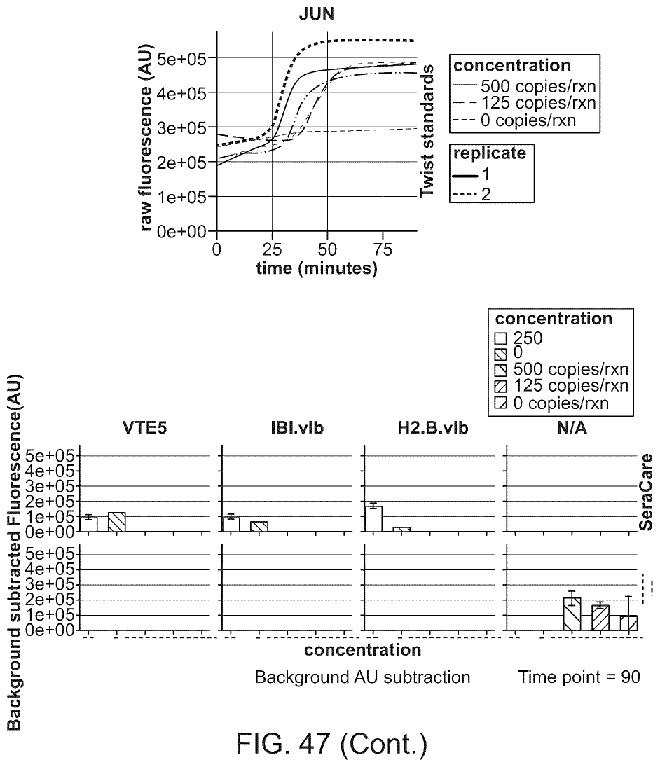

In some embodiments, crude lysis buffer is used in a one-pot assay with Cas14a.1. Results can be seen in FIG. 47.

In some embodiments, the enzyme Cas12MO8 is used with the DETECR assay. In some embodiments, the sequence of Cas12MO8 is:

(SEQ ID NO: 50)
MKKIDNFVGCYPVSKTLRFKAIPIGKTQENIEKKRLVEEDEVRAKD
YKAVKKLIDRYHREFIEGVLDNVKLDGLEEYYMLFNKSDREESDN
KKIEIMEERFRRVISKSFKNNEEYKKIFSKKIIEEILPNYIKDEE
EKELVKGFKGFYTAFVGYAQNRENMYSDEKKSTAISYRIVNENMP
RFITNIKVFEKAKSILDVDKINEINEYILNNDYYVDDFFNIDFFN
YVLNQKGIDIYNAIIGGIVTGDGRKIQGLNECINLYNQENKKIRL
PQFKPLYKQILSESESMSFYIDEIESDDMLIDMLKESLQIDSTIN
NAIDDLKVLFNNIFDYDLSGIFINNGLPITTISNDVYGQWSTISD
GWNERYDVLSNAKDKESEKYFEKRRKEYKKVKSFSISDLQELGGK
DLSICKKINEIISEMIDDYKSKIEEIQYLFDIKELEKPLVTDLNK
IELIKNSLDGLKRIERYVIPFLGTGKEQNRDEVFYGYFIKCIDAI
KEIDGVYNKTRNYLTKKPYSKDKFKLYFENPQLMGGWDRNKESDY
RSTLLRKNGKYYVAIIDKSSSNCMMNIEEDENDNYEKINYKLLPG
PNKMLPKVFFSKKNREYFAPSKEIERIYSTGTFKKDTNFVKKDCE
NLITFYKDSLDRHEDWSKSFDFSFKESSAYRDISEFYRDVEKQGY
RVSFDLLSSNAVNTLVEEGKLYLFQLYNKDFSEKSHGIPNLHTMY
FRSLFDDNNKGNIRLNGGAEMFMRRASLNKQDVTVHKANQPIKNK
NLLNPKKTTTLPYDVYKDKRFTEDQYEVHIPITMNKVPNNPYKIN
HMVREQLVKDDNPYVIGIDRGERNLIYVVVVDGQGHIVEQLSLNE
IINENNGISIRTDYHTLLDAKERERDESRKQWKQIENIKELKEGY
ISQVVHKICELVEKYDAVIALEDLNSGFKNSRVKVEKQVYQKFEK
MLITKLNYMVDKKKDYNKPGGVLNGYQLTTQFESFSKMGTQNGIM
FYIPAWLTSKMDPTTGFVDLLKPKYKNKADAQKFFSQFDSIRYDN
QEDAFVFKVNYTKFPRTDADYNKEWEIYTNGERIRVFRNPKKNNE
YDYETVNVSERMKELFDSYDLLYDKGELKETICEMEESKFFEELI
KLFRLTLQMRNSISGRTDVDYLISPVKNSNGYFYNSNDYKKEGAK
YPKDADANGAYNIARKVLWAIEQFKMADEDKLDKTKISIKNQEWL
EYAQTHCE*.
The * indicates a stop codon.

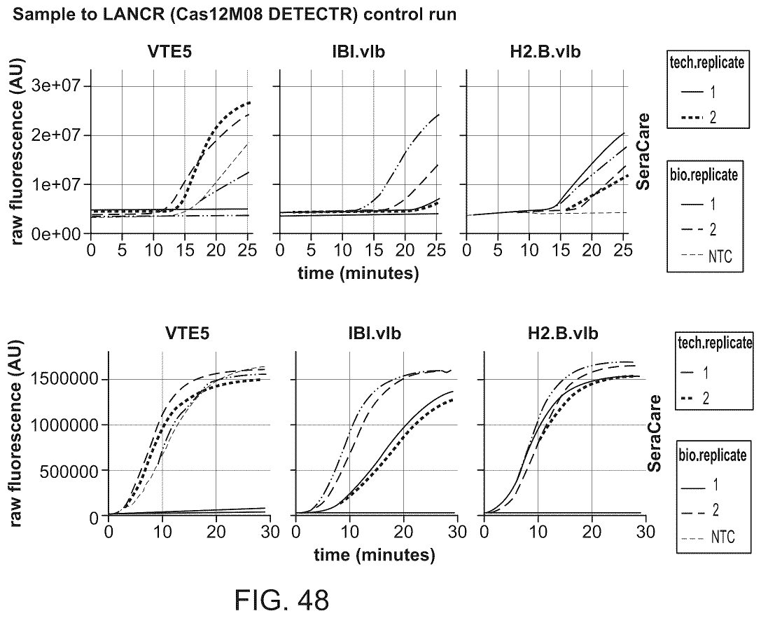

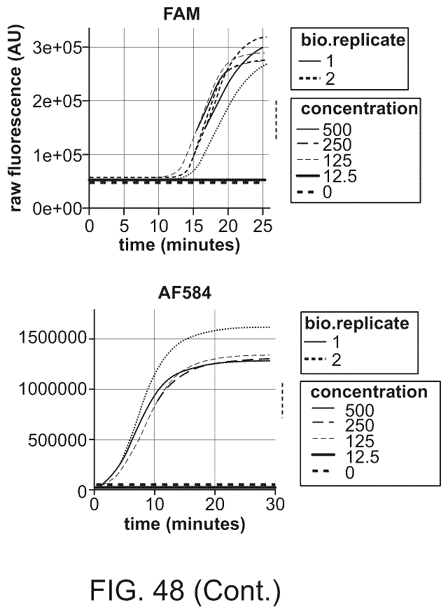

FIG. 48 presents results for a control study involving sample preparation optimization of the LANCR (Cas12MO8 DETECTR) assay. In such embodiments, crude lysis involves: 25 uL sample+25 uL lysis buffer and incubation at 25 C for 1 minute. In some embodiments, the LANCR reaction is run as follows: 5 uL sample in a 25 uL reaction volume (standard conditions). In some embodiments the DETECTR reaction is run as follows: 2 uL LANCR product in 20 uL reaction volume (standard conditions). Sample: 250 copies/rxn SeraCare.

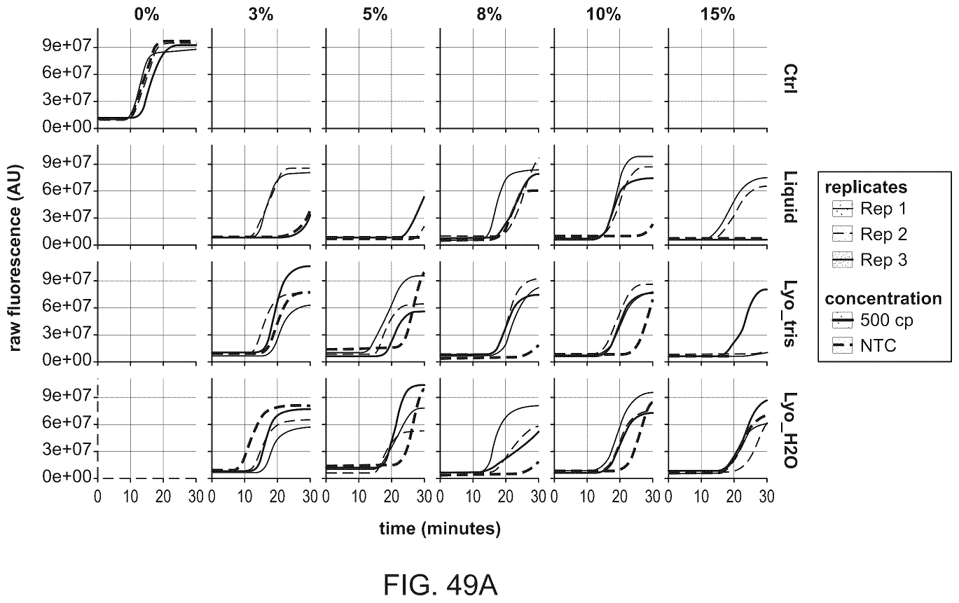

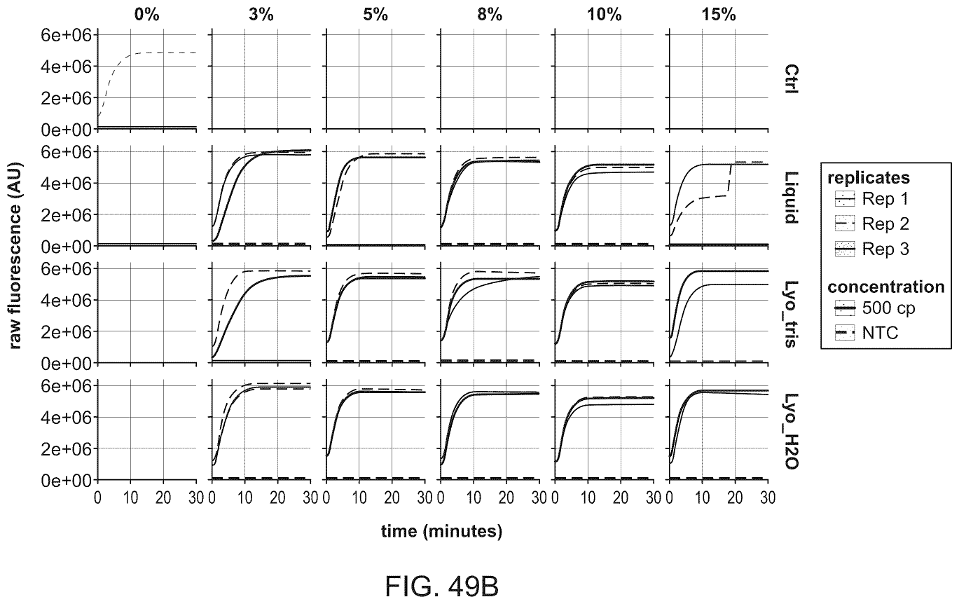

For some embodiments, two groups of conditions were evaluated for lyophilization performance. For one embodiment, Group I, Trehalose is used. FIGS. 49A-49B presents lyophilization optimization results for the Group 1 using RT-LAMP assay is presented in FIG. 49A and the DETECTR assay as seen in FIG. 49B.

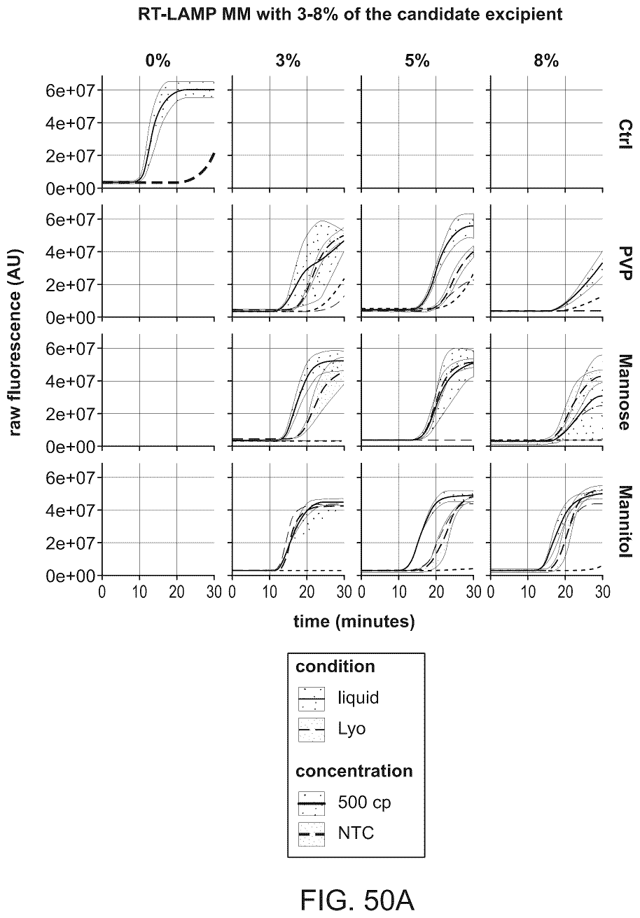

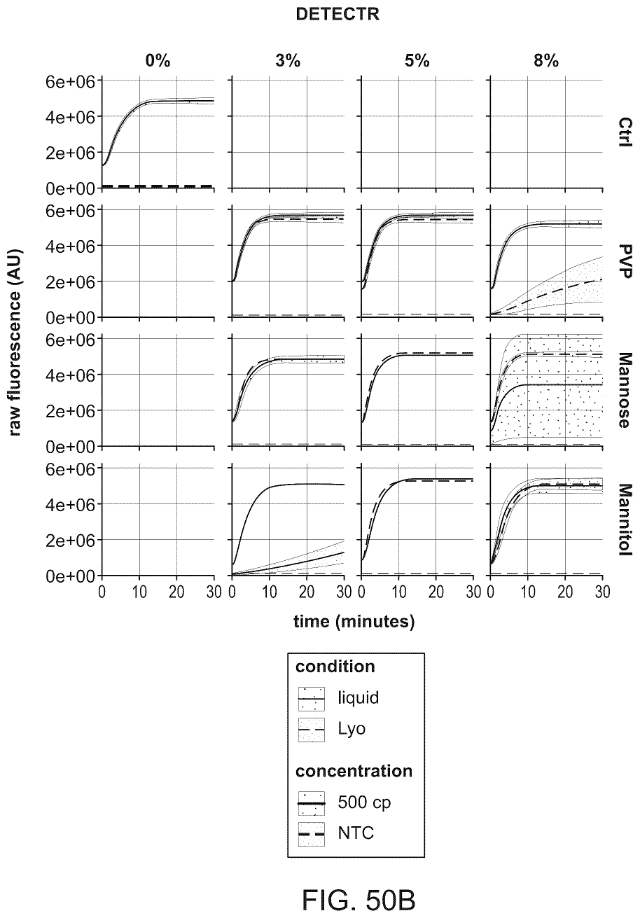

In another embodiment, Group II comprising: PVP 40; sorbitol; Mannitol; and Mannosse are used. FIGS. 50A-50B presents lyophilization optimization results for Group 2 evaluated using RT-LAMP 3-8% of the candidate excipient as shown in FIG. 50A and for Group 2 using the DETECTR assay conditions as seen in FIG. 50B.

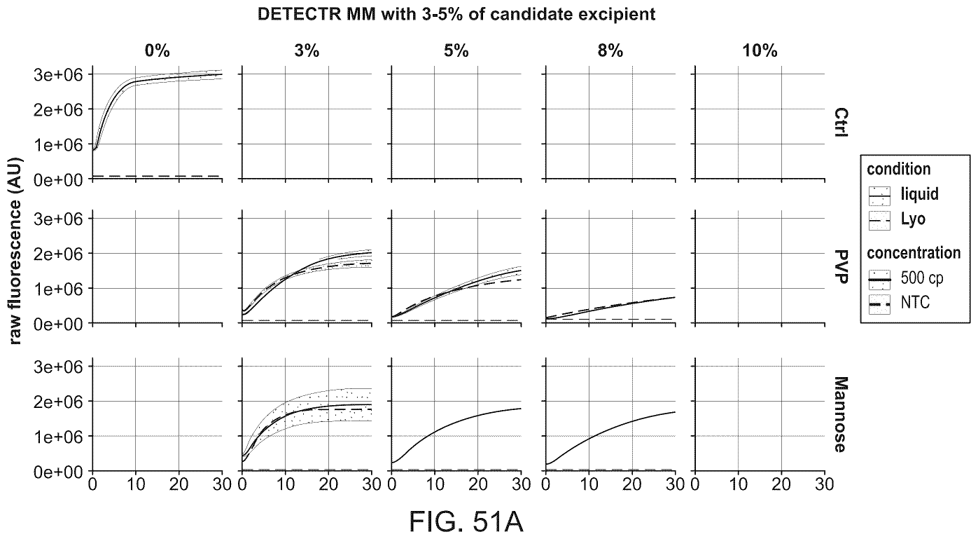

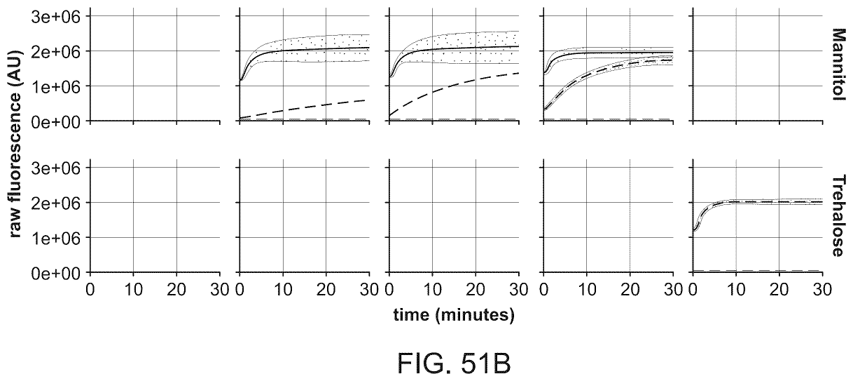

FIGS. 51A-51B presents lyophilization optimization results for an embodiment of Group 2: PVP 40, Sorbitol, Mannitol, Mannose, using DETECTR MM with 3-5% of candidate excipient.

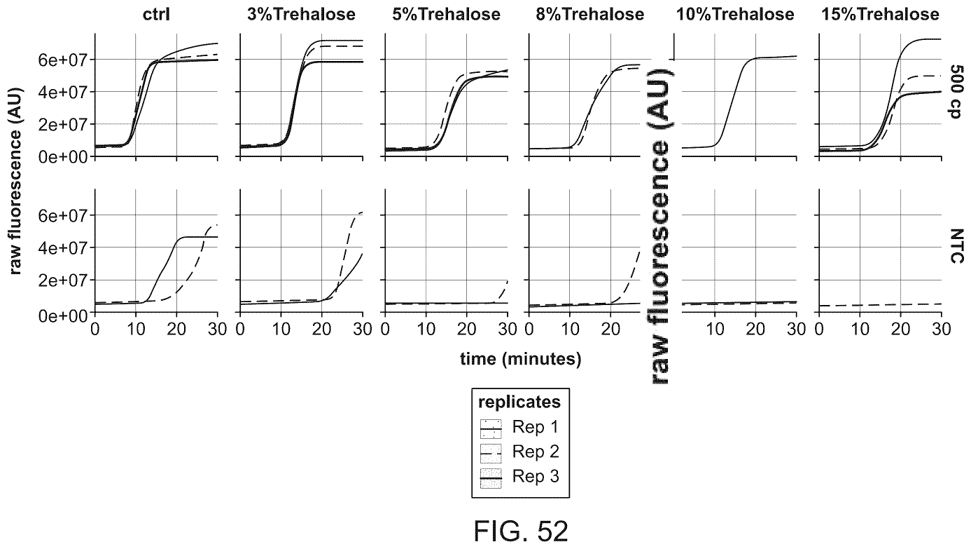

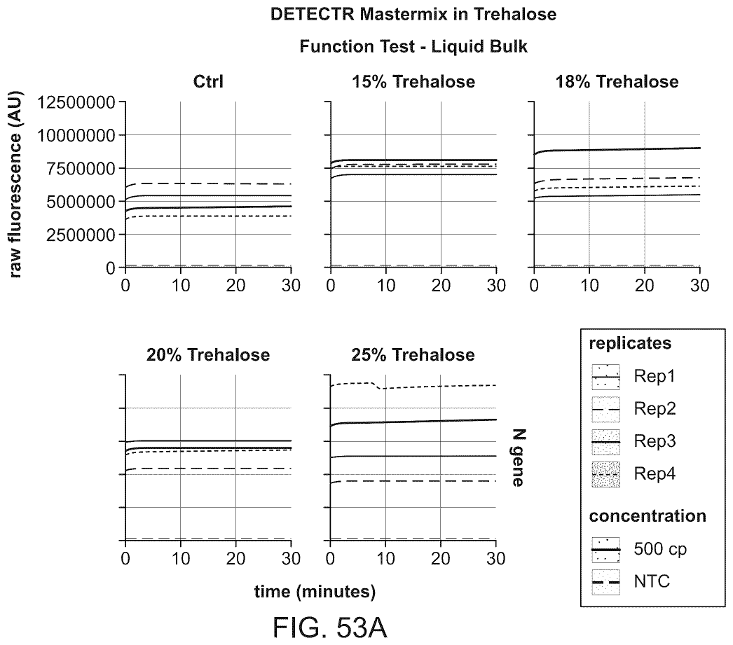

In some embodiments, Trehalose is used to control the rate of the reaction. FIG. 52 presents results from a study involving such embodiments, where RT-LAMP reaction components and various percentages of Trehalose were used.

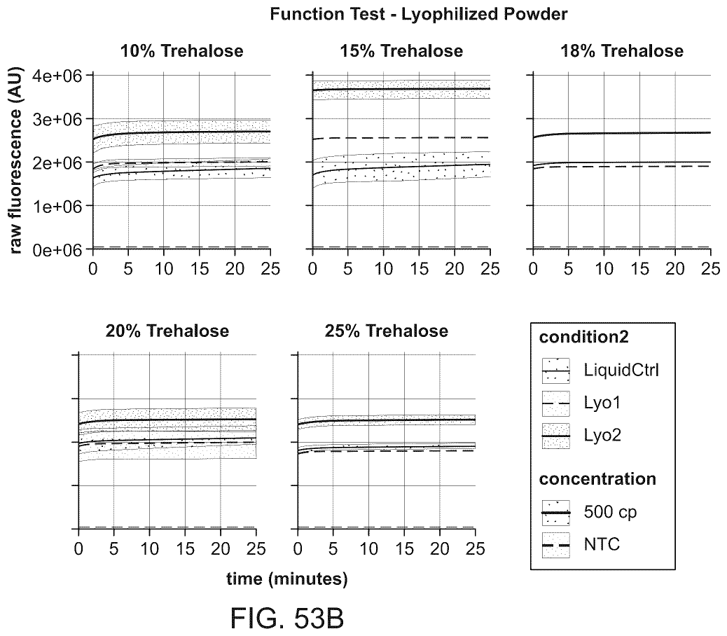

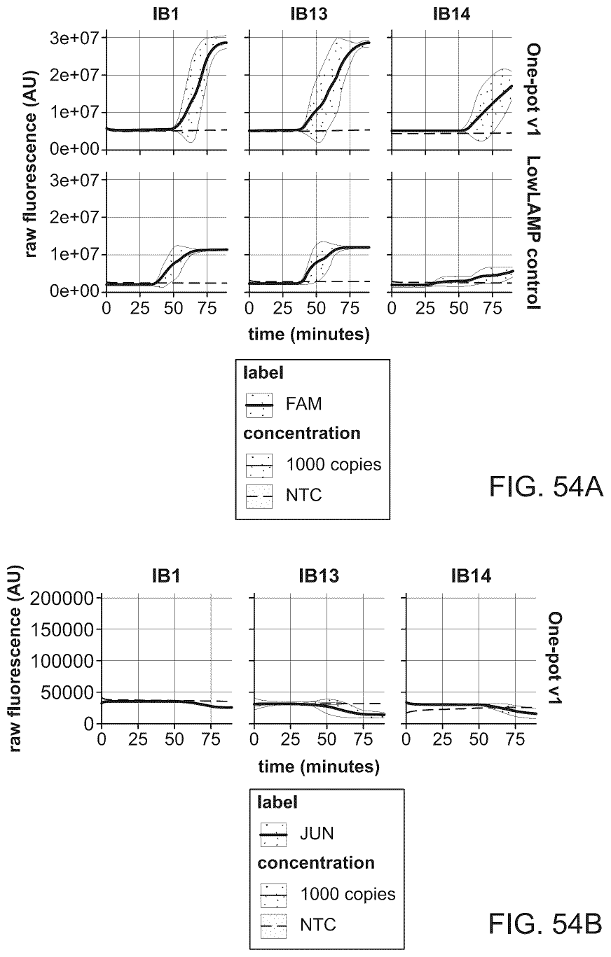

FIGS. 53A-53B presents results of a study involving embodiments where DETECTR reaction components and Trehalose are used. The top row of subplots show results for increasing Trehalose percentages in liquid bulk solution. The bottom row show results for increasing Trehalose in lyophilized powder form. In such embodiments, primary drying lasts 90 hours, fill volume is 250 uL in a 2 mL vial; reconstituted with 250 uL water; secondary drying is 6 hours; and temperature ramp is 0.5° C. per minute.

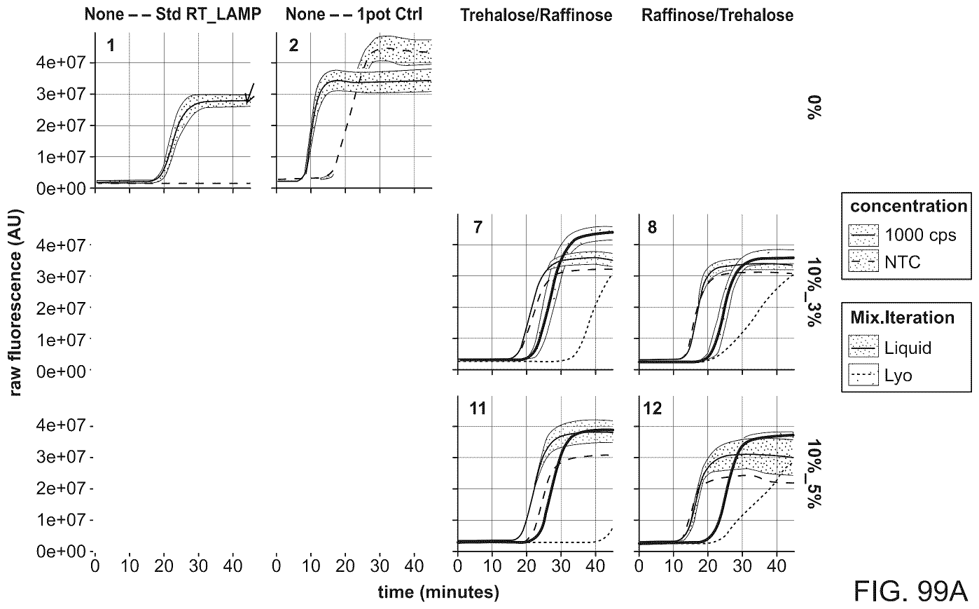

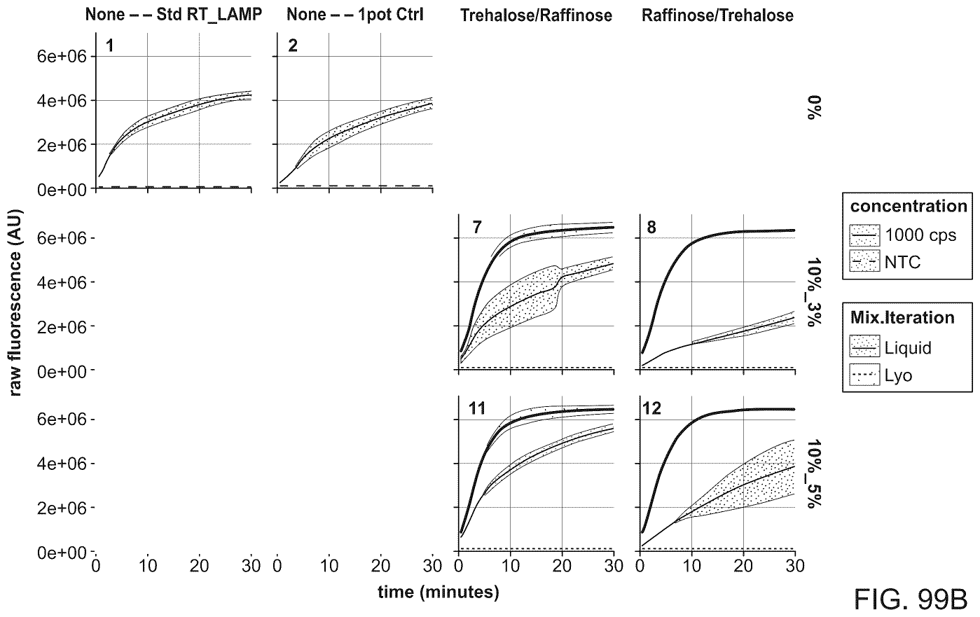

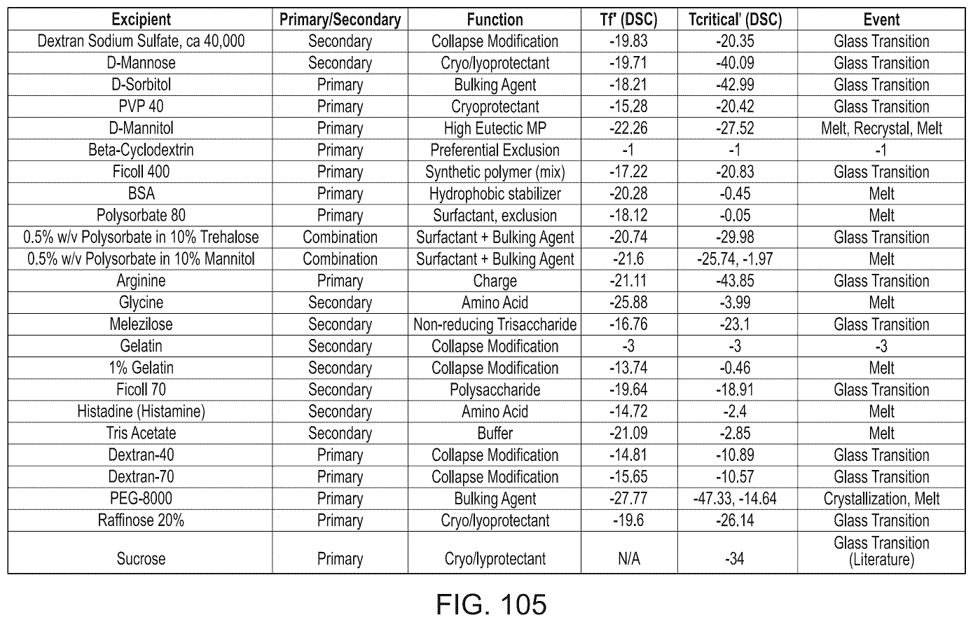

Described herein are various methods and devices for carrying out DETECTR assays. In some embodiments, DETECTR assays utilize the Cas12M08 protein. In some embodiments, RT-LAMP and DETECTR master mixes of reagents are lyophilized in the same combined master mix. In some embodiments, RT-LAMP and DETECTR master mixes of reagents and target are lyophilized in the same combined master mix. FIG. 99A presents results for a one-pot RT-LAMP assay. In some embodiments, one-pot refers to the combination of both RT-LAMP and DETECTR reaction reagents in one sample. FIG. 99B presents results for a Cas12M08-based DETECTR assay. The left-hand y-axis plots raw fluorescence. The x-axis plots time in minutes. Subplot 1 presents results where master mixes were not lyophilized and did not contain excipients. Subplot 2 presents results where master mixes were not lyophilized but did contain excipients. In some embodiments, an excipient is used confirm reagent stability throughout the lyophilization process, comprising freezing and drying steps. In some embodiments, excipients are sugars. In some embodiments, excipients comprise one or more of the compounds listed in FIG. 105. Subplots 7 and 11 of FIG. 99A presents results of an embodiment of a master mix containing the excipients Trehalose and Raffinose, wherein Trehalose and Raffinose are included at various percentages as described on the right-hand y-axis. Subplot 7 presents results for a mixture with 10% Trehalose and 3% Raffinose. Subplot 11 exhibits data for a mixture with 10% Trehalose and 5% Raffinose. Subplot 8 exhibits data for a mixture with 10% Raffinose and 3% Trehalose. Subplot 12 exhibits data for a mixture with 10% Raffinose and 5% Trehalose.

In some embodiments, the reagents and target from both the RT-LAMP and DETECTR assays were lyophilized in one sample. FIG. 99B shows results for such an embodiment ran through a Cas12M08-based DETECTR assay and can be interpreted as FIG. 99A has been described.

In some embodiments, the sample mixtures include multiple target molecules. In some embodiments, the sample mixtures contain multiple copies of nucleic acid targets. In some embodiments, the 1000 copies (cps) of the nucleic acid target are present in one sample. In the legends on the right-hand sides of FIGS. 99A-99B, “1000 cps” refers to 1000 copies of the target and is represented as a solid line. NTC refers to no-target-control and is represented as a large dashed line. The thin solid lines bounding all data series of FIGS. 99A-99B, 100, 101, 102A-102B and 103 represent bounds of the data set. Additionally, in some embodiments, “mix. iteration” refers to the form of the master mix, wherein “Liquid” refers to master mixes that were not lyophilized, represented as a solid line and “Lyo” refers to master mixes that were lyophilized and is represented as a small dashed line.

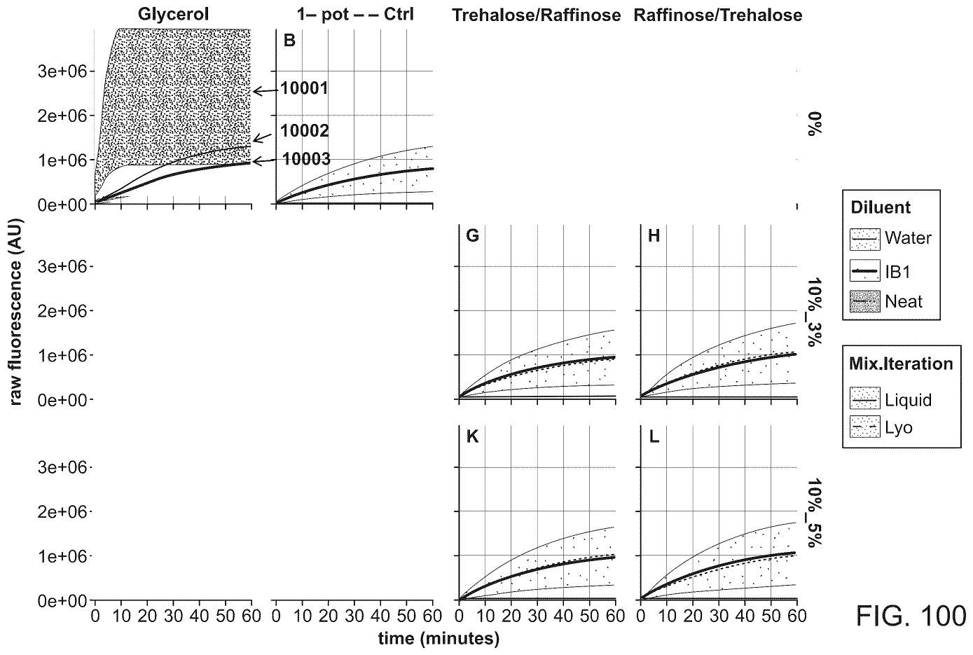

In some embodiments, a master mix of assay reagents are reconstituted after lyophilization. In some embodiments, a master mix of DETECTR assay reagents are reconstituted after lyophilization. In some embodiments, a master mix of DETECTR assay reagents, including Cas12M08 protein are reconstituted after lyophilization. In some embodiments, a master mix of amplification and DETECTR assay reagents, including Cas12M08 protein are reconstituted after lyophilization. In some embodiments, a master mix of RT-LAMP and DETECTR assay reagents, including Cas12M08 protein are reconstituted after lyophilization. The results of a reconstituted lyophilized Cas12M08 DETECTR master mix are shown in FIG. 100. The left-hand, y-axis plots raw fluorescence. The x-axis plots time in minutes. Subplot A contains results for a glycerol control (10001), IB1 buffer (10003) and water (10002), with no excipients present. Subplot B contains results for the one-pot control. Subplot G shows results for a sample containing 10% Trehalose and 3% Raffinose. Subplot K shows results for a sample containing 10% Trehalose and 5% Raffinose. Subplot H shows Raffinose and Trehalsose at 10% and 3%, respectively, and subplot L shows Raffinose and Trehalose at 10% and 5%, respectively. The bold solid trace shows the experimental series where IB1 reaction buffer was used. The thin solid lines, see FIGS. 99A-99B (9901), located on either side of the bold solid line representing the IB1 buffer, show the boundaries of the data.

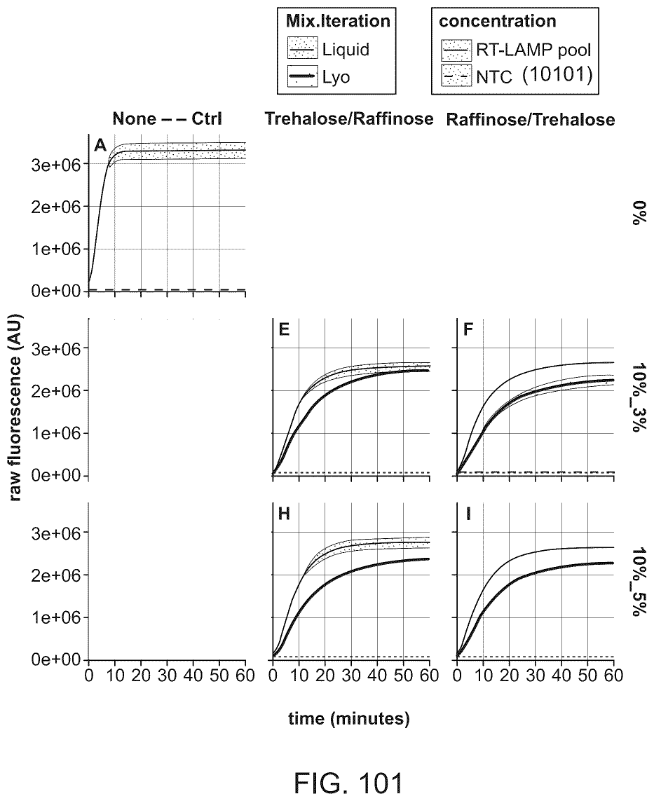

FIG. 101 presents results for a Cas14a1-based DETECTR assay. The left-hand, y-axis plots raw fluorescence. The x-axis plots time in minutes. Subplot A presents results for non-lyophilized control master mix comprising an RT-LAMP pool as the target, seen as the solid line with speckled background for the data boundaries. The thin solid line with speckled background series) contains a non-lyophilized control with no target present. Subplots E, F, H and I present results for master mixes, with RT-LAMP pool as the target. Lyophilized master mixes with RT-LAMP pool as the target are represented by the solid line, while non-lyophilized master mixes are represented by the thin solid line with speckled background series. Lyophilized master mixes without target are represented by the bold line series and non-lyophilized samples without target are represented by the thin solid line with speckled background series. Additionally, subplot E contains excipients 10% Trehalose and 3% Raffinose. Subplot H contains 10% Trehalose and 5% Raffinose. Subplot F contains 10% Raffinose and 3% Trehalose and subplot I contains 10% Raffinose and 5% Trehalose.

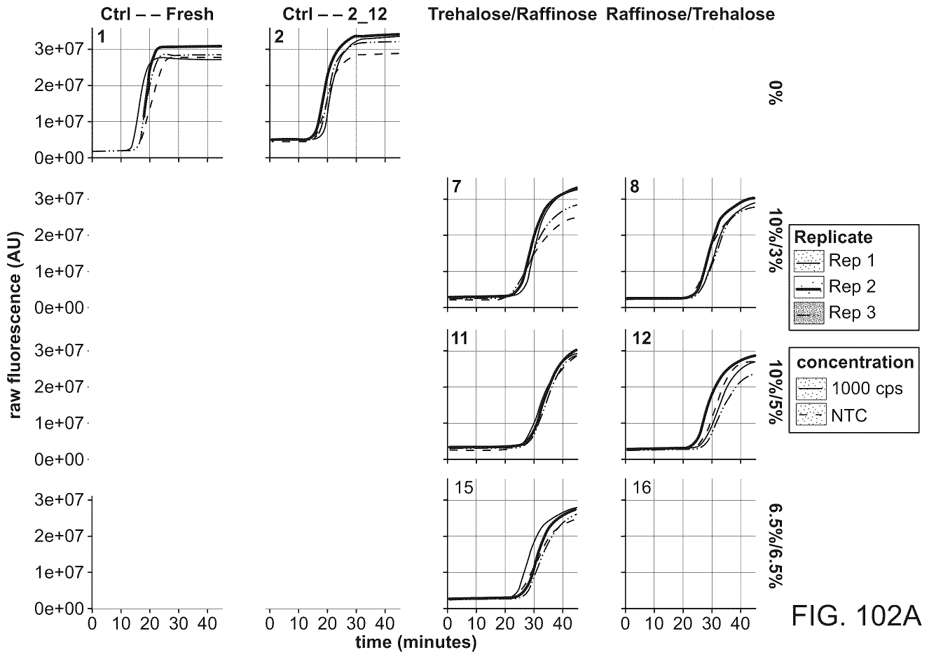

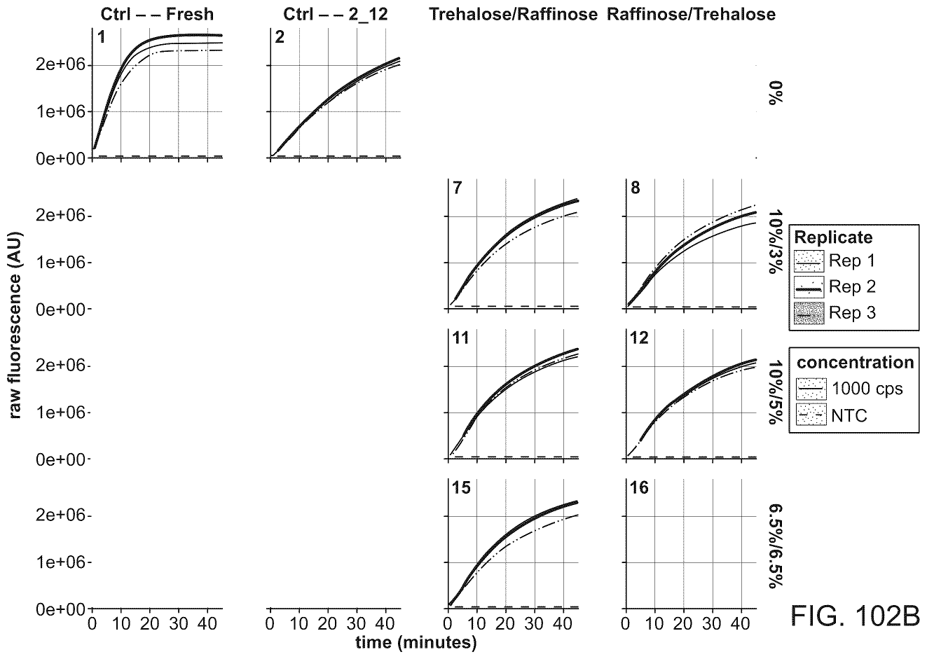

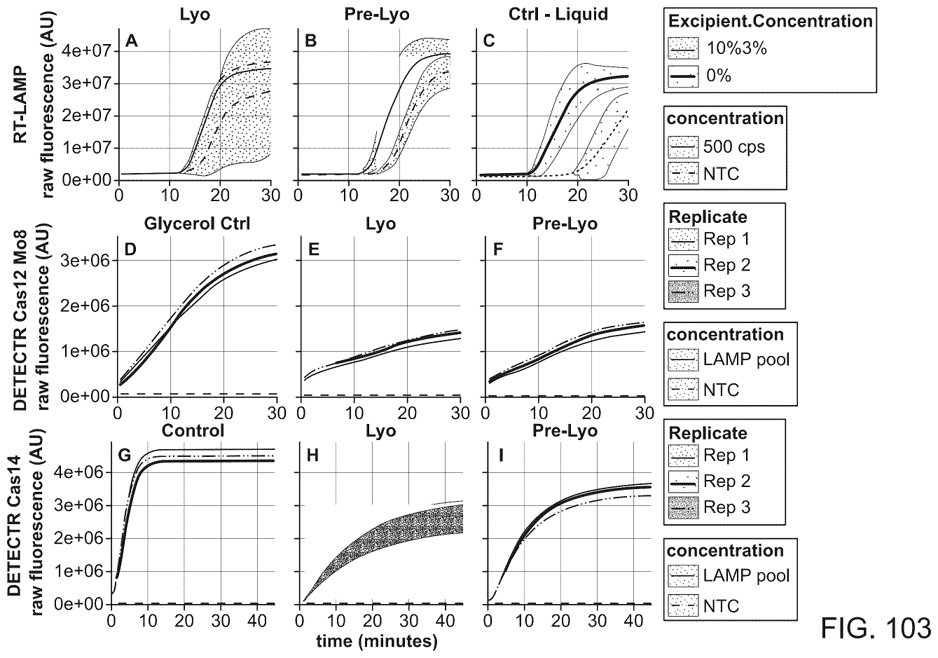

In some embodiments, the master mix of reagents and target for one assay is lyophilized. In some embodiments, the master mixes from more than one assay are pooled and lyophilized. In some embodiments, a duration of time occurs between mixing and lyophilization. FIG. 102A and FIG. 102B present results of an embodiment where the master mixes for both RT-LAMP and Cas12M08-based DETECTR assays were each made, mixed together, and stored for two weeks prior to lyophilization. Subplot 1 of FIG. 102A presents results for a sample without excipient and was not lyophilized but was prepared immediately prior to running the RT-LAMP assay. Subplot 2 presents results for a sample that did not contain excipient and was lyophilized two weeks after initial preparation. Subplots 7, 8, 11, 12, 15 and 16 of FIG. 102A show results for samples containing various types and amounts of excipient as described on the axes. Each color line represents one of three replicates. The solid lines represent a concentration of 1000 copies of target per sample and the dashed line contain no targets. FIG. 102B shows similar results for the DETECTR assay ran on an aliquot of the same sample.

In some embodiments, lyophilized master mixes of reagents from more than one assay are prepared in volumes of less than 1 mL. In some embodiments, lyophilized master mixes of reagents from more than one assay are prepared in volumes of less than 250 uL. In some embodiments, lyophilized master mixes of reagents from more than one assay are prepared in volumes of less than 25 uL. In some embodiments, lyophilized master mixes of reagents from more than one assay are prepared in volumes of less than 10 uL. FIG. 103 presents results for lyophilized reactions at 25 uL in volume. Subplots A, B, C show results for an RT-LAMP assay. Subplot A shows RT-LAMP results for lyophilized sample with excipients, where the solid line represents a sample with 500 copies per sample of target and the dashed line represents a no target control. Subplot B presents results for non-lyophilized sample with excipients, where the solid line represents a sample with 500 copies per sample of target and the dashed line represents a no target control. Subplot C presents results for samples without excipients. Subplots D, E, and F show results for a Cas12M08-based DETECTR assay. Subplots G, H and I show results for a Cas14-based DETECTR assay. For both assays, the solid lines show results for samples with the same targets used in the RT-LAMP assay, with three replicates. The dashed lines represent no target controls.

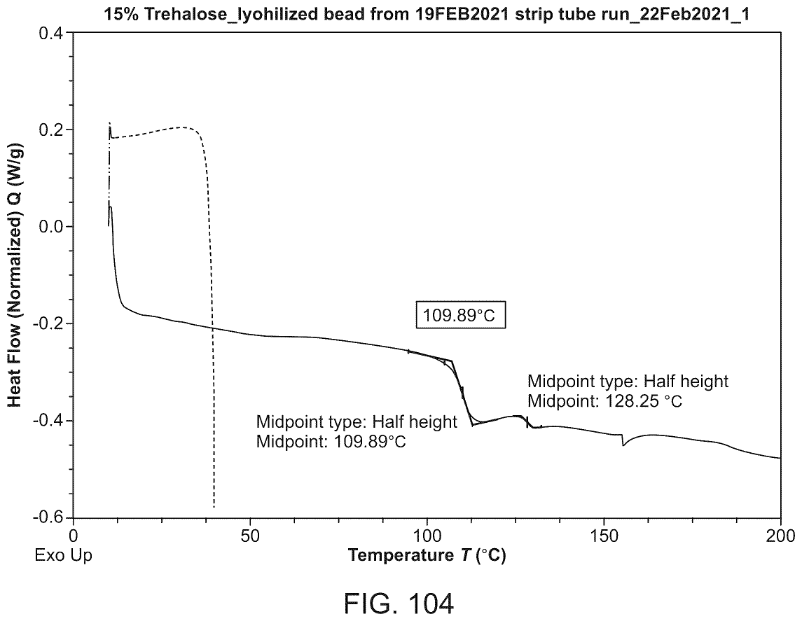

In some embodiments, lyophilized master mixes of assay reagents are analyzed by dynamic scanning calorimetry (DSC). FIG. 104 plots heat flow versus temperature in degrees Celsius for such an embodiment. In some embodiments, such measurements provide a measure of quality of the master mix throughout the lyophilization process, specifically the freeze dry steps. In some embodiments, DSC measurements yield a glass transition temperature which can be indicative of long-term stability of a sample.

In some embodiments, an excipient is used to stabilize the sample throughout the lyophilization process that may comprise freezing and drying steps. FIG. 105 presents a list of excipients as well as each excipient's primary/secondary status, function, freezing temperature (T_{f}) as determined by DSC, critical temperature (T_{critical}) as determined by DSC, and event. In some embodiments, T_{critical} is the temperature required to ensure that the sample is fully frozen.

In some embodiments, the hygroscopicity of enzymes and reagents is optimized to improve lyophilization performance.

LAMP Amplification with Cas14a DETECTR in Single Reaction Volume (One-Pot)

Described herein are various methods of sample amplification and detection in a single reaction volume. Any of the devices described herein may be configured to perform amplification and detection in a same well, chamber, channel, or volume in the device. In certain instances, methods include simultaneous amplification and detection in the same volume. In certain instances, methods include sequential amplification and detection in the same volume. In some embodiments, sample amplification may comprise LAMP amplification.

FIGS. 54A-54B presents results for an embodiment of HotPot involving LAMP amplification with Cas14a DETECTR in single reaction volume (one-pot) In such an embodiment, Cas14 fails to function as a one-pot in standard LAMP conditions. In some embodiments, Cas14a was tested to see if it can function in a one-pot reaction using LowLAMP at 50 C using Klenow(exo-) as the DNA polymerase. (a) The signal from SYTO9, a DNA binding dye, indicates the production of DNA by LAMP. It was confirmed that LowLAMP was able to generate DNA amplicon in the presence of Cas14 in three different buffers. (b) The signal from the Cas14 FQ reporter in the one-pot reaction condition shown in (a). No signal was detected in the one-pot reaction, even though DNA was generated. For this embodiment, results suggest that Cas14 is inhibited in the LAMP reaction.

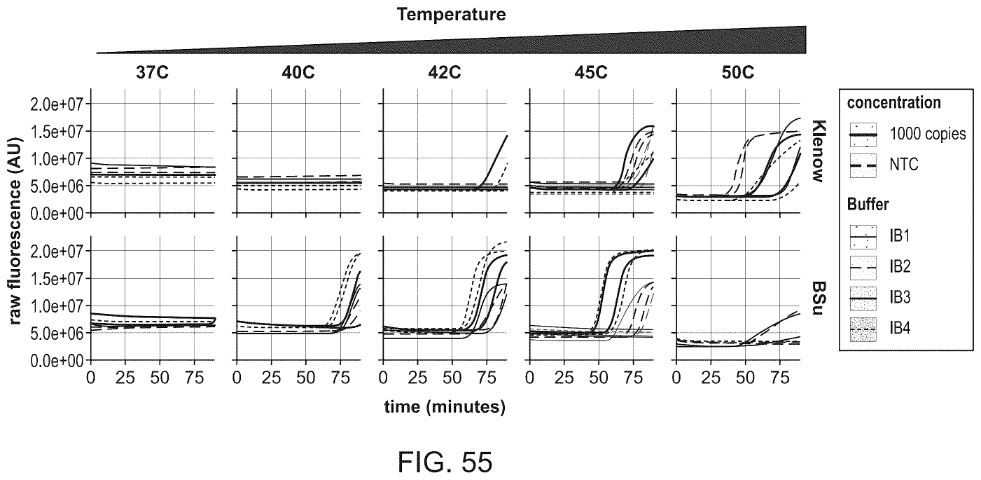

For some embodiments, RT-LAMP can be performed at lower temperatures by using Klenow(exo-) or Bsu polymerases: LowLAMP. RT-LAMP is normally performed at temperatures between 55 C-70 C. Results can be seen in FIG. 55. This temperature range is influenced by the polymerase used. In standard RT-LAMP, a Bst or Bsm polymerase is used which show peak activity above 55 C. In this embodiment, the performance of RT-LAMP at temperatures from 37 C to 50 C. By using Klenow(exo-) and Bsu polymerases was evaluated and demonstrated functional RT-LAMP on an RNA target of SARS-CoV-2 at temperatures as low as 40 C. For this embodiment, the performance of these enzymes in 4 different buffers was tested and peak activity for Klenow(exo-) was observed at 50 C, and peak activity for Bsu was at 45 C. For some embodiments, this method is called LowLAMP, as it functions at lower temperatures than standard RT-LAMP.

The Cas14a1 sequence is:

(SEQ ID NO: 1)
MAKNTITKTLKLRIVRPYNSAEVEKIVADEKNNREKIALEKNKDK
VKEACSKHLKVAAYCTTQVERNACLFCKARKLDDKFYQKLRGQFP
DAVFWQEISEIFRQLQKQAAEIYNQSLIELYYEIFIKGKGIANAS
SVEHYLSDVCYTRAAELFKNAAIASGLRSKIKSNFRLKELKNMKS
GLPTTKSDNFPIPLVKQKGGQYTGFEISNHNSDFIIKIPFGRWQV
KKEIDKYRPWEKFDFEQVQKSPKPISLLLSTQRRKRNKGWSKDEG
TEAEIKKVMNGDYQTSYIEVKRGSKIGEKSAWMLNLSIDVPKIDK
GVDPSIIGGIDVGVKSPLVCAINNAFSRYSISDNDLFHFNKKMFA
RRRILLKKNRHKRAGHGAKNKLKPITILTEKSERFRKKLIERWAC
EIADFFIKNKVGTVQMENLESMKRKEDSYFNIRLRGFWPYAEMQN
KIEFKLKQYGIEIRKVAPNNTSKTCSKCGHLNNYFNFEYRKKNKF
PHFKCEKCNFKENADYNAALNISNPKLKSTKEEP.

In some embodiments, a tracrRNA, known as R1518 is used and is native for the system and has the sequence of:

(SEQ ID NO: 51)
CUUCACUGAUAAAGUGGAGAACCGCUUCACCAAAAGCUGUCCCUU
AGGGGAUUAGAACUUGAGUGAAGGUGGGCUGCUUGCAUCAGCCUA
AUGUCGAGAAGUGCUUUCUUCGGAAAGUAACCCUCGAAACAAAUU
CAUUU.

In some embodiments, Cas14a.1 uses two RNA components, a tracrRNA and a crRNA. In some embodiments, the native crRNA repeat that occurs with this system is used.

In some embodiments, crRNAs used are:

R3297-SARS-CoV-2 N-gene having the sequence of:

(SEQ ID NO: 52)
GUUGCAGAACCCGAAUAGACGAAUGAAGGAAUGCAACCCCCCAGC
GCUUCAGCGUUC;

R3298-Mammuthus—having the sequence of:

(SEQ ID NO: 53)
GUUGCAGAACCCGAAUAGACGAAUGAAGGAAUGCAACGCCGAUAA
UGAUGUAGGGAU;

R4336-SARS-CoV-2—having the sequence of:

(SEQ ID NO: 54)
GUUGCAGAACCCGAAUAGACGAAUGAAGGAAUGCAACUGGCACUG
AGAAUUUGACUA;
and

R4783-OC43—having the sequence of:

(SEQ ID NO: 55)
GUUGCAGAACCCGAAUAGACGAAUGAAGGAAUGCAACUACAAGUG
CCUGUAGGUAUA.

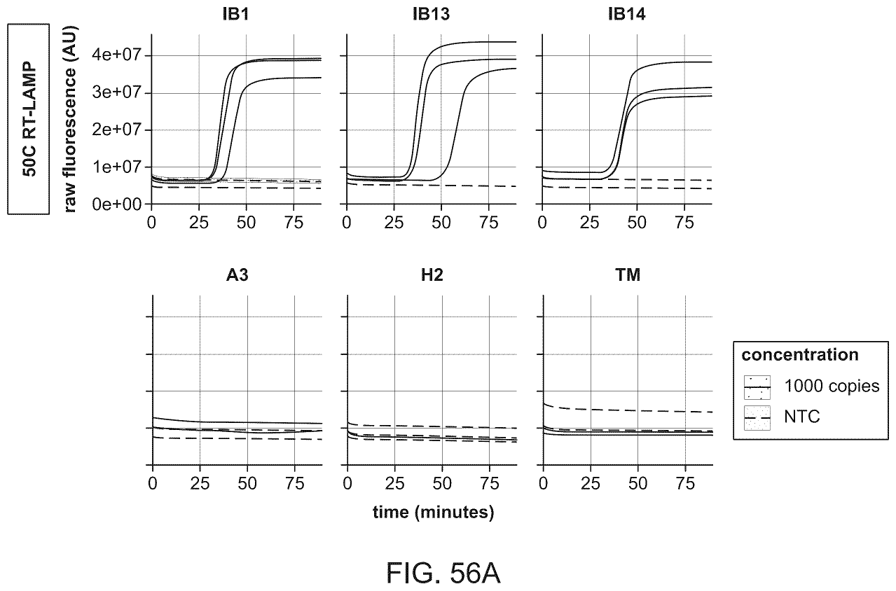

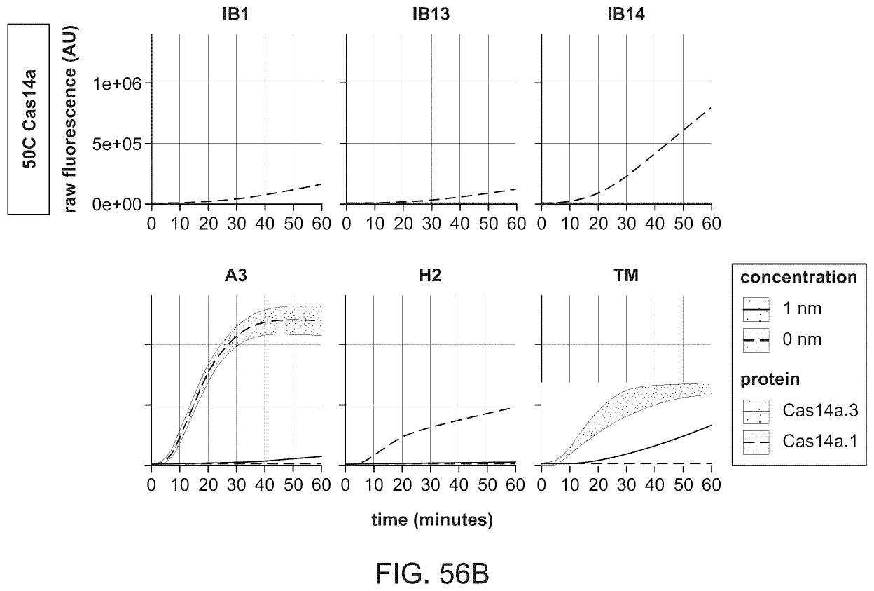

For some embodiments, buffers that were compatible with Cas14a and low temperature RT-LAMP (LowLAMP) were identified. Results are shown in FIGS. 56A-56B. For this embodiment, LowLAMP and Cas14a were found to be optimally functional in different buffers. For this embodiment, the performance of LowLAMP at 50 C in buffers that are optimal for LowLAMP (IB1, IB13, IB14) and buffers that are optimal for Cas14a (A3, H2, TM) was evaluated as well as the performance of Cas14a in the same buffers at 50 C. Results in FIGS. 56A-56B show that both LowLAMP and Cas14a are functional in the LowLAMP optimal buffers, but Cas14a is most compatible with buffer IB14. This work enables the identification of a buffer condition that is compatible with both isothermal amplification and Cas14a DETECTR, a key step in moving towards a one-pot reaction.

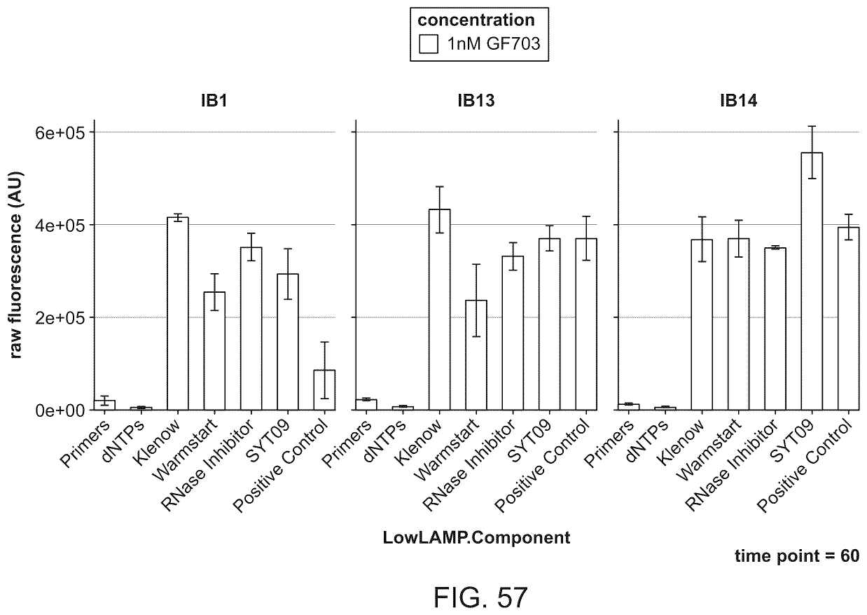

In some embodiment, primers and dNTPs have the greatest inhibitory effect on Cas14 performance as seen in the results presented in FIG. 57. Here the impact of the individual components of LowLAMP on the performance of Cas14 were tested to determine which components might be responsible for the inhibition seen when attempting one-pot reactions. Each component was added to the Cas14a reaction with 1 nM of target dsDNA and the reaction was run for 60 minutes at 50 C. The results indicate that primers and dNTPs contribute to the inhibition of LAMP.

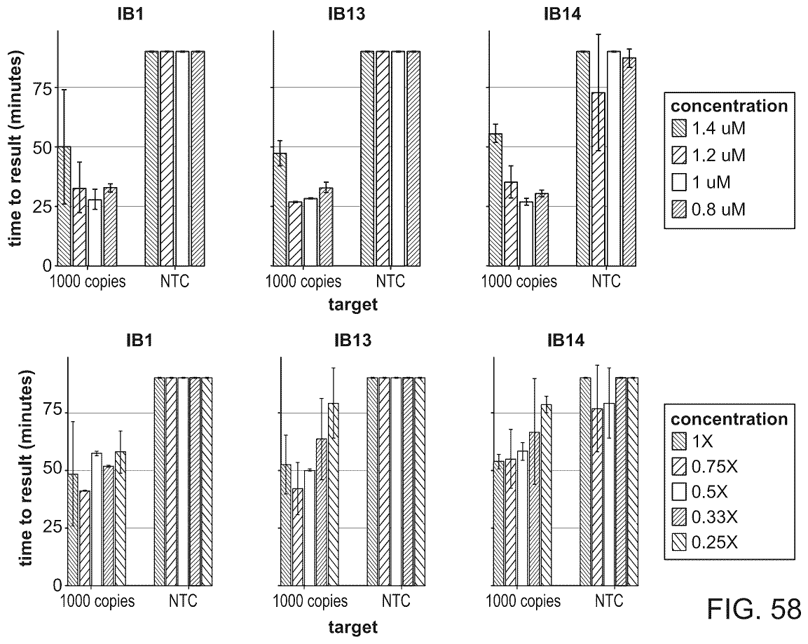

In some embodiments, LAMP functions with lower concentrations of dNTPs and primers as shown in FIG. 58. In some embodiments, both the dNTPs and primers in LAMP have an inhibitory effect on Cas14. In this embodiment, the performance of lower concentrations of dNTPs and primers was tested to see the impact on the performance of LowLAMP using Klenow(exo-) at 50 C. For this embodiment, results show a time-to-result where a lower value indicates faster amplification. (a) Titration of dNTPs shows that LowLAMP functions down to 0.8 mM dNTPs. For this embodiment, the performance of the assay was improved at 1.2 mM and less versus the standard 1.4 mM. (b) Titration of primers in LowLAMP. The results of this assay show that going below 0.5× of primers can have an inhibitory effect. Together these results suggest that the dNTPs and primers concentrations can be reduced without negatively impacting LowLAMP performance, which may help relieve the observed inhibition of Cas14.

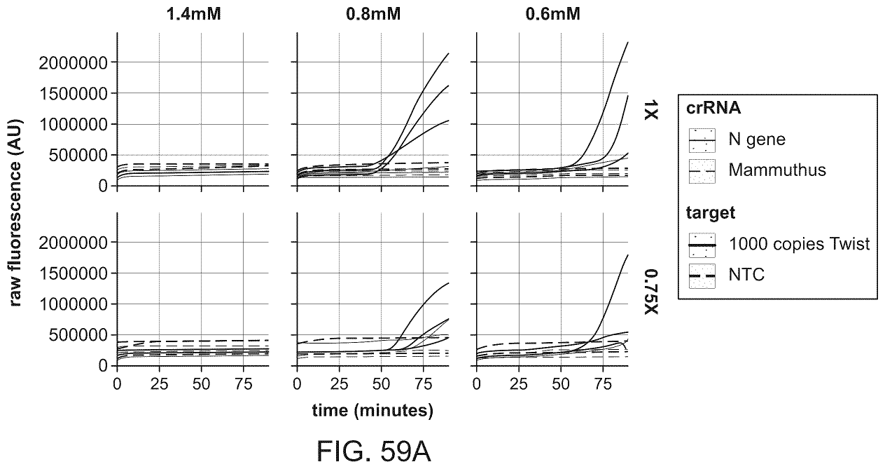

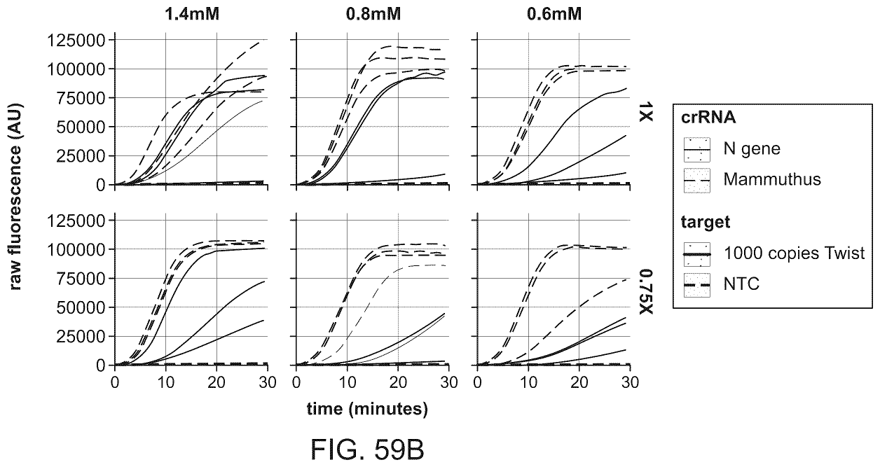

In some embodiments, One-pot Cas14 with LowLAMP was tested at 50 C. For such embodiments, one-pot DETECTR using Cas14 and LowLAMP at 50 C using a Klenow(exo-) DNA polymerase was shown to be functional as seen in FIGS. 59A-59B. For this embodiment, lower primer and dNTP concentrations were tested. Other embodiments have suggested that Cas14 is inhibited at the standard 1.4 mM and 1× primer concentration. (a) Signal from Cas14a one-pot. For this embodiment, one-pot DETECTR reactions using a Cas14 were either complexed with a SARS-CoV-2 N-gene crRNA that targeted the amplicon generated by LowLAMP, or complexed with a non-target crRNA that targeted a gene in the Mammuthus primigenius mtDNA. Results from this embodiment, showed a signal only in the reaction that were run with target RNA and the crRNA targeting the amplicon. No signal was seen when there was no target present, or the Cas14 crRNA was targeting the Mammuthus gene. (b) To confirm the generation of the target of interest by LAMP, amplicons generated in the one-pot reaction were run in a Cas12 DETECTR reaction using a SARS-CoV-2 N-gene crRNA. These results demonstrated that the amplicon was generated when the target RNA was present. Furthermore, the results indicate that the one-pot reaction is functional at 0.8 and 0.6 mM dNTPS, but not at 1.4 mM dNTPs due to Cas14 inhibition at this concentration of dNTPs.

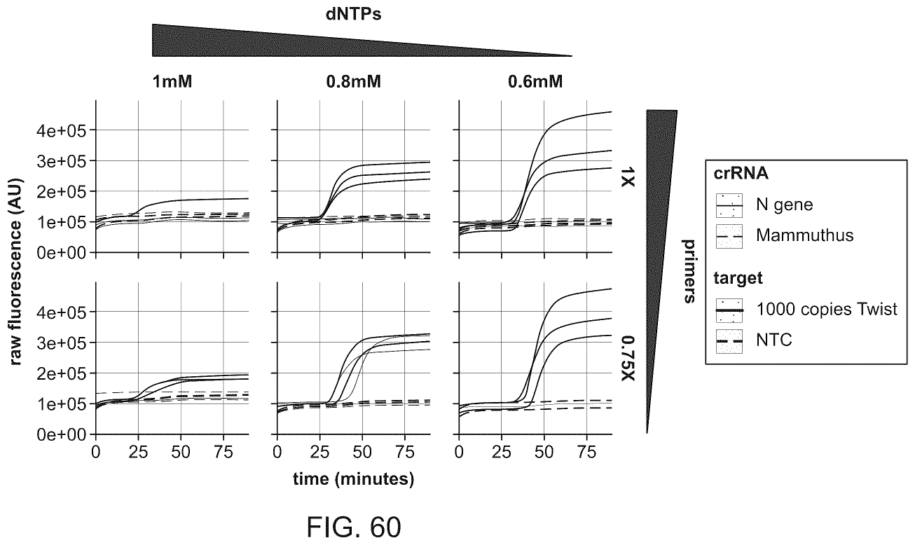

In some embodiments, Cas14 is used with a polymerase (e.g., a Bsm DNA polymerase, a Bst DNA polymerase, a Klenow(exo-) DNA polymerase, or a Bsu DNA polymerase) (55 C) for the OnePot assay. The one-pot reaction may be faster if the reaction temperature is increased from 50 C to 55 C. However, the DNA polymerase used in LowLAMP is not functional at 55 C, so here Bsm DNA polymerase was used which works more robustly at 55 C than other LAMP polymerases such as Bst. Several different concentrations of dNTPs and primers were tested and performance of the assay was assessed. Results shown in FIG. 60 indicate that a signal is generated by Cas14 when Cas14 has the crRNA that matches the target amplicon (N-gene) and when the target RNA is present in the reaction. The speed of the reaction was fastest with 1 mM dNTPs, but the overall signal was lower than when 0.8 and 0.6 mM dNTPs were used.

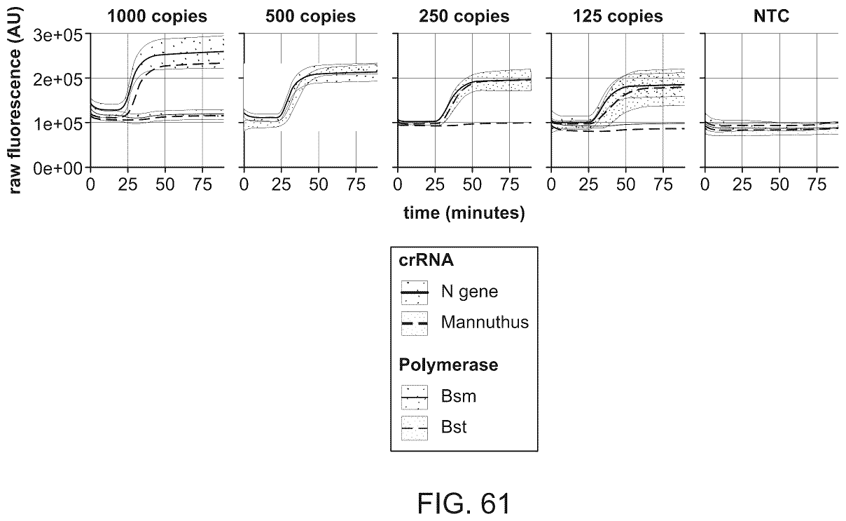

For some embodiments, the initial performance of the one-pot DETECTR reaction, called HotPot was evaluated. Results are shown in FIG. 61. In this experiment, a limit of detection experiment for two different DNA polymerases at 55 C was performed. Cas14 crRNAs targeting either the SARS-CoV-2 N-gene or an off-target gene were included in the experiment. The assay at 1000, 500, 250, or 125 copies/rxn of SARS-CoV-2 RNA genomes was tested. Robust performance of the assay at the lowest tested concentration of 125 copies/rxn for 3/3 replicates as shown in FIG. 61.

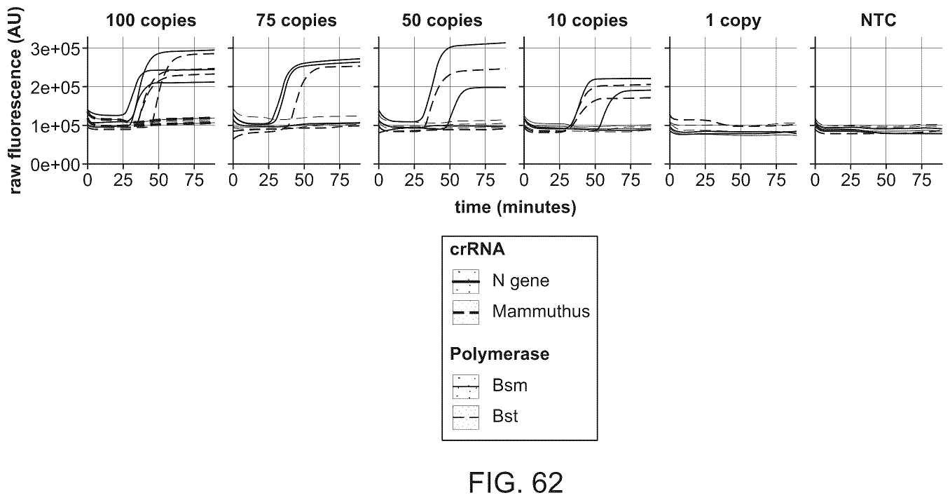

For some embodiments, the limit of detection experiments were performed two different DNA polymerases at 55 C. Results are shown in FIG. 62. For these embodiments, Cas14 crRNAs targeting either the SARS-CoV-2 N-gene or an off-target gene were included in the experiments and the assay at 100, 75, 50, 10, or 1 copies/rxn of SARS-CoV-2 RNA genomes were tested. For these embodiments, robust performance of the assay at 100 copies/rxn for 3/3 replicates was observed. At 75 copies and below several replicates did not show up, but detection down to 10 copies/rxn for 2/3 replicates was observed

For various embodiments described herein, the assay turn-around time is 5 minutes. In some embodiments, the assay turn-around time is 10 minutes. In some embodiments, the turn-around time is 15 minutes. In some embodiments, the turn-around time is 20 minutes. In some embodiments, the turn-around time is 30 minutes. In some embodiments, the turn-around time is 40 minutes or less.

### One-Pot NECTR: NEAR Amplification+Cas14a DETECTR in Single Reaction Volume

Described herein are various methods of sample amplification and detection in a single reaction volume. Any of the devices described herein may be configured to perform amplification and detection in a same well, chamber, channel, or volume in the device. In certain instances, methods include simultaneous amplification and detection in the same volume. In certain instances, methods include sequential amplification and detection in the same volume. In some embodiments, sample amplification may comprise NEAR amplification.

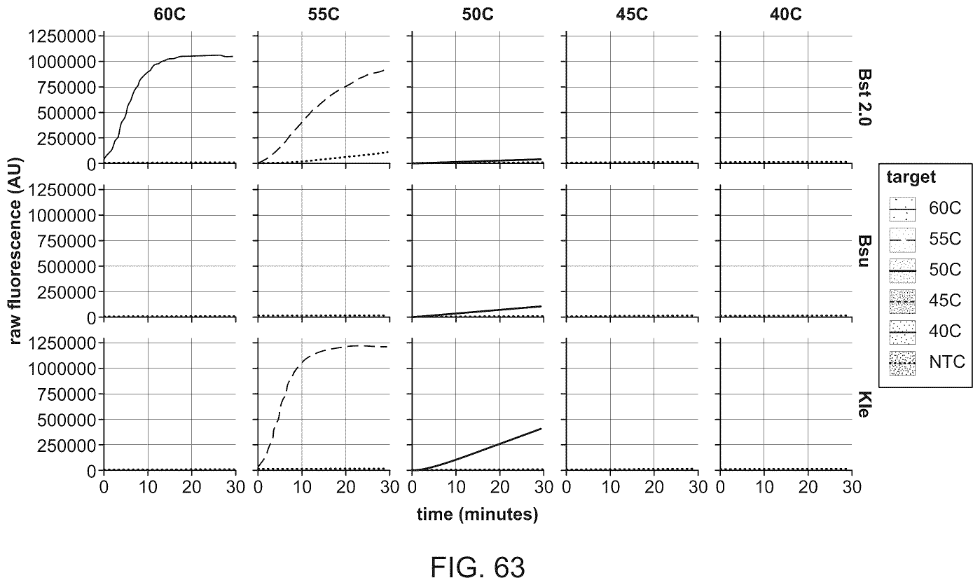

For some embodiments, replacing Bst polymerase in NEAR can enable SARS-CoV-2 detection at lower temperatures as shown in FIG. 63. In these embodiments, NEAR protocol uses Bst 2.0 to generate an amplicon from SARS-CoV-2 at 60 C. This polymerase also functions at 55 C, but at reduced capacity. The performance of Bsu and Klenow(-exo) polymerases at lower temperatures was evaluated for these embodiments. For these embodiments, it was found that Klenow(-exo) polymerase enables robust amplification at 55 C and 50 C. Data shown in FIG. 63 is Cas12M08 DETECTR following a NEAR amplification reaction. For some embodiments, amplification reactions were performed at the indicated temperature for 10 minutes in a buffer that was composed of a mixture of 1× IsoAmp buffer+0.5× CutSmart buffer. It was observed in this embodiment that Cas14a shows peak activity at 55 C and 50 C, but not at 60 C.

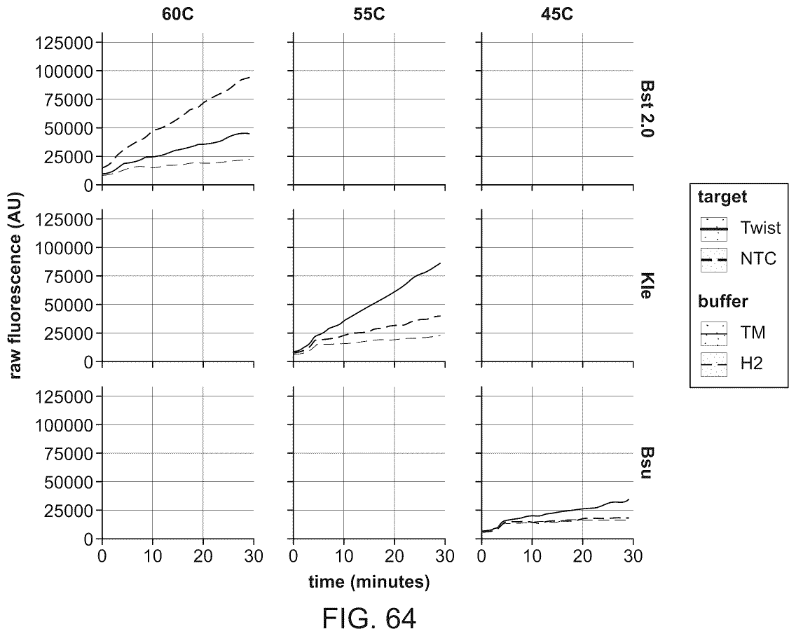

For some embodiments, NEAR amplification functions in Cas14a optimal buffers as shown in FIG. 64. The optimal buffer for Cas14a has less salt and higher Mg2+ than the optimal buffer for NEAR amplification. In this embodiment, the performance of NEAR amplification in two buffers for Cas14a: 1× TM Buffer and 1× H2 Buffer. In this embodiment, amplification reactions were performed with the indicated polymerase, buffer, and reaction temperature with Nt.BstNBI, Omniscript RT, M2805 FWD, M2811 REV. Data shown in FIG. 64 is the result of a Cas12M08 DETECTR reaction to evaluate the amount of amplicon produced. For this embodiment, results indicate that TM Buffer and H2 Buffer may function for NEAR amplification. However, results suggest that the reactions are producing ˜10 times less amplicon than an optimal NEAR reaction. This suggests room for further buffer optimization. In some embodiments: 1× TM Buffer: 20 mM Tricine, pH 8.5; 2 mM KOAc; 100 μg/mL BSA; 15 mM MgOAc. In some embodiments: 1× H2 Buffer: 20 mM Tris-HCl, pH 8.8; 2 mM KOAc; 0.1% Tween-20; 17.5 mM MgOAc.

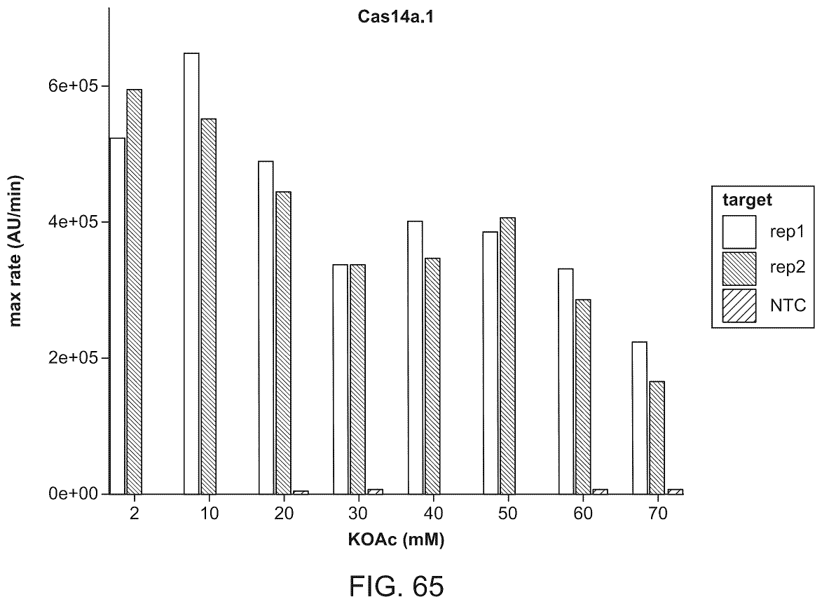

In some embodiments, Cas14a functions in a range of KOAc salt concentrations as shown in FIG. 65. In some embodiments, Cas14a reaction buffer (H2 Buffer) contains 2 mM KOAc. In some embodiments, amplification reactions require higher concentrations for optimal efficiency. In some embodiments, the performance of a Cas14a DETECTR reaction in increasing concentrations of KOAc. Cas14a DETECTR reaction was evaluated with tracrRNA R1518, crRNA R2424, at a target concentration of 1 nM, and a reaction temperature of 50 C. Results shown in FIG. 65 indicate that Cas14a is maximally active at 2-10 mM KOAc, but remains robustly active up to 60 mM. At 70 mM KOAc performance of Cas14a begins to drop off. In some embodiments, 1× H2 Buffer is composed of: 20 mM Tris-HCl; pH 8.8; 2 mM KOAc; 0.10% Tween-20; and 17.5 mM MgOAc.

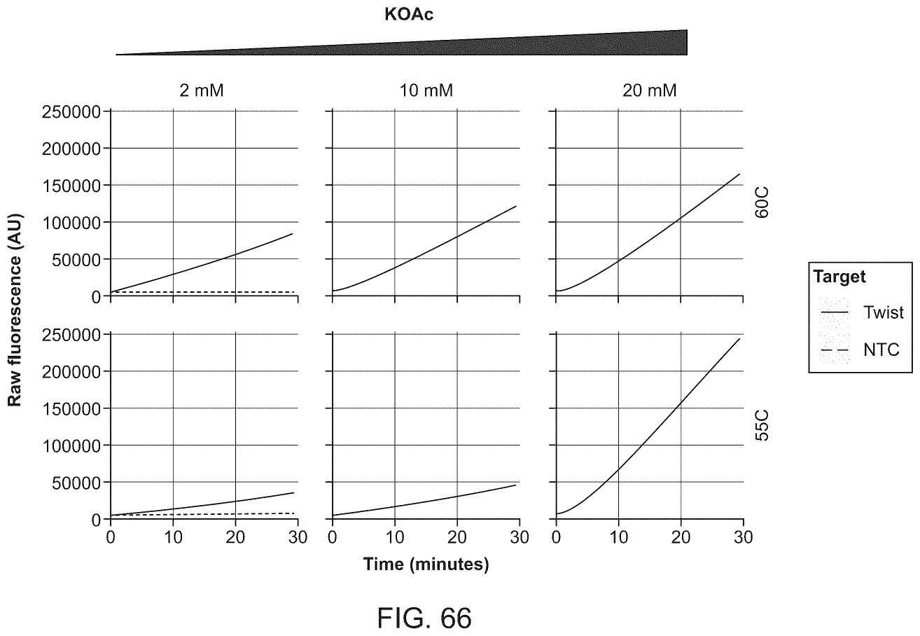

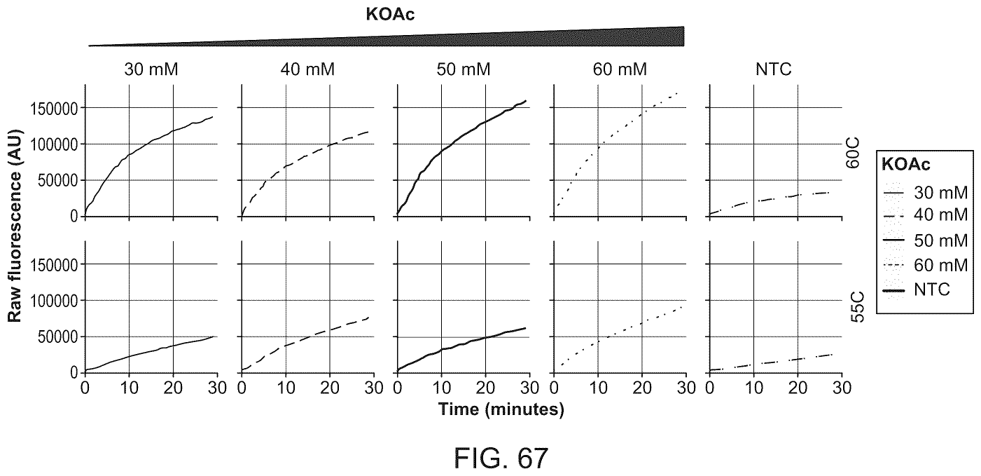

In some embodiments, increasing concentrations of KOAc improve NEAR performance in Cas14a optimal buffers, as seen in FIG. 66. NEAR amplification in Cas14a optimal reaction buffers was initially seen to be less efficient than when amplification is performed in the optimal NEAR buffer (1× IsoAmp+0.5× CutSmart). In some embodiments, the impact of increasing amounts of KOAc on the performance of NEAR amplification in the background of a Cas14a optimal amplification buffer (H2 Buffer). As seen in FIG. 66 results show how Cas12M08 DETECTR assay performed after NEAR amplification to measure amplification efficiency. Results indicate that increasing KOAc to up to 60 mM improves the amount of NEAR amplicon generated. From 2 mM to 60 mM KOAc are functional ranges for Cas14a. In some embodiments: 1× H2 Buffer: 20 mM Tris-HCl, pH 8.8; 2 mM KOAc; 0.1% Tween-20; 17.5 mM MgOAc. In some embodiments, increasing concentrations of KOAc improve NEAR performance in Cas14a optimal buffers as seen in FIG. 67.

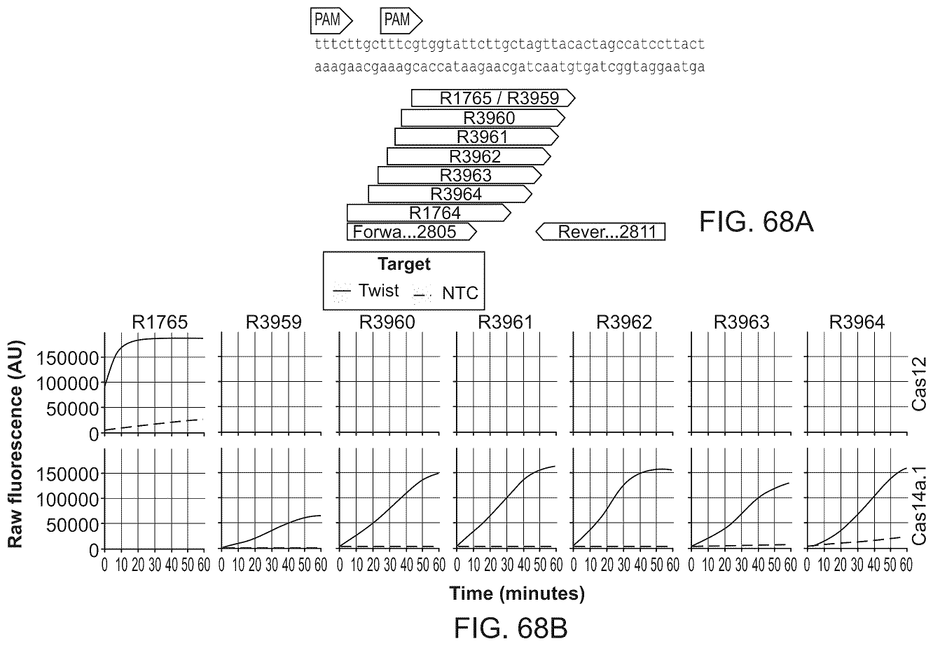

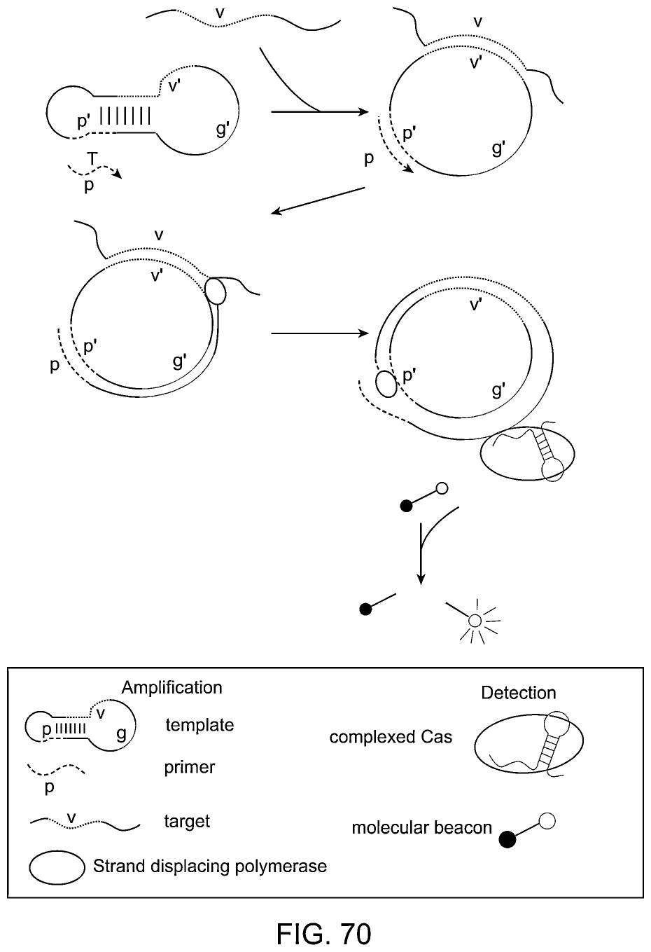

Performance of Cas14a.1 crRNAs on SARS-CoV-2 E-gene amplicon are shown in FIG. 70 for some embodiments. In some embodiments, Cas14a crRNAs, designed to target the SARS-CoV-2 E-gene NEAR amplicon generated by primers M2805/M2811. Results are shown in FIGS. 68A-68B. FIG. 68A shows the position of the crRNAs. R1765 and R1764 are Cas12M08 crRNAs that target the amplicon. PAM sequences for Cas12M08 are indicated, but there are no TTTR PAMs for Cas14 to use within this amplicon. FIG. 68B shows the performance of Cas14a.1 crRNAs on NEAR amplicon (generated at 60 C for 15 minutes). All tested crRNAs worked robustly with the least amount of background from R3960, R3961, and R3962.

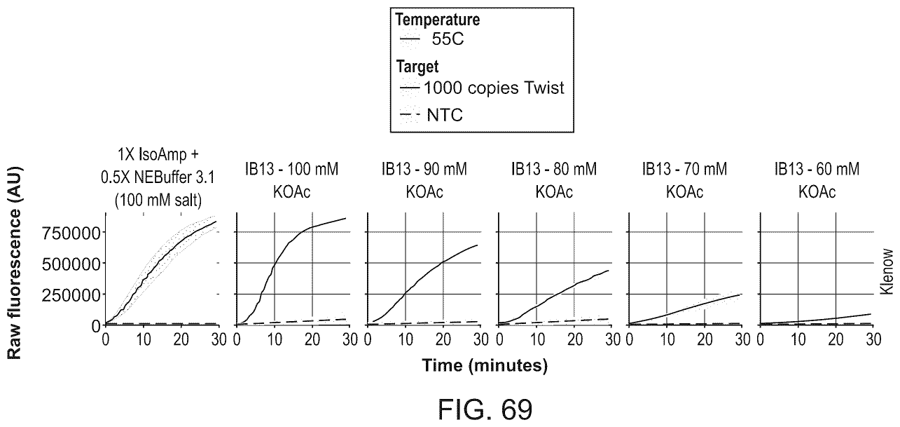

In some embodiments, the performance of Klenow(exo-) NEAR assay in IB13 buffer at decreasing salt concentrations was evaluated as shown in FIG. 69. In these embodiments, standard NEAR reaction buffer was composed a mixture of 1× IsoAmp buffer and 0.5× NEBuffer 3.1, which gives a final salt concentration of 100 mM. In these embodiments, variations of the IB13 buffer with various concentrations of KOAc as the salt were tested. In these buffer variations the NEAR assay using Klenow(exo-) as the polymerase was run at 55 C for 20 minutes. To readout the amount of amplicon produced, a Cas12M08 DETECTR assay on 2 μL of the NEAR amplicon was performed. The results indicate that the best NEAR performance was with 100 mM KOAc, similar to the standard NEAR reaction buffer, and that the amount of amplicon produced was reduced as the salt is reduced. The assay has acceptable performance at 80-70 mM KOAc. In some embodiments, the IB13 buffer has a composition of: 20 mM Tris-HCl, pH 8.8; 10 mM (NH4)2SO4; 50 mM KOAc; 5 mM MgOAc; 1% Tween-20; 1 mg/mL BSA.

One-Pot sRCA: Rolling Circle Amplification with Cas14a DETECTR in Single Reaction Volume

Described herein are various methods of sample amplification and detection in a single reaction volume. Any of the devices described herein may be configured to perform amplification and detection in a same well, chamber, channel, or volume in the device. In certain instances, methods include simultaneous amplification and detection in the same volume. In certain instances, methods include sequential amplification and detection in the same volume. In some embodiments, sample amplification may comprise RCA.

FIG. 70 presents an overview of sRCA. In this system, a target nucleic acid is added to a system that contains components for a one-pot RCA+Cas protein reaction. The RCA portion of the system is composed of a dumbbell-shaped DNA template, a primer, and a DNA polymerase. For some embodiments, the DETECTR portion of the reaction is composed of a Cas protein, such as Cas12 or Cas14, a crRNA that targets the amplicon generated by RCA, but not the dumbbell-shaped DNA template, and a FQ ssDNA reporter. A target nucleic acid (e.g., viral RNA), which is capable of binding to the dumbbell DNA template, is added to the system. The target nucleic acid base pairs with the DNA template. The more extensive base pairing between the target and the DNA template causes the internal base pairing of the dumbbell to be disrupted, which opens up a binding site for the primer. The DNA polymerase can then use this primer to begin RCA. In some embodiments, As RCA proceeds, the amplicon is generated which contains the target site for the Cas protein. The Cas protein recognizes this site through base pairing with the crRNA and initiates trans-cleavage of the FQ ssDNA reporter. In some embodiments, the system contains fewer components than other one-pot approaches and does not require a RT enzyme.

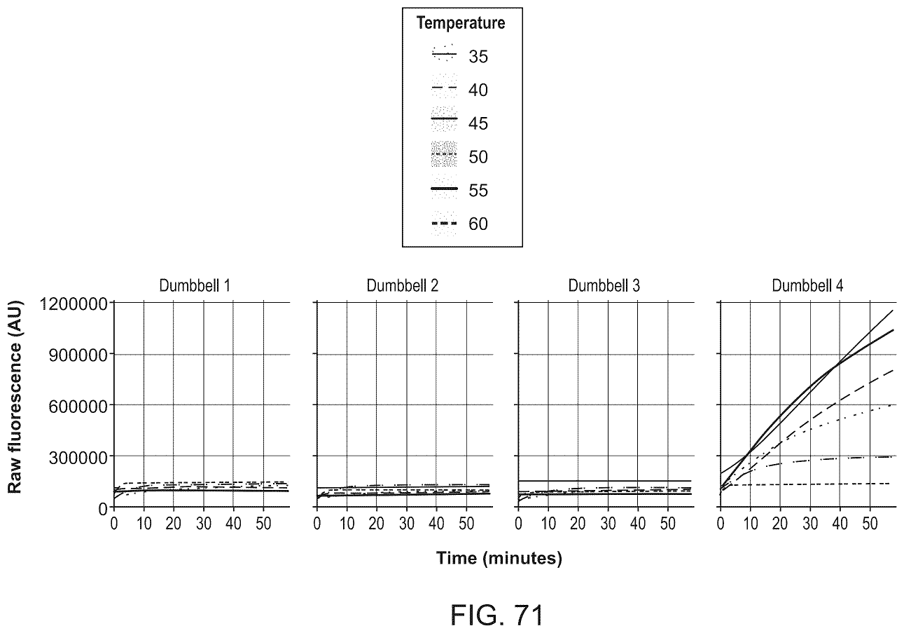

In some embodiments, screening dumbbell DNA templates are screened for sRCA performance, as shown in FIG. 71. In some embodiments, four dumbbell DNA templates for RCA contain a Cas12 or Cas14 target sites. For these embodiments, a DNA binding dye, SYTO9, is used to monitor whether these dumbbells are functional in RCA at a variety of temperatures for 1 hour. Results as seen in FIG. 71 indicate that only dumbbell 4 was functional in generating DNA by RCA. The peak performance of the system was seen at 35-45 C.

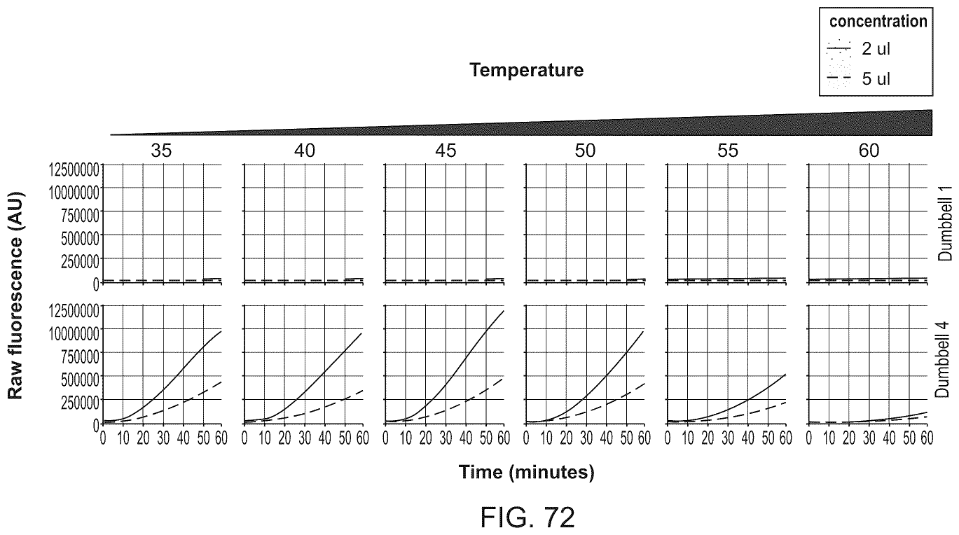

For some embodiments, the performance of Cas14a to detect product of RCA reaction was monitored, as seen in FIG. 72. In other embodiments, it was shown that Dumbbell 4 was functional in RCA and that Dumbbell 1 was not functional. In some embodiments, either 2 μL or 5 μL of the RCA reactions were added to a 20 μL Cas14a DETECTR reaction that contained a crRNA that is capable of detecting the amplicon generated by RCA, but not the Dumbbell DNA template. The results indicate that Cas14a is capable of detecting the amplicon as expected, and that increased performance is seen with lower amounts of RCA added to the reaction.

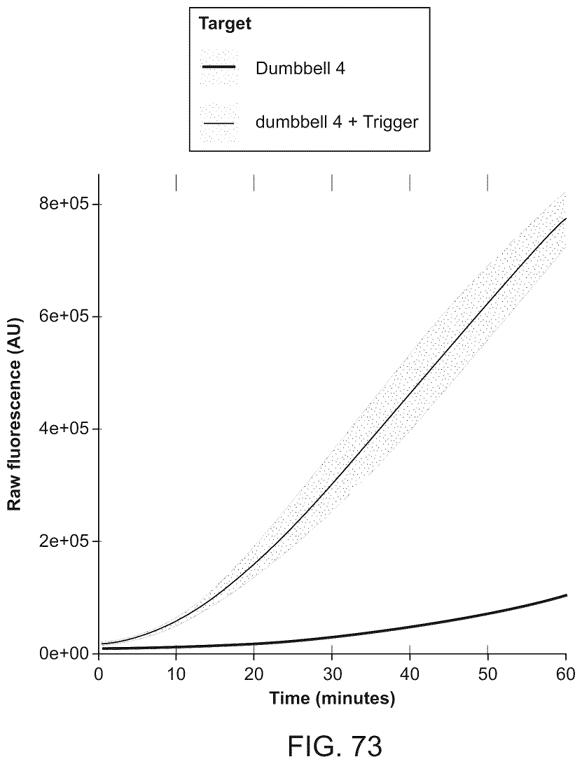

In some embodiments, of the One-Pot assay sRCA, Cas14 is used. Functional results for such embodiments are shown in FIG. 73. The reaction was performed at 45 C in two conditions as two embodiments. In one embodiment, the DNA template, Dumbbell 4, does not contain a trigger oligo and in the other embodiment, the DNA template does have a trigger oligo. In the embodiment with the trigger oligo, which initiates RCA, higher signal is observed as compared to the other embodiment without the trigger oligo. This demonstrates that Cas14 is capable of detecting the RCA amplicon in a one-pot reaction, and that the sRCA reaction is controlled by the presence of the trigger oligo.

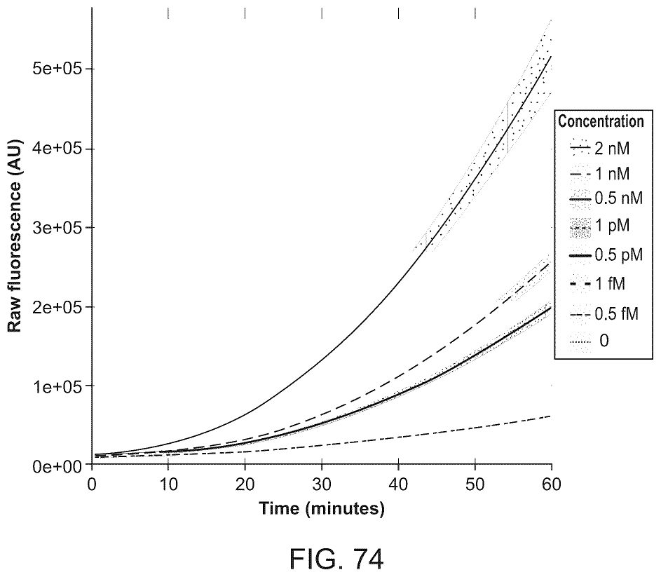

In some embodiments, a trigger oligo is titrated for a Cas14 One-Pot sRCA assay. For this embodiment, the minimal concentration of trigger oligo that is required to initiate the one-pot Cas14 sRCA reaction was determined. Results shown in FIG. 74 indicate that at least 0.5 nM of trigger oligo is required to initiate the sRCA reaction.

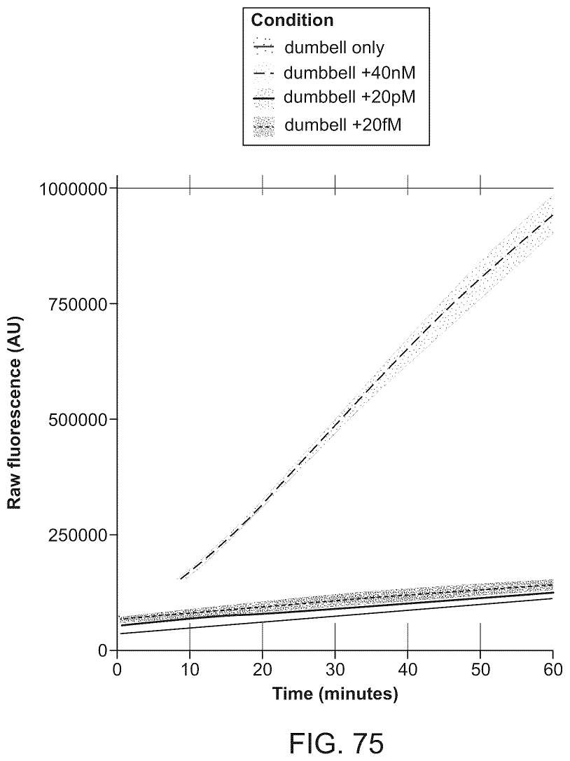

In some embodiments, the Cas12M08 enzyme is used in the one-pot sRCA assay. In other embodiments, it has been shown that Cas14 is capable of functioning in a one-pot sRCA reaction. In this embodiment, it was shown that Cas12M08 is also capable of functioning in this assay at 45 C. The results from the cleavage of ssDNA FQ reporter included in the sRCA reaction are shown in FIG. 75. The results for such an embodiment indicate that Cas12M08 is capable of functioning in the one-pot sRCA format. In some embodiments, the concentration of stock trigger oligo is: (1) 40 nM stock=2 nM final conc; (2) 20 μM stock=1 μM final conc.; and (3) 20 fm stock=1 fM final conc.

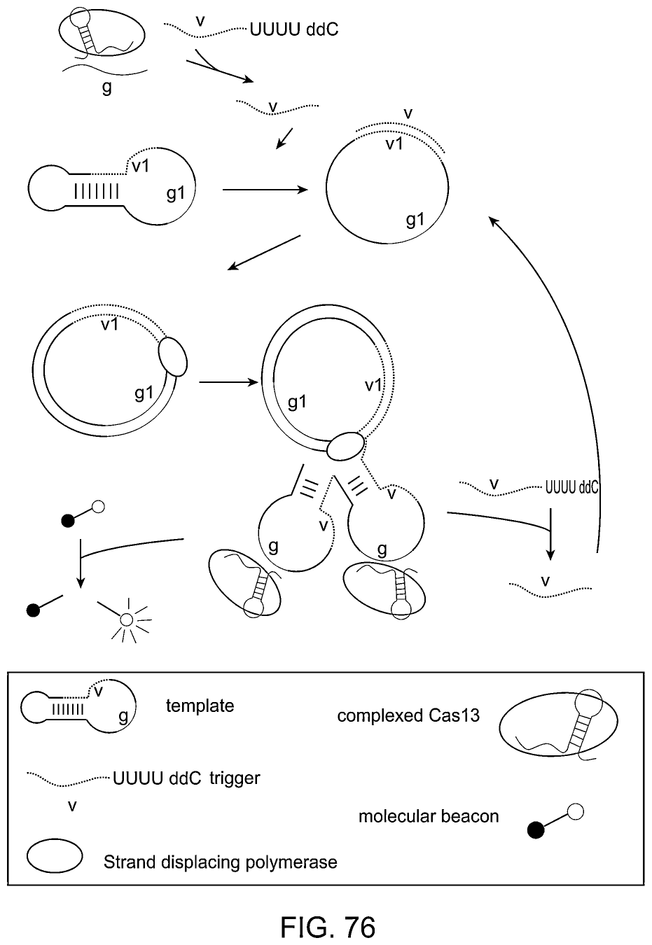

FIG. 76 presents an overview of RCA positive feedback for Cas13. In some embodiments, Cas13 is programmed to recognize an RNA target (g). When the viral RNA target is present, a blocking motif on the 3′ end of the primer (v) is removed. After removal of this blocking motif, the primer can then serve to open up the circular template and allow for amplification by RCA using a DNA polymerase. As the amplicon is generated the same target sequence as the original RNA (g) is generated. This ssDNA target sequence is then capable of being recognized by Cas13 which can either remove additional blocking groups from the primer (v) or cleave a FQ reporter that generates a fluorescent signal. This system forms a positive feedback loop for Cas13.

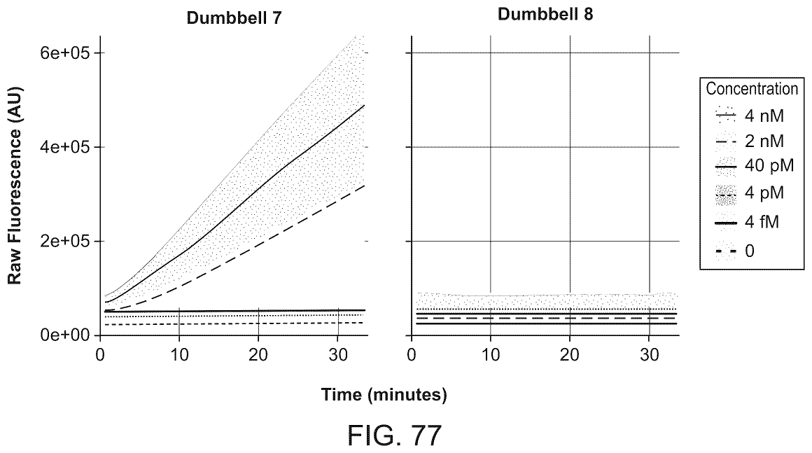

FIG. 77 presents results for the evaluation of Cas13-compatible DNA templates for RCA. In some embodiments, two dumbbell DNA templates for RCA that contain a Cas13 ssDNA targeting site. In some embodiments, a DNA binding dye, SYTO9, is used to monitor whether these two dumbbells are functional in RCA at 30 C. The performance of the two templates was monitored a titration of the primers that are used to trigger the amplification. The results indicate that Dumbbell 7, but not Dumbbell 8 is compatible with RCA as shown in FIG. 77.

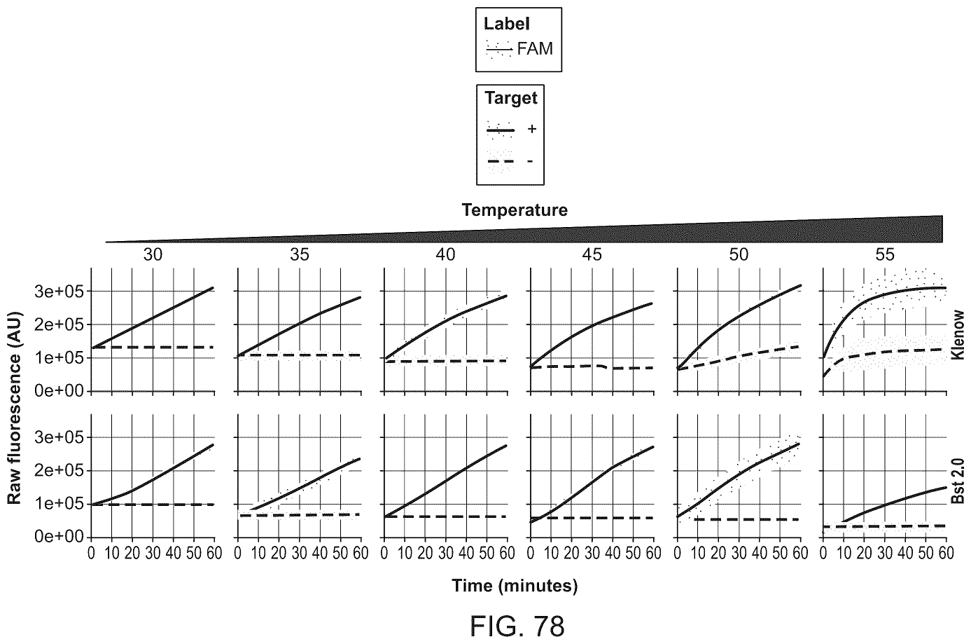

In some embodiments, Cas13-compatible DNA template is used for RCA. FIG. 78 presents results for such embodiments, to determine whether Cas13-compatible DNA template is functional in RCA. In some embodiments a circular DNA dumbbell for sRCA has a Cas13 ssDNA target site, known as dumbbell 7. In some embodiments, a DNA binding dye, SYTO9, is used to monitor whether the DNA template is functional in RCA at various temperatures for two different polymerases using 2 nM of trigger oligo (the primer for sRCA). The results shown in FIG. 78 indicate that dumbbell 7 is capable of generating amplicon at temperatures from 30 to 55 C. Such an embodiment, consisting of the utilization of dumbbell 7 for generating amplicons from 30 to 55 C, enables for the use of the Cas13 enzyme in the OnePot reaction.

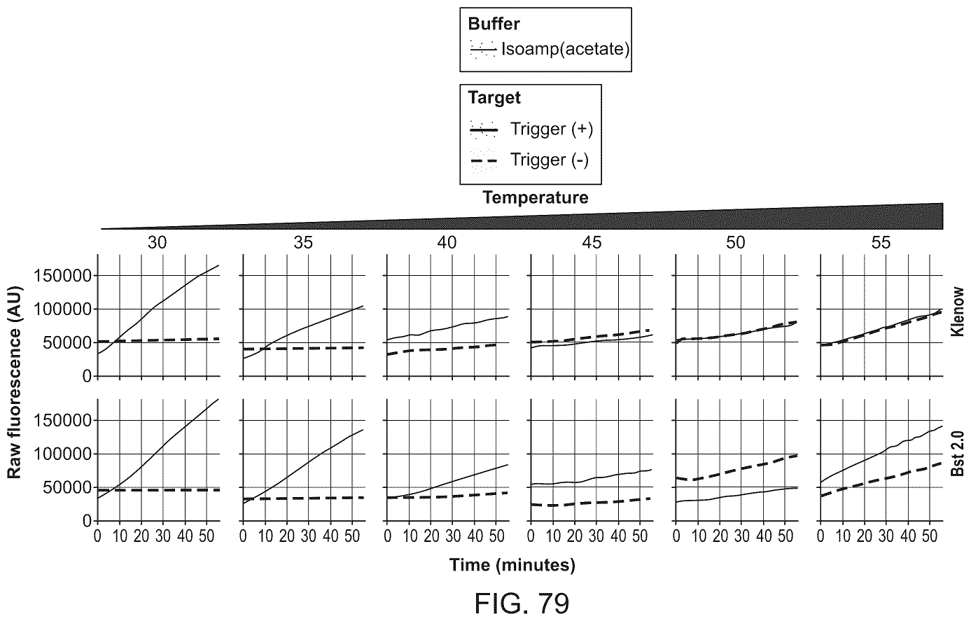

In some embodiments, the Cas13M26 is used in the one-pot sRCA reaction. FIG. 79 presents results for such an embodiment, where the amplicon generated by RCA contains a ssDNA region that is capable of being recognized by a Cas13 gRNA. As the reaction proceeds, additional ssDNA target is produced, while the activated the Cas13M26 cleaves a FQ RNA reporter to generate a signal. The performance of this reaction at a variety of temperatures was evaluated and it was shown that Cas13M26 is capable of detecting this ssDNA region of the RCA amplicon from 30-40 C, which aligns with the previously established active temperatures for Cas13M26. The performance of two different polymerases at the temperatures of interest was also compared. The results shown in FIG. 79 suggest that Cas13 can be used in a one-pot reaction where RCA is the amplification method, and that Cas13's ability to detect ssDNA is preserved in this embodiment.

CasPin: Cas13 Positive-Feedback Loop Leveraging Cas13 ssDNA Targeting

Described herein are various methods of signal amplification. Any of the devices described herein may be configured to perform signal amplification after the reporter has been cleaved by the programmable nuclease. Signal amplification may improve detection of rare targets in a complex sample. In certain instances, methods include leveraging ssDNA targeting of the programmable nuclease (e.g., Cas13) to create a positive feedback loop upon biding of the programmable nuclease to the target nucleic acid to cleave additional reporters and amplify the signal generated by the presence of the target nucleic acid.

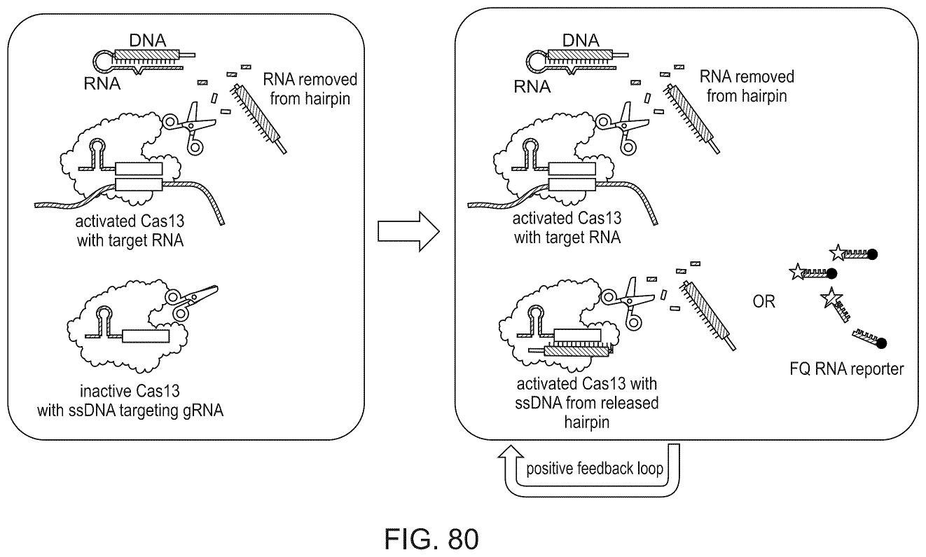

FIG. 80 presents an overview of CasPin. In some embodiments, the CasPin system uses two populations of Cas13. One is programmed with a crRNA that targets an RNA of interest, such as a viral genome. The other population is programmed with a crRNA that is optimal for ssDNA detection. In some embodiments, the systems also contains a hairpin-shaped oligo that is composed of both DNA and RNA. Finally, in some embodiments, there is a FQ RNA reporter that is used to readout the result of the assay. When Cas13 detects the RNA of interest, it can either cleave the FQ RNA reporter or the RNA on the hairpin oligo. When the RNA on the hairpin oligo is cleaved, it dissociates from the DNA revealing a ssDNA target site that can be recognized by the other population of Cas13 RNPs. This initiates a positive feedback loop where Cas13 recognizes the ssDNA target and cleaves more hairpin molecules, which increases the overall amount of target in the system, and leads to further activation of the system. As this process continues more and more FQ RNA reporter is cleaved, which is the ultimate readout of the assay.

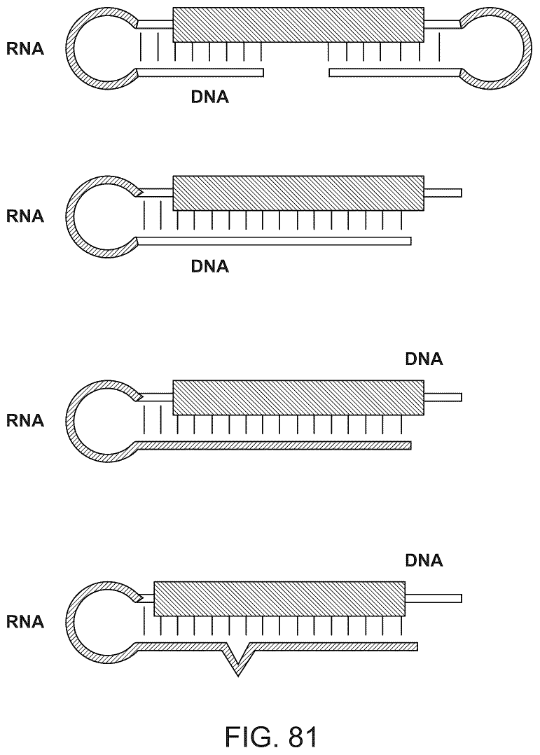

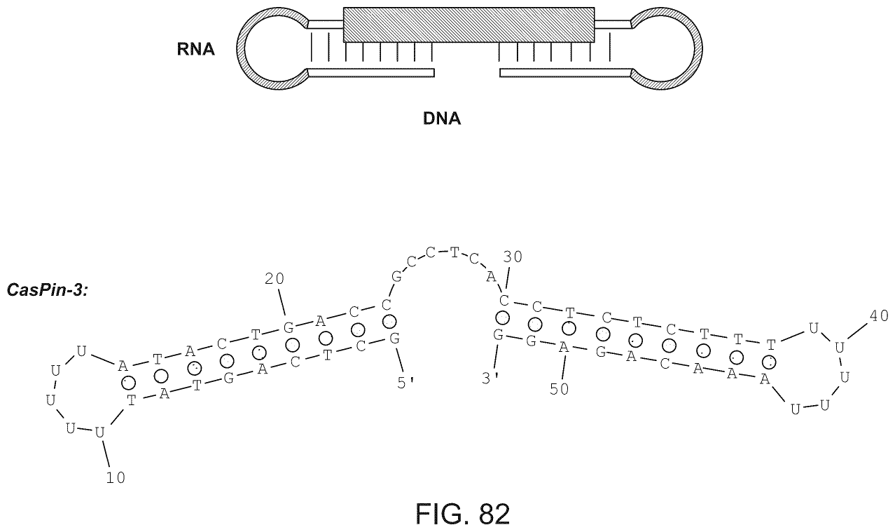

FIG. 81 presents potential structures of hairpins for CasPin. In some embodiments, the target ssDNA sequence is indicated by the purple rectangle. RNA loop structures could occur on either side of the target strand (either 5′, 3′ or both). The strand that is complementary to the target site could be DNA or RNA. The strand that is complementary to the target site could also be a perfect match to the target site, be shorter than the target site, or contain mismatches to help destabilize or promote trans-cleavage by Cas13.

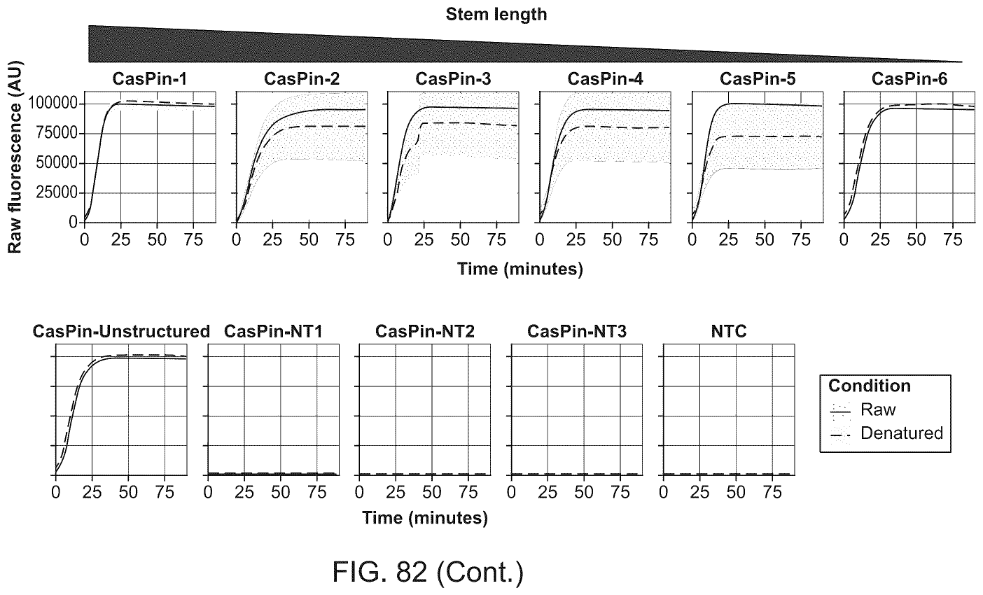

In some embodiments, two hairpins are used on either end of the target site. FIG. 82 presents results for such an embodiment and indicates capability for blocking Cas13 from recognizing the ssDNA target site. In some embodiments, CasPin oligos have varying lengths of hairpin stems. In some embodiments, CasPin oligo do not have stem structures. In some embodiments, CasPin oligos contain another DNA sequence. Such embodiments were evaluated and found to not be recognized by the crRNA. Both raw oligos from the manufacture and those that had been denatured and refolded at 25 C in a Cas13 DETECTR reaction were tested. The results of this experiment, shown in FIG. 84, demonstrate that Cas13 was able to recognize the target site regardless of the stem length. In some embodiments, longer stem length oligos block Cas13 recognition without RNA cleavage to release the structure. One-pot DETECTR on handheld microfluidic device

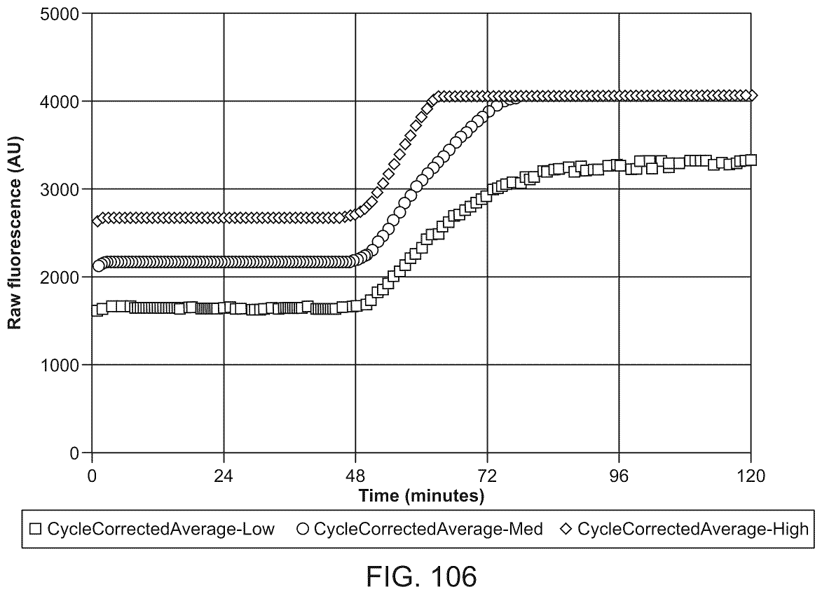

Described herein are various devices and methods for running one-pot DETECTR assays on a handheld device. Any of the devices described herein may be configured to perform a one-pot DETECTR assay. For example, the device shown in FIGS. 10A-12 may be configured to run a one-pot DETECTR assay. Results for such an embodiment are shown in FIG. 106. The y-axis displays raw fluorescence (AU) and the x-axis shows time in minutes. The three traces show the same data collected at three different acquisition settings. The line trace represented by squares, a low cycle corrected average, shows a setting that did not saturate the detector and thus shows the full dynamic range of the signal throughout the assay.

### Multiplexed DETECTR Assay-Based Lateral Flow Assay

Described herein are various methods of multiplexing detection. Any of the devices described herein may be configured for multiplexing (e.g., detecting multiple target nucleic acids). In certain instances, multiplexed detection may utilize one or more lateral flow assay strips.

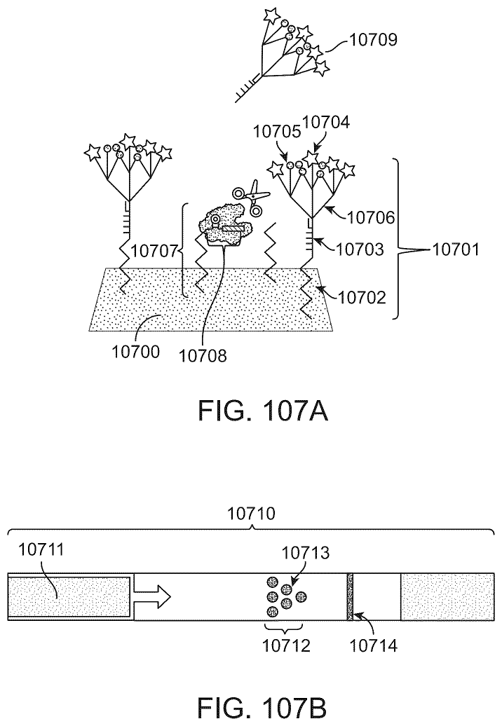

Described here are various devices and methods for a DETECTR™ assay based multiplex lateral flow strip as illustrated in FIG. 107. In some embodiments, reporters (10701) are immobilized to a surface (10700) of a solid support. In some embodiments, programmable nuclease (e.g., Cas-complex) probes (10707) are immobilized to a surface (10700). In some embodiments, programmable nuclease probes (10707) comprise guide nucleic acid such as a single guide RNA (sgRNA) (10708). In some embodiments, a programmable nuclease probe (10707) may comprise a sgRNA (10708) that is designed to be a compliment for a target nucleic acid of a sample. In some embodiments, programmable nuclease probes (10707) and reporters (10701) are both immobilized to a surface (10700). In some embodiments, programmable nuclease probes (10707) and reporters (10701) are both immobilized to a surface (10700) in close enough proximity that the reporter (10701) can be cleaved by the programmable nuclease of the programmable nuclease probe (10707). In some embodiments, programmable nuclease probes (10707) and reporters (10701) are both immobilized to a surface (10700) in close enough proximity that the reporter (10701) can be cleaved (10709) by the programmable nuclease of the programmable nuclease probe (10707) upon binding of a target nucleic acid to an sgRNA (10708) of the programmable nuclease probe (10707) when target nucleic acid and sgRNA (10708) are compliments. In such an embodiment, this indicates the presence of and is a “hit” for the target. In some embodiments, binding of a target nucleic acid that is complimentary to a sgRNA (10708) of the programmable nuclease probe (10707) results in the programmable nuclease of the programmable nuclease probe (10707) initiating cleavage of nucleic acids within a close enough proximity of the programmable nuclease. In some embodiments, the surface (10700) is in the bottom of a well. In some embodiments, a collection of a first programmable nuclease probe (10707) and a first reporter (10701) are immobilized to a surface at one location of the surface (10700).

In some embodiments, as illustrated in FIG. 107A, the reporter (10701) may comprise a surface linker (10702), a nucleic acid (10703), a second linker (10706), a detection moiety (e.g., a label) (10704), and an affinity molecule (e.g., a binding moiety) (10705). In some embodiments, the binding moiety (10705) is biotin. In some embodiments, there is more than one copy of the same reporter (10701) immobilized to the surface.

In some embodiments, lateral flow assay strips (10710) are used to detect cleaved reporters (10709). In some embodiments, cleaved reporters (10709) are contacted to the sample pad (10711) of the lateral flow strip (10710). In some embodiments, the cleaved reporters (10709) bind to conjugate particles present in the sample pad. In some embodiments, the conjugate particles are gold nanoparticles. In some embodiments, the gold nanoparticles are functionalized with anti-biotin. In some embodiments, the anti-biotin functionalized gold nanoparticles bind to the cleaved reporter which contains one or more biotins in the binding moiety (10705).

In some embodiments, the reporter contains a second linker. In some embodiments, the second linker links one or more binding moieties to the nucleic acid. In some embodiments, the second linker links one or more labels to the nucleic acid. In some embodiments, the second linker links both one or more binding moieties and one or more labels to the nucleic acid of the reporter. In some embodiments, the reporter is a dendrimer or trebler molecule.

In some embodiments, the reporter contains a label. In some embodiments, label is FITC, DIG, TAMRA, Cy5, AF594, Cy3, or any appropriate label for a lateral flow assay.

In some embodiments, the reporter may comprise chemical functional group for binding. In some embodiments, the chemical functional group is biotin. In some embodiments, the chemical functional group is complimentary to a capture probe on the flowing capture probe (e.g. conjugate particle or capture molecule). In some embodiments, the flowing capture probe is a gold nanoparticle functionalized with anti-biotin. In some embodiments, the flow capture probe is located in the sample pad. In some embodiments, the flowing capture probe is located in a conjugate pad in contact with the sample pad, wherein both lateral flow assay strip may comprise both the sample pad and conjugate pad, further wherein both the sample pad and the conjugate pad are in fluid communication with the detection region.

In some embodiments, the lateral flow assay strip (10710) contains a detection region (10712). In some embodiments, the detection region (10712) may comprise one or more detection spots. In some embodiments, the detection spots contain a stationary capture probe (e.g., capture molecule). In some embodiments, the stationary capture probe may comprise one or more capture antibodies. In some embodiments, the capture antibodies are anti-FITC, anti-DIG, anti-TAMRA, anti-Cy5, anti-AF594, or any other appropriate capture antibody capable of binding the detection moiety or conjugate.

In some embodiments, the flowing capture probe comprising FITC is captured by a stationary capture probe comprising anti-FITC antibody. In some embodiments, the flowing capture probe comprising TAMRA is captured by a stationary capture probe comprising anti-TAMRA antibody. In some embodiments, the flowing capture probe comprising DIG is captured by the stationary capture probe comprising anti-DIG antibody. In some embodiments, the flowing capture probe comprising Cy5 is captured by the stationary capture probe comprising anti-Cy5 antibody. In some embodiments, the flowing capture probe comprising AF574 is captured by the stationary capture probe comprising anti-AF594 antibody.

In some embodiments, the lateral flow assay strip (10710) may comprise a control line (10714). In some embodiments, the control line (10714) may comprise anti-IgG that is complimentary to all flowing capture probes. In some embodiments, when a flowing capture probe does not bind to a reporter the flowing capture probe will be captured by the anti-IgG on the control line, ensuring the user that the device is working properly even no signal is read from the test line.

In some embodiments, the lateral flow assay strip (10710) may comprise a sample pad. In some embodiments, the flowing capture probe may comprise anti-biotin. In some embodiments, the flowing capture probe may comprise HRP. In some embodiments, the flowing capture probe may comprise HRP-anti-biotin. In some embodiments, the flowing capture probe is HRP-anti-biotin DAB/TMB.

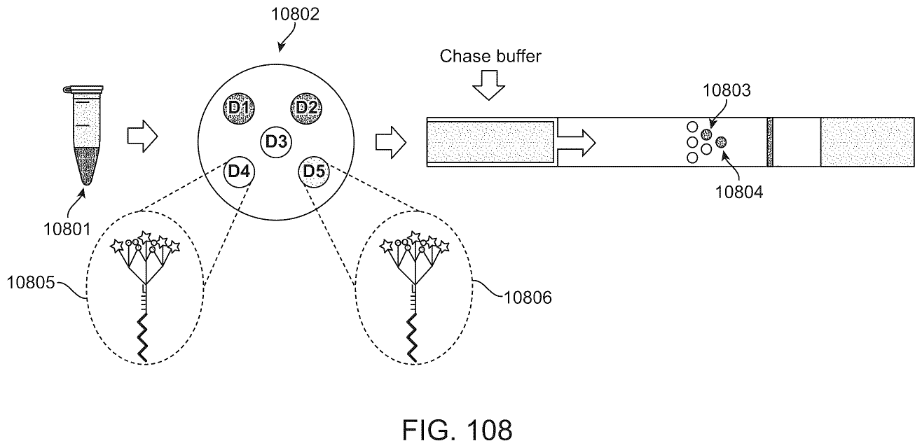

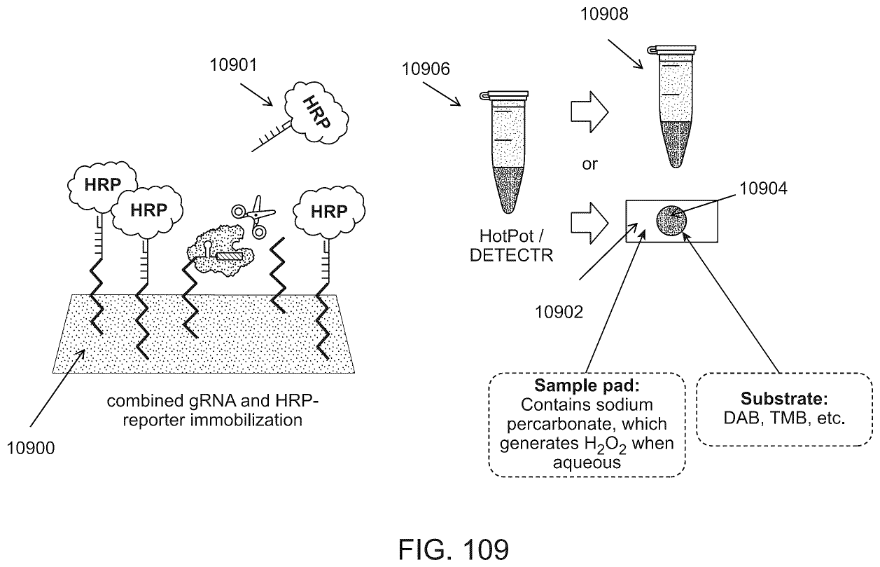

Described here are various devices and methods for a DETECTR™ assay based multiplex lateral flow strip as illustrated in FIG. 108. FIG. 108 depicts a non-limiting exemplary workflow for a DETECTR™ assay read out on a lateral flow assay strip. In some embodiments, a sample (10801) contains one or more target nucleic acid sequences. In some embodiments, a sample (10801) (e.g., a sample solution) contains at least first and second target nucleic acid sequences. In some embodiments, the sample (10801) is introduced into a well (10802) (e.g., D1-D5) where at one or more locations there are different guide nucleic acids such as sgRNAs immobilized to the surface of the well. In some embodiments, the sgRNAs are part of a programmable nuclease probe immobilized to a surface. In some embodiments, a sgRNA is designed to specifically bind to a target nucleic acid in the sample. In some embodiments, there are different sgRNAs corresponding to different locations (e.g., locations D1-D5) on the surface of the well, where each different sgRNA is complimentary for a different target nucleic acid sequence that may or may not be present in the sample. In some embodiments, in addition to the programmable nuclease probes containing sgRNAs, each location is functionalized with one or more reporter probes having distinct functional groups. In some embodiments, the reporter probes are in close enough proximity to be cleaved by the programmable nuclease probes. In some embodiments, as described in example 13, binding between a particular sgRNA and the target nucleic acid to which the sgRNA is designed to specifically bind allows for a section of one or more reporters are cleaved from a corresponding nucleic acid and released into the sample solution. In some embodiments, the reporter is functionalized with a label. In some embodiments, the lateral flow assay strip contains a detection region comprising detection spots (e.g., 10803, 10804), where each detection spot contains a different type of capture antibody. In some embodiments, each capture antibody type specifically binds to a particular label type of a reporter. In some embodiments, a first detection spot (10803) contains the capture antibody anti-FITC. In some embodiments, location D5 on the surface of the well (10802) contains a first immobilized programmable nuclease probe including the sgRNA specific to the first target nucleic acid sequence. In some embodiments, D5 additionally contains the immobilized first reporter (10806), which is labeled with FITC. In some embodiments, upon binding of the first target nucleic acid sequence to the programmable nuclease probe causes the cleavable nucleic acid of the first reporter (10806) to be cleaved and released into solution. Alternatively, or in combination, in some embodiments, a second detection spot (10804) contains the capture antibody anti-DIG. In some embodiments, a second location D4 contains the immobilized programmable nuclease probe including the sgRNA specific to the second target nucleic acid sequence. In some embodiments, D4 additionally contains the immobilized second reporter (10805), which is labeled with DIG. Therefore, in some embodiments, upon binding of the second target nucleic acid sequence, the immobilized second reporter (10805) is cleaved and released into solution. In some embodiments, the solution containing cleaved first and second reporters (10805) and (10806) is contacted to the sample pad of the lateral flow assay strip along with chase buffer. In some embodiments, the sample pad has one or more flowing capture probes (e.g., anti-biotin-AuNP) disposed thereon. In some embodiments, the sample solution containing the cleaved first and second reporters, along with the chase buffer, flow across the sample pad, where the reporters are bound to conjugates (e.g., anti-biotin-gold nanoparticles). In some embodiments, the solution containing the cleaved reporters is contacted to the sample pad by manually pipetting. In some embodiments, the solution containing the cleaved reporters is contacted to the sample pad being drawn from a chamber in fluid connection with the sample pad. In some embodiments, the solution containing the cleaved reporters is contacted to the sample pad by being drawn from a chamber in which the assay resulting in the cleaved reporter solution occurs. In some embodiments, the reporters are cleaved in the sample pad. In some embodiments, the reporters are cleaved in the sample pad by a DETECTR™ assay. In some embodiments, the solution is drawn into and out of the sample pad by capillary action, or wicking. In some embodiments, the capillary action or wicking is caused by the liquid being drawn into an absorption pad. In some embodiments, the capillary action or wicking is caused by the liquid being drawn into an absorption pad, not requiring electrical power. In some cases, the solution is drawn into or out of the sample pad by a pressure gradient. In some embodiments, the gold nanoparticle-reporter conjugates having reporter (10806) labeled with FITC will selectively bind to the first detection spot (10803) containing the capture antibody anti-FITC, thus indicating the presence of the first target nucleic acid sequence in the sample. In some embodiments, the AuNP-reporter conjugates having mostly reporter (10805) labeled with DIG will selectively bind to the second detection spot (10804) containing the capture antibody anti-DIG, thus indicating the presence of the second target nucleic acid sequence in the sample. In this manner, for some embodiments, parallel detection of two or more target nucleic acid sequences present in a multiplexed sample is enabled.

Described herein are various embodiments of lateral flow-based detection as illustrated in FIG. 109. In some embodiments, horse radish peroxidase (HRP) (10901) is used to enhance detection in lateral flow based DETECTR™ assays. In some embodiments, a sample containing a target(s) nucleic acid sequence is exposed to a surface (10900) upon which programmable nuclease probes and reporter probes are immobilized on the surface. In some embodiments, the reporter probes contain HRP molecules. In some embodiments, upon cleavage of the reporter by the programmable nuclease following a specific binding event between the target and the guide RNA, the cleaved portion of the reporter is released into the sample solution (10906). In some embodiments, the sample solution is then exposed to a lateral flow assay strip (10902) comprising or adjacent to a sample pad containing sodium percarbonate (10904), which generates H₂O₂ when exposed to an aqueous solution. In some embodiments, the rehydration of the sodium percarbonate to form H₂O₂ occurs when the sample is wicked through the region. In some embodiments, the substrate contains DAB, TMB, or any other sufficient substrate. In some embodiments, the “spot” changes from blue to red, indicating the presence of HRP, and in turn a “hit” for the target nucleic acid sequence. In some embodiments, the readout is accomplished in solution, upon a color change of the sample solution (10908).

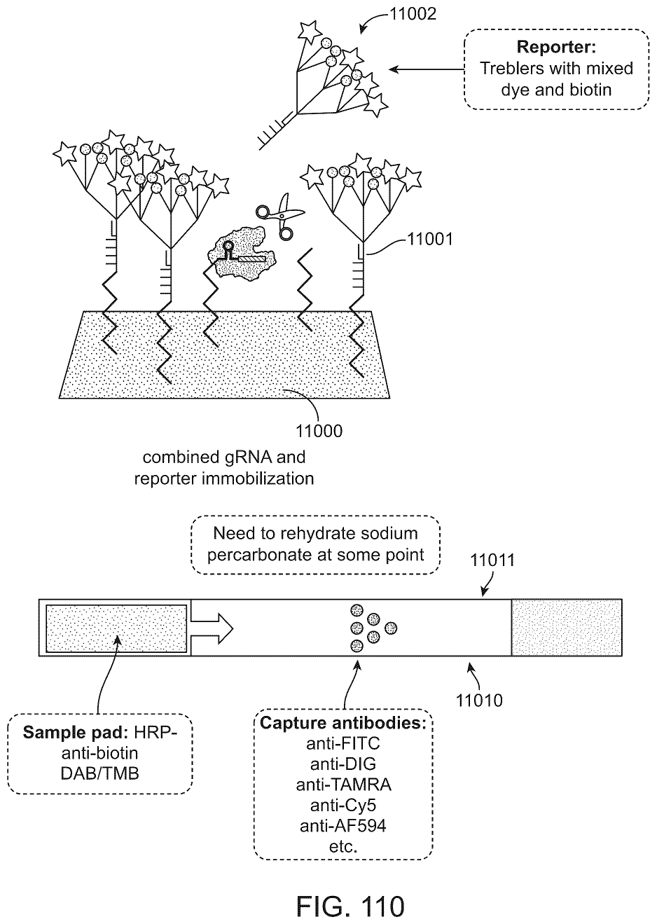

Described here are various methods and devices utilizing HRP-enhanced multiplexed DETECTR™ assays utilizing lateral flow assay strips for readout. In some embodiments, an HRP-signal enhanced multiplexed lateral flow assay as illustrated in FIG. 110. In some embodiments, the immobilized surface (11000) of a support medium and detection on the lateral flow assay strip (11010) are carried out as described in FIGS. 107A-110 with the exception that signal transduction is not carried out by gold nanoparticles scattering light. Instead, in some embodiments, the anti-biotin labeled AuNP are supplanted by HRP-anti-biotin DAB/TMB. In some embodiments, the HRP is activated by sodium percarbonate present in the lateral flow assay strip which is rehydrated by the reaction and or chase buffer. In some embodiments, HRP allows for strong enough signal so as not to require sample amplification such as PCR.

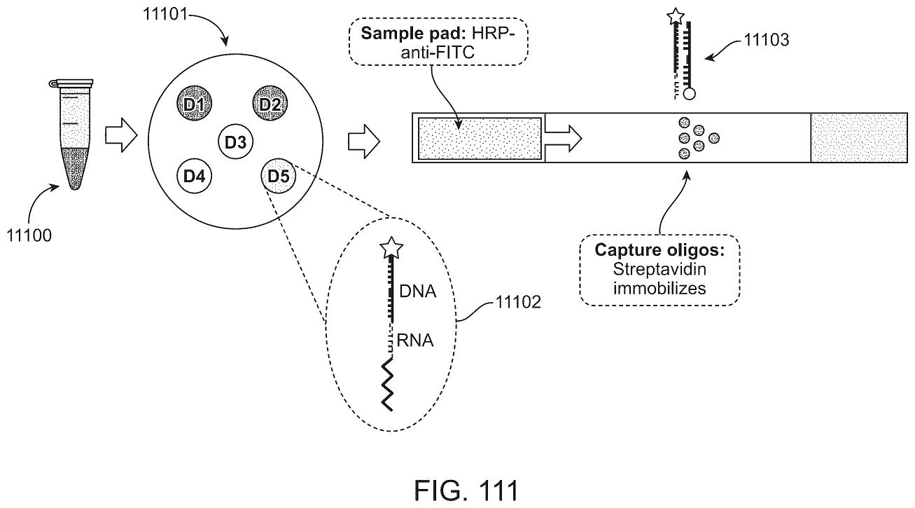

Described herein are various embodiments for multiplexed target nucleic acid detection utilizing Cas13 RNA cleaving specificity over DNA, HRP-signal enhancement, and capture oligo probe specificity. In some embodiments, as shown in FIG. 111, the sample (11100) contains different target nucleic acids. In some embodiments, the sample (11100) is then contacted to the surface of the well (11101) that is functionalized at one or more locations (e.g., five locations, D1-D5). In some embodiments, there are one or more locations. In some embodiments, Cas13 enzyme is present in the programmable nuclease probe. In some embodiments, Cas13 cleaves RNA but not DNA, enabling the use of a reporter (11102) that contains nucleic acid sequences with both DNA and RNA strands. In some embodiments, upon binding of the target nucleic acid to the sgRNA, the RNA of the reporter is cleaved by the Cas13 enzyme and a fragment containing a portion of the RNA, the complete DNA sequence, and a (FITC label is released into solution. In some embodiments, this action is repeated in parallel at each location, or spot with different reporters. In some embodiments, this action is repeated in parallel at locations D1 through D5 for five different target nucleic acids, producing five distinct reporter fragments. In some embodiments, the solution is then contacted to the sample pad of the lateral flow assay strip, where the sample pad contains HRP-anti-FITC. In some embodiments, the FITC-labeled reporter fragment then binds to the HRP-anti-FITC, forming a complex (11103) and is carried downstream across the detection region, binding specifically to the detection spot containing a capture oligo that has been designed to be the compliment for the oligo in the complex (11103).

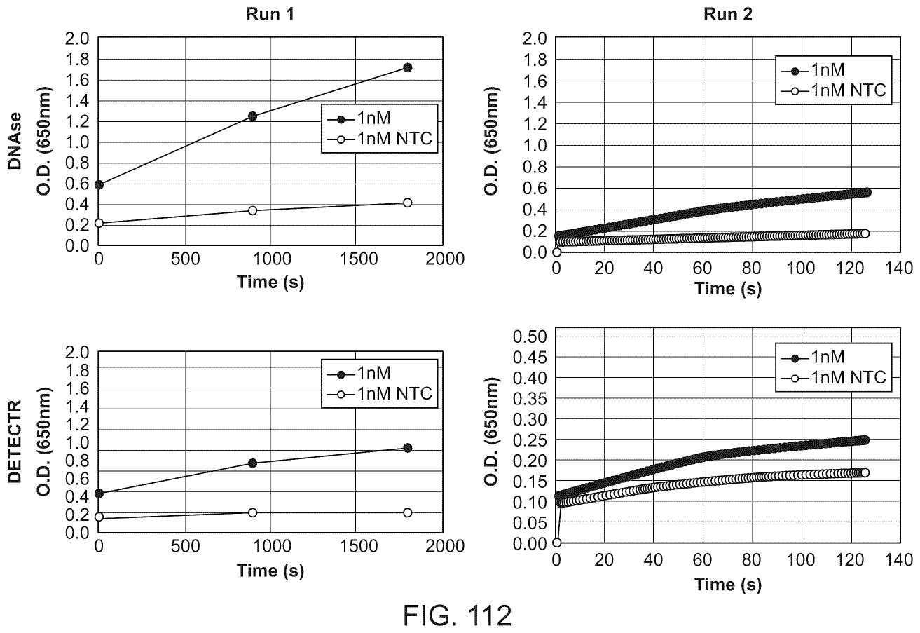

FIG. 112 shows results for both DNAse and DETECTR based assays for two replicate runs performed a week apart.

### Guide RNA Pooling for Signal Enhancement

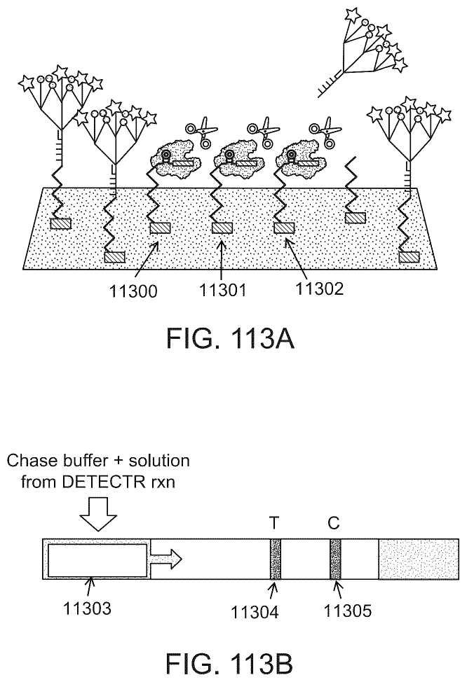

In some embodiments, one or more programmable nuclease probes (11300-11302) are used for guide pooling to achieve enhanced signal detection in lateral flow assays as shown in FIG. 113A. In some embodiments, a first programmable nuclease probe (11300) may comprise a first sgRNA that is complimentary for a first segment of a target nucleic acid. In some embodiments, a second programmable nuclease probe (11301) may comprise a second sgRNA that is complimentary for a second segment of a same target nucleic acid. In some embodiments, a third programmable nuclease probes (11302) may comprise a third sgRNA that is complimentary for a third segment of the same target nucleic acid. In some embodiments, the first programmable nuclease probe, the second programmable nuclease probe, and the third programmable nuclease probe are all located close enough to allow for sufficient cleaving of a reporter that is labeled to indicate the presence of the target nucleic acid. FIG. 113B shows a typical lateral flow assay strip comprising a sample pad (11303), a test line (11304), and a control line (11305).

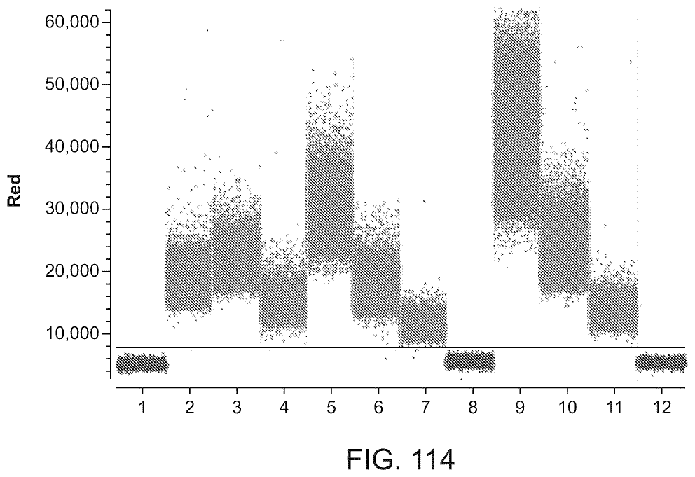

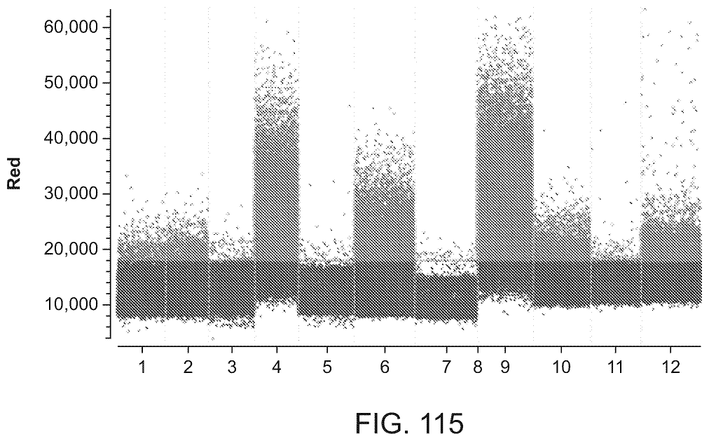

Described herein are various embodiments of guide pooling to achieve enhanced signal detection in lateral flow assays. For some embodiments as described herein, guide pooling shows enhanced Cas12a activity. FIG. 114 (described in Example 20) depicts results of a DETECTR™ assay showing enhanced Cas12a-based detection of the GF184 target using a pooled-guide (pooled-gRNA) format compared to DETECTR™ Cas12a-based assay using an individual gRNA format. In FIG. 114 the y-axis, labeled “Red” displays units of intensity and the x-axis shows the chamber number wherein a different DETECTR™ reaction occurred. FIG. 115 (described in Example 20) depicts results of a DETECTR™ assay showing enhanced sensitivity of the Cas13a-based detection of the SC2 target using a pooled-guide format compared to the Cas13a-based assays using an individual guide format.

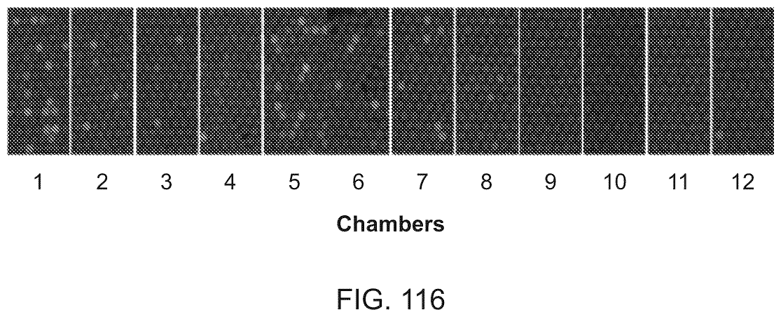

FIG. 116 (described in Example 20) shows images corresponding to each chamber, used to count the number of positive droplets, showing that the Cas13a-DETECTR™ assay samples containing the pooled guide RNAs generated more crystals containing the amplified products per starting copy of the target RNA than the Cas13a-DETECTR™ assay samples containing the guide RNAs in individual format.

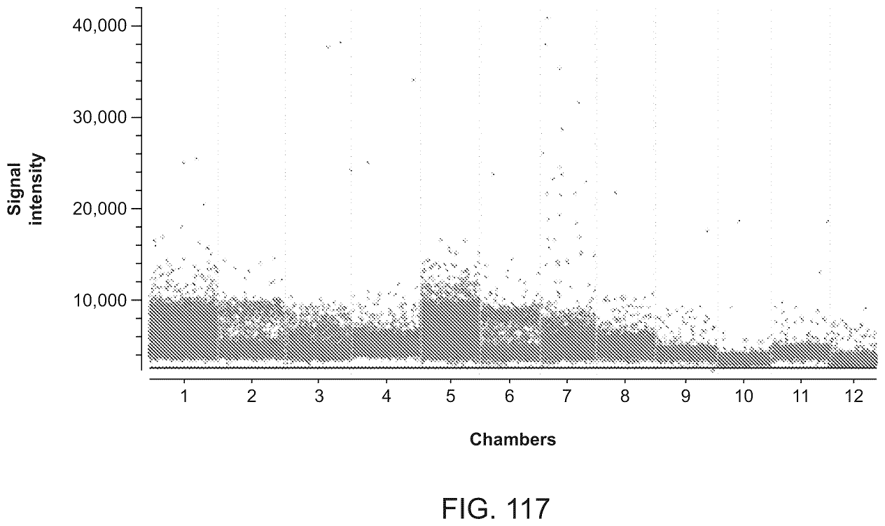

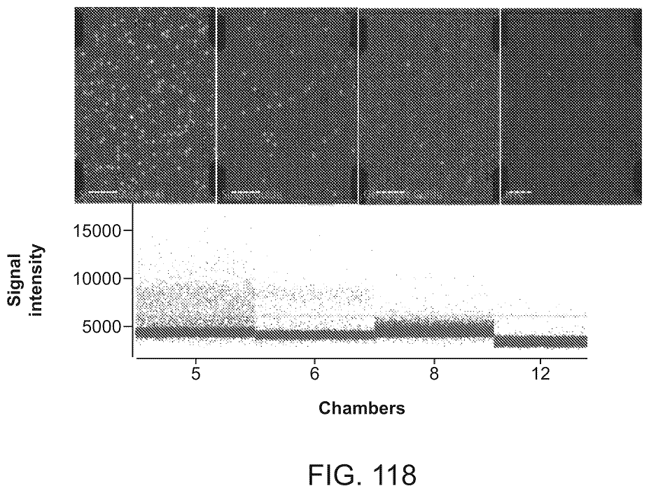

FIG. 117 (described in Example 20) shows that measurement of signal intensity following amplification showed that the Cas13a-DETECTR™ assay samples containing the pooled guide RNAs generated more signal intensity per starting copy of the target template RNA than the Cas13a-DETECTR™ assay samples containing the guide RNAs in individual format. FIG. 118 (described in Example 20) shows that measurement of signal intensity following amplification showed that the Cas13a-DETECTR™ assay samples containing the pooled guide RNAs generated more signal intensity per starting copy of the target template RNA than the Cas13a-DETECTR™ assay samples containing the guide RNAs in individual format. FIG. 118 also shows that relative quantification performed by counting the number of positive droplets showed that the Cas13a-DETECTR™ assay samples containing the pooled guide RNAs generated more crystals containing the amplified products per starting copy of the target template RNA than the Cas13a-DETECTR™ assay samples containing the guide RNAs in individual format.

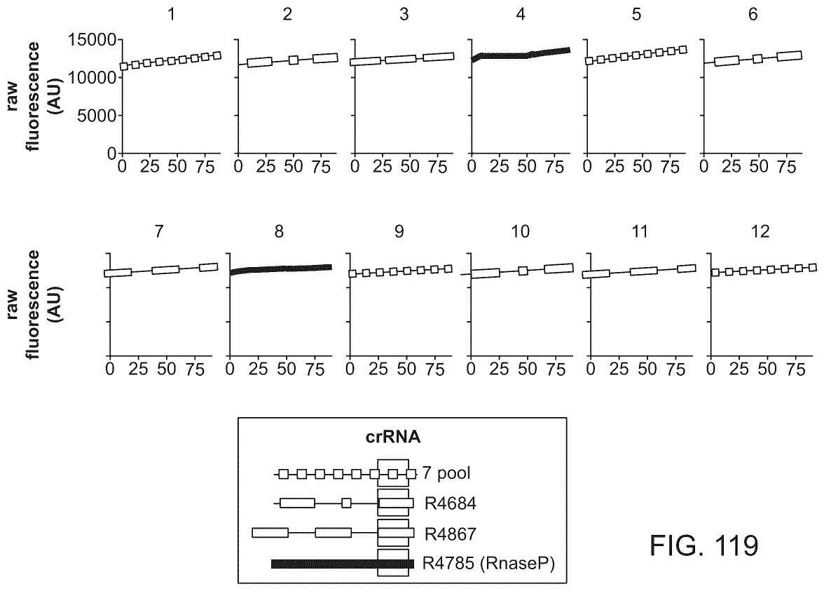

FIG. 119 (described in Example 20) shows that Cas13a DETECTR™ assay samples containing the pooled guides (R4637, R4638, R4667, R4676, R4684, R4689, R4691) did not exhibit higher target detection sensitivity per starting copy of the target than the Cas13a DETECTR™ samples containing the single guides R4684, R4667, or R4785 (RNAseP guide) in individual format.

### DETECTR Based Multiplexed Lateral Flow PON Device

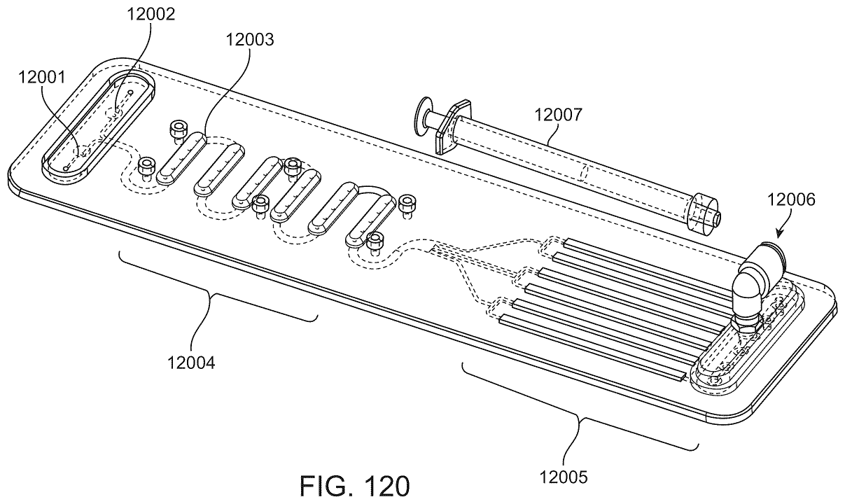

Described herein are various methods and devices for a programmable nuclease (e.g., DETECTR) assay based multiplex lateral flow assay as illustrated in FIG. 120. FIG. 120 depicts a non-limiting exemplary handheld device for performing a multiplexed programmable nuclease (e.g., DETECTR) assay. In some embodiments, a sample contains one or more target nucleic acid sequences. In some embodiments, the sample is introduced into a sample input (12001) (also referred to herein as a sample interface) and loaded into one or more zones (e.g., heating regions or reaction chambers, 12003) comprising one or more dried or lyophilized programmable nuclease reagents (e.g., immobilized to a glass bead). In some embodiments, the sample interface may be configured to concentrate, filter, lyse, or otherwise prepare the sample as described herein. In some embodiments, negative pressure is applied to a negative pressure port (12006) by a negative pressure source (e.g., a syringe, 12007) in order to load the DETECTR zones with the sample. In some embodiments, the sample is a lysis sample. In some embodiments, a plurality of DETECTR zones (12003) are coupled to one another along a serpentine fluid path (12004). In some embodiments, the fluid path may be spiral as described herein. In some embodiments, amplification of one or more target nucleic acids is performed in an amplification region comprising the one or more DETECTR zones (12003). The amplification region (12004) may be heated (e.g., to 55 C for 20 minutes or less) to amplify the one or more target nucleic acids as described herein. In some embodiments, the amplification region (12004) may comprise interspersed polymers. Heating can include liquid-phase chemical heating, solid phase chemical heating, and electric heating (including, but not limited to, resistance/Joule heating, induction heating, Peltier heat pumping, etc.). In some embodiments, the device includes multiple lateral flow strips (12005). In some embodiments, a diluent is introduced into a diluent input (12002). Those skilled in the art will understand that the detection region (e.g., lateral flow assay region (12005)) may include any compatible assay or a lateral flow strip as described herein.

In some embodiments, the amplification region (12004) may be coated. In some embodiments, a coating may be a hydrophilic coating, a hydrophobic coating, an inorganic coating, or an organic coating. In some embodiments, a coating may comprise a polymer coating. In some embodiments, a coating may comprise a polyethylene glycol coating. In some embodiments, a coating may comprise a streptavidin coating. In some embodiments, the interspersed polymers may be a crowding agent. In some embodiments, the crowding agent may comprise polyethylene glycol.

In some embodiments, the one or more reaction or heating zones may comprise guide nucleic acids (e.g., sgRNAs) immobilized to a surface (e.g., a glass bead disposed within a DETECTR zone). In some embodiments, the guide nucleic acids are part of a programmable nuclease (e.g., Cas-complex) probe immobilized to a surface. In some embodiments, a guide nucleic acid is designed to specifically bind to a target nucleic acid in the sample. In some embodiments, there are different guide nucleic acids corresponding to different locations on the surface and/or different surfaces in the one or more zones, where each different guide nucleic acid is complimentary for a different target nucleic acid sequence that may or may not be present in the sample. In some embodiments, in addition to the programmable nuclease probes containing guide nucleic acids, each surface location is functionalized with one or more reporters having distinct functional groups. In some embodiments, the reporters may be in close enough proximity to be cleaved by the programmable nuclease probes. In some embodiments, as described in example 13, reporters are cleaved and released into the solution upon binding between a particular guide nucleic acid and the target nucleic acid to which the guide nucleic acid is designed to specifically bind. In some embodiments, reporters are functionalized with a detection moiety (e.g., a label).

In some embodiments, chemical heating may be used. In some embodiments, chemical heating may be used to supply energy to initiate and run reactions. In some embodiments, chemical heating may be used to supply energy to initiate and run programmable nuclease assay reactions. In some embodiments, chemical heating may be used to heat reaction or heating zones. In some embodiments, chemical heating may be used to heat regions, chambers, volumes, zones, surfaces, or areas of a device.

In some embodiments, the device may comprise one or more lateral flow assay strips in a detection region disposed downstream of the amplification region. Each lateral flow assay strip contains one or more detection regions or spots, where each detection region or spot contains a different type of capture antibody. In some embodiments, each lateral flow assay strip contains a different type of capture antibody. In some embodiments, each capture antibody type specifically binds to a particular label type of a reporter. In some embodiments, a first lateral flow assay strip contains the capture antibody anti-FITC. In some embodiments, a first DETECTR region or surface location (e.g., within a reaction chamber or heating region) contains the immobilized programmable nuclease (e.g., Cas-complex) including the guide nucleic acid (e.g., sgRNA) specific to the first target nucleic acid sequence. In some embodiments, the first DETECTR region or surface location additionally contains a first immobilized reporter which is labeled with a first detection moiety (e.g., FITC). In some embodiments, upon binding of the first target nucleic acid sequence, the first immobilized reporter is cleaved and released into solution. In some embodiments, the first detection moiety is released into solution and the remainder of the first reporter remains immobilized on the surface. Alternatively, or in combination, in some embodiments, a second lateral flow assay strip contains the capture antibody anti-DIG. In some embodiments, a second DETECTR region or surface location (e.g., within a reaction chamber or heating region) contains the immobilized programmable nuclease (e.g., Cas complex) including the guide nucleic acid (e.g., sgRNA) specific to the second target nucleic acid sequence. In some embodiments, the second DETECTR region or surface location additionally contains a second immobilized reporter which is labeled with a second detection moiety (e.g., DIG). Therefore, in some embodiments, upon binding of the second target nucleic acid sequence, the second immobilized reporter is cleaved and released into solution. In some embodiments, the second detection moiety is released into solution and the remainder of the second reporter remains immobilized on the surface.

In some embodiments, the solution containing the first and second cleaved reporters is transferred from the amplification region to the lateral flow region comprising the first lateral flow assay strip and the second lateral flow assay strip. In some embodiments, a chase buffer or diluent is introduced into a diluent input and negative pressure is applied to the negative pressure port to contact the solution containing the first and second cleaved reporters to the lateral flow assay strips of the lateral flow region, where the reporters are bound to conjugate molecules e.g., anti-biotin-AuNPs. In some embodiments, the AuNP-reporter conjugates having the first reporter labeled with the first detection moiety (e.g., FITC) will selectively bind to a first detection region or spot containing the first capture antibody (e.g., anti-FITC) on the first lateral flow assay strip, thus indicating the presence of the first target nucleic acid sequence in the sample. In some embodiments, the AuNP-reporter conjugates having mostly the second reporter labeled with the second detection moiety (e.g., DIG) will selectively bind to a second detection region or spot containing the second capture antibody (e.g., anti-DIG) on the second lateral flow assay strip, thus indicating the presence of the second target nucleic acid sequence in the sample. In this manner, for some embodiments, parallel detection of two or more target nucleic acid sequences present in a multiplexed sample is enabled.

In some embodiments, the amplification region is configured to hold about 200 μL of liquid (e.g., sample solution and reagent(s)). In some embodiments, each lateral flow assay strip is configured to hold about 80 μL of liquid (e.g., sample solution and/or chase buffer). In some embodiments, the device may comprise more than one lateral flow assay strip. For example, the device may comprise two, three, four, five, six, seven, eight, nine, ten, or more lateral flow assay strips. In some embodiments, one or more lateral flow assay strips are configured to detect a control sequence instead of or in addition to a target sequence. For example, a device comprising six lateral flow assay strips may comprise five lateral flow assay strips configured to detect one or more target sequences (e.g., five different target sequences) and one lateral flow assay strip configured to detect a control sequence.

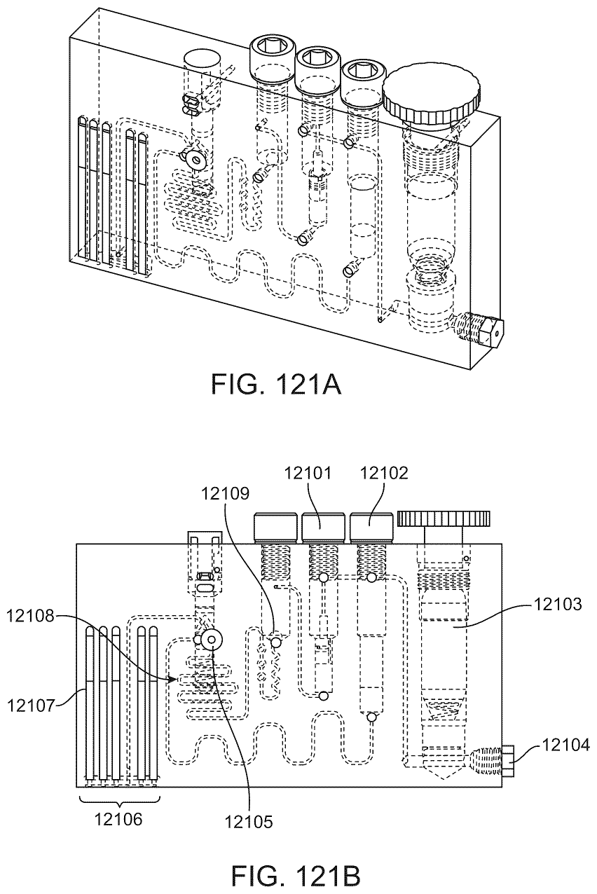

Described herein are various methods and devices for a programmable nuclease assay based multiplex lateral flow assay as illustrated in FIGS. 121A-121B. FIGS. 121A-121B illustrate a non-limiting example of a point of care device for parallel detection of two or more target nucleic acid sequences. In some embodiments, a sample contains one or more target nucleic acid sequences. In some embodiments, the sample is introduced into a sample input chamber (12101) (also referred to herein as a sample interface). The sample input chamber (12101) may be configured to receive a swab comprising the sample. In some embodiments, the sample input chamber (12101) may comprise a solution/buffer (e.g., lysis buffer) to retrieve the sample from the swab. In some embodiments, the sample interface may be configured to concentrate, filter, lyse, or otherwise prepare the sample as described herein. In some embodiments, positive pressure is applied to the sample input chamber (12101) to move the sample solution from the sample input chamber (12101) to a reaction chamber (12108) (e.g., within a heating region) containing one or more dried or lyophilized programmable nuclease (e.g., DETECTR) reagents (12109) (e.g., immobilized to a glass bead) as described herein. In some embodiments, positive pressure may be applied by a positive pressure source (e.g., a compressed gas tank, 12103) in order to load the reaction chamber with the sample solution. In some embodiments, pressure is regulated by a pressure valve (12104) coupled to the positive pressure source (12103). In some embodiments, application of positive pressure (e.g., by operating an actuator to pierce a positive pressure source 12103 comprising a compressed gas tank) moves the sample solution and dried or lyophilized reagent(s) (12109) into a serpentine amplification region of the reaction chamber (12108). In some embodiments, the amplification region may be spiral as described herein. In some embodiments, amplification of one or more target nucleic acids is performed in the amplification region (12108). The amplification region (12108) may be heated (e.g., to 55 C for 20 minutes or less) to amplify the one or more target nucleic acids as described herein. In some embodiments, application of sufficient positive pressure opens the pressure valve (12105) between the amplification region and the detection region, e.g., a lateral flow assay region (12106). In some embodiments, the valve (12105) between the amplification region (12108) and the lateral flow assay region (12106) comprising lateral flow strips (12107) may be opened by pressing a button connected to the valve (12105). Heating can include liquid-phase chemical heating, solid phase chemical heating, and electric heating (including, but not limited to, resistance/Joule heating, induction heating, Peltier heat pumping, etc.). In some embodiments, a diluent or chase buffer may be introduced to the system via the diluent input (12102). In some embodiments, opening the valve (12105) between the amplification region (12108) and the lateral flow assay region (12106) transfers the sample from the amplification region (12108) to the lateral flow assay region (12106). In some embodiments, the sample is mixed with diluent as it moves from the amplification region (12108) to the lateral flow assay region (12106). In some embodiments, the chase buffer pushes the sample from the amplification region (12108) to the lateral flow assay region (12106) without significantly diluting the sample.

In some embodiments, the one or more programmable nuclease reagent(s) comprise guide nucleic acids immobilized to a surface (e.g., a glass bead). In some embodiments, the guide nucleic acids are part of a programmable nuclease (e.g., Cas-complex) probe immobilized to a surface. In some embodiments, a guide nucleic acid may be designed to specifically bind to a target nucleic acid in the sample. In some embodiments, there are different guide nucleic acids corresponding to different locations on the surface and/or different surfaces in the one or more zones, where each different guide nucleic acid is complimentary for a different target nucleic acid sequence that may or may not be present in the sample. In some embodiments, in addition to the programmable nuclease probes containing guide nucleic acids, each surface location may be functionalized with one or more reporters having distinct functional groups. In some embodiments, the reporters are in close enough proximity to be cleaved by the programmable nuclease probes. In some embodiments, as described in example 13, reporters are cleaved and released into the solution upon binding between a particular sgRNA and the target nucleic acid to which the guide nucleic acid is designed to specifically bind. In some embodiments, reporters are functionalized with a detection moiety (e.g., a label).

In some embodiments, the device may comprise one or more lateral flow assay strips in a detection region disposed downstream of the amplification region. Each lateral flow assay strip contains one or more detection regions or spots, where each detection region or spot contains a different type of capture antibody. In some embodiments, each lateral flow assay strip may contain a different type of capture antibody. In some embodiments, each capture antibody type specifically binds to a particular label type of a reporter. In some embodiments, a first lateral flow assay strip contains a first capture antibody (e.g., anti-FITC). In some embodiments, a first surface (e.g., bead) contains the immobilized programmable nuclease (e.g., Cas-complex) including the guide nucleic acid (e.g., sgRNA) specific to the first target nucleic acid sequence. In some embodiments, the first surface additionally contains a first immobilized reporter which is labeled with a first detection moiety (e.g., FITC). In some embodiments, upon binding of the first target nucleic acid sequence, the first immobilized reporter is cleaved and released into solution as described herein. Alternatively, or in combination, in some embodiments, a second lateral flow assay strip contains a second capture antibody (e.g., anti-DIG). In some embodiments, a second surface (e.g., bead) contains the second immobilized programmable nuclease including the guide nucleic acid specific to the second target nucleic acid sequence. In some embodiments, the second surface additionally contains a second immobilized reporter which is labeled with a second detection moiety (e.g., DIG). Therefore, in some embodiments, upon binding of the second target nucleic acid sequence, the second immobilized reporter is cleaved and released into solution.

In some embodiments, the solution containing the first and second cleaved reporters is transferred from the amplification region to the lateral flow region comprising the first lateral flow assay strip and the second lateral flow assay strip. In some embodiments, a chase buffer or diluent is introduced into a diluent input or reservoir and negative pressure is applied to the negative pressure port to contact lateral flow region. A pressure valve may be disposed between the amplification region and the lateral flow region in order to regulate flow of the sample solution from the amplification region to the lateral flow region before amplification has occurred. Actuation of the pressure valve enables the solution containing the first and second cleaved reporters to contact the lateral flow assay strips of the lateral flow region, where the reporters are bound to congugate molecules, e.g., anti-biotin-AuNPs. In some embodiments, the AuNP-reporter conjugates having the first reporter labeled with the first detection moiety (e.g., FITC) will selectively bind to a first detection region or spot containing a first capture antibody (e.g., anti-FITC) on the first lateral flow assay strip, thus indicating the presence of the first target nucleic acid sequence in the sample. In some embodiments, the AuNP-reporter conjugates having mostly the second reporter labeled with the second detection moiety (e.g., DIG) will selectively bind to a second detection region or spot containing a second capture antibody (e.g., anti-DIG) on the second lateral flow assay strip, thus indicating the presence of the second target nucleic acid sequence in the sample. In this manner, for some embodiments, parallel detection of two or more target nucleic acid sequences present in a multiplexed sample is enabled.

In some embodiments, the amplification region is configured to hold about 200 μL of liquid (e.g., sample solution and reagent(s)). In some embodiments, each lateral flow assay strip is configured to hold about 80 μL of liquid (e.g., sample solution and/or chase buffer). In some embodiments, the device may comprise more than one lateral flow assay strip. For example, the device may comprise two, three, four, five, six, seven, eight, nine, ten, or more lateral flow assay strips. In some embodiments, one or more lateral flow assay strips are configured to detect a control sequence instead of or in addition to a target sequence. For example, a device comprising six lateral flow assay strips may comprise five lateral flow assay strips configured to detect one or more target sequences (e.g., five different target sequences) and one lateral flow assay strip configured to detect a control sequence.

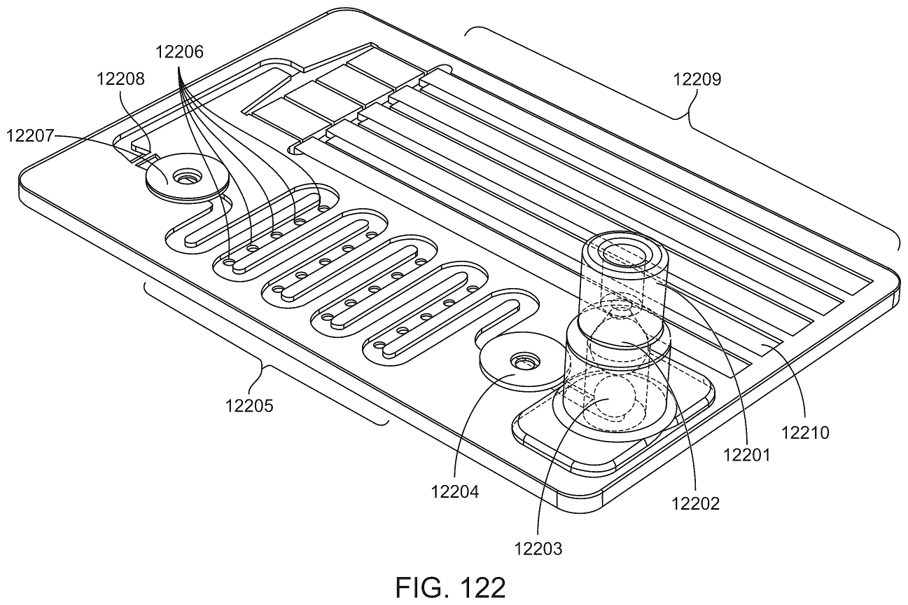

Described herein are various methods and devices for a DETECTR assay based multiplex lateral flow assay as illustrated in FIG. 122. FIG. 122 illustrates an embodiment of a point of care device comprising a lateral flow assay which is substantially similar to the embodiment of FIG. 120 but utilizes positive pressure to drive fluid flow instead of negative pressure. In some embodiments, the device may comprise an input port (12201) that receives a sample. In some embodiments, the input port may be configured to interface with a luer lock syringe containing the sample (e.g., containing a sample solution and/or a swab disposed therein). In some embodiments, the input port (12201) may comprise a ball check valve (12202) configured to seal the input port (12201) until a sample is received. In some embodiments, the device may comprise lyophilized reagents (12203). In some embodiments, the lyophilized reagents (12203) may be stored in the sealed sample interface chamber before the sample is received. In some embodiments, the device may comprise hydrophobic filters that are permeable to gases (12204 and 12207) and configured to permit liquid movement through the device and facilitate release of displaced gases therefrom. In some embodiments, the device may comprise a serpentine channel region (12205). In some embodiments, the serpentine channel region (12205) is heated. In some embodiments, the serpentine channel region (12205) may comprise one or more reaction zones (12206) within a heating region. In some embodiments, the reaction zones (12206) may be configured to perform one or more DETECTR assays. In some embodiments, the reaction zones (12206) comprise amplification enzymes or reagents. In some embodiments, the lyophilized reagents (12203) comprise one or more amplification enzymes or reagents which are reconstituted upon introduction of the sample into the sample interface and which, following reconstitution, may be transferred into the reaction chamber along with the sample. In some embodiments, the sample interface may be configured to concentrate, filter, lyse, or otherwise prepare the sample as described herein. In some embodiments, the reaction zones (12206) comprise visual assays that change color in the presence of a chemical. In some embodiments, the serpentine channel region (12205) is a nucleic acid amplification region. Alternatively, or in combination, the serpentine channel region may be configured to perform one or more programmable nuclease-based detection assays. In some embodiments, a valve may separate the detection region (12209) from the heating region (12205). In some embodiments, the device may comprise a pressure cracking valve (12208) that breaks under sufficient positive pressure and enable fluid movement between the two regions once broken (and, conversely, prevent fluid movement between the regions prior to positive pressure application). In some embodiments, a diluent or chase buffer may be injected into the device via the input port (12201) in order to break the pressure cracking valve (12208) and transfer the sample fluid from the heating/reaction region 12205 to the detection region 12209. In some embodiments, the device may receive positive gas pressure at the input port (12201). In some embodiments, breaking the pressure cracking valve (12208) allows the passage of liquids from the serpentine channel region (12205) to the lateral flow assay region (12209).

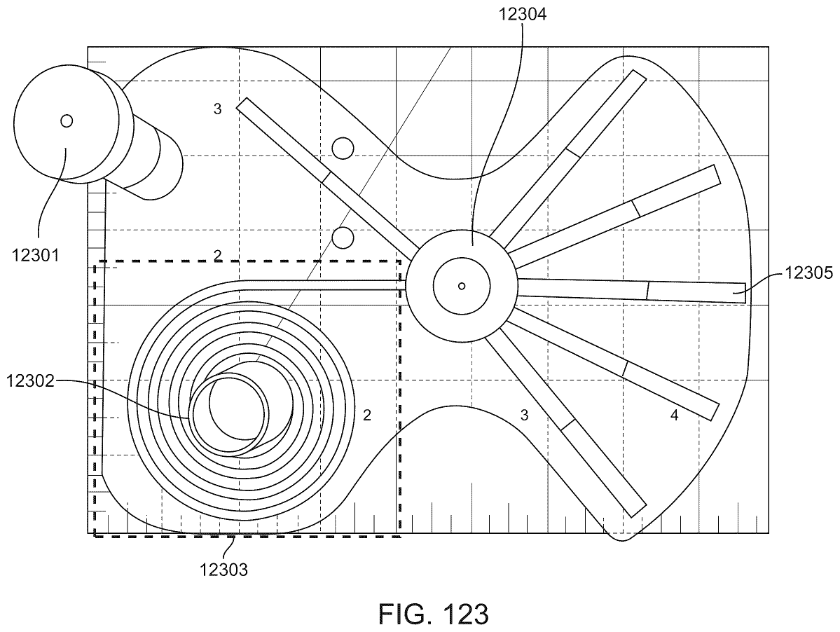

Described herein are various methods and devices for a handheld assay device as illustrated in FIG. 123. FIG. 123 illustrates an embodiment of a handheld assay device comprising an input port (12302), a spiral channel heating or reaction region (12303), a first actuator (12301), a second actuator (12304), and lateral flow assay region (12305). In some embodiments, a nucleic acid sample is delivered at the input port (12302) as described herein, and the sample flows through the spiral channel region (12303). In some embodiments, the sample interface 12302 may be configured to concentrate, filter, or otherwise prepare the sample as described herein prior to transfer into the reaction region 12303. In some embodiments, the first actuator (12301) stores mechanical energy. In some embodiments, the stored mechanical energy is stored as pressurized gas. In some embodiments, the stored mechanical energy is stored in compressed springs. In some embodiments, actuating the first actuator (12301) release the stored mechanical energy. In some embodiments, releasing the stored mechanical energy pushes fluid from the sample interface 12302 through the spiral channel region (12303). In some embodiments, releasing the stored mechanical energy pulls fluid through the spiral channel region (12303). In some embodiments, the spiral channel region may comprise amplification enzymes and reagents. In some embodiments, the sample interface may comprise amplification enzymes and reagents which can be transferred into the spiral channel region with the sample as described herein. In some embodiments, the nucleic acid sample is amplified in the spiral channel region (12303) as described herein. In some embodiments, the spiral channel region is heated. In some embodiments, the second actuator (12304) stores mechanical energy. In some embodiments, the stored mechanical energy is stored as pressured gas. In some embodiments, actuating the second actuator (12304) release the stored mechanical energy. In some embodiments, releasing the stored mechanical energy pushes the sample through the detection (e.g., lateral flow assay) region (12305). In some embodiments, releasing the stored mechanical energy pulls the sample through the lateral flow assay region (12305). The detection region may comprise one or more lateral flow assay strips as described herein.

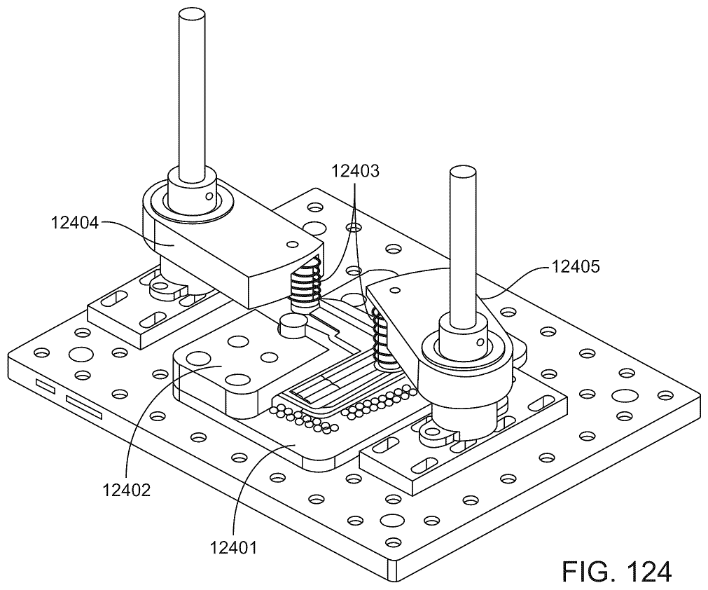

Described herein are various methods and devices for a handheld assay device as illustrated in FIG. 124. FIG. 124 illustrates an embodiment of a handheld assay device breadboard comprising actuator triggers (12404, 12405), actuators (12403), a heating plate (12401), or a platen (12402), which may be configured to be used in conjunction with the fluid flow scheme described in FIG. 123, for example. In some embodiments, the handheld assay device may comprise a single-use, disposable assay component. In some embodiments, the handheld assay device may comprise reusable hardware framework that houses the single-use, disposable assay components (e.g., cartridges). In some embodiments, the single-use, disposable assay components are held by a platen (12402). In some embodiments, the disposable assay components comprise actuators storing mechanical energy as described herein. In some embodiments, the actuators are actuated by actuator triggers (12404, 12405). In some embodiments, the disposable assay components are heated by a heating plate (12401). In some embodiments, the entire device, including triggers, actuators, heating plate, platen, and fluidic components (including lateral flow assay strips, etc.) are coupled together (e.g., within a housing) in a single disposable unit.

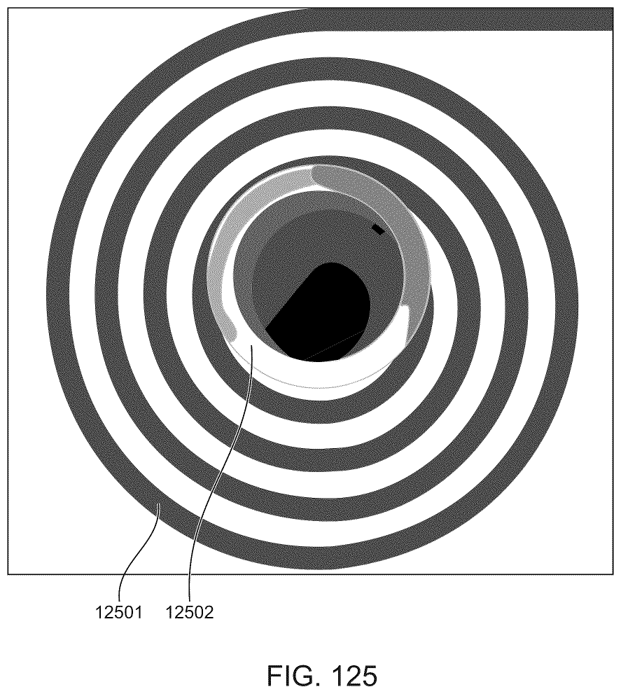

FIG. 125 illustrates an embodiment of a fluid channel (12501) of a handheld assay device. Any of the devices described herein may comprise a spiral fluid channel as shown (e.g., the heating region or reaction region may comprise a spiral fluid channel). In some embodiments, the fluid channel (12501) is substantially spiral in shape. In some embodiments, the fluid channel (12501) is coupled to a port (12502). In some embodiments, the port (12502) is a input sample receiver. In some embodiments, the port (12502) is an input pressure receiver. In some embodiments, the port (12502) is an input diluent receiver. In some embodiments, the fluid channel (12501) may comprise enzymes or reagents. In some embodiments, the fluid channel (12501) is an amplification region. In some embodiments, the fluid channel (12501) is heated. In some embodiments, the fluid channel (12501) may comprise one or more DETECTR assay reagents as described herein.

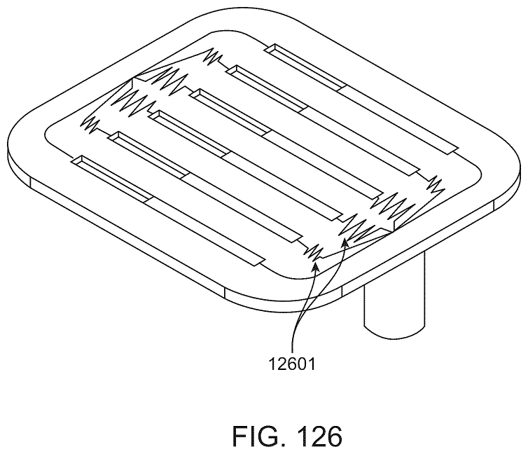

FIG. 126 illustrates an embodiment of a lateral flow assay region of a handheld assay device which may be implemented in any of the devices described herein. In some embodiments, the path length leading to each lateral flow strip is substantially equal in fluid path length (12601) in order to ensure substantially equal fluidic resistances between channels for substantially equal transfer of fluids from the reaction region to the lateral flow assay strips of the lateral flow assay detection region. The fluid path length is defined as the length of the path that is taken by a fluid that moves from one end of a channel to the other end of the channel.

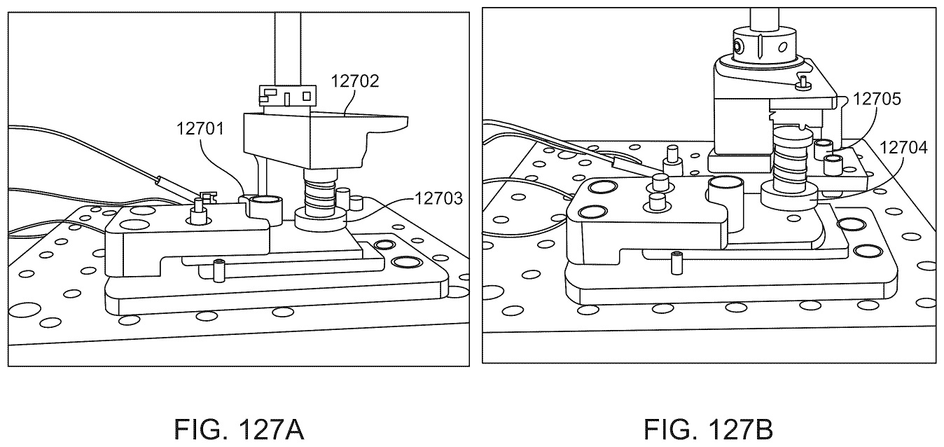

FIGS. 127A-127B show the breadboard device of FIG. 124 in action with the fluidic device of FIG. 123. In FIG. 127A, the spring-based actuator 12703 is loaded and the cartridge is ready for the addition of the sample into the sample interface. In FIG. 127B, the spring-based actuator is partially released and the sample has been pulled from the sample interface into the spiral reaction region. The spring is configured to stay in this position during the amplification and/or programmable nuclease-based detection reaction.

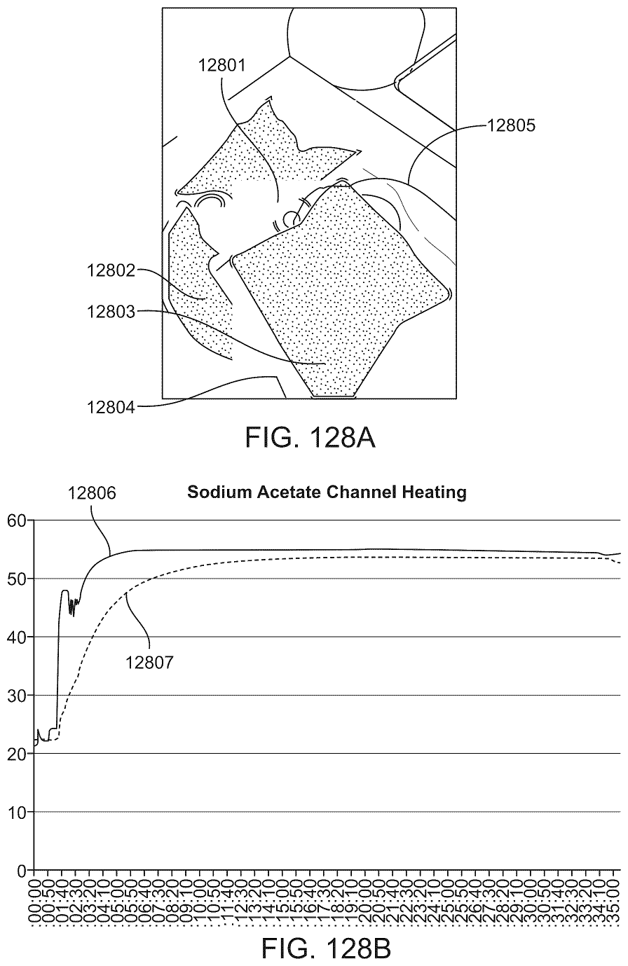

FIG. 128A depicts a chemical heating pouch disposed on the device of FIG. 123 for proof of concept testing. In some embodiments, the chemical heating pouch contains a solution comprising sodium acetate and a metal button, wherein pressing the metal button starts a crystallization reaction that is exothermic. The sample chamber (12801) of the disposable was exposed to allow for sample addition. The sample chamber disposable was nested between heated pouches (12802 and 12803). A temperature probe (12804) nested between the two pouches was used to measure the external temperature. A second temperature probe (12805) was used to measure fluid temperature inside the sample chamber. FIG. 128B depicts the temperature as a function of time measured at the chemical heating pouch (12806) and measured at the second temperature probe (12807). The temperature of the pouch was a couple of degrees hotter than the fluid temperature. In some embodiments, the temperature of the fluid approximately maintained a temperature of 53° C. for about 35 minutes. In some embodiments, the temperature of the fluid approximately maintained a temperature of 55° C. for about 30 minutes.

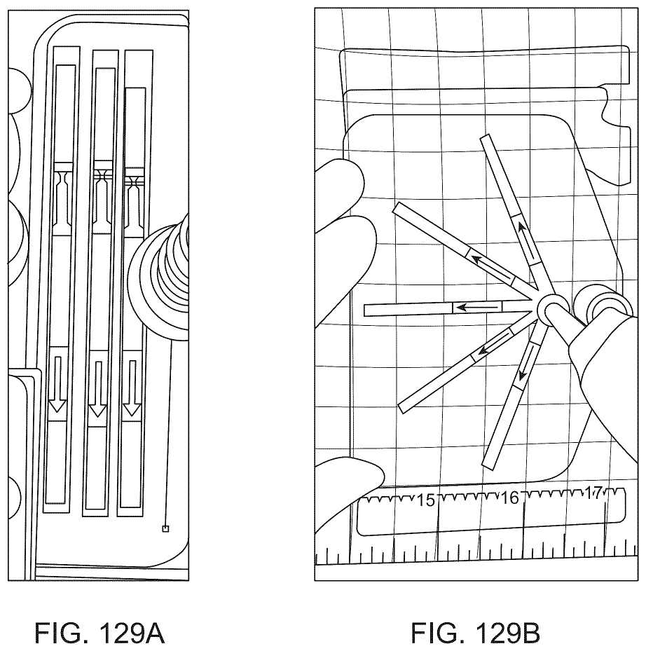

FIG. 129A-129B illustrate additional exemplary embodiments of a lateral flow assay region. In some embodiments, the sample fluid from the heated chamber may be transferred to the lateral flow assay region through aspiration, e.g., created by a syringe vacuum. In some embodiments, the lateral flow assay strips may be arranged in serial geometries as shown in FIG. 129A. In some embodiments, the lateral flow assay strips may be arranged in radial geometries as shown in FIG. 129B. In some embodiments, lateral flow assay strips may be arranged in parallel geometries as shown in FIG. 126. In some embodiments, the lateral flow strips may be used to capture programmable nuclease-mediated reporter cleavage as described herein.

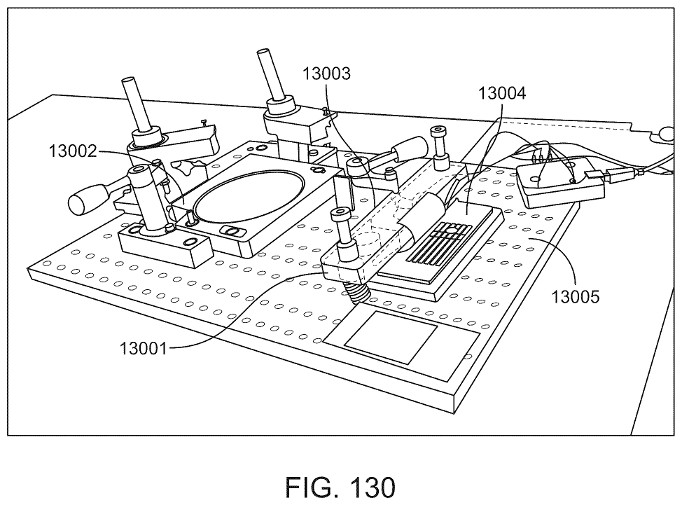

FIG. 130 depicts an embodiment of a breadboard device for testing DETECTR reactions. The breadboard holds 3D printed disposable chips with reaction chamber and lateral flow strips. This embodiment is compatible for testing DETECTR reactions with chemical and electrical heating. In some embodiments, the device may comprise a fluidic drive (13001). In some embodiments, the device may comprise a chemical heating element (13002). In some embodiments, the chemical heating element (13002) may comprise a pouch containing sodium acetate solution and a metal disk, wherein pressing the metal disk triggers the crystallization in the sodium acetate solution. In some embodiments, the temperature of the heating chamber is brought to at least about 50 to at least about 60° C. In some embodiments, the device may comprise a polyamide resistive heater. In some embodiments, the temperature of the heating chamber is brought to at least about 54 to at least about 56° C. In some embodiments, the device may comprise clamps (13003). In some embodiments, the device may comprise lateral flow strips (13004). In some embodiments, the device may comprise electric heating elements (13005). In some embodiments, the device may comprise a polyamide resistive heater.

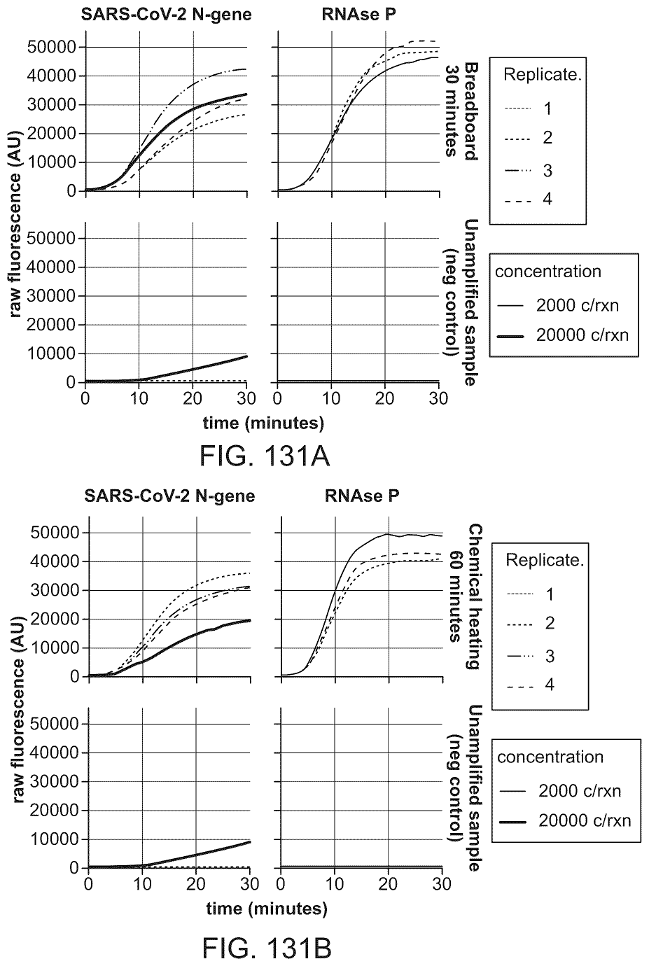

Described herein are various methods and devices for a handheld assay device as illustrated in FIGS. 131A-131B. In FIG. 131A, DETECTR analysis confirmed successful amplification of a duplex assay performed on an embodiment of a handheld device using electric heating. A 2-plex RT-LAMP assay was assembled using 2000 copies of HeLa RNA and 20,000 copies of a synthetic SARS-CoV-2 RNA. The experiment was performed with an assay temperature of 62° C. for 30 minutes. The amplification products were recovered from the chip and a DETECTR assay was performed using a Tecan plate reader to collect the results. A portion of the unamplified mastermix plus sample was stored on ice and used as the negative control for the DETECTR reaction. The resulting DETECTR curves confirm robust amplification had occurred on a spiral breadboard device using electric heating. In FIG. 131B, DETECTR analysis confirmed successful amplification of a duplex assay on an embodiment of a handheld device using chemical heating. A 2-plex RT-LAMP assay was assembled using 2000 copies of HeLa RNA and 20,000 copies of a synthetic SARS-CoV-2 RNA. The experiment was performed with an assay temperature of 55° C. for 60 minutes. The amplification products were recovered from the chip and a DETECTR™ assay was performed using a Tecan plate reader to collect the results. A portion of the unamplified mastermix plus sample was stored on ice and used as the negative control for the DETECTR reaction. The resulting DETECTR curves confirmed robust amplification had occurred on a spiral breadboard device using chemical heating.

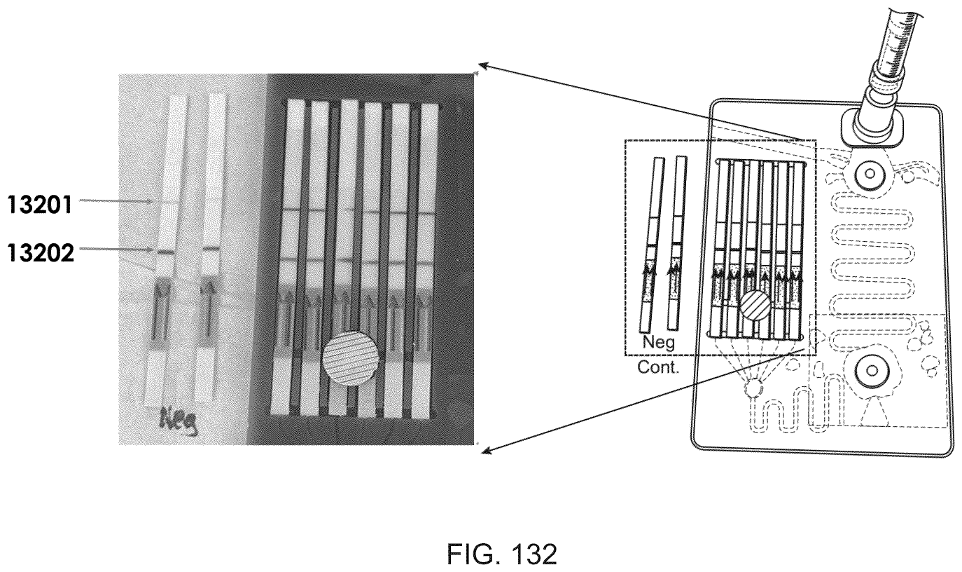

FIG. 132 depicts a DETECTR reaction with lateral flow readout performed on the breadboard device of FIG. 130 with a disposable chip similar to the device shown in FIG. 122. The disposable chip was loaded with 200 μL of DETECTR mastermix containing RT-LAMP amplified SARS-CoV-2 N-gene. The disposable chip was incubated on the breadboard device for 20 minutes using the electric heater. A ‘chase’ buffer applied to the sample input port to dilute and move the DETECTR reaction onto the lateral flow strips. The strips were allowed to absorb the DETECTR reaction for four minutes before the results were photographed. A portion of the reaction was removed prior to incubating on the device and used as the negative control ‘off device.’ For testing Milenia HybridDetect lateral flow strips were used, which placed the control band in the lower position (13201) and the SARS-CoV-2 N-gene band distal to the control (13202).

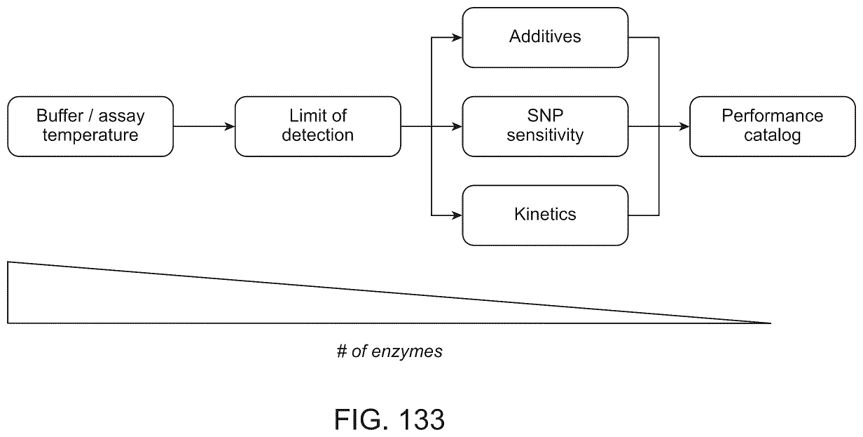

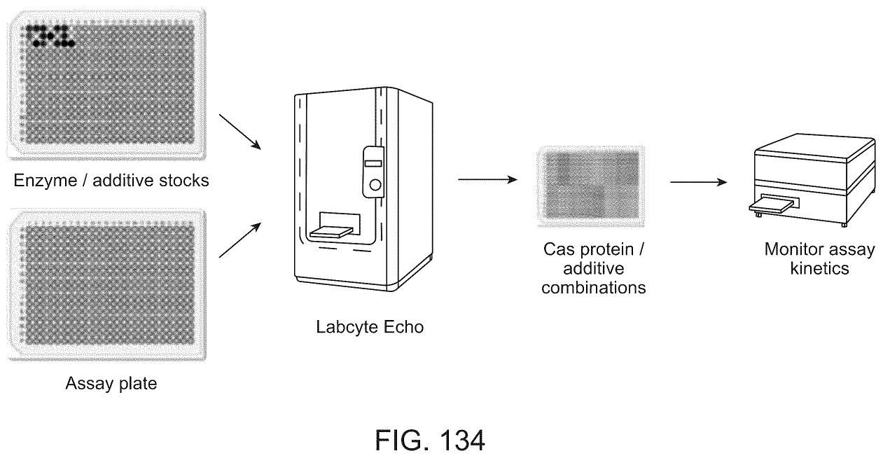

Disclosed herein are methods for optimizing proteins and formulations for diagnostic applications. An ideal Cas protein for CRISPR diagnostics is one that is fast, robust, and sensitive. A fast protein enables reduced turnaround times for diagnostic assays. Robust enzymes are more likely to be successful when combined with other molecular processes, such as in the one-pot DETECTR assay. Sensitive enzymes enable lower limits of detection from either small amounts of amplified product or when eliminating pre-amplification and doing direct detection of target nucleic acids. FIG. 133 illustrates a flow diagram of a process used to evaluate, characterize, and optimize proteins for diagnostic applications. Proteins may be evaluated based on thermostability, compatibility with various assay buffers, and sensitivity. FIG. 134 illustrates a schematic showing a workflow for the process using Labcyte Echo. Using Labcyte echo enables 4.5× faster evaluation and characterization of enzymes in comparison to manual or semi-automated setup.

Initial optimization of programmable nuclease systems (e.g., the combination of Cas protein and guide nucleic acids) involved screening the performance of candidate systems in a variety of buffers and at temperatures from 35° C. to 60° C. The buffers selected for screening included buffers used in DETECTR assays and other Cas activity buffers. The buffers may comprise a range of salt types, salt concentrations, counter ion types, and various additives important for polymerase performance.

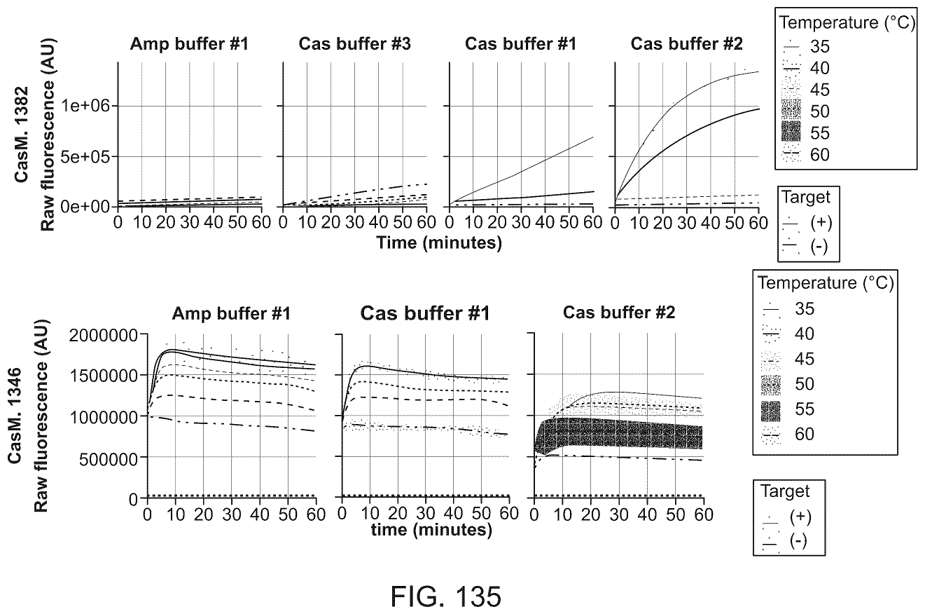

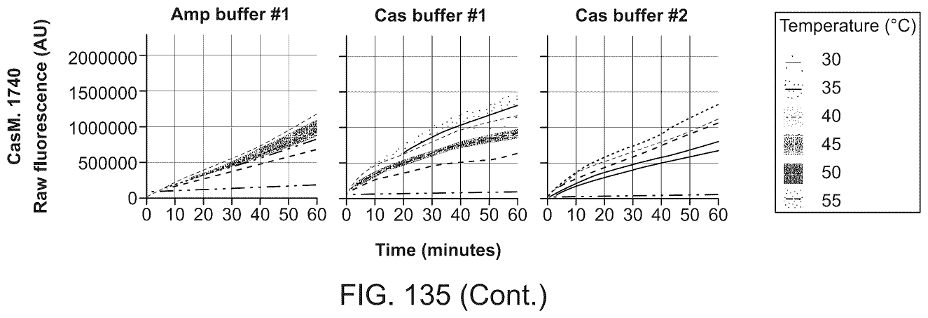

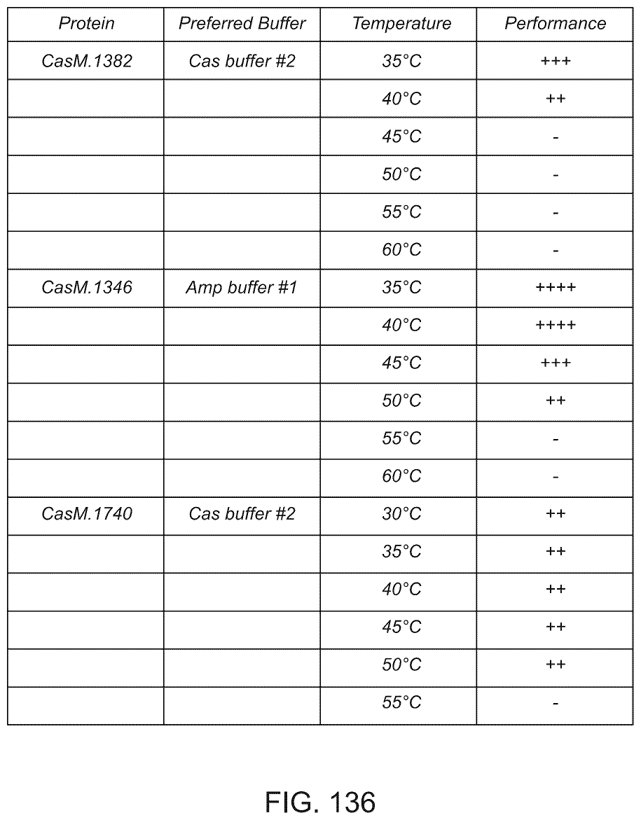

FIG. 135 shows the results of three candidate Cas enzymes trials. The results of trans-cleavage assays for the Cas enzymes exemplified the differences that were observed between enzyme performance. For example, CasM. 1382 performed strongly in Cas buffer #2, and had little activity in Amp buffer #1. In Cas buffer #3, which had high levels of Mg⁺², elevated background signals were observed in the no-target controls. CasM. 1346 showed strong performance in various buffers. The kinetic curves from this enzyme suggest that it was fast and sensitive, which lead to a large initial signal increase at multiple temperatures, but the enzyme quickly denatured and did not demonstrate sustained activity at temperatures over 40° C. CasM. 1740 showed slower activity in comparison to CasM. 1346. CasM. 1740 showed robust and continued activity at temperatures as high as 50° C. but not at 55° C., suggesting that CasM. 1740 is a thermostable Cas protein and may be compatible with a variety of assay designs for direct detection or when coupled to other signal amplification processes.

FIG. 136 shows the performance of three candidate Cas enzymes at different temperatures and buffers.

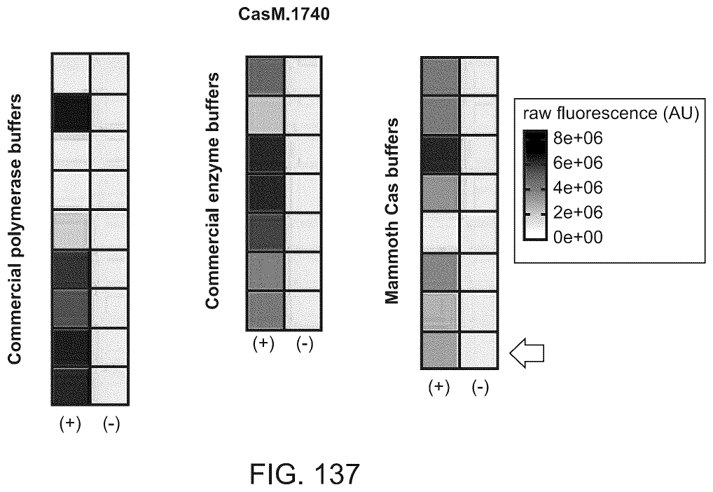

FIG. 137 shows the results of testing conducted with CasM. 1740 with three additional buffers at 35° C. CasM. 1740 was robustly active in some polymerase buffers and many enzyme activity buffers. The results show that the performance of the enzymes may be optimized or enhanced by using certain buffers.

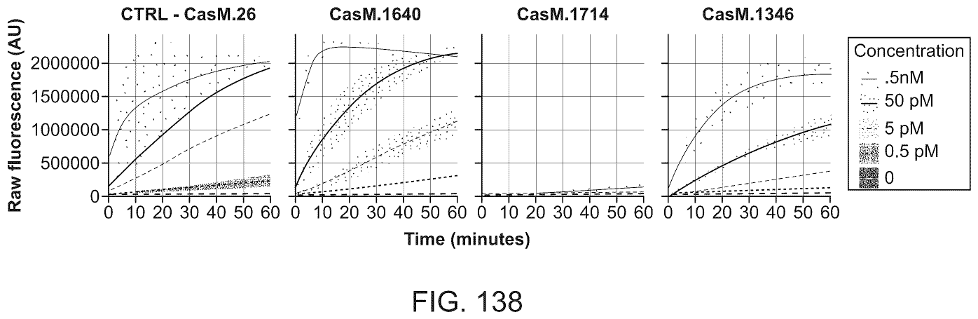

FIG. 138 shows the results of experiments investigating the limits of detection on single-strand oligo or synthetic dsDNA target at 35° C. Some systems, although functional, did not have robust limits of detection such as CasM. 1714. Some performed similarly or potentially outperformed the control system CasM. 26.

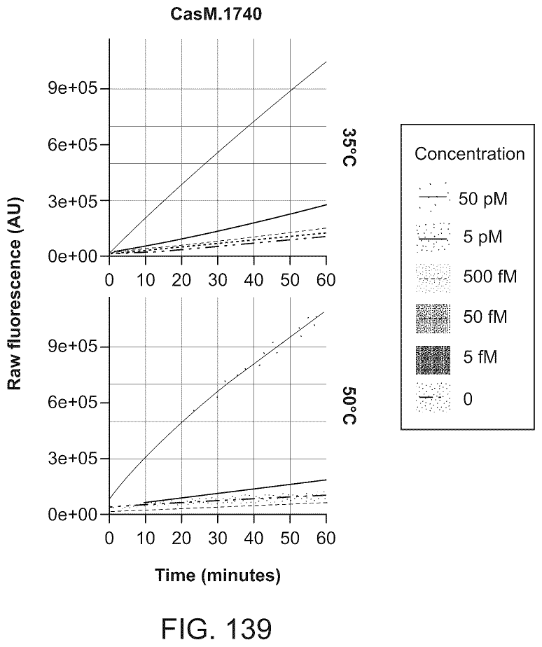

FIG. 139 shows the results of experiments evaluating the limit of detection at both 35° C. and the highest temperature that the protein was demonstrated to function at for CasM. 1740. At both 35° C. and 50° C., the performance of the enzyme was similar with the ability to detect 5 μM of a target.

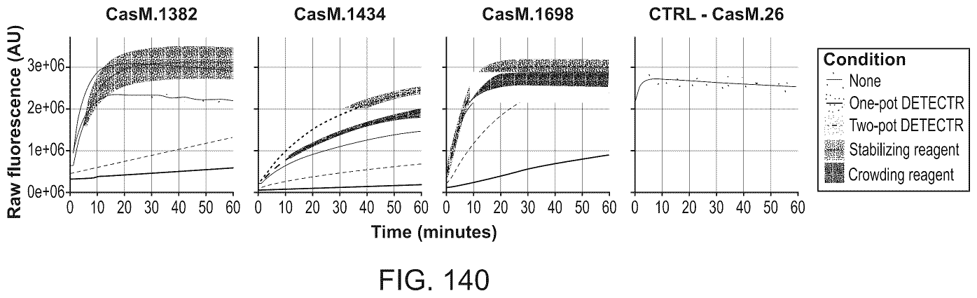

FIG. 140 shows the results of experiments investigating the effects of additives and assay formulations on the performance of the proteins. Additives included those used for lyophilization (e.g., trehalose), for stabilizing protein performance (e.g., BSA), and for reducing water content and crowding reactions (e.g., PEG). Assay formulations comprised a buffer, enzyme, primer, and dNTP concentrations used in one-pot DETECTR and two-pot DETECTR reactions. In some cases, the additives had little or no effect (e.g., CasM. 1382 and CasM. 1698), and in some cases, the additives boost the performance of the enzyme (e.g., CasM. 1434). Some enzymes were more or less tolerant of assay formulations. For example, CasM. 1698 performed well in the two-pot DETECTR assay formulation and had reduced activity in the one-pot DETECTR assay formulation. In contrast, CasM. 1382 showed similar performance to CasM. 1698 when no additives are used and was almost completely inhibited in the one-pot DETECTR assay formulation. This suggest that CasM. 1382 may be more robust in a range of assay conditions.

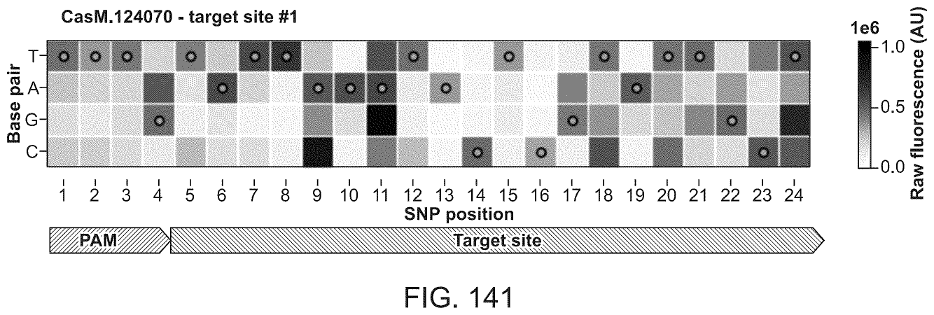

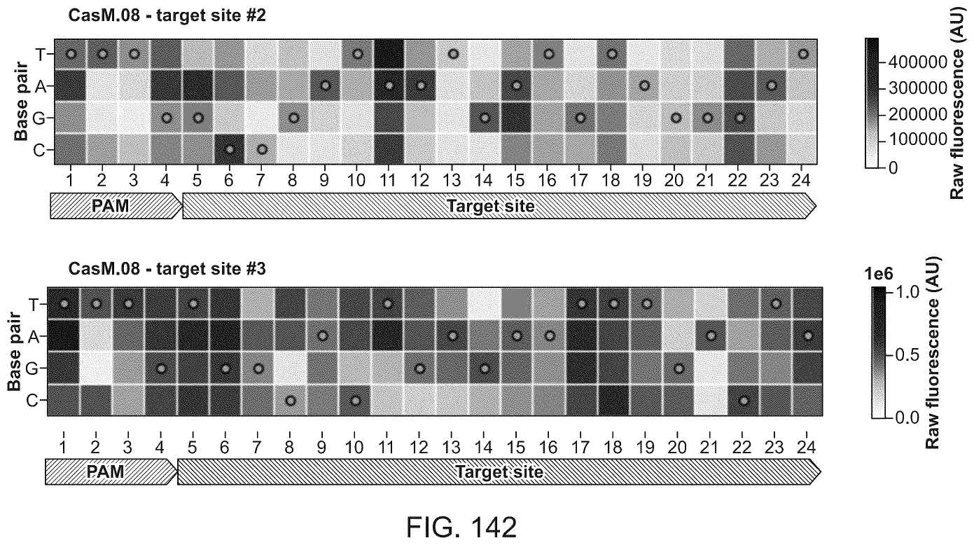

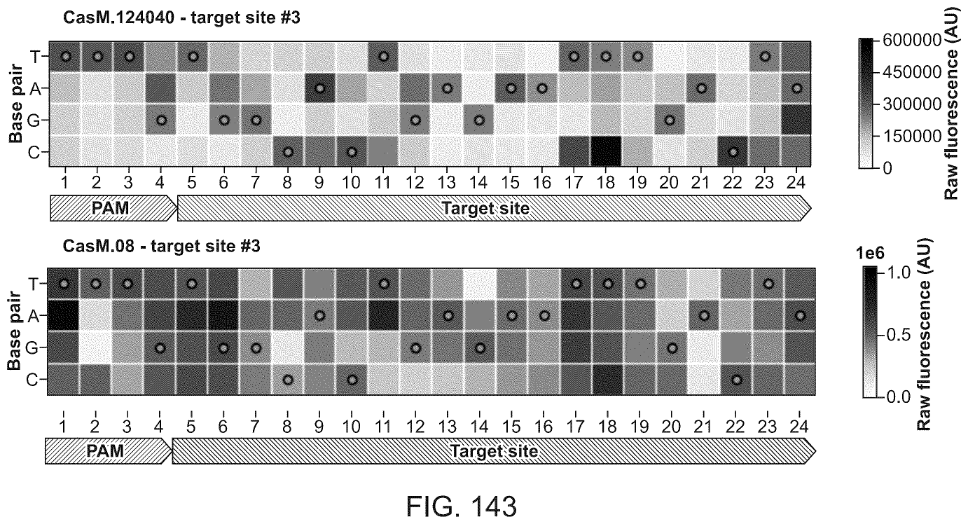

FIG. 141, FIG. 142, and FIG. 143 show the results of experiments investigating the sensitivity of Cas proteins to single-nucleotide mutations. Proteins that have high sensitivity to mutations may be useful for genotyping applications, such as the detection of viral strains or somatic genotyping for disease alleles. Meanwhile, proteins that have low sensitivity to mutations may be useful for assays where robust detection is most important. To evaluate single nucleotide mutation sensitivity of each protein, a series of synthetic dsDNA target molecules that contained all 4 potential mutations at every position along the target site and in the PAM region were synthesized. Each protein is screened in optimal conditions, as previously determined. For proteins with strong sensitivity to single nucleotide mutations, a high signal from the DETECTR assay was observed only for the nucleotides that match those in the gRNA. For proteins with low sensitivity to single nucleotide mutations, a high signal from the DETECTR assay was observed for one more nucleotides, regardless of whether the nucleotide matches those in the gRNA or not. FIG. 141 shows the results for CasM. 124070 which showed strong single nucleotide mutation sensitivity. FIG. 142 shows the results for CasM. 08, which shows that the sensitivity depended on the target site. FIG. 143 shows the results for CasM. 124070 and CasM. 08 with the same target site, showing that different proteins had different sensitivity to the same target site.

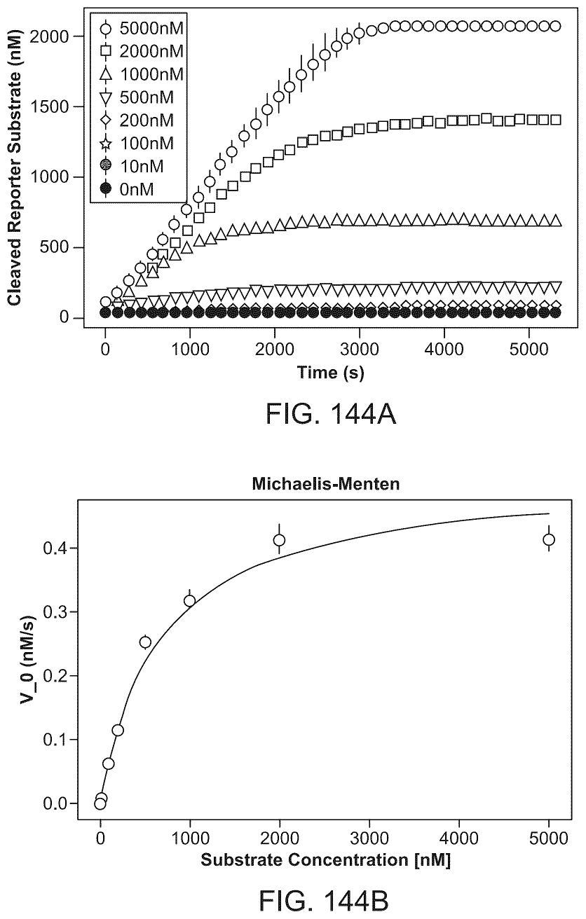

FIGS. 144A-144B show the results of experiments investigating the kinetics of trans-cleavage for proteins that have the best performance in terms of sensitivity, specificity, or thermostability. RNP complexes were prepared and incubated with target DNA or RNA for 30 minutes at the optimal temperature for the system. Various concentrations of quenched fluorescent reporters were added to the system and a plate reader was used to measure the fluorescence over time. The results were normalized across machines and temperatures for each protein. Michaelis-Menten kinetics were analyzed for each system. The results showed striking differences in the catalytic efficiency between Cas effectors. The results suggest that systems with the greatest catalytic efficiency tend to have the lowest limit of detection.

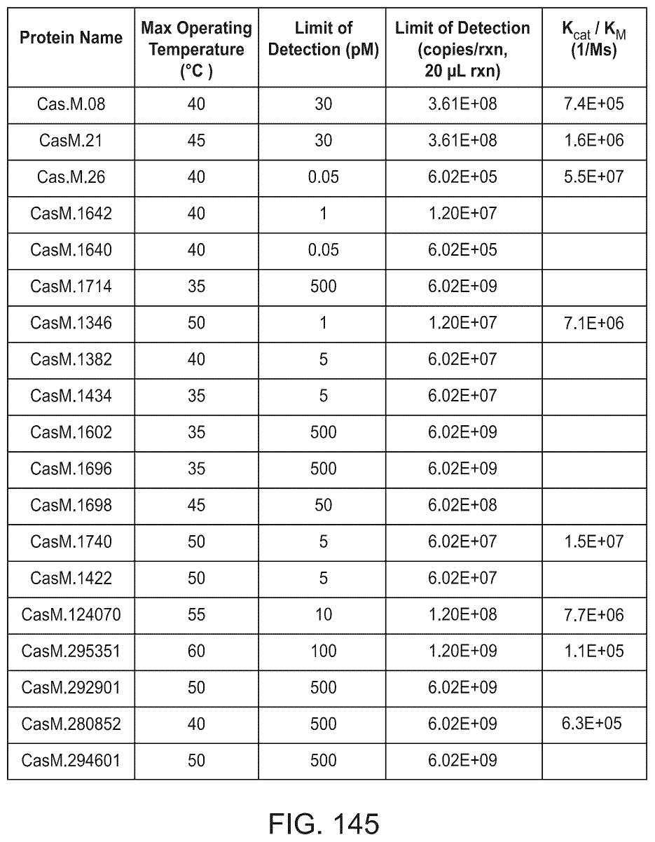

FIG. 145 summarizes the performance results for various Cas enzymes.

The terms “sample interface”, “sample input”, “input port”, “input”, “port” as used herein, generally refers to a portion of a device that is configured to receive a sample.

The terms “heating region”, “heated region”, “heat chambers”, “heat volumes”, “heat zones”, “heat surfaces”, “heat areas”, and the like, as used herein, generally refers to a portion of a device that is in thermal communication with a heating unit.

The terms “heater”, “heating unit”, “heating element”, “heat source”, and the like, as used herein, generally refers to an element that is configured to produce heat and is in thermal communication with a portion of a device.

The term “reagent mix”, “reagent master mix”, “reagents”, and the like, as used herein, generally refers to a formulation comprising one or more chemicals that partake in a reaction that the reagent mix is formulated for.

The term “non-cycled temperature profile,” as used herein, generally refers to a temperature profile that is cyclical or sinusoidal in that the temperature profile has an initial temperature, a target temperature, and a final temperature.

The term “capture probe”, “capture molecule”, and the like, as used herein, generally refers to a molecule that selectively binds to a target molecule and only nonspecifically binds to other molecules that can be washed away.

The term “collection tube,” as used herein, generally refers to a compartment that is used to collect a sample and deliver the sample to the sample interface of a device. In some embodiments, the collection tube may be portable. In some embodiments, the collection tube is a syringe.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Electrochemical Detection of the HERC2 Gene Using the DETECTR Reaction

DETECTR has previously been demonstrated to be a powerful technology for detection of pathogens such as SARS-CoV-2 (Broughton et al., 2020). Electrochemical detection has been demonstrated to show a lower limit of detection than fluorescence-based assays by roughly two orders of magnitude (Lou et al., 2015). In this example, an electrochemical probe is incorporated into the DETECTR assay.

The following electrochemical probe: 5′-2XXTTATTXX-3′; Where 2=5′ 6-FAM; X=ferrocene dT and 3=3′ Biotin TEG was used. Additionally, both cyclic voltammetry and square wave voltammetry with the probe, using a DropSens pSTAT. ECL instrument and DropSens screen-printed carbon electrodes were used. A difference in signal between the initial and final timepoints of the DETECTR reaction was observed. A detection of 50 fM. HERC2 target, which is much lower than has been observed with the fluorescence assay was achieved. FIG. 13: presents an oxidation curve for HERC2 DETECTR reaction with electrochemical reporters. Error bars represent standard deviation of two measurements of the same solution, using three traces from each measurement. A 40 nA difference in signal was observed indicating presence of 50 fM of HERC2 target. FIG. 14: presents a reduction curve for HERC2 DETECTR reaction with electrochemical reporters. Error bars represent standard deviation of two measurements of the same solution, using three traces from each measurement. A 30 nA difference in signal indicates the presence of 50 nM of HERC2 after 33 min of reaction. FIG. 15: presents a resultant cyclic voltammogram taken before and after HERC2 DETECTR reaction using 24 μM electrochemical reporter. Each trace is the average of three scans of the same solution and error bars represent standard deviations and the discernable difference between the two voltammograms indicates the presence of 24 μM of reporter.

### Example 2: SARS-CoV-2 Electrochemical DETECTR Reaction

The following electrochemical probe: 5′-2XXTTATTXX-3′; where 2=5′ 6-FAM; X=ferrocene dT; and 3=3′ Biotin TEG was used with a DNA sequence, part of the genome of SARS-CoV-2. Square wave voltammetry measurements were performed using a DropSens pSTAT ECL instrument and DropSens screen-printed carbon electrodes. A difference in signal between the initial and final timepoints of the DETECTR reaction, using 500 fM of target, was detected. FIG. 16 presents an oxidation curve for SARS-CoV-2 DETECTR reaction with electrochemical reporters. Error bars represent standard deviation of three traces from each measurement. The detailed experimental conditions are as follows. A DETECTR mix was prepared according to FIG. 18. Diluted stock reporter 1/10 (1 μL reporter plus 9 μL nuclease-free water) was used to make 240 μM stock. After 30 minute incubation of DETECTR master mix at 37° C. and adding reporter, the reaction mixture was split into two aliquots of 152 μL of master mix plus 8 μL of target (either 1× TE or 10 μM stock concentration gene fragment). The final target concentration was 500 fM. A 55 μL aliquot for measurement was removed at t=0 and then incubated the rest on a heat block at 37° C. for 20 minutes prior to electrochemical measurement. Square wave voltammetry with parameters according to FIG. 19 were used. One 50 μL drop of solution for each measurement was used. Prior to use, each electrode was rinsed with 1 mL of 1× TE and dried with compressed air.

Data Analysis was carried out as follows. The first 2 scans were discarded because they typically have anomalous signals from debris on the electrode that is cleared by voltage application. The remaining 3 scans are averaged for each sample, and the standard deviation and coefficient of variance is calculated. The mean current traces were then imported into PeakFit software for a baseline correction. The corrected traces were then exported and the original CV values were used to calculate standard deviations for the error. It should be noted that the aliquot of reporter used had experienced multiple freeze-thaw cycles. The data from the measurements with the original parameter set were noisy and were not fully analyzed. The location of this peak was different from previous experiments, where a peak was observed around 0.15 V. It is worth noting that the aliquot of reporter had experienced multiple freeze thaw cycles, so it may have degraded. As a control, LAMP buffer to determine the relative signal was also run. The LAMP buffer was already activated with magnesium in a previous experiment, then refrozen). The LAMP buffer was run neat, without any dilution. The signal from the LAMP buffer was much higher than the signal from the DETECTR reactions, as is visible in FIG. 17. It was also found that the signal from the negative control at t=0 was higher than the signal from the other DETECTR reactions. However, this measurement was the second one on that sample and electrode, after a first measurement with the original parameters. In conclusion, SARS-CoV-2 electrochemical DETECTR enabled detectable results for the SARS-CoV-2 virus.

### Example 3: Evaluating Function of Biotin Modified gRNAs for Cas13

The purpose of this experiment was to evaluate biotinylated gRNA functionality with Cas13a both in solution and immobilized on a surface. In this experiment three replicate runs of a biotin-modified gRNA (mod023) and three replicate runs of a non-biotin modified gRNA (R0003) were carried out. Three replicate “no target control,” or NTC runs were carried out for both the mod023 reporter and R0003 control. The procedure was carried out as follows:
(1) gRNAs were diluted to 20 uM.
(2) Cas12M08 complexing reactions using gRNAs were prepared. The testing and control complexes were diluted to 100 μM final concentrations. Complexing reactions were carried out in two conditions with 3 replicates each resulting in 6 reactions per gRNA. Please see FIG. 16 for complexing mix details.
(3) Sample was incubated for 30 min at 37 C.
(4) Reporter substrate was added.
(5) Reaction was kept on ice until the next step.
(6) 13 uL of 1× MBuffer 1 was added in wells on 384-well plates.
(7) 5 μL of complexing reaction was added.
(8) In a post-amp hood, 2 uL of 1 nM target (respective) was transferred to and kept in a 384 well plate on ice.
(9) Sealed plate with optically clear seal.
(10) Spun down for 30 sec at 2000 rcf.
(11) Read on plate reader with extended gain settings for 30 min at 37 C.

FIGS. 32A and 32B show results, in solution, for mod023, the biotin modified reporter, and R003 the non-biotin-modified reporter, respectively. It was shown that the biotin-modified gRNA had similar performance to the non-biotin-modified gRNA in solution. FIG. 32C shows results for gRNA that was modified with biotin and immobilized to the surface. FIG. 32D shows results for gRNA that was not modified with biotin but was deposited on the surface in the same manner as C. FIG. 32E, similar to FIGS. 32A and 32B, shows results for gRNA that was unmodified and in solution. Together these results showed that with biotin modification and surface immobilization functionality was maintained and DETECTR assay performance was not adversely affected.

### Example 4: Optimizing Reporter Incubation Time on Streptavidin Slides

The objective of this experiment was to verify if a 30 min incubation time was sufficient to produce a strong signal assay signal. Two concentrations were run. The procedure used is as follows:
1. Dilutions of REP072 rep106 at 1 uM and 5 uM in 1× Wash Buffer were prepared. 1× Wash Buffer is composed of 25 mM Tris, 150 mM NaCl; pH 7.2; 0.1% BSA, and 0.05% Tween®-20 Detergent.
2. The wells of a fresh streptavidin slide were marked as shown in FIG. 33 and FIGS. 34A and 34B using a hydrophobic pen.
3. Three uL of each dilution were spotted on the streptavidin coated slide (in triplicate).
4. The slide was incubated for 30 min at room temperature and covered from light.
5. Three uL of the dilution were removed from each spot.
6. Five uL of 1× wash buffer was added to each spot and mixed up and down 3 times. This wash step was repeated three times.
7. 50 μL of 1× wash buffer was added and incubated for 5 min.
8. The slide was then flicked over a kim wipe to remove all the solution then dabbed gently on all four sides against a kim wipe to clean up the edges.
9. The slides were then imaged on GelDoc (SYBR Blue setting, autoexposure with adjusted gain settings).

FIG. 33 shows results for the test reporter, Rep072, and the negative control, Rep106. The replicates of Rep072 at 5 μM show the strongest signal and the three replicates of Rep072 at 1 μM concentration show the next strongest signal. The negative control reporter, rep106 shows the same low signal (on none at all) for both 5 μM and 1 μM concentrations. This result shows specific binding of a FAM-bioinylated reporter with a 30 minute incubation time at both 5 μM and 1 μM concentrations. FIGS. 34A-34B show similar results with reporters at 5 mM in FIG. 34A and 2.5 mM in FIG. 34B. The top row of FIGS. 34A and 34B shows spots exhibiting bright fluorescence and the bottom row of FIGS. 34A and 34B show spots exhibiting similarly low fluorescence.

### Example 5: Quencher-Based Reporter Testing for Immobilization

Fluorescent quencher-based reporters were tested in an immobilized DETECTR assay. Streptavidin functionalized plates and biotin labeled reporters were used. FIG. 36 shows sequence and other details for reporters used in this experiment. The following procedure was used:
1. Stocks of the reporters rep072, rep104, rep105, rep117 and rep118 were prepared for binding to the reader plate. Reporter binding details can be seen in FIG. 37A.
2. Complexing reactions were then prepared using the mod018 sequence that is 5′ modified with biotin TEG. See FIG. 36 for more details on sequence mod018. Complexing mix details can be seen in FIG. 37B.
3. Complexing reactions were incubated for 30 min at 37 C.
4. Grid of dilutions of RNP and reporter were prepared with (50:50 ratio) with enough material for 2 reactions each.
5. Wells of a 96-well streptavidin coated plate were pre-rinsed with 100 μL of 1× MBuffer1, twice.
6. 25 μL of complex and 25 μL reporter mix were then added.
7. Sealed plate with foil seal.
8. Binding was then carried out at 25 C for 30 minutes with intermittent shaking (1000 rpm 15 sec every 2 min on Thermomixer).
9. Plates were then spun down briefly.
10. Supernatant was removed.
11. Washed once with 100 μL 1× MBuffer-1. 1× MBuffer-1 is composed of 20 mM Imidazole 7.5, 25 mM KCl, 5 mM MgCl2, 10 μg/mL BSA, 0.01% Igepal Ca-630, and 5% Glycerol.
12. Washed once with 100 μL 1× MBuffer-3. 1× MBuffer-3 is composed of 20 mM HEPES pH 7.5, 2 mM KOAc, 5 mM MgOAc, 1% Glycerol, and 0.00016% Triton-X 100.
13. Added 50 μl of 1× MBuffer3 to each well.
14. Added 5 μL of target/no-target in 1× MBuffer3 a. target volume=5 μL per reaction (GF577 PCR product 1:10).
15. Sealed plate with foil seal.
16. Incubated at 37 C for 90 minutes with intermittent shaking in plate reader measuring FAM intensity.
17. Spun down briefly.
18. Transferred 20 μL of supernatant to wells of 384-well plate and measured FAM fluorescent intensity (single-read).

Results are illustrated in FIGS. 27A-27E. As predicted the positive control shows a positive slope indicating increased binding over the course of the reaction. This is due to the release of FAM dye into solution upon binding and transcleavage as seen in FIG. 27B. In rep104 the cleavage point is between the FAM and the biotin, while the biotin in all reporters is the attachment point to the streptavidin surface. FIG. 27C plots the control, target binding kinetic plot for rep105. Rep105 is composed of biotin-FAM-T16-FQ (SEQ ID NO: 68). In this case the streptavidin coated surface emits fluorescence because the region between the FAM dye and the quencher is cleaved upon binding and the quencher is released. FIG. 27D plots the control, target binding kinetic plot for rep117. Rep117 is composed of biotin-FAM-T20-FQ (SEQ ID NO: 71). In this embodiment, where the reporter is cleaved between the FAM dye and the quencher, thus allowing for release of the quencher in the solution upon binding and transcleavage. This in turn, allows the surface emits fluorescence. FIG. 27E plots the control, target binding kinetic plot for rep118. Rep118 is composed of FAM-T20-biotin-FQ (SEQ ID NO: 72). In this embodiment, the solution emits fluorescence because upon binding the nucleic acid region between the biotin and the FAM is transcleaved, thus releasing the FAM into solution.

### Example 6: Immobilization Optimization—Complex Formation Step

The objective of this experiment was to determine whether binding both the gRNA and reporter to a plate allows the DETECTR assay to be as effective as binding the CAS protein-gRNA complex and reporter. This removes the need to functionalize the surface with the pre-complex of gRNA and CAS protein, allowing for an easier manufacturing process. Additionally, greater specificity can be achieved by allowing for more stringent washes. The following procedure was used.
1. The experiment was designed as shown in FIG. 38A.
2. A stock solution of reporter rep117 was bound to the plate according to the conditions presented in FIG. 38B.
3. Complexing reactions for mod018 (5′ biotin-TEG R1763 SARS-CoV-2 N-gene) and R1763 CDC-N2-Wuhan were then prepared according to the conditions presented in FIG. 39A.
4. Two sets of full complexing mix were made for each and two mixes without Cas12M08 according to FIG. 39B.
5. Incubated complexing reactions for 30 min at 37 C
6. Pre-rinsed wells of 96-well streptavidin coated plate with 50 μL of 1× MBuffer1, twice
7. Added 25 μL reporter to each well.
8. Added 25 μL of complex to A1-D2, 25 μ L 1× MB1 to A3-D4, and 25 μ L cRNA mix A5-D6.
9.Sealed plate with foil seal.
10. Ran binding reaction at 25 C for 30 minutes with intermittent shaking, 1000 rpm 15 sec every 2 min on Thermomixer
11. Spun streptavidin plate down briefly.
12.Removed supernatant.
13. Washed twice with 100 μ L 1× MBuffer-1.
14. Washed once with 100 μ L 1× MBuffer-3.
15. Added 50 μ l of 1× MBuffer3 to wells A1-D2.
16. Added 25 μ L 1XMB3 and 25 μ L of complex to “in-solution” wells A3-D4.
17. Added 47.5 μ L 1XMB3 and 2.5 μ L Cas12M08 (50 uL MM) to each “prot after” well A5-D6.
18. Added 5 μ L of 1:10 diluted purified LAMP product to (+) target wells.
19.Sealed plate with optically clear seal.
20. Read on plate reader—FAM, 37 C, 90 min.

The results of this experiment are shown in FIGS. 29A-29F show that is it possible to add CAS protein with the target and still achieve complexing and signal. FIGS. 29A-29C illustrate results for a first replicate of tests. FIGS. 29D-29F illustrate results for a second replicate of tests. FIGS. 29A and 29D show results where both a biotinylated reporter and a complex of biotinylated RNA and CAS protein were immobilized. Here activity buffer and target were then added. FIGS. 29B and 29E illustrate results where the biotinylated reporter is immobilized and all other reaction components including gRNA and CAS protein are introduced in solution. FIGS. 29C and 29F illustrate results where the biotinylated reporter and biotinylated gRNA are immobilized and then buffer, CAS protein and target are added. In these results it is observed that complexation of CAS protein and gRNA and a reporter signal upon binding can be detected when only gRNA and reporter are immobilized as shown in FIG. 29F.

### Example 7: Demonstration of Immobilized Target Discrimination

The purpose of this experiment was to demonstrate target discrimination for immobilized reporters for the DETECTR reaction. The experiment design used in this experiment is shown in FIG. 40A. The following procedure was used.
1. Experiment planned as shown in FIG. 40A. The experiment included 3 gRNAs including mod018, mod025 and mod024. Two targets and two controls were used. The two targets were N-gene and RNaseP. The two controls were: (1) no target with all other reaction components and (2) water.
2. Stock solution of reporter rep117, later bound to plate, was prepared as shown in FIG. 40B.
3. Complexing reactions were prepared for the three gRNAs: mod018, mod024 and mod025 with reporters:
(1) biotin-TEG R1763 SARS-CoV-2 N-gene,
(2) 5′ biotin-TEG R777 Mammuthus,
(3) 5′ biotin-TEG R1965 RNase P, respectively. The reaction conditions are shown FIG. 41A.
4. Pre-rinsed wells of 96-well streptavidin coated plate with 50 μ L of 1× MBuffer1, twice.
5. Added 25 μ L reporter to each well.
6. Added 25 μ L complexing mix to wells.
7. Sealed plate with foil seal.
8. Ran binding reaction at 25 C for 30 minutes with intermittent shaking (1000 rpm 15 sec every 2 min on Thermomixer).
9. Ran FASTR protocol as follows:
a. Primers used:
   I. SARS-CoV-2: M2062 CDC N2-FWD/M2063 CDC N2-REV.
   II. RNase P: POP7 8F/6R. Please see FIG. 41B for reaction conditions.
   II. a. Pipette 4 μ L of master mix into wells of MBS 96-well plate.
b. Added 1 uL twist RNA dilution.
c. 1000 copies/uL: 7.8 uL of 6400c/uL in 42.2 uL H2O.
d. Sealed plate with foil seal at 165 C for 1.5 seconds.
e. Ran the following PCR protocol on the MBS NEXTGENPCR thermocycler according to conditions shown in FIG. 42.
f. Removed plate from thermocycler.
g. Spun down at 2000 rpm for 30 sec.
h. Kept on ice until ready to use.
10. Spun streptavidin plate down briefly.
11. Removed supernatant.
12. Washed twice with 100 μ L 1× MBuffer-1.
13. Washed once with 100 μ L 1× MBuffer-3.
14. Added 50 uL 1XMB3 15 mM Mg2+.
15. Added 4 μ L of target from FASTR to target wells.
16. Sealed plate with optically clear seal.
17. Read on plate reader—FAM, 37 C, 90 min.

Results are shown FIGS. 31A-31C. FIG. 31A presents results for reporter mod018 showing specificity for the N-gene target. FIG. 31B presents results for reporter mod025 showing specificity for the RNaseP target. FIG. 31C presents results for mod024 showing no signal as predicted since no target was present.

### Example 8: Functional Testing for One-Pot RT-LAMP

The purpose of this experiment was to test the functionality of a one-pot reaction composed of both RT-LAMP and Cas12M08 enzyme-based DETECTR master mixes. Both master mixes were co-lyophilized together into one master mix. The reactions are functionally incompatible due to optimal reaction temperature differences, so they were evaluated independently. The RT-LAMP master mix shows reaction characteristics like the liquid controls. Results are shown for the RT-LAMP assay in FIG. 99A and the Cas12M08 assay in FIG. 99B. The results shown in in FIG. 100 demonstrate a ‘one pot’ reaction, composed of RT-LAMP and Cas12M08 DETECTR master mixes, co-lyophilized into one master mix and reconstituted. The results shown in FIG. 100 demonstrate activity based on the reaction curves.

### Example 9: Cas14a1 DETECTR

The purpose of this experiment was to investigate Cas14a1 which is functional at higher temperatures than RT-LAMP assay requires. In this example the temperatures were run up to 55° C. The results shown in FIG. 101 demonstrate that Cas14a1 was able to be lyophilized and reconstituted with activities comparable to the control.

### Example 10: Functional Testing of a One-Pot Cas12M08-Based DETECTR Assay

The purpose of this experiment was to investigate the performance of pooled and lyophilized master mixes containing both RT-LAMP and DETECTR reaction reagents together. Further this study showed that the pre-lyophilized mixture is stable for two weeks prior to lyophilization. The master mix of RT-LAMP and Cas12M08-based DETECTR lyophilized together, was functionally tested separately, since the Cas12M08 is not compatible with the RT-LAMP amplification conditions. These data show robust RT-LAMP and Cas12M08-based DETECTR activity, comparable to the master mix that was stored at 4° C. for two weeks prior to lyophilization. Results are shown in FIGS. 102A and 102B.

### Example 11: Smaller Volume Lyophilization Reactions

Previous to this example, lyophilized sample volume was demonstrated at 250 μL and performed in glass vials. This example provides data for lyophilization of 25 μL samples in 8-well plastic strip tubes. Additionally, the pre-lyophilization (3 weeks at 4° C.) and values are comparable showing stability of the master mix as seen in FIG. 103. Differential scanning calorimetry results of a lyophilized master mix of reaction reagents, including in 15% Trehalose, are shown in FIG. 104. A half-height mid-point of 109.89° C. was observed. These results taken together demonstrate that the master mix of reaction reagents are stable throughout the lyophilization process at smaller volumes.

### Example 12: One-Pot DETECTR on Handheld Microfluidic Device

In this experiment, the performance of the one-pot DETECTR assay in a handheld microfluidic device was evaluated. Here, one-pot refers to both the RT-LAMP and DETECTR reagents lyophilized as one master mix of reagents. The functions performed on the handheld, microfluidic device included: sample intake; RNA extraction from sample; mixing of the sample with CRISPR reactants; and the transfer of the mixture to a well to heat the mixture to the reaction temperature. In the device, the mixture was heated, and fluorescence was monitored continuously over time. FIG. 106 displays results plotted at 3 different data acquisition settings. The series made of squares shows a setting that did not saturate the detector and therefore displayed the full dynamic range of the signal throughout the life of the assay. From this data it was determined that the DETECTR assay is functional when ran on a miniaturized handheld, microfluidic device.

### Example 13: DETECTR-Based Lateral Flow Assay Strip

The purpose of this example is to demonstrate a DETECTR™-based multiplexed assay using a lateral flow assay (LFA) strip for parallel readout as illustrated in FIG. 107A-107B. To perform the DETECTR™ assay, a surface (10700) is immobilized with programmable nuclease probes (10707) and reporter probes (10701). In this example, the surface is the bottom of a well, separate from the LFA strip. The reporter probes (10701) contain a surface linker (10702), a cleavable nucleic acid sequence (10703), a label (10704) and binding moiety for the flowing capture probe (10705) of the strip. In this example the binding moiety is biotin. The label and binding moiety are attached to the nucleic acid (10703) by a second linker (10706), where in this example the linker is a dendrimer or trebler molecule. The programmable nuclease probe (10707) contains a surface linker and a programmable nuclease (e.g., Cas enzyme) that in turn contains an sgRNA (10708). The sgRNA contains a repeat unit (or hair pin) and a recognition sequence. The recognition sequence is the compliment for a target nucleic acid of the sample. Anti-biotin labeled gold nanoparticles are located in the sample pad (10711) of the LFA strip (10710) as shown in FIG. 107B. In this example, the first step is to contact the surface (10700) with a sample containing target nucleic acids. Upon binding of a target nucleic acid that is complimentary to the sgRNA (10708) of the immobilized programmable nuclease probe (10707), the reporters (10701) immobilized in near proximity, are cleaved by the programmable nuclease, releasing a cleaved section of the reporter (10709) into solution. The sample solution now containing cleaved reporters corresponding to target nucleic acids that were present in the sample, is then contacted to the sample pad (10711) of the lateral flow assay strip (10710). In this example, the sample pad has flowing capture probes (e.g., anti-biotin labeled gold nanoparticles) disposed thereon. Once the sample solution containing released sections of reporters is contacted with the sample pad, said sample solution then flows across the sample pad (for e.g., by being wicked). The cleaved reporters in the sample solution contact and bind to the anti-biotin gold nanoparticle flowing capture probes upon contact in solution. The complex of reporter and nanoparticle is then carried downstream with the rest of the liquid sample by capillary action through the detection region (10712) of the lateral flow assay strip. In this example, the detection region (10712) may comprise six detection spots (10713), where each detection spot (10713) contains a different capture antibody type that is specific for a reporter's dye (10704). In this example, the dye (10704) is FITC and the stationary capture probe is an anti-FITC antibody functionalized to the detection spot (10713) allowing for the specific detection of the FITC labeled reporter among other reporters that are specific for other target nucleic acids. A control line (10714) is present, functionalized with anti-IgG so that all flowing capture probes, not bound to a FITC labeled reporter fragment, (e.g. detection moiety) are captured and detected. It should be noted that in this example, multiple labels and binding moieties were present via the dendritic linker of the detection moiety (10706) to amplify the signal. In this example, multiple detections spots (10713) are present, allowing for the possibility parallel detection of multiplexed samples, as explained in Example 14.

### Example 14: Multiplexed DETECTR-Based Lateral Flow Assay Strip

The purpose of this example is to demonstrate a lateral flow assay strip workflow utilizing a multiplex “Hotpot” assay as illustrated in FIG. 108. In this example, a sample (10801) contains target nucleic acid sequences 1 and 2. The sample (10801) is contacted to the surface (10802) of a well where at each of five locations, D1 through D5, there are different sgRNA's immobilized to the surface. For example, each of the 5 different sgRNA's are part of 5 different programmable nuclease probes (e.g., see FIG. 107) immobilized in the five different locations D1 through D5, as depicted in FIG. 108. Additionally, each of the 5 different sgRNA's are designed to specifically bind to different target nucleic acids in the sample, thus allowing for sample multiplexing. In addition to the immobilized programmable nuclease probes containing sgRNAs, each location, D1 through D5, is functionalized with reporter probes having distinct functional groups. The reporter probes are in close enough proximity to be cleaved by the programmable nuclease probes. Therefore, as described in example 13, reporters are cleaved and released into the solution upon binding between a sgRNA and the target nucleic acid that the sgRNA is designed to bind specifically to. In this example, D4 and D5 each contain reporters labeled with two different labels or capture antibody recognition elements. Once the sample has contacted with the wells (D1 to D5), the respective cleaved sections of reporters are released into the sample solution (as described in Example 13). The sample solution with the released reporters are then contacted with a sample pad, wherein in this example, is situated on lateral flow assay. In this example each detection spot contains a different type of capture antibody, where each capture antibody type specifically binds to a particular label of a reporter. For this example, detection spot (10803) contains the capture antibody anti-FITC, whereas well D5 contains 1) the immobilized Cas-complex including the sgRNA specific to a first target nucleic acid sequence, and 2) the immobilized reporter (10806), which is labeled with FITC. Therefore, upon binding of the first target nucleic acid sequence with the corresponding sgRNA, the linkage between the corresponding immobilized reporter (10806) and corresponding nucleic acid (for example see ref. char. 10701 in FIG. 107) is cleaved, thereby releasing the reporter into solution. By contrast, for this example, detection spot (10804) contains the capture antibody anti-DIG, whereas well D4 contains 1) the immobilized Cas complex including the sgRNA specific to a second target nucleic acid sequence, and 2) the immobilized reporter (10805), which is labeled with DIG. Therefore, upon binding of the second target nucleic acid sequence with the corresponding sgRNA, the linkage between the corresponding immobilized reporter (10805) and corresponding nucleic acid (for example see ref. char. 10701 in FIG. 107) is cleaved, thereby releasing the reporter the solution. The solution now containing cleaved reporters (10805 and 10806) is then contacted to the sample pad of the LFA strip along with chase buffer, where the reporters bind with and pick up flowing capture probes (e.g., anti-biotin-AuNPs) that are disposed on the sample pad. The AuNP-reporter conjugates having reporter (10806) labeled with FITC will selectively bind to detection spot (10803) containing the capture antibody anti-FITC, thus indicating the presence of the first target nucleic acid sequence in the sample. The AuNP-reporter conjugates having reporter (10805) labeled with DIG will selectively bind to detection spot (10804) containing the capture antibody anti-DIG, thus indicating the presence of the second target nucleic acid sequence in the sample. In this manner, parallel detection of 2 or more target nucleic acid sequences present in a multiplexed sample is enabled. In some examples, the detection spots are spaced apart from each other in prescribed locations, such that detection of a reporter at a given detection spot will correlate with a specific target nucleic acid.

### Example 15: HRP-Enhanced DETECTR-Based Lateral Flow Assay Strip

The purpose of this example is to demonstrate horse radish peroxidase (HRP) paper-based detection as illustrated in FIG. 109. Here, “paper-based” refers to a lateral flow assay strip. As in examples 7 and 8, a liquid, blue color sample, containing a target(s) nucleic acid sequence is exposed to a surface upon which CRISPR-Cas/gRNA probes and reporter probes are immobilized. In this example, the reporter probes contain HRP molecules. Upon cleavage of the reporter by the Cas enzyme (e.g. programmable nuclease) following a specific binding event between the target and the guide RNA, the cleaved portion of the reporter is released into the sample solution. In this example, the sample solution having the released sections of reported molecules is then contacted with a lateral flow assay strip comprising a sample pad containing sodium percarbonate, which generates H₂O₂ when hydrated. The rehydration of the sodium percarbonate to form H₂O₂ occurs when the sample is wicked through the sample pad and the lateral flow assay to a detection spot, as described in the previous examples 7 and 8. In this example, the lateral flow assay strip contains the chemical substrates DAB and TMB which activate a color change from blue to red, indicating the presence of HRP, and in turn a “hit” for the target nucleic acid sequence. The chemical catalytic nature of HRP enables signal amplification. Alternatively, the readout can also be accomplished in solution, upon a color change of the sample solution from blue to red.

### Example 16: HRP-Enhanced Multiplexed DETECTR-Based Lateral Flow Assay Strip

The purpose of this example is to demonstrate an HRP-signal enhanced multiplexed lateral flow assay as illustrated in FIG. 110. The immobilized surface (11000) and detection on the lateral flow assay strip (11010) are carried out as described in the previous examples with the exception that signal transduction is not carried out by gold nanoparticles scattering light. Instead, the anti-biotin labeled AuNP are supplanted by HRP-anti-biotin DAB/TMB. The HRP is activated by sodium percarbonate present in the LFA strip which is rehydrated by the reaction and or chase buffer. In this manner, HRP allows for strong enough signal so as not to require sample amplification such as PCR. Multiplexed detection is accomplished in the same manner as described in Example 14.

### Example 17: Cas13 Based, HRP-Enhanced DETECTR LFA with Capture Oligo Probe Specificity

The purpose of this example is to demonstrate multiplexed target nucleic acid detection utilizing Cas13 RNA cleaving specificity over DNA, HRP-signal enhancement, and capture oligo probe specificity as shown in FIG. 111. In this example, the sample (11100) contains different target nucleic acids. The sample (11100) is then contacted to the surface (11101) of the well that is functionalized at five locations, D1-D5, as described in Example 14. The difference here is that the Cas13 enzyme is present in the Cas-complex probe. Cas13 cleaves RNA but not DNA. This enables the use of a reporter (11102) that contains nucleic acid sequences, one composed of DNA and the other composed of RNA. Upon binding of the target nucleic acid to the sgRNA, the RNA of the reporter is cleaved by the Cas13 enzyme and a fragment containing: a portion of the RNA; the complete DNA sequence; and the FITC label is released into solution. This action is repeated in parallel at each spot D1 through D5 for five different target nucleic acids, producing 5 distinct reporter fragments. The solution is then contacted to the sample pad of the LFA strip, where the sample pad contains HRP-anti-FITC. The FITC labeled reporter fragment then binds to the HRP-anti-FITC, forming a complex (11103) and is carried downstream across the detection region, binding specifically to the detection spot containing a capture oligo that has been designed to be the compliment for the oligo in the complex (11103). In this manner, parallel detection of multiplexed samples as described in Example 14 is possible.

### Example 18: HRP-Enhanced DETECTR™-Based Lateral Flow Assay Strip Utilizing Cas14

The purpose of this example is to demonstrate horse radish peroxidase (HRP) paper-based detection as illustrated in FIG. 109. Here, “paper-based” refers to a lateral flow assay strip. As in examples 13 and 14, a liquid, blue color sample, containing a target(s) nucleic acid sequence is exposed to a surface upon which programmable nuclease probes and reporter probes are immobilized. In this example, the reporter probes contain HRP molecules. Upon cleavage of the reporter by the Cas14 enzyme following a specific binding event between the target and the guide nucleic acid, the cleaved portion of the reporter is released into the sample solution. In this example, the sample solution is then exposed to a lateral flow assay strip comprising a sample pad containing sodium percarbonate, which generates H₂O₂ when hydrated. The rehydration of the sodium percarbonate to form H₂O₂ occurs when the sample is wicked through the region, as described in the previous examples 13 and 14. Since the substrate contains DAB, TMB, etc. . . . the “spot” changes from blue to red, indicating the presence of HRP, and in turn a “hit” for the target nucleic acid sequence. In this manner HRP enables signal amplification. Alternatively, as shown in FIG. 109, the readout can also be accomplished in solution, upon a color change of the sample solution from blue to red.

### Example 19: HRP-T20-Biotin (SEQ ID NO: 73) DETECTR™-Based Assay

The purpose of this example was to demonstrate an HRP and DETECTR™-based assay. In this example, reporters were cleaved by a Cas complex, or a DNAse enzyme in solution. The cleaved reporter was reacted to HRP-T20-biotin (SEQ ID NO: 73). The supernatant solution was then added to a reaction volume that contained TMB and H₂O₂ to generate a color signal. The cleaved reporter-HRP conjugate was then detected by optical density measurement of the solution. Optical density measurements were acquired from the beginning of the reaction. The experiment was performed in two sets comprising 2 runs each, where each set was run 1 week apart. DNase and DETECTR™ were used separately in each run of each set. In the DNase runs, 1 nM of HRP target oligo was used in the filled in circle series. Results are shown in FIG. 112. The significance of this example is that multiple turnovers of both HRP and DETECTR™ enable alternative signal amplification to sample amplification such as PCR.

### Example 20: Guide Pooling for Enhanced Target Detection Signal in DETECTR Assays

Guide RNAs that were designed to bind to a different region within a single target molecule were pooled as a strategy for enhancing the target detection signal from DETECTR assays. For examples, in this strategy, each DETECTR™ reaction contained a pool of CRISPR-Cas RNP complexes each of which targeted a different region within a single molecule. As discussed in the paragraphs below, this strategy resulted in increased sensitivity to target detection by using increased number of complexes/single target such that the signal is strong enough to detect within a Poisson distribution (sub-one copy/droplet) and provide a quantitative evaluation of target numbers within a sample.

To test the effect of guide pooling on target detection using the Cas12a nuclease, first, a Cas12a complexing mix was prepared. The R1965 (off-target guide), R1767, R3164, R3178 guides were present in the Cas12a complexing mix in either a pooled-gRNA format (a pool of two or more of the three guides selected from R1767, R3164, or R3178) or in a single-gRNA format (wherein R1767, R3164, R3178 were present individually) and the mix was incubated for 20 minutes at 37° C. A 2-fold dilution series for the template RNA (GF184) was created from a starting dilution concentration (wherein 5.4 μl of GF184 at 0.1 ng/μL was added to 44.6 μl of nuclease-free water). DETECTR master mixes which included the Cas12 complex, Reporter substrate, Fluorescein, Buffer, and diluted template (GF184 or off-target template GF577) were then assembled as shown in Table 2. The DETECTR mixes were then loaded into a Stilla Sapphire chip and placed into the Naica Geode. Crystals were created from thousands of droplets from each sample. No amplification step was performed. The signal from the Sapphire chips was measured in the Red channel. The results of the DETECTR assay showed enhanced Cas12a-based detection of the GF184 target using a pooled-guide format compared to DETECTR Cas12a-based assay using an individual guide format. For example, the DETECTR assays showed an enhanced signal from chamber 5 containing a pool of two guides R1767 and R3178, compared to the signal from chamber 2 or chamber 4 which contained the R1767 and R3178 in individual guide format, respectively, as shown in FIG. 114. Similarly, the DETECTR assays showed an enhanced signal from chamber 9 containing a pool of three guides (R1767, R3164, and R3178), compared to the signal from chamber 5 which contained a pool of two guides (R1767 and R3178) and compared to the signal from chamber 2, chamber 3, or chamber 4 which contained the R1767, R3164, and R3178 in individual guide format, respectively, as shown in FIG. 114.

### Table 2 Displays Conditions, Copies per Chamber, Number of Droplets and Copies/Droplet per Chamber

| | | Copies/ | | copies/ |
|---|---|---|---|---|
| Chamber | Condition | Chamber | # Droplets | droplet |
| 1 | Off Target | 2.5 × 10⁷ | 29336 | 852 |
| | Guide (1965) | | | |
| 2 | Single R1767 | 2.5 × 10⁷ | 26838 | 931 |
| 3 | Single R3164 | 2.5 × 10⁷ | 29590 | 845 |
| 4 | Single R3178 | 2.5 × 10⁷ | 27769 | 900 |
| 5 | 2x pool | 2.5 × 10⁷ | 27929 | 895 |
| | (R1767, | | | |
| | R3178) | | | |
| 6 | 2x pool | 1.25 × 10⁷ | 28787 | 434 |
| | (R1767, | | | |
| | R3178) | | | |
| 7 | 2x pool | 6.125 × 10⁶ | 27503 | 223 |
| | (R1767, | | | |
| | R3178) | | | |
| 8 | 2x pool | 0 | 28814 | 0 |
| | (R1767, | | | |
| | R3178) | | | |
| 9 | 3x Pool | 2.5 × 10⁷ | 27881 | 897 |
| | (R1767, | | | |
| | R3164, R3178) | | | |
| 10 | 3x Pool | 1.25 × 10⁷ | 29523 | 423 |
| | (R1767, | | | |
| | R3164, R3178) | | | |
| 11 | 3x Pool | 6.125 × 10⁶ | 28957 | 211 |
| | (R1767, | | | |
| | R3164, R3178) | | | |
| 12 | 3x Pool | 0 | 29087 | 0 |
| | (R1767, | | | |
| | R3164, R3178) | | | |

Enhanced sensitivity to target detection with guide-pooling was observed in the case of Cas13a nuclease also. In these assays, a Cas13a complexing mix was prepared wherein the R002(off-target guide), R4517, R4519, R4530 guides were present in either a pooled-gRNA format (a pool of two or more of the three guides R4517, R4519, and R4530) or single-gRNA format (wherein R4517, R4519, and R4530 were present individually) and the mix was incubated for 20 minutes at 37 C. DETECTR master mixes which included the Cas13a complex, FAM-U5 Reporter substrate, Buffer, and diluted template SC2 RNA (or off-target template 5S-87) was then assembled as shown in Table 3. The DETECTR mixes were then loaded into a Stilla Sapphire chip and placed into the Naica® Geode system. Crystals were generated from the droplets from each of the samples and incubated at 37° C. (no amplification step was performed). The signal from the Sapphire chips was measured in the Cy5 channel. The results of the DETECTR assay showed enhanced Cas13a-based detection of the SC2 target RNA using a pooled-guide format compared to a Cas13a-based detection of the SC2 target RNA using a single-guide format. For example, the DETECTR assays showed an enhanced signal from chamber 8 (saturated—not displayed), containing the template at a concentration of 1×10⁶ copies, and a pool of the three guides R4517, R4519, and R4530, compared to the signal from chamber 2, chamber 4, or chamber 6 which contained the template at a concentration of 1×10⁶ copies, and the guides R4517, R4519, and R4530 in individual guide format, respectively, as shown in FIG. 115. Similarly, the DETECTR assays showed an enhanced signal from chamber 9 which contained the template at a concentration of 1×10⁵ copies and a pool of three guides (R1767, R3164, and R3178), compared to the signal from chamber 2, chamber 6, or chamber 4, which contained the template at a concentration of 1×10⁶ copies, and which contained the R1767, R3164, and R3178 in individual guide format, respectively, as shown in FIG. 115.

### Table 3 Displays Conditions, Copies per Chamber, Number of Droplets and Copies per Droplet per Chamber

| | | Copies/ | | copies/ |
|---|---|---|---|---|
| Chamber | Condition | Chamber | # Droplets | droplet |
| 1 | Off Target | 1 × 10⁶ | 19960 | 50 |
| | Guide (R002) | | | |
| 2 | Single R4517 | 1 × 10⁶ | 18102 | 55 |
| 3 | Single R4517 | 0 | 19146 | 0 |
| 4 | Single R4519 | 1 × 10⁶ | 18289 | 55 |
| 5 | Single R4519 | 0 | 23324 | 0 |
| 6 | Single R4530 | 1 × 10⁶ | 25402 | 39 |
| 7 | Single R4530 | 0 | 26285 | 0 |
| 8 | 3 pool | 1 × 10⁶ | saturated | ~40 |
| 9 | 3 pool | 1 × 10⁵ | 23209 | 4.3 |
| 10 | 3 pool | 1 × 10⁴ | 24064 | 0.41 |
| 11 | 3 pool | 0 | 21137 | 0 |
| 12 | 3 pool | 1 × 10⁶ | 24885 | 40 |

Next, the sensitivity of a target detection in Cas13a digital droplet DETECTR assays containing guide RNA in either a pooled-guide format versus a single guide format was assayed. DETECTR reaction master-mixes was prepared for each gRNA (R4637, R4638, R4667, R4676, R4684, R4689, R4691, or R4785 (RNaseP)) and included, in addition to the gRNA, the Cas13a nuclease, and the reporter substrate. After complexing, 2 μL of each RNP was combined in either a pooled-gRNA format (a pool of the seven gRNAs, i.e., R4637, R4638, R4676, R4689, R4691, R4667, and R4684) or remained in the single-gRNA format (wherein R4667, R4684, and R4785 (RNAse P were present individually). The template RNAs (Twist SC2, ATCC SC2, and 5s-87 off-target) were diluted to obtain a series of template concentrations. DETECTR reactions directed to the detection of the template RNAs (Twist SC2, ATCC SC2, and 5s-87 off-target template RNAs) were assembled by combining the Cas13a-gRNA RNPs with the diluted template RNA from the previous step as shown in Table 4. The assembled DETECTR reactions were loaded into chambers on a Stilla Sapphire Chip. The Chips were placed into the Naica® Geode system and crystals were generated using the droplet generation program and imaged to reveal droplets that contain detected targets.

The sensitivity of target detection by the DETECTR assays containing the pooled guides (R4637, R4638, R4667, R4676, R4684, R4689, R4691) was compared with the sensitivity of target detection by the DETECTR assays containing the single guides R4684, R4667, R4785 (RNAseP guide) in individual format. Relative quantification performed by counting the number of these positive droplets showed that the samples containing the pooled guide RNAs generated more crystals containing the amplified products per copy of starting target RNA than the samples containing the guide RNAs in individual format as shown in FIG. 116. For example, the number of positive droplets from chamber 1 is higher than the number of droplets in chamber 2 and 3; and the number of droplets from chamber 5 is higher than the number of droplets in chambers 6 and 7 as shown in FIG. 116. Measurement of the target detection signal intensity from the chips also confirmed that the sensitivity of target detection per copy of starting target RNA by the DETECTR assays containing the pooled guides (R4637, R4638, R4667, R4676, R4684, R4689, R4691) was higher than the sensitivity of target detection by the DETECTR assays containing the single guides R4684, R4667, R4785 (RNAseP guide) in individual format as shown in FIG. 117. For example, signal intensity from chamber 1 (containing the seven-guide pool and the Twist SC2 template RNA is higher than the signal intensity in chamber 2 and 3 (containing the R4684, and the R4667 gRNAs in individual format respectively in the presence of the Twist SC2 RNA); and the signal intensity from chamber 5 (containing the seven-guide pool and the ATCC SC2 template RNA) is higher than the signal intensity in chambers 6 and 7 (containing the R4684, and the R4667 gRNAs in individual format respectively, in the presence of the ATCC SC2 RNA) FIG. 117. Similarly, the signal intensity from chamber 5 (containing the seven-guide pool and the ATCC SC2 template RNA) is higher than the signal intensity in chamber 6 (containing the gRNA R4684 in individual format and the ATCC SC2 RNA), the signal intensity from chamber 8 (containing the control RNaseP gRNA in individual format with the ATCC SC2 template RNA) and the signal intensity from chamber 12 (containing the seven pooled gRNAs with no template RNA) FIG. 117.

Relative quantification of the number of droplets containing amplified target (per copy of starting target RNA) observed in chamber 5 (containing the seven-guide pool and the ATCC SC2 template RNA) is higher than the number of droplets observed in chamber 6 (containing the gRNA R4684 in individual format and the ATCC SC2 RNA), the number of droplets observed in chamber 8 (containing the control RNaseP gRNA in individual format with the ATCC SC2 template RNA) and the number of droplets observed in chamber 12 (containing the seven pooled gRNAs with no template RNA) as shown in FIG. 118. The sensitivity of target detection by the assays containing the pooled guides (R4637, R4638, R4667, R4676, R4684, R4689, R4691) was compared with the sensitivity of target detection by the assays containing the single guides R4684, R4667, R4785 (RNAseP guide) in individual format, when the assays were conducted in a benchtop assay format FIG. 119. Results from the bench top assay showed that the samples containing the pooled guides (R4637, R4638, R4667, R4676, R4684, R4689, R4691) was not higher than the sensitivity of target detection by the in the samples containing the single guides R4684, R4667, or R4785 (RNAseP guide) in individual format as shown in FIG. 119.

### Table 4 Displays the Guide Pool and Template per Chamber

| Chamber | Guide | Template |
|---|---|---|
| 1 | 7 pool | 5000 copies Twist SC2 |
| 2 | R4684 | 5000 copies Twist SC2 |
| 3 | R4667 | 5000 copies Twist SC2 |
| 4 | R4785(RNaseP) | 5000 copies Twist SC2 |
| 5 | 7 pool | 5000 copies ATCC SC2 |
| 6 | R4684 | 5000 copies ATCC SC2 |
| 7 | R4667 | 5000 copies ATCC SC2 |
| 8 | R4785(RNaseP) | 5000 copies ATCC SC2 |
| 9 | 7 pool | 5000 copies 5s-87 |
| 10 | R4684 | 5000 copies 5s-87 |
| 11 | R4667 | 5000 copies 5s-87 |
| 12 | 7 pool | NTC |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein can be employed. It is intended that the following claims define the scope of the invention and that methods and structures within the scope of these claims and their equivalents be covered thereby.

1. A device for detecting a target nucleic acid, comprising:
a. a sample interface configured to receive a sample comprising a target nucleic acid;
b. a heating region in fluid communication with the sample interface and configured to amplify the sample received via the sample interface;
c. a detection region in fluid communication with the heating region;
d. a programmable nuclease probe disposed within the sample interface, the heating region, or the detection region, wherein the programmable nuclease probe comprises a programmable nuclease and a guide nucleic acid; and
e. a reporter;
wherein the programmable nuclease is activated by selective binding between the guide nucleic acid and the target nucleic acid within the heating region, the sample interface, or the detection region;
wherein the reporter is configured to release a detection moiety upon cleavage by the activated programmable nuclease;
wherein the detection region is configured to detect a signal produced by the released detection moiety and corresponding to a presence of the target nucleic acid; and
wherein the presence or absence of the target nucleic acid is determined within a time of less than 30 minutes after the sample is received at the sample interface.

2. The device of claim 1, further comprising a reagent mix comprising amplification reagents.

3.-7. (canceled)

8. The device of claim 1, wherein the heating region is configured to maintain an isothermal, or non-cycled temperature profile.

9.-10. (canceled)

11. The device of claim 1, wherein the sample interface a) comprises a compartment configured to receive a swab containing the sample; b) is configured to receive the sample from a swab via pipetting; c) comprises a compartment configured to receive the sample from a container containing the sample; or d) is configured to receive the sample as a fluid.

12. The device of claim 11, wherein the compartment comprises a scraper configured to transfer the sample from the swab to the device.

13.-22. (canceled)

23. The device of claim 1, wherein the heating region comprises one or more channels for fluid movement therethrough.

24.-26. (canceled)

27. The device of claim 23, wherein each channel of the heating region comprises one or more movable mechanisms.

28.-30. (canceled)

31. The device of claim 1, further comprising a chemical heating element.

32. (canceled)

33. The device of claim 1, wherein the heating region comprises a chamber in fluid communication with the sample interface and the detection region.

34. (canceled)

35. The device of claim 1, wherein the reporter is immobilized in the heating region via a support that is immobilized on a surface of the heating region, wherein the support comprises a bead, a coating, and an interspersed polymer.

36.-38. (canceled)

39. The device of claim 1, further comprising a plurality of channels to move the sample from the sample interface to the detection region, wherein the plurality of channels comprises at least one set of channels arranged in series or at least one set of channels arranged in parallel.

40.-47. (canceled)

48. The device of claim 1, further comprising at least one actuator.

49.-50. (canceled)

51. The device of claim 48, wherein a first actuator of the at least one actuator is configured to move the sample from the sample interface to the heating region via manual actuation of the first actuator or wherein a second actuator of the at least one actuator is configured to move the detection moiety from the heating region to the detection region via manual actuation of the second actuator.

52. (canceled)

53. The device of claim 1, wherein the device is configured to be operated manually without electrical power, or wherein the device further comprises a power source.

54.-62. (canceled)

63. The device of claim 1, wherein the programmable nuclease comprises a Cas enzyme, optionally wherein the Cas enzyme is selected from the group consisting of Cas12, Cas13, Cas14, Cas14a, Cas14a1, and CasPhi.

64. (canceled)

65. The device of claim 1, wherein the target nucleic acid comprises a single nucleotide polymorphism (SNP); or wherein the target nucleic acid is indicative of a respiratory disorder, a respiratory pathogen, a sexually transmitted infection (STI), or an infection related to a woman's health.

66.-73. (canceled)

74. The device of claim 1, further comprising a physical filter configured to filter one or more particles from the sample that do not comprise the target nucleic acid.

75. (canceled)

76. The device of claim 1, wherein the programmable nuclease, guide nucleic acid, or the reporter are immobilized to a device surface by a linkage, wherein the linkage comprises a covalent bond, a non-covalent bond, an electrostatic bond, a bond between streptavidin and biotin, an amide bond, or non-specific absorption.

77.-84. (canceled)

85. A device for detecting a target nucleic acid in a sample, comprising:
a. a sample interface for receiving the sample;
b. a heating region in fluid communication with the sample interface, the heating region comprising:
i. a programmable nuclease comprising a guide nucleic acid, and
ii. a reporter, wherein the programmable nuclease is activated by selective binding between the guide nucleic acid and a target nucleic acid, wherein the reporter is configured to release a detection moiety upon cleavage by the activated programmable nuclease;
c. a heating element configured to heat to the heating region;
d. a detection region in fluid communication with the heating region, wherein the detection region is configured to detect a signal produced by the released detection moiety;
e. a first manual actuator configured to transfer the sample from the heating region to the detection region; and
f. a reagent mix comprising amplification reagents, wherein the reagent mix is disposed within the sample interface, the heating region, the detection region, and/or between the sample interface and the heating region,
wherein the device is configured to determine the presence or absence of the target nucleic acid within a time of less than 30 minutes via the produced signal.

86.-176. (canceled)

177. A method for the detection of a target nucleic acid in a sample, the method comprising:
a. providing a device configured to determine a presence or absence of a target nucleic acid in less than 30 minutes after a sample is introduced into the device, the device comprising a sample interface, a heating region in fluid communication with the sample interface, and a detection region in fluid communication with the heating region;
b. introducing the sample into the sample interface of the device;
c. mixing the sample with a reagent mix comprising amplification reagents to generate a mixed sample solution;
d. transferring the mixed sample solution from the sample interface to the heating region;
e. amplifying the sample by heating the mixed sample solution in the heating region;
f. performing a programmable nuclease-based assay, wherein selective binding between a guide nucleic acid and the target nucleic acid activates a programmable nuclease probe configured to cleave a reporter probe, thereby releasing a detection moiety into the mixed sample solution when the target nucleic acid is present;
g. transferring the mixed sample solution with the amplified sample from the heating region to the detection region; and
h. determining the presence or absence of the target nucleic acid in the sample via capture of the released detection moiety in the detection region.

178.-187. (canceled)
